(19) United States
(12) Patent Application Publication
(10) Pub. No.: US 2004/0138462 A1
(43) Pub. Date: Jul. 15, 2004

(54) Aminoalcohol derivatives

(76) Inventors: Minoru Sakurai, Osaka-shi Osaka (JP); Kenichi Washizuka, Osaka (JP); Hitoshi Hamashima, Osaka (JP); Yasuyo Tomishima, Osaka-shi Osaka (JP); Masashi Imanishi, Osaka-shi Osaka (JP); Hiroshi Kayakiri, Osaka-shi Osaka (JP); Kiyoshi Taniguchi, Osaka-shi Osaka (JP); Fujiko Takamura, Osaka-shi Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/477,751

(22) PCT Filed: May 20, 2002
(86) PCT No.: PCT/JP02/04865

(30) Foreign Application Priority Data
May 24, 2001 (AU) PR 5232
Dec. 28, 2001 (AU) PR 9780
Feb. 28, 2002 (AU) PS 0799

(51) Int. Cl. C 07D 4 3/02; C07D 213/36

(52) U.S. Cl. 546/264; 546/276.7; 546/277.4; 546/329

(57) ABSTRACT

The present invention relates to a compound formula wherein R¹ is phenyl, pyridyl, etc., each of which may be substituted with one or two substituent(s); R² is hydrogen, an amino protective group, etc.; R³ and R⁴ are each independently hydrogen, lower alkyl or hydroxy(lower)alkyl; R⁵ is aryl, ar(lower)alkyl, etc., each of which may be substituted with one, two or three substituent(s); R⁸ is hydrogen or halogen, X is a single bond or O—CH₂—, and n is 0, 1 or 2, or a salt thereof. The compound [I] of the present invention and pharmaceutically acceptable salts thereof are useful for the prophylactic and/or the therapeutic treatment of pollakiurea or urinary incontinence.

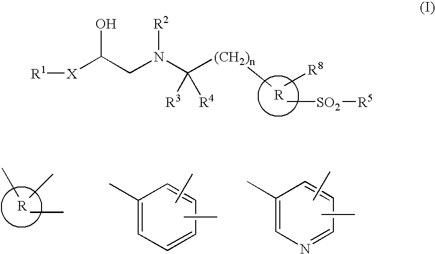

### TECHNICAL FIELD

This invention relates to new aminoalcohol derivatives and salts thereof which are beta-3 (β₃) adrenergic receptor agonists and useful as a medicament.

### DISCLOSURE OF INVENTION

This invention relates to new aminoalcohol derivatives which are β₃ adrenergic receptor agonists and salts thereof.

More particularly, it relates to new aminoalcohol derivatives and salts thereof which have gut sympathomimetic, anti-ulcerous, anti-pancreatitis, lipolytic, anti-urinary incontinence, anti-pollakiuria activities, anti-diabetes and anti-obesity, to processes for the preparation thereof, to a pharmaceutical composition comprising the same and to a method of using the same therapeutically in the treatment and/or prevention of gastro-intestinal disorders caused by smooth muscle contractions in a human being or an animal.

One object of this invention is to provide new and useful aminoalcohol derivatives and salts thereof which have gut sympathomimetic, anti-ulcerous, lipolytic, anti-urinary incontinence, anti-pollakiuria activities, anti-diabetes and anti-obesity.

Another object of this invention is to provide processes for the preparation of said aminoalcohol derivatives and salts thereof.

A further object of this invention is to provide a pharmaceutical composition comprising, as an active ingredient, said aminoacohol derivatives and salts thereof.

Still further object of this invention is to provide a therapeutical method for the treatment and/or prevention of aforesaid diseases in a human being or an animal, using said aminoalcohol derivatives and salts thereof.

The object aminoalcohol derivatives of this invention are new and can be represented by compound of the following formula [I]:

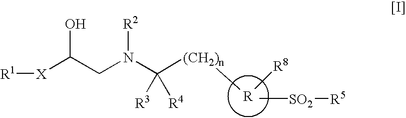

wherein

R¹ is phenyl, pyridyl, indolyl or carbazolyl, each of which may be substituted with one or two same or different substituent(s) selected from a group consisting of halogen; hydroxy; benzyloxy; nitro; cyano; mono(or di or tri)halo(lower)alkyl; and (lower alkylsulfonyl)amino,

R² is hydrogen, [5-(lower alkyl)-2-oxo-1,3-dioxol-4-yl](lower)alkoxycarbony or an amino protective group,

R³ and R⁴ are each independently hydrogen, lower alkyl or hydroxy(lower)alkyl,

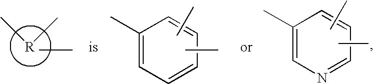

R⁵ is aryl, ar(lower)alkyl, a heterocyclic group or lower alkyl, each of which may be substituted with one, two or three same or different substituent(s) selected from a group consisting of halogen; hydroxy; cyano; amino(hydroxyimino)methyl; phenyl optionally substituted with carboxy or lower alkoxycarbonyl; phenoxy optionally substituted with halogen; lower alkoxy optionally substituted with hydroxy, amino, cyano, carboxy, carbamoyl, mono(or di)(lower)alkylcarbamoyl, lower alkoxycarbonyl, cyclo(lower)alkyloxycarbonyl, hydroxy(lower)alkoxycarbonyl, di[(lower)alkoxy](lower)alkoxycarbonyl, pyridyl(lower)alkoxycarbonyl, phenyl or tetrazolyl; mono(or di or tri)halo(lower)alkoxy; lower alkyl optionally substituted with carboxy, lower alkoxycarbonyl, dioxothiazolidinyl or dioxothiazolidinylidene; lower alkenyl optionally substituted with carboxy or lower alkoxycarbonyl; oxadiazolyl optionally substituted with lower alkyl; tetrazolyl; triazolylthio; lower alkanoyl; carboxy; lower alkoxycarbonyl; carbamoyl optionally substituted with one or two same or different substituent(s) selected from a group consisting of lower alkyl, lower alkoxy, carboxy(lower)alkyl, lower alkoxycarbonyl(lower)alkyl, tetrazolyl, thiazolyl optionally substituted with lower alkyl, oxazolyl optionally substituted with lower alkyl, oxadiazolyl, lower alkylsulfonyl and phenylsulfonyl; (hydroxypiperidino)carbonyl; (2,4-dioxo-1,3-thiazolidin-5-ylindene)methyl; and amino optionally substituted with one or two same or different substituent(s) selected from a group consisting of lower alkyl, lower alkanoyl, benzoyl, pyridylcarbonyl, lower alkylsulfonyl, phenylsulfonyl, carbamoyl, lower alkylcarbamoyl, phenylcarbamoyl, lower alkoxycarbonyl and phenoxycarbonyl, or

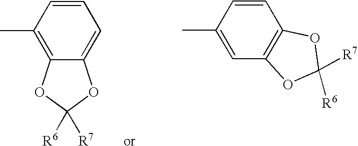

in which R⁶ and R⁷ are each independently hydrogen, carboxy or lower alkoxycarbonyl,

R⁸ is hydrogen or halogen,

X is a single bond or —O—CH₂—, and

n is 0, 1 or 2,

or a salt thereof.

According to this invention, the object compounds can be prepared by processes which are illustrated in the following schemes.

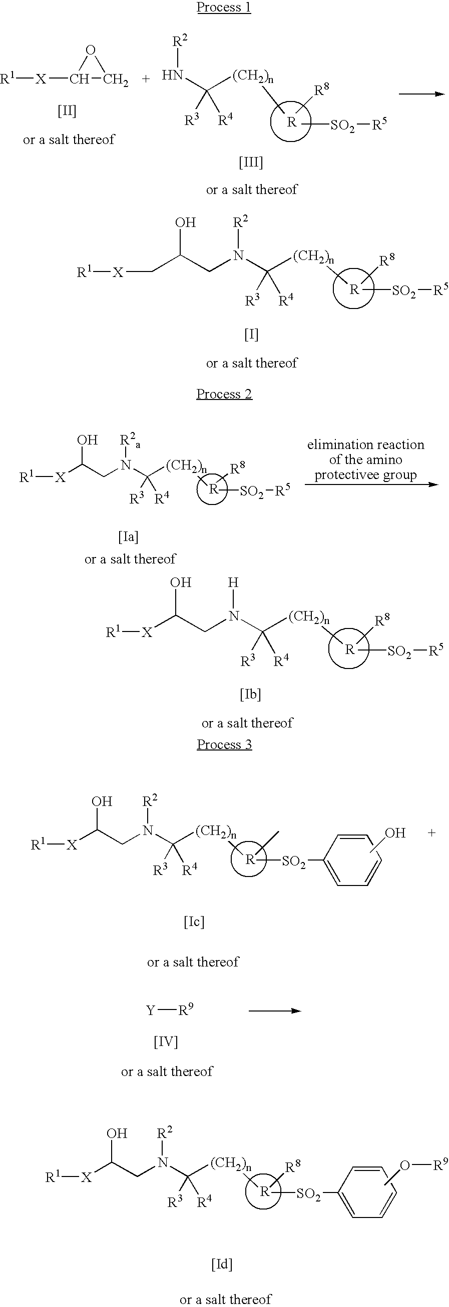

wherein R¹, R², R₃, R⁴,

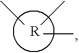

R⁵, R⁸, X and n are each as defined above,

R²ₐ is [5-(lower alkyl)-2-oxo-1,3-dioxol-4-yl](lower)alkoxycarbonyl or an amino protective group,

R⁹ is lower alkyl optionally substituted with hydroxy, amino, cyano, carboxy, carbamoyl, mono(or di)(lower)alkylcarbamoyl, lower alkoxycarbonyl, cyclo(lower)alkyloxycarbonyl, hydroxy(lower)alkoxycarbonyl, di[(lower)alkoxy](lower)alkoxycarbonyl, pyridyl(lower)alkoxycarbonyl, phenyl or tetrazolyl, and

Y is halogen.

As to the starting compounds [II], [III], [Ia], [Ic] and [IV], some of them are novel and can be prepared by the procedures described in the Preparations and Examples mentioned later or a conventional manner.

In the above and subsequent description of the present specification, suitable examples of the various definition to be included within the scope of the invention are explained in detail in the following.

The term “lower” is intended to mean a group having 1 to 6, preferably 1 to 4, carbon atom(s), unless otherwise indicated.

Suitable “lower alkyl” and “lower alkyl” moiety in the terms of “(lower alkylsulfonyl)amino”, “di(lower)alkyl-carbamoyl”, etc. may include straight or branched one having 1 to 6 carbon atom(s), such as methyl, ethyl, propyl, isopropyl, butyl, isobutyl, sec-butyl, tert-butyl, pentyl, 1-methylpentyl, tert-pentyl, neo-pentyl, hexyl, isohexyl and the like, in which more preferable one is C₁-C₄ alkyl, and the most preferable one is methyl.

Suitable “lower alkenyl” may include vinyl, 1-(or 2-)-propenyl, 1-(or 2- or 3-)butenyl, 1-(or 2- or 3- or 4-)-pentenyl, 1-(or 2- or 3- or 4- or 5-)hexenyl, methylvinyl, ethylvinyl, 1-(or 2- or 3-)methyl-1-(or 2-)propenyl, 1-(or 2- or 3-)ethyl-1-(or 2-)propenyl, 1-(or 2- or 3- or 4-)methyl-1-(or 2- or 3-)butenyl and the like, in which more preferable one may be C₂-C₄ alkenyl.

Suitable “cyclo(lower)alkyl” moiety in the term of “cyclo(lower)alkyloxycarbonyl” may include cyclopropyl, cyclobutyl, cyclopentyl, and cyclohexyl, in which more preferable one is cyclo(C₃-C₆)alkyl, and the most preferable one is cyclohexyl.

Suitable “lower alkoxy” and “lower alkoxy” moiety in the terms of “mono(or di or tri)(lower)alkoxy” and “lower alkoxycarbonyl” may include methoxy, ethoxy, propoxy, isopropoxy, butoxy, iso-butoxy, t-butoxy, pentyloxy, t-pentyloxy, hexyloxy and the like, in which preferable one is C₁-C₄ alkoxy, and the most preferable one is methoxy or ethoxy.

Suitable “lower alkanoyl” may include formyl, acetyl, propanoyl, butanoyl, 2-methylpropanoyl, pentanoyl, 2,2-dimethylpropanoyl, hexanoyl and the like, in which preferable one is C₂-C₄ alkanoyl, and the most preferable one is formyl.

Suitable “halogen” may be fluoro, chloro, bromo and iodo, in which preferable one is chloro.

Suitable “aryl” and “aryl” moiety in the term of “ar(lower)alkyl” may include phenyl, naphthyl, anthryl-and the like, in which the preferred one may be phenyl.

Suitable example of “heterocyclic group” may include

unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 1H-1,2,4-triazolyl, 4H-1,2,4-triazolyl, 1H-1,2,3-triazolyl or 2H-1,2,3-triazolyl), tetrazolyl (e.g. 1H-1,2,3,4-tetrazolyl, 2H-1,2,3,4-tetrazolyl, etc.), etc.;

saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, azetidinyl, etc.;

unsaturated condensed heterocyclic group containing 1 to 4 nitrogen atom(s), for example, indolyl, isoindolyl, indolinyl, indolizinyl, benzimidazolyl, quinolyl, isoquinolyl, indazolyl, benzotriazolyl, etc.;

unsaturated 3 to 8-membered (more preferably 5 or 6-membered) heteromonocyclic group containing 1 or 2 oxygen atom(s) and 1 to 3 nitrogen atom(s), for example, oxazolyl, isoxazolyl, oxadiazolyl (e.g., 1,2,4-oxadiazolyl, 1,3,4-oxadiazolyl, 1,2,5-oxadiazolyl, etc.), etc.;

saturated 3 to 8-membered (more preferably 5 or 6-membered) heteromonocyclic group containing 1 or 2 oxygen atom(s) and 1 to 3 nitrogen atom(s), for example, morpholinyl, sydnonyl, morpholino, etc.;

unsaturated condensed heterocyclic group containing 1 or 2 oxygen atom(s) and 1 to 3 nitrogen atom(s), for example, benzoxazolyl, benzoxadiazolyl, etc.;

unsaturated 3 to 8-membered (more preferably 5 or 6-membered) heteromonocyclic group containing 1 or 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;

saturated 3 to 8-membered (more preferably 5 or 6-membered) heteromonocyclic group containing 1 or 2 sulfur atom(s) and 1 to 3 nitrogen atom(s), for example thiazolidinyl, thiomorpholinyl, thiomorpholino, etc.;

unsaturated 3 to 8-membered (more preferably 5 or 6-membered) heteromonocyclic group containing 1 or 2 sulfur atom(s), for example, thienyl, dihydrodithiinyl, dihydrodithionyl, etc.;

unsaturated condensed heterocyclic group containing 1 or 2 sulfur atom(s) and 1 to 3 nitrogen atom(s), for example, benzothiazolyl, benzothiadiazolyl, imidazothiadiazolyl, etc.;

unsaturated 3 to 8-membered (more preferably 5 or 6-membered) heteromonocyclic group containing an oxygen atom, for example, furyl etc.;

saturated 3 to 8-membered (more preferably 5 or 6-membered) heteromonocyclic group containing 1 or 2 oxygen atom(s), for example, tetrahydrofuran, tetrahydropyran, dioxacyclopentane, dioxacyclohexane, etc.;

unsaturated 3 to 8-membered (more preferably 5 or 6-membered) heteromonocyclic group containing an oxygen atom and 1 or 2 sulfur atom(s), for example, dihydrooxathiinyl, etc.;

unsaturated condensed heterocyclic group containing 1 or 2 sulfur atom(s), for example benzothienyl, benzodithiinyl, etc.;

unsaturated condensed heterocyclic group containing an oxygen atom and 1 or 2 sulfur atom(s), for example, benzoxathiinyl, etc.; and the like.

Suitable “mono(or di or tri)halo(lower)alkoxy” may include chloromethoxy, dichloromethoxy, trichloromethoxy, bromomethoxy, dibromomethoxy, tribromomethoxy, fluoromethoxy, difluoromethoxy, trifluoromethoxy, 1 or 2-chloroethoxy, 1 or 2-bromoethoxy, 1 or 2-fluoroethoxy, 1,1-difluoroethoxy, 2,2-difluoroethoxy and the like, in which more preferable one is mono(or di or tri)halo(C₁-C₄)alkoxy, and the most preferable one is difluoromethoxy.

Suitable example of “amino protective group” moiety may be common amino protective group such as substituted or unsubstituted lower alkanoyl [e.g. formyl, acetyl, propionyl, trifluoroacetyl, etc.], phthaloyl, lower alkoxycarbonyl [e.g. tert-butoxycarbonyl, tert-amyloxycarbonyl, etc.], substituted or unsubstituted aralkyloxycarbonyl [e.g. benzyloxycarbonyl, p-nitrobenzyloxycarbonyl, etc.], substituted or unsubstituted arenesulfonyl [e.g. benzenesulfonyl, tosyl, etc.], nitrophenylsulfenyl, ar(lower)alkyl [e.g. trityl, benzyl, etc.], and the like, in which preferable one is benzyl.

Suitable salts of the object aminoalcohol derivative [I] are pharmaceutically acceptable salts and include conventional non-toxic salts such as an inorganic acid addition salt [e.g. hydrochloride, hydrobromide, sulfate, phosphate, etc.], an organic acid addition salt [e.g. formate, acetate, trifluoroacetate, oxalate, maleate, fumarate, tartrate, citrate, methanesulfonate, benzenesulfonate, toluenesulfonate, etc., an alkali metal salt [e.g. sodium salt, potassium salt, etc.] or the like.

The Processes 1 to 3 for preparing the object compounds of the present invention are explained in detail in the following.

Process 1

The object compound [I] or a salt thereof can be prepared by reacting a compound [II] or a salt thereof with a compound [III] or a salt thereof.

Suitable salt of the compounds [II] and [III] may be the same as those exemplified for the compound [I].

The reaction is preferably carried out in the presence of a base such as an alkali metal carbonate [e.g. sodium carbonate, potassium carbonate, etc.], an alkaline earth metal carbonate [e.g. magnesium carbonate, calcium carbonate, etc.], an alkali metal bicarbonate [e.g. sodium bicarbonate, potassium bicarbonate, etc.], tri(lower)alkylamine [e.g. trimethylamine, triethylamine, etc.], picoline or the like.

The reaction is usually carried out in a conventional solvent, such as an alcohol [e.g. methanol, ethanol, propanol, isopropanol, etc.], diethyl ether, tetrahydrofuran, dioxane, or any other organic solvent which does not adversely influence the reaction.

The reaction temperature is not critical, and the reaction can be carried out under cooling to heating.

Process 2

The object compound [Ib] or a salt thereof can be prepared by subjecting a compound [Ia] or a salt thereof to elimination reaction of the amino protective group.

Suitable salts of the compounds [Ia] and [Ib] may be the same as those exemplified for the compound [I].

This reaction can be carried out in a similar manner to that of Example 8 or 9 mentioned below.

Process 3

The object compound [Id] or a salt thereof can be prepared by reacting a compound [Ic] or a salt thereof with a compound [IV] or a salt thereof.

Suitable salts of the compounds [Ic] and [IV] may be the same as those exemplified for the compound [I].

This reaction can be carried out in a similar manner to that of Example 19 or 21.

The compounds obtained by the above processes can be isolated and purified by a conventional method such as pulverization, recrystallization, column chromatography, reprecipitation, or the like, and converted to the desired salt in conventional manners, if necessary.

It is to be noted that the compound [I] and the other compounds may include one or more stereoisomers due to asymmetric carbon atoms, and all of such isomers and mixture thereof are included within the scope of this invention.

It is further to be noted that isomerization or rearrangement of the object compound [I] may occur due to the effect of the light, acid base or the like, and the compound obtained as the result of said isomerization or rearrangement if also included within the scope of the present invention.

It is also to be noted that the solvating form of the compound [I] (e.g. hydrate, etc.) and any form of the crystal of the compound [I] are included within the scope of the present invention.

The object compound [I] or a salt thereof possesses gut sympathomimetic, anti-ulcerous, anti-pancreatitis, lipolytic, anti-urinary incontinence and anti-pollakiuria activities, and are useful for the treatment and/or prevention of gastro-intestinal disorders caused by smooth muscle contractions in human beings or animals, and more parcitularly for the treatment and/or prevention of spasm or hyperanakinesia in case of irritable bowel syndrome, gastritis, gastric ulcer, duodenal ulcer, enteritis, cholecystopathy, cholantitis, urinary calculus and the like; for the treatment and/or prevention of ulcer such as gastric ulcer, duodenal ulcer, peptic ulcer, ulcer causes by non steroidal anti-inflammatory drags, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis, prostatic hypertrophy or the like; for the treatment and/or prevention of pancreatitis, obesity, diabetes, glycosuria, hyperlipidemia, hypertension, atherosclerosis, glaucoma, melancholia, depression or the like; for the treatment and/or prevention of diseases as the result of insulin resistance (e.g. hypertension, hyperinsulinemia, etc.); for the treatment and/or prevention of neurogenetic inflammation; and for reducing a wasting condition, and the like.

Additionally, β₃ adrenergic receptor agonists are known to lower triglyceride and cholesterol levels and to raise high density lipoprotein levels in mammals (U.S. Pat. No. 5,451,677). Accordingly, the object compound [I] in useful in the treatment and/or prevention of conditions such as hyper-triglyceridaemia, hypercholesterolaemia and in lowering high density lipoprotein levels as well as in the treatment of atherosclerotic and cardiovascular diseases and relates conditions.

Moreover, the object compound [I] is useful for inhibiting uterine contractions, preventing premature labor, and treating and preventing dysmenorrhea.

In order to show the usefulness of the compound [I] for the prophylactic and therapeutic treatment of above-mentioned disease in human being or animals, the pharmacological test data of a representative compound thereof are shown in the following.

Test

Effect on the increase in intravesical pressure induced by carbachol in anesthetized dog

Test Compound

(1) (2S)-2-[((2S)-2-Hydroxy-3-phenoxypropyl)amino]-3-[4-(phenylsulfonyl)phenyl]-1-propanol (the object compound of Example 8 mentioned below)

Test Method

Female Beagle dogs weighing 8.0-15.0 kg were fasted for 24 hours and maintained under halothane anesthesia. A 12F Foley catheter was lubricated with water soluble jelly, inserted into the urethral orifice and advanced approximately 10 cm until the balloon tip was placed well inside the bladder. The balloon was then inflated with 5 ml of room air and catheter slowly withdrawn just part the first resistance that is felt at the bladder neck. Urine was completely drained out through the catheter, and 30 ml of biological saline was infused. The catheter was connected to pressure transducer, and intravesical pressure was continuously recorded. Intraduodenal administration of test compound (I) inhibited carbachol (1.8 μg/kg)-induced increase in intravesical pressure (IVP).

Test Results

| | % inhibition of |
|---|---|
| | carbachol-induced |
| Treatment | increase in IVP |
| Test Compound (1) | 30% |
| (0.32 mg/kg) | |

Preferred embodiments of the object compound [I] are as follows:

R¹ is phenyl, pyridyl, indolyl or carbazolyl, each of which may be substituted with one or two same or different substituent(s) selected from a group consisting of halogen (more preferably fluoro or chloro); hydroxy; benzyloxy; nitro; cyano; mono(or di or tri)halo(lower)alkyl (more preferably mono(or di or tri)halo(C₁-C₄)alkyl, most preferably trifluoromethyl) and (lower alkylsulfonyl)amino (more preferably (C₁-C₄ alkylsulfonyl)amino, most preferably (methanesulfonyl)amino),

R² is hydrogen, [5-(lower alkyl)-2-oxo-1,3-dioxol-4-yl](lower)alkoxycarbonyl (more preferably [5-(C₁-C₄ alkyl)-2-oxo-1,3-dioxol-4-yl](C₁-C₄)-alkoxycarbonyl, most preferably (5-methyl-2-oxo-1,3-dioxol-4-yl)methoxycarbonyl), lower alkoxycarbonyl (more preferably C₁-C₄ alkoxycarbonyl, most preferably tert-butoxycarbonyl) or ar(lower)alkyl (more preferably ar(C₁-C₄)alkyl, most preferably benzyl),

R³ and R⁴ are each independently hydrogen, lower alkyl (more preferably C₁-C₄ alkyl, most preferably methyl) or hydroxy(lower)alkyl (more preferably hydroxy(C₁-C₄)-alkyl, most preferably hydroxymethyl),

R⁵ is aryl (more preferably phenyl), ar(lower)alkyl (more preferably ar(C₁-C₄)alkyl, most preferably benzyl), a heterocyclic group (more preferably unsaturated 3 to 8-membered (more preferably 5 or 6-membered) heteromonocyclic group containing 1 to 4 nitrogen atom(s), unsaturated condensed heterocyclic group containing 1 to 4 nitrogen atom(s), unsaturated 3 to 8-membered (more preferably 5 or 6-membered) heteromonocyclic group containing 1 or 2 sulfur atom(s) and 1 to 3 nitrogen atom(s) or unsaturated 3 to 8-membered (more preferably 5 or 6-membered) heteromonocyclic group containing 1 or 2 sulfur atom(s), most preferably triazolyl (most preferably 1H-1,2,4-triazolyl), tetrazolyl (most preferably 1H-1,2,3,4-tetrazolyl), quinolyl, thiazolyl or thienyl) or lower alkyl (more preferably C₁-C₄ alkyl, most preferably propyl), each of which may be substituted with one, two or three same or different substituent(s) selected from a group consisting of halogen (more preferably fluoro or chloro); hydroxy; cyano; amino(hydroxyimino)methyl; phenyl optionally substituted with carboxy or lower alkoxycarbonyl (more preferably C₁-C₄ alkoxycarbonyl, most preferably ethoxycarbonyl); phenoxy optionally substituted with halogen (more preferably fluoro); lower alkoxy (more preferably C₁-C₄ alkoxy, most preferably methoxy, ethoxy or isopropoxy) optionally substituted with hydroxy, amino, cyano, carboxy, carbamoyl, mono(or di)(lower)alkylcarbamoyl (more preferably mono(or di)(C₁-C₄)alkylcarbamoyl, most preferably methylcarbamoyl or dimethylcarbamoyl), lower alkoxycarbonyl (more preferably C₁-C₄ alkoxycarbonyl, most preferably methoxycarbonyl, ethoxycarbonyl, propoxycarbonyl, isopropoxycarbonyl or tert-butoxycarbonyl), cyclo(lower)alkyloxycarbonyl (more preferably cyclo(C₃-C₆)alkyloxycarbonyl, most preferably cyclohexyloxycarbonyl), hydroxy(lower)alkoxycarbonyl (more preferably hydroxy(C₁-C₄)alkoxycarbonyl, most preferably 2-hydroxyethoxycarbonyl), di[(lower)alkoxy](lower)-alkoxycarbonyl (more preferably di[(C₁-C₄)alkoxy]-(C₁-C₄)alkoxycarbonyl, most preferably 2-ethoxy-1-(ethoxymethyl)ethoxycarbonyl), pyridyl(lower)alkoxycarbonyl (more preferably pyridyl(C₁-C₄)alkoxycarbonyl, most preferably 2-pyridylmethoxycarbonyl), phenyl or tetrazolyl (more preferbaly 1H-1,2,3,4-tetrazolyl); mono(or di or tri)halo(lower)alkoxy (more preferably mono(or di or tri)halo(C₁-C₄)alkoxy, most preferably fluoromethoxy, difluoromethoxy or trifluoromethoxy); lower alkyl (more preferably C₁-C₄ alkyl, most preferably methyl or ethyl) optionally substituted with carboxy, lower alkoxycarbonyl (more preferably C₁-C₄ alkoxycarbonyl, most preferably ethoxycarbonyl), dioxothiazolidinyl (more preferably 2,4-dioxothiazolidinyl) or dioxothiazolidinylidene (more preferably 2,4-dioxothiazolidinylidene); lower alkenyl (more preferably C₂-C₄ alkenyl, most preferably vinyl) optionally substituted with carboxy or lower alkoxycarbonyl (more preferably C₁-C₄ alkoxycarbonyl, most preferably ethoxycarbonyl); oxadiazolyl (more preferably 1,2,4-oxadiazolyl) optionally substituted with lower alkyl (more preferably C₁-C₄ alkyl, most preferably methyl); tetrazolyl (more preferably 1H-1,2,3,4-tetrazolyl); triazolylthio (more preferably 1H-1,2,4-triazol-3-ylthio); lower alkanoyl (more preferably C₁-C₄ alkanoyl, most preferably formyl); carboxy; lower alkoxycarbonyl (more preferably C₁-C₄ alkoxycarbonyl, most preferably ethoxycarbonyl); carbamoyl optionally substituted with one or two same or different substituent(s) selected from a group consisting of lower alkyl (more preferably C₁-C₄ alkyl, most preferably methyl), lower alkoxy (more preferably C₁-C₄ alkoxy, most preferably methoxy), carboxy(lower)alkyl (more preferably carboxy(C₁-C₄)-alkyl, most preferably carboxymethyl or 2-carboxyethyl), lower alkoxycarbonyl(lower)alkyl (more preferably C₁-C₄ alkoxycarbonyl(C₁-C₄)alkyl, most preferably ethoxycarbonylmethyl or 2-(ethoxycarbonyl)ethyl), thiazolyl optionally substituted with lower alkyl (more preferably C₁-C₄ alkyl, most preferably methyl), oxazolyl optionally substituted with lower alkyl (more preferably C₁-C₄ alkyl, most preferably methyl), oxaziazolyl (more preferably 1,2,4-oxaziazolyl), lower alkylsulfonyl (more preferably C₁-C₄ alkylsulfonyl, most preferably methanesulfonyl) and phenylsulfonyl; (hydroxypiperidino)carbonyl; (2,4-dioxo-1,3-thiazolidin-5-ylidene)methyl; and amino optionally substituted with one or two same or different substituent(s) selected from a group consisting of lower alkyl (more preferably C₁-C₄ alkyl, most preferably methyl), lower alkanoyl (more preferably C₁-C₄ alkanoyl, most preferably acetyl), benzoyl, pyridylcarbonyl, lower alkylsulfonyl (more preferably C₁-C₄ alkylsulfonyl, most preferably methanesulfonyl), phenylsulfonyl, carbamoyl, lower alkylcarbamoyl (more preferably C₁-C₄ alkylcarbamoyl, most preferably methylcarbamoyl), phenylcarbamoyl, lower alkoxycarbonyl (more preferably C₁-C₄ alkoxycarbonyl, most preferably methoxycarbonyl) and phenoxycarbonyl, or

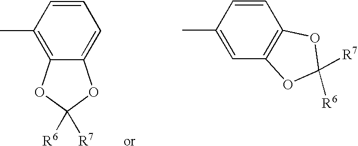

in which R⁶ and R⁷ are each independently hydrogen, carboxy or lower alkoxycarbonyl (more preferably C₁-C₄ alkoxycarbonyl, most preferably ethoxycarbonyl), and

R⁸ is hydrogen or halogen (more preferably chloro).

More preferred embodiments of the object compound [I] are as follows:

R¹ is phenyl which may be substituted with one or two same or different substituent(s) selected from a group consisting of halogen (more preferably fluoro or chloro); hydroxy; benzyloxy; nitro and (lower alkylsulfonyl)amino (more preferably (C₁-C₄ alkylsulfonyl)amino, most preferably (methanesulfonyl)amino),

R² is hydrogen or [5-(lower alkyl)-2-oxo-1,3-dioxol-4-yl](lower)alkoxycarbonyl (more preferably [5-(C₁-C₄ alkyl)-2-oxo-1,3-dioxol-4-yl](C₁-C₄)alkoxycarbonyl, most preferably (5-methyl-2-oxo-1,3-dioxol-4-yl)methoxycarbonyl),

R³ and R⁴ are each independently hydrogen, lower alkyl (more preferably C₁-C₄ alkyl, most preferably methyl) or hydroxy(lower)alkyl (more preferably hydroxy(C₁-C₄)-alkyl, most preferably hydroxymethyl),

R⁵ is phenyl, benzyl, triazolyl (more preferably 1H-1,2,4-triazolyl), tetrazolyl (more preferably 1H-1,2,3,4-tetrazolyl), quinolyl, thiazolyl, thienyl or lower alkyl (more preferably C₁-C₄ alkyl, most preferably propyl), each of which may be substituted with one, two or three same or different substituent(s) selected from a group consisting of halogen (more preferably fluoro or chloro); hydroxy; cyano; amino(hydroxyimino)methyl; phenyl optionally substituted with carboxy or lower alkoxycarbonyl (more preferably C₁-C₄ alkoxycarbonyl, most preferably ethoxycarbonyl); phenoxy optionally substituted with halogen (more preferably fluoro); lower alkoxy (more preferably C₁-C₄ alkoxy, most preferably methoxy, ethoxy or isopropoxy) optionally substituted with hydroxy, amino, cyano, carboxy, carbamoyl, mono(or di)(lower)alkylcarbamoyl (more preferably mono(or di)(C₁-C₄)alkylcarbamoyl, most preferably methylcarbamoyl or dimethylcarbamoyl), lower alkoxycarbonyl (more preferably C₁-C₄ alkoxycarbonyl, most preferably methoxycarbonyl, ethoxycarbonyl, propoxycarbonyl, isopropoxycarbonyl or tert-butoxycarbonyl), cyclo(lower)alkyloxycarbonyl (more preferably cyclo(C₃-C₆)alkyloxycarbonyl, most preferably cyclohexyloxycarbonyl), hydroxy(lower)alkoxycarbonyl (more preferably hydroxy(C₁-C₄)alkoxycarbonyl, most preferably 2-hydroxyethoxycarbonyl), di[(lower)alkoxy]-(lower)alkoxycarbonyl) (more preferably di[(C₁-C₄)-alkoxy](C₁-C₄)alkoxycarbonyl, most preferably 2-ethoxy-1-(ethoxymethyl)ethoxycarbonyl), pyridyl(lower)alkoxycarbonyl (more preferably pyridyl(C₁-C₄)alkoxycarbonyl, most preferably 2-pyridylmethoxycarbonyl), phenyl or tetrazolyl (more preferbaly 1H-1,2,3,4-tetrazolyl); mono(or di or tri)halo(lower)alkoxy (more preferably mono(or di or tri)halo(C₁-C₄)alkoxy, most preferably fluoromethoxy, difluoromethoxy or trifluoromethoxy); lower alkyl (more preferably C₁-C₄ alkyl, most preferably methyl or ethyl) optionally substituted with carboxy, lower alkoxycarbonyl (more preferably C₁-C₄ alkoxycarbonyl, most preferably ethoxycarbonyl), dioxothiazolidinyl (more preferably 2,4-dioxothiazolidinyl) or dioxothiazolidinylidene (more preferably 2,4-dioxothiazolidinylidene); lower alkenyl (more preferably C₂-C₄ alkenyl, most preferably vinyl) optionally substituted with carboxy or lower alkoxycarbonyl (more preferably C₁-C₄ alkoxycarbonyl, most preferably ethoxycarbonyl); oxadiazolyl (more preferably 1,2,4-oxadiazolyl) optionally substituted with lower alkyl (more preferably C₁-C₄ alkyl, most preferably methyl); tetrazolyl (more preferably 1H-1,2,3,4-tetrazolyl); triazolylthio (more preferably 1H-1,2,4-triazol-3-ylthio); lower alkanoyl (more preferably C₁-C₄ alkanoyl, most preferably formyl); carboxy; lower alkoxycarbonyl (more preferably C₁-C₄ alkoxycarbonyl, most preferably ethoxycarbonyl); carbamoyl optionally substituted with one or two same or different substituent(s) selected from a group consisting of lower alkyl (more preferably C₁-C₄ alkyl, most preferably methyl), lower alkoxy (more preferably C₁-C₄ alkoxy, most preferably methoxy), carboxy(lower)alkyl (more preferably carboxy(C₁-C₄)-alkyl, most preferably carboxymethyl or 2-carboxyethyl), lower alkoxycarbonyl(lower)alkyl (more preferably C₁-C₄ alkoxycarbonyl(C₁-C₄)alkyl, most preferably ethoxycarbonylmethyl or 2-(ethoxycarbonyl)ethyl), thiazolyl optionally substituted with lower alkyl (more preferably C₁-C₄ alkyl, most preferably methyl), oxazolyl optionally substituted with lower alkyl (more preferably C₁-C₄ alkyl, most preferably methyl), oxaziazolyl (more preferably 1,2,4-oxaziazolyl), lower alkylsulfonyl (more preferably C₁-C₄ alkylsulfonyl, most preferably methanesulfonyl) and phenylsulfonyl; (hydroxypiperidino)carbonyl; (2,4-dioxo-1,3-thiazolidin-5-ylidene)methyl; and amino optionally substituted with one or two same or different substituent(s) selected from a group consisting of lower alkyl (more preferably C₁-C₄ alkyl, most preferably methyl), lower alkanoyl (more preferably C₁-C₄ alkanoyl, most Preferably acetyl), benzoyl, pyridylcarbonyl, lower alkylsulfonyl (more preferably C₁-C₄ alkylsulfonyl, most preferably methanesulfonyl), phenylsulfonyl, carbamoyl, lower alkylcarbamoyl (more preferably C₁-C₄ alkylcarbamoyl, most preferably methylcarbamoyl), phenylcarbamoyl, lower alkoxycarbonyl (more preferably C₁-C₄ alkoxycarbonyl, most preferably methoxycarbonyl) and phenoxycarbonyl, or

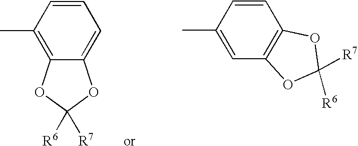

in which R⁶ and R⁷ are each independently hydrogen, carboxy or lower alkoxycarbonyl (more preferably C₁-C₄ alkoxycarbonyl, most preferably ethoxycarbonyl), and

R⁸ is hydrogen or halogen (more prefeably chloro).

More preferred embodiments of the object compound [I] are as follows:

R¹ is phenyl which may be substituted with halogen,

R² is hydrogen,

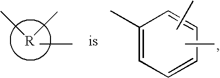

R⁵ is phenyl which may be substituted with one, two or three same or different substituent(s) selected from a group consisting of halogen; hydroxy; cyano; amino(hydroxyimino)methyl; phenyl optionally substituted with carboxy or lower alkoxycarbonyl; phenoxy optionally substituted with halogen; lower alkoxy optionally substituted with hydroxy, amino, cyano, carboxy, carbamoyl, mono(or di)(lower)alkoxycarbamoyl, lower alkoxycarbonyl, cyclo(lower)alkyloxycarbonyl, hydroxy(lower)alkoxycarbonyl, di[(lower)alkoxy](lower)alkoxycarbonyl, pyridyl(lower)alkoxycarbonyl, phenyl or tetrazolyl; mono(or di or tri)halo(lower)alkoxy; lower alkyl optionally substituted with carboxy, lower alkoxycarbonyl, dioxothiazolidinyl or dioxothiazolidinylidene; lower alkenyl optionally substituted with carboxy or lower alkoxycarbonyl; oxadiazolyl optionally substituted with lower alkyl; tetrazolyl; triazolylthio; lower alkanoyl; carboxy; lower alkoxycarbonyl; carbamoyl optionally substituted with one or two same or different substituent(s) selected from a group consisting of lower alkyl, lower alkoxy, carboxy, lower alkoxycarbonyl, thiazolyl optionally substituted with lower alkyl, oxazolyl optionally substituted with lower alkyl, oxadiazolyl, lower alkylsulfonyl or phenylsulfonyl; and amino optionally substituted with one or two same or different substituent(s) selected from a group consisting of lower alkyl and lower alkanoyl,

R⁸ is hydrogen,

X is a single bond, and

n is 1.

More preferred embodiments of the object compound [I] are as follows:

R¹ is phenyl which may be substituted with halogen,

R² is hydrogen,

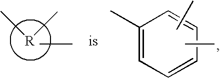

R³ and R⁴ are each hydrogen,

R⁵ is phenyl substituted with lower alkoxy optionally substituted with a substituent selected from a group consisting of hydroxy, amino, cyano, carboxy, carbamoyl, mono(or di)(lower)alkoxycarbamoyl, lower alkoxycarbonyl, cyclo (lower) alkyloxycarbonyl, hydroxy(lower)alkoxycarbonyl, di[(lower)alkoxy](lower)alkoxycarbonyl, pyridyl(lower)alkoxycarbonyl, phenyl and tetrazolyl,

R⁸ is hydrogen,

X is a single bond, and

n is 1.

The following Preparations and Examples are given for the purpose of illustrating this invention.

Preparation 1

Under nitrogen, to a solution of tert-butyl (S)-2-hydroxy-1-(4-hydroxybenzyl)ethylcarbamate (24 g) in dichloromethane (500 ml) were added 2,2-dimethoxypropane (34 ml) and p-toluenesulfonic acid monohydrate (1.7 g) at room temperature, and the mixture was stirred at the same temperature for 60 hours. The resulting mixture was poured into saturated aqueous sodium hydrogencarbonate and the aqueous mixture was extracted with ethyl acetate. The organic layer was washed with brine, dried over anhydrous magnesium sulfate and evaporated under reduced pressure to get a solid. Trituration with hexane followed by collection and dryness in vacuo gave tert-butyl (S)-4-(4-hydroxybenzyl)-2,2-dimethyl-1,3-oxazolidine-3-carboxylate (22 g).

NMR (DMSO-d₆, δ): 1.3-1.55 (15H, m), 2.4-2.6 (1H, m), 2.8-2.95 (1H, m), 3.6-4.0 (3H, m), 6.69 (2H, d, J=8.2 Hz), 6.98 (2H, d, J=8.4 Hz)

Preparation 2

Under nitrogen, to a solution of tert-butyl (S)-4-(4-hydroxybenzyl)-2,2-dimethyl-1,3-oxazolidine-3-carboxylate (10 g) in dichloromethane (100 ml) were added 2,6-lutidine (4.2 ml) and trifluoromethanesulfonic anhydride (6.0 ml) at 5° C. and the mixture was stirred at the same temperature for 80 minutes. The resulting mixture was poured into ice-cold 0.1N hydrochloric acid and the aqueous mixture was extracted with ethyl acetate. The organic layer was washed successively with saturated aqueous sodium hydrogen carbonate, water and brine, dried over anhydrous magnesium sulfate, and evaporated in vacuo. The residue was purified by column chromatography on silica gel (hexane:ethyl acetate=10:1) to give tert-butyl (S)-2,2-dimethyl-4-[4-[[(trifluoromethyl)sulfonyl]oxy]benzyl]-1,3-oxazolidine-3-carboxylate (13 g).

NMR (CDCl₃, δ): 1.35-1.7 (15H, m), 2.65-2.85 (1H, m), 3.05-3.3 (1H, m), 3.7-4.2 (3H, m), 7.15-7.4 (4H, m)

Preparation 3

Under nitrogen, to a solution of benzenethiol (0.94 ml) in tetrahydrofuran (30 ml) was added dropwise butyllithium (1.52M in hexane, 6.0 ml) in acetone-dry ice bath, and the mixture was stirred at the same temperature for 20 minutes. Under nitrogen, to a solution of tert-butyl (S)-2,2-dimethyl-4-[4-[[(trifluorometyl)sulfonyl]oxy]benzyl]-1,3-oxazolidine-3-carboxylate (3.6 g), lithium chloride (770 mg) and tetrakis(triphenylphosphine)palladium(0) (1.9 g) in tetrahydrofuran (40 ml) was added the above prepared solution at room temperature, and the mixture was refluxed for 40 minutes. The mixture was poured into water and the aqueous mixture was extracted with ethyl acetate. The organic layer was washed with brine, dried over anhydrous magnesium sulfate, and evaporated in vacuo. The residue was purified by column chromatography on silica gel (hexane:ethyl acetate=20:1 to 10:1) to give tert-butyl (S)-2,2-dimethyl-4-[4-(phenylthio) benzyl]-1,3-oxazolidine-3-carboxylate (1.8 g).

NMR (CDCl₃, δ): 1.4-1.7 (15H, m), 2.55-2.75 (1H, m), 3.0-3.25 (1H, m), 3.7-4.2 (3H, m), 7.1-7.4 (9H, m)

Preparation 4

The following compounds were obtained according to a similar manner to that of Preparation 3.

(1) tert-Butyl (S)-4-[4-[(4-methoxyphenyl)thio]benzyl]-2,2-dimethyl-1,3-oxazolidine-3-carboxylate

NMR (CDC₁₃, δ): 1.4-1.6 (15H, m), 2.5-2.8 (1H, m), 3.0-3.3 (1H, m), 3.7-4.2 (6H, m), 6.85-7.5 (8H, m) (+)ESI-MS (m/z): 452 (M+Na)⁺

(2) tert-Butyl (S)-4-[4-[(4-fluorophenyl)thio]benzyl]-2,2-dimethyl-1,3-oxazolidine-3-carboxylate

NMR (CDCl₃, δ): 1.4-1.65 (15H, m), 2.6-2.75 (1H, m), 3.0-3.25 (1H, m), 3.7-4.2 (3H, m), 6.95-7.5 (8H, m) (+)ESI-MS (m/z): 440 (M+Na)⁺

(3) tert-Butyl (S)-2,2-dimethyl-4-[4-(2-thienylthio)benzyl]-1,3-oxazolidine-3-carboxylate

NMR (CDCl₃, δ): 1.4-1.7 (15H, m), 2.55-2.7 (1H, m), 2.95-3.25 (1H, m), 3.65-4.15 (3H, m), 7.05-7.5 (7H, m) (+)ESI-MS (m/z): 428 (M+H)⁺

Preparation 5

Under nitrogen at 5° C., to a solution of tert-butyl (S)-2,2-dimethyl-4-[4-(phenylthio)benzyl]-1,3-oxazolidine-3-carboxylate (230 mg) in dichloromethane (10 ml) were added sodium hydrogen carbonate (170 mg) and m-chloroperbenzoic acid (300 mg) and the mixture was stirred at the same temperature for 1 hour. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (hexane:ethyl acetate=10:1 to 4:1) to give tert-butyl (S)-2,2-dimethyl-4-[4-(phenylsulfonyl)benzyl]-1,3-oxazolidine-3-carboxylate (250 mg).

NMR (CDCl₃, δ): 1.35-1.6 (9H, m), 2.65-2.8 (1H, m), 3.05-3.3 (1H, m), 3.6-3.8 (2H, m), 3.9-4.2 (1H, m), 7.25-7.6 (5H, m), 7.8-8.0 (4H, m) (+)ESI-MS (m/z): 454 (M+Na)⁺

Preparation 6

The following compounds were obtained according to a similar manner to that of Preparation 5.

(1) tert-Butyl (S)-4-[4-[(4-methoxyphenyl)sulfonyl]benzyl]-2,2-dimethyl-1,3-oxazolidine-3-carboxylate

NMR (CDCl₃, δ): 2.65-2.8 (1H, m), 3.05-3.3 (1H, m), 3.65-3.85 (2H, m), 3.84 (3H, s), 3.9-4.2 (1H, m), 6.9-7.05 (2H, m), 7.3-7.5 (2H, m), 7.75-7.9 (4H, m) (+)ESI-MS (m/z): 484 (M+Na)⁺

(2) tert-Butyl (S)-4-[4-[(4-fluorophenyl)sulfonyl]benzyl]-2,2-dimethyl-1,3-oxazolidine-3-carboxylate

NMR (CDCl₃, δ): 1.4-1.7 (15H, m), 2.7-2.85 (1H, m), 3.05-3.3 (1H, m), 3.65-3.85 (2H, m), 3.9-4.15 (1H, m), 7.1-7.45 (4H, m), 7.8-8.0 (4H, m) (+)ESI-MS (m/z): 472 (M+Na)⁺

(3) tert-Butyl (S)-2,2-dimethyl-4-[4-(2-thienylsulfonyl)benzyl]-1,3-oxazolidine-3-carboxylate

NMR (CDCl₃, δ): 1.4-1.65 (15H, m), 2.7-2.85 (1H, m), 3.05-3.3 (1H, m), 3.65-3.85 (2H, m), 3.9-4.2 (1H, m), 7.05-7.1 (1H, m), 7.3-7.45 (1H, m), 7.6-7.75 (1H, m), 7.85-7.95 (1H, m) (+)ESI-MS (m/z): 460 (M+Na)⁺

Preparation 7

To a solution of (S)-2,2-dimethyl-4-[4-(phenylsulfonyl)benzyl]-1,3-oxazolidine-3-carboxylate (230 mg) in a mixture of 1,4-dioxane (1 ml) and methanol (1 ml) was added 4N hydrogen chloride in 1,4-dioxane (2 ml) at room temperature, and the mixture was stirred at the same temperature for 2.5 hours. After evaporation under reduced pressure, the residue was dried in vacuo to give (S)-2-amino-3-[4-(phenylsulfonyl)phenyl]-1-propanol hydrochloride (190 mg).

NMR (DMSO-d₆, δ): 2.9-3.0 (2H, m), 3.2-3.6 (3H, m), 7.5-8.2 (9H, m) (+)ESI-MS (m/z): 292 (M-HCl+H)⁺

Preparation 8

The following compounds were obtained according to a similar manner to that of Preparation 7.

(1) (S)-2-Amino-3-[4-[(4-methoxyphenyl)sulfonyl]phenyl]-1-propanol hydrochloride

NMR (DMSO-d₆, δ): 2.9-2.95 (2H, m), 3.25-3.6 (3H, m), 3.83 (3H, s), 7.13 (2H, d, J=8.9 Hz), 7.50 (2H, d, J=8.2 Hz), 7.85-7.95 (4H, m) (+)APCI-MS (m/z): 322 (M-HCl+H)⁺

(2) (S)-2-Amino-3-[4-[(4-fluorophenyl)sulfonyl]phenyl]-1-propanol hydrochloride

NMR (DMSO-d₆, δ): 2.8-3.1 (2H, m), 3.2-3.6 (3H, m), 7.4-7.65 (4H, m), 7.9-8.3 (4H, m) (+)APCI-MS (m/z): 310 (M-HCl+H)⁺

(3) (S)-2-Amino-3-[4-(2-thienylsulfonyl)phenyl]-1-propanol hydrochloride

NMR (DMSO-d₆, δ): 2.9-3.2 (2H, m), 3.25-3.6 (3H, m), 7.24 (1H, dd, J=3.8, 4.9 Hz), 7.57 (2H, d, J=8.3 Hz), 7.86 (1H, dd, J=1.3, 3.8 Hz), 7.94 (2H, d, J=8.3 Hz), 8.10 (1H, dd, J=1.3, 4.9 Hz) (+)ESI-MS (m/z): 298 (M-HCl+H)⁺

(4) (S)-2-Amino-3-[4-(4-quinolinylsulfonyl)phenyl]-1-propanol dihydrochloride

NMR (DMSO-d₆, δ): 2.9-3.0 (2H, m), 3.3-3.8 (3H, m), 7.55 (2H, d, J=8.3 Hz), 7.75-8.1 (7H, m), 8.15-8.25 (1H, m), 8.26 (1H, d, J=4.4 Hz), 8.55-8.65 (1H, m), 9.23 (1H, d, J=4.4 Hz) (+)APCI-MS (m/z): 343 (M-2HCl+H)⁺

Preparation 9

Under nitrogen at 5° C., to a solution of (S)-2-amino-3-[4-(phenylsulfonyl)phenyl]-1-propanol hydrochloride (410 mg) in methanol (10 ml) was added sodium methoxide (28% in methanol, 0.24 ml), and the mixture was stirred at the same temperature for 20 minutes. After removal of the insoluble materials by filtration, the filtrate was evaporated and dried in vacuo. A mixture of the residue and benzaldehyde (0.13 ml) in toluene (10 ml) in the presence of a catalytic amount of p-toluenesulfonic acid monohydrate was refluxed for 1.5 hours to remove water as the toluene azeotrope. After removal of toluene by evaporation, to a solution of the residue in methanol (5 ml) was added sodium borohydride (47 mg) under nitrogen at 5° C., and the mixture was stirred at room temperature for 1.5 days. The resulting mixture was poured into water and the aqueous mixture was extracted with ethyl acetate. The organic layer was washed successively with saturated aqueous sodium hydrogen carbonate and brine, dried over anhydrous magnesium sulfate and evaporated under reduced pressure. The residue was purified by column chromatography on silica gel (chloroform:methanol=100:1 to 30:1) to give (S)-2-(benzylamino)-3-[4-(phenylsulfonyl)-phenyl]-1-propanol (270 mg).

NMR (CDCl₃, δ): 2.7-3.0 (3H, m), 3.25-3.35 (1H, m), 3.55-3.7 (1H, m), 3.76 (2H, s), 7.1-7.35 (7H, m), 7.45-7.65 (3H, m), 7.8-8.0 (4H, m) (+)APCI-MS (m/z): 382 (M+H)⁺

Preparation 10

To an ice-cooled mixture of (R)-1-(4-benzyloxy-3-nitrophenyl)-2-bromoethanol (140.86 g, 87.3% ee), pyridine (65 ml) and 4-(dimethylamino)pyridine (2.44 g) in toluene (705 ml) was added (1S)-(−)-camphanic chloride (95.21 g) in portions over 15 minutes. The mixture was stirred at room temperature for 22 hours. The mixture was cooled with an ice bath and partitioned between toluene and water. The organic layer was separated, washed twice with water (430 ml), once with sodium hydrogen carbonate solution (430 ml), once with brine (430 ml), dried over magnesium sulfate, and filtered. The filtrate was concentrated and the residual oil was crystallized from ethyl acetate (107 ml)—2-propanol (1070 ml) to give crude (1S,4R)-camphanic acid (R)-2-bromo-1-(4-benzyloxy-3-nitrophenyl)ethyl ester (193.48 g) as a white powder.

NMR (CDCl₃, δ): 1.02 (3H, s), 1.07 (3H, s), 1.13 (3H, s), 1.60-1.80 (1H, m), 1.85-2.12 (2H, m), 2.32-2.56 (1H, m), 3.52-3.80 (2H, m, AB of ABX), 5.25 (2H, s), 6.07 (1H, dd, J=8, 5 Hz, X of ABX), 7.15 (1H, d, J=9 Hz), 7.28-7.52 (5H, m), 7.57 (1H, dd, J=9, 2 Hz), 7.89 (1H, d, J=2 Hz)

MS (m/z): 554, 556 (M+Na)⁺

Preparation 11

The crude powder of (245.78 g) of (1S,4R)-camphanic acid (R)-2-bromo-1-(4-benzyloxy-3-nitrophenyl)ethyl ester, the object compound in Preparation 10 was recrystallized from ethyl acetate (490 ml)—hexane (740 ml) to give pure ester (186.23 g) as white crystals. The diastereomeric excess of the product was determined to be 98.2% de by HPLC analysis using a chiral stationary phase column (Daicel CHIRALPAK AD, 4.6×250 mm, hexane/2-propanol=50/50). The second crop was obtained from the mother liquor by the same method (37.84 g, 97.6% de).

Mp: 149-150° C.

Preparation 12

To an ice-cooled solution of (1S,4R)-camphanic acid (R)-2-bromo-1-(4-benzyloxy-3-nitrophenyl)ethyl ester (229.14 g, 98% de) in tetrahydrofuran (460 ml)—methanol (460 ml) was added dropwise 6N sodium hydroxide solution (158 ml) over 10 minutes. The mixture was stirred at room temperature for 1 hour. The mixture was cooled with an ice bath and partitioned between toluene and water. The organic layer was separated, washed twice with water (460 ml), once with brine (460 ml), dried over magnesium sulfate, and filtered. The filtrate was concentrated to give a solid. The solid was recrystallized from ethyl acetate (120 ml)—hexane (820 ml) to give (R)-(4-benzyloxy-3-nitrophenyl)oxirane (110.80 g) as a white powder. The enantiomeric excess of the product was determined to be 98.2% ee by HPLC analysis using a chiral stationary phase column (Daicel CHIRALPAK AS, 4.6×250 mm, hexane/2-propanol=70/30).

NMR (CDCl₃, δ): 2.76 (1H, dd, J=5, 2 Hz), 3.16 (1H, dd, J=5, 4 Hz), 3.85 (1H, dd, J=4, 2 Hz), 5.24 (2H, s), 7.10 (1H, d, J=9 Hz), 7.25-7.52 (6H, m), 7.78 (1H, d, J=2 Hz)

MS (m/z): 294 (M+Na)⁺

Preparation 13

Under nitrogen, to a solution of triisopropylsilane (0.48 g) in tetrahydrofuran (10 ml) was added dropwise butyllithium (1.54M in hexane, 1.6 ml) in acetone-dry ice bath, and the mixture was stirred at the same temperature for 15 minutes. After removal of the cooling bath, to this one were added a solution of tert-butyl (S)-2,2-dimethyl-4-[4-[[(trifluoromethyl)sulfonyl]oxy]benzyl]-1,3-oxazolidine-3-carboxylate (1.0 g) in tetrahydrofuran (4 ml) and tetrakis(triphenylphosphine)palladium(0) (0.26 g), and the mixture was refluxed for 4.5 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=20:1 to 10:1) to give tert-butyl (S)-2,2-dimethyl-4-[4-[(triisopropylsilyl)thio]benzyl]-1,3-oxazolidine-3-carboxylate (280 mg).

NMR (CDCl₃, δ): 1.0-1.35 (21H, m), 1.45-1.7 (15H, m), 2.5-2.7 (1H, m), 3.0-3.25 (1H, m), 3.65-4.2 (3H, m), 7.0-7.15 (2H, m), 7.35-7.5 (2H, m) (+)ESI-MS (m/z): 346 (M−ⁱPr₃Si+2H)⁺

Preparation 14

Under nitrogen at room temperature, to a solution of tert-butyl (S)-2,2-dimethyl-4-[4-[(triisopropylsilyl)thio]-benzyl]-1,3-oxazolidine-3-carboxylate (270 mg) in N,N-dimethylformamide (5 ml) were added cesium fluoride (92 mg) and 4-chloroquinoline (99 mg), and the mixture was stirred at the same temperature for 12 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=3:1 to 2:1) to give tert-butyl (S)-2,2-dimethyl-4-[4-(4-quinolinylthio)benzyl]-1,3-oxazolidine-3-carboxylate (180 mg).

NMR (CDCl₃, δ): 1.45-1.7 (15H, m), 2.7-2.9 (1H, m), 3.1-3.3 (1H, m), 3.7-4.3 (3H, m), 6.76 (1H, d, J=4.6 Hz), 7.25-7.4 (2H, m), 7.45-7.8 (4H, m), 8.09 (1H, d, J=8.3 Hz), 8.22 (1H, d, J=7.6 Hz), 8.58 (1H, d, J=4.8 Hz) (+)ESI-MS (m/z): 451 (M+H)⁺

Preparation 15

Under nitrogen, to a solution of tert-butyl (S)-2,2-dimethyl-4-[4-(4-quinolinylthio)benzyl]-1,3-oxazolidine-3-carboxylate (140 mg) in dichloromethane (2 ml) were added acetic acid (1 ml) and m-chloroperbenzoic acid (110 mg) at 5° C., and the mixture was stirred at the same temperature for 30 minutes. The mixture was poured into a mixture of water and ethyl acetate and the mixture was made alkaline with aqueous 5N sodium hydroxide. After separation, the organic layer was washed successively with water and brine, dried over anhydrous magnesium sulfate and evaporated under reduced pressure. The residue was purified by column chromatography on silica gel (hexane:ethyl acetate=1:1) to give tert-butyl (S)-2,2-dimethyl-4-[4-(4-quinolinyl-sulfonyl)benzyl]-1,3-oxazolidine-3-carboxylate (51 mg).

NMR (CDCl₃, δ): 1.3-1.6 (15H, m), 2.7-2.95 (1H, m), 3.05-3.3 (1H, m), 3.8-4.15 (3H, m), 7.3-7.5 (2H, m), 7.6-7.85 (2H, m), 7.9-8.05 (2H, m), 8.1-8.25 (2H, m), 8.67 (1H, d, J=8.4 Hz), 9.12 (1H, d, J=4.4 Hz) (+)ESI-MS (m/z): 505 (M+Na)⁺

Preparation 16

Under nitrogen at room temperature, to a solution of 4-fluorobenzaldehyde (3.0 g) in N,N-dimethylformamide (60 ml) was added 4-methoxybenzenethiol (3.3 ml) and potassium carbonate (3.7 g), and the mixture was stirred at 120° C. for 6 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=10:1) to give 4-[(4-methoxyphenyl)thio]benzaldehyde (4.9 g).

NMR (CDCl₃, δ): 3.86 (3H, s), 6.95-7.0 (2H, m), 7.1-7.2 (2H, m), 7.45-7.5 (2H, m), 7.65-7.7 (2H, m), 9.89 (1H, s) (+)APCI-MS (m/z): 245 (M+H)⁺

Preparation 17

The following compounds were obtained according to a similar manner to that of Preparation 16.

(1) 4-[(3-Methoxyphenyl)thio]benzaldehyde

NMR (CDCl₃, δ): 3.81 (3H, s), 6.9-7.0 (1H, m), 7.05-7.15 (2H, m), 7.25-7.4 (3H, m), 7.7-7.8 (2H, m), 9.92 (1H, s) (+)APCI-MS (m/z): 245 (M+H)⁺

(2) 4-[(2-Methoxyphenyl)thio]benzaldehyde

NMR (CDCl_{3, δ):} 3.82 (3H, s), 6.95-7.1 (2H, m), 7.15-7.25 (2H, m), 7.4-7.55 (2H, m), 7.65-7.75 (2H, m), 9.90 (1H, s) (+)APCI-MS (m/z): 245 (M+H)⁺

(3) 4-[(3,4-Dimethoxyphenyl)thio]benzaldehyde

NMR (CDCl₃, δ): 3.87 (3H, s), 3.94 (3H, s), 6.94 (1H, d, J=8.3 Hz), 7.05 (1H, d, J=2.0 Hz), 7.1-7.25 (3H, m), 7.65-7.8 (2H, m), 9.89 (1H, s) (+)ESI-MS (m/z): 297 (M+Na)⁺

Preparation 18

Under nitrogen at 5° C., to a solution of 4-[(4-methoxyphenyl)thio]benzaldehyde (4.8 g) in dichloromethane (100 ml) was added m-chloroperbenzoic acid (11 g), and the mixture was stirred at the same temperature for 2.5 hours. The resulting mixture was poured into saturated aqueous sodium hydrogen carbonate and the aqueous mixture was extracted with ethyl acetate. The organic layer was washed successively with saturated aqueous sodium hydrogen carbonate and brine, dried over anhydrous magnesium sulfate and evaporated under reduced pressure followed by dryness to give 4-[(4-methoxyphenyl)sulfonyl]benzaldehyde (5.3 g).

NMR (CDCl₃, δ): 3.85 (3H, s), 6.95-7.05 (2H, m), 7.85-8.1 (6H, m), 10.07 (1H, s) (+)APCI-MS (m/z): 277 (M+H)⁺

Preparation 19

Under nitrogen at 5° C., to a solution of tert-butyl N-benzyl-N-[2-[4-[(4-methoxyphenyl)thio]phenyl]ethyl]-carbamate (1.3 g) in dichloromethane (25 ml) was added m-chloroperbenzoic acid (1.5 g), and the mixture was stirred at the same temperature for 45 minutes. The mixture was poured into aqueous sodium thiosulfate and the aqueous mixture was extracted with ethyl acetate. The organic layer was washed successively with saturated aqueous sodium hydrogen carbonate twice and brine, dried over anhydrous magnesium sulfate and evaporated under reduced pressure followed by dryness to give t-butyl N-benzyl-N-[2-[4-[(4-methoxyphenyl)sulfonyl]phenyl]ethyl]carbamate (1.5 g).

(+)ESI-MS (m/z): 504 (M+Na)⁺

Preparation 20

The following compounds were obtained according to a similar manner to that of Preparation 19.

(1) tert-Butyl N-benzyl-N-[2-[4-[[4-(4-fluorophenoxy)phenyl]sulfonyl]phenyl]ethyl]carbamate

(+)ESI-MS (m/z): 584 (M+Na)⁺

(2) tert-Butyl N-benzyl-N-[2-[4-[(3-methoxyphenyl)sulfonyl]phenyl]ethyl]carbamate

(+)ESI-MS (m/z): 504 (M+Na)⁺

(3) tert-Butyl N-benzyl-N-[2-[4-[(3-hydroxyphenyl)sulfonyl]phenyl]ethyl]carbamate

NMR (CDCl₃, δ): 1.38 (9H, br s), 2.7-2.9 (2H, m), 3.25-3.5 (2H, m), 4.37 (2H, br s), 6.95-7.05 (1H, m), 7.15-7.5 (10H, m), 7.75-7.85 (2H, m) (+)ESI-MS (m/z): 490 (M+Na)⁺

(4) tert-Butyl N-benzyl-N-[2-[4-[[3-(4-fluorophenoxy)phenyl]sulfonyl]phenyl]ethyl]carbamate

(+)ESI-MS (m/z): 584 (M+Na)⁺

(5) tert-Butyl N-benzyl-N-[2-[4-[(2-methoxyphenyl)sulfonyl]phenyl]ethyl]carbamate

(+)ESI-MS (m/z): 504 (M+Na)⁺

(6) tert-Butyl N-benzyl-N-[2-[4-[(2-hydroxyphenyl)sulfonyl]phenyl]ethyl]carbamate

(+)ESI-MS (m/z): 490 (M+Na)⁺

(7) Ethyl [2-[[4-[2-[N-benzyl-N-(tert-butoxycarbonyl)-amino]ethyl]phenyl]sulfonyl]phenoxy]acetate

NMR (CDCl₃, δ): 1.23 (3H, t, J=7.1 Hz), 1.43 (9H, s), 2.7-2.95 (2H, br s), 3.25-3.5 (2H, br s), 4.19 (2H, q, J=7.1 Hz), 4.25-4.45 (1H, m), 4.59 (2H, s), 6.75-6.85 (1H, m), 7.1-7.35 (8H, m), 7.45-7.55 (1H, m), 7.9-8.0 (2H, m), 8.15-8.2 (1H, m) (+)ESI-MS (m/z): 576 (M+Na)⁺

(8) tert-Butyl N-benzyl-N-[2-[4-[[2-(4-fluorophenoxy)phenyl]sulfonyl]phenyl]ethyl]carbamate

NMR (CDCl₃, δ): 1.42 (9H, br s), 2.65-2.9 (2H, m), 3.25-3.5 (2H, m), 4.25-4.5 (2H, m), 6.65-6.8 (3H, m), 6.85-7.0 (2H, m), 7.05-7.5 (9H, m), 7.8-7.95 (2H, m), 8.2-8.3 (1H, m) (+)APCI-MS (m/z): 462 (M-Boc+2H)⁺

(9) tert-Butyl N-benzyl-N-[2-[4-[(3,4-dimethoxyphenyl)sulfonyl]phenyl]ethyl]carbamate

NMR (CDCl₃, δ): 1.39 (9H, br s), 2.7-2.95 (2H, m), 3.25-3.5 (2H, m), 3.90 (3H, s), 3.91 (3H, s), 4.25-4.5 (2H, m), 6.91 (1H, d, J=8.5 Hz), 7.1-7.4 (8H, m), 7.5-7.6 (1H, m), 7.75-7.85 (2H, m) (+)APCI-MS (m/z): 534 (M+Na)⁺

Preparation 21

Under nitrogen at 5° C., to a suspension of (methoxymethyl)triphenylphosphonium chloride (2.5 g) in tetrahydrofuran (10 ml) was added potassium tert-butoxide (0.74 g) by portions with care, and the mixture was stirred at room temperature for 30 minutes. To this one was added 4-[(4-methoxyphenyl)sulfonyl]benzaldehyde (0.91 g) in tetrahydrofuran (10 ml), and the mixture was stirred at room temperature for 12 hours. The resulting mixture was poured into water and the aqueous mixture was extracted in ethyl acetate. The organic layer was washed with brine, dried over anhydrous magnesium sulfate and evaporated under reduced pressure. The residue was purified by column chromatography on silica gel (hexane:ethyl acetate=3:1 to 1:1) to give 1-methoxy-4-[[4-(2-methoxyethenyl)phenyl]sulfonyl]benzene (0.89 g).

(+)APCI-MS (m/z): 305 (M+H)⁺

Preparation 22

Under nitrogen at room temperature, to a solution of 1-methoxy-4-[[4-(2-methoxyethenyl)phenyl]sulfonyl]benzene (400 mg) in dichloromethane (4 ml) was added formic acid (2 ml), and the mixture was refluxed for 10 hours. The resulting mixture was poured into water and the aqueous mixture was extracted with ethyl acetate. The organic layer was washed successively with water and brine, dried over anhydrous magnesium sulfate and evaporated under reduced pressure followed by dryness to give crude [4-[(4-methoxyphenyl)sulfonyl]phenyl]acetaldehyde which was used in the next step.

Preparation 23

A mixture of (S)-2-(phenoxymethyl)oxirane (3.0 g) and 28% ammonium hydroxide (15 ml) in ethanol (30 ml) was sealed at room temperature for 12 hours. After evaporation under reduced pressure, the residue was dissolved into a mixture of ethyl acetate and methanol followed by addition of 4N hydrogen chloride in 1,4-dioxane. After being stirred for 12 hours, the precipitates were collected by filtration followed by being washed with ethyl acetate and dryness to give (S)-1-amino-3-phenoxy-2-propanol hydrochloride (3.4 g).

(+)ESI-MS (m/z): 168 (M-HCl+H)⁺

Preparation 24

Under nitrogen at room temperature, to a solution of 4-[(4-methoxyphenyl)thio]benzaldehyde (5.1 g) in methanol (51 ml) were added nitromethane (1.7 ml), acetic acid (0.60 ml) and butylamine (1.0 ml), and the mixture was stirred at the same temperature overnight to give a precipitate. Water (51 ml) was poured into the resulting mixture and the mixture was stirred for 30 minutes. The deposit was collected by filtration and the filter cake was washed with water followed by air-drying to give 1-methoxy-4-[[4-(2-nitroethenyl)phenyl]thio]benzene (5.4 g).

NMR (CDCl₃, δ): 3.86 (3H, s), 6.9-7.15 (4H, m), 7.3-7.6 (5H, m), 7.85-7.95 (1H, m) (+)ESI-MS (m/z): 310 (M+Na)⁺

Preparation 25

The following compounds were obtained according to a similar manner to that of Preparation 24.

(1) 1-Methoxy-3-[[4-(2-nitroethenyl)phenyl]thio]benzene

NMR (CDCl₃, δ): 3.80 (3H, s), 6.85-7.15 (3H, m), 7.2-7.55 (6H, m), 7.9-8.0 (1H, m) (+)ESI-MS (m/z): 310 (M+Na)⁺

(2) 1-Methoxy-2-[[4-(2-nitroethenyl)phenyl]thio]benzene

NMR (DMSO-d₆, δ): 3.78 (3H, s), 6.95-7.25 (4H, m), 7.35-7.55 (2H, m), 7.7-7.85 (2H, m), 8.0-8.25 (2H, m) (+)APCI-MS (m/z): 288 (M+H)⁺

(3) 1,2-Dimethoxy-4-[[4-(2-nitroethenyl)phenyl]thio]benzene

NMR (CDCl₃, δ): 3.88 (3H, s), 3.94 (3H, s), 6.85-7.0 (1H, m), 7.0-7.25 (4H, m), 7.35-7.4 (2H, m), 7.45-7.6 (1H, m), 7.9-8.0 (1H, m) (+)ESI-MS (m/z): 340 (M+Na)⁺

Preparation 26

Under nitrogen at 5° C., to a suspension of lithiumaluminum hydride (3.2 g) in tetrahydrofuran (80 ml) was added dropwise 1-methoxy-4-[[4-(2-nitroethenyl)phenyl]-thio]benzene (4.8 g) in tetrahydrofuran (50 ml), and the mixture was refluxed for 6.5 hours. The resulting mixture was cooled to 5° C., and to this one was added sodium fluoride (14 g) followed by water (4.5 ml) dropwise carefully. The mixture was vigorously stirred at room temperature for 30 minutes. The precipitate was removed by filtration, and the filter cake was washed with a mixture of ethyl acetate and ethanol (95:5). The filtrate was evaporated under reduced pressure. The residue was dissolved in ethyl acetate (40 ml) and cooled to 5° C. To this one was added 4N hydrogen chloride in 1,4-dioxane (8.4 ml) and the mixture was stirred at room temperature for 30 minutes to deposit the corresponding salt followed by collection by filtration. The filter cake was washed with ethyl acetate and dissolved in a mixture of ethyl acetate and 1N sodium hydroxide. After separation, the organic layer was dried over anhydrous magnesium sulfate, evaporated under reduced pressure and dried to give 2-[4-[(4-methoxyphenyl)thio]phenyl]ethylamine (2.0 g).

NMR (CDCl₃, δ): 2.69 (2H, t, J=6.8 Hz), 2.93 (2H, t, J=6.8 Hz), 3.81 (3H, s), 6.85-6.95 (2H, m), 7.05-7.2 (4H, m), 7.35-7.45 (2H, m) (+)APCI-MS (m/z): 260 (M+H)⁺

Preparation 27

The following compounds were obtained according to a similar manner to that of preparation 26.

(1) 2-[4-[(3-Methoxyphenyl)thio]phenyl]ethylamine

NMR (CDCl₃, δ): 2.74 (2H, t, J=6.9 Hz), 2.97 (2H, t, J=6.9 Hz), 3.75 (3H, s), 6.7-6.9 (3H, m), 7.1-7.4 (5H, m) (+)ESI-MS (m/z): 260 (M+H)⁺

(2) 2-[4-[(2-Methoxyphenyl)thio]phenyl]ethylamine

NMR (CDCl₃, δ): 2.74 (2H, t, J=6.6 Hz), 2.9-3.05 (2H, m), 3.88 (3H, s), 6.8-7.4 (8H, m) (+)APCI-MS (m/z): 260 (M+H)⁺

(3) 2-[4-[(3,4-Dimethoxyphenyl)thio]phenyl]ethylamine

NMR (DMSO-d₆, δ): 2.45-2.8 (4H, m), 3.72 (3H, s), 3.77 (3H, s), 6.9-7.2 (7H, m) (+)ESI-MS (m/z): 290 (M+H)⁺

Preparation 28

Under nitrogen at room temperature, to a solution of 2-[4-[(4-methoxyphenyl)thio]phenyl]ethylamine (2.0 g) in dichloromethane (20 ml) was added benzaldehyde (0.78 ml), and the mixture was stirred at the same temperature for 20 minutes. To this one was added toluene and evaporated under reduced pressure. Under nitrogen at 5° C., to a solution of the residue in tetrahydrofuran (20 ml) was added sodium borohydride (0.32 g) followed by methanol (10 ml) dropwise and the mixture was stirred at room temperature for 40 minutes. The resulting mixture was poured into a mixture of ethyl acetate and water and stirred for 10 minutes. After separation, the organic layer was washed with brine, dried over anhydrous magnesium sulfate and evaporated under reduced pressure. The residue was purified by column chromatography on silica gel (chloroform:methanol=100:1 to 20:1) to give N-benzyl-N-[2-[4-[(4-methoxyphenyl)thio]-phenyl]ethyl]amine (2.0 g).

NMR (CDCl₃, δ): 2.7-2.9 (4H, m), 3.81 (2H, s), 3.83 (3H, s), 6.85-6.95 (2H, m), 7.05-7.45 (11H, m) (+)APCI-MS (m/z): 350 (M+H)⁺

Preparation 29

The following compounds were obtained according to a similar manner to that of Preparation 28.

(1) N-Benzyl-N-[2-[4-[(3-methoxyphenyl)thio]phenyl]ethyl]-amine

NMR (CDCl₃, δ): 2.75-3.0 (4H, m), 3.78 (3H, s), 3.80 (2H, s), 6.7-6.95 (3H, m), 7.1-7.4 (10H, m) (+)APCI-MS (m/z): 350 (M+H)⁺

(2) N-Benzyl-N-[2-[4-[(2-methoxyphenyl)thio]phenyl]ethyl]-amine

NMR (CDCl₃, δ): 2.75-2.95 (4H, m), 3.84 (2H, s), 3.87 (3H, s), 6.75-6.9 (2H, m), 6.95-7.05 (1H, m), 7.15-7.4 (10H, m) (+)APCI-MS (m/z): 350 (M+H)⁺

(3) N-Benzyl-N-[2-[4-[(3,4-dimethoxyphenyl)thio]-phenyl]ethyl]amine

NMR (CDCl₃, δ): 2.7-2.95 (4H, m), 3.79 (2H, s), 3.82 (3H, s), 3.88 (3H, s), 6.84 (1H, d, J=8.3 Hz), 6.95-7.4 (11H, m) (+)ESI-MS (m/z): 380 (M+H)⁺

Preparation 30

Under nitrogen at room temperature, to a solution of N-benzyl-N-[2-[4-[(4-methoxyphenyl)thio]phenyl]ethyl]amine (1.0 g) in tetrahydrofuran (10 ml) was added di-tert-butyl dicarbonate (0.69 g) in tetrahydrofuran (2 ml), and the mixture was stirred at the same temperature for 1.5 hours. The resulting mixture was poured into saturated aqueous sodium hydrogen carbonate and the aqueous mixture was extracted with ethyl acetate. The organic layer was washed with brine, dried over anhydrous magnesium sulfate and evaporated under reduced pressure followed by dryness to give tert-butyl N-benzyl-N-[2-[4-[(4-methoxyphenyl)thio]-phenyl]ethyl]carbamate (1.3 g).

(+)ESI-MS (m/z): 472 (M+H)⁺

Preparation 31

The following compounds were obtained according to a similar manner to that of Preparation 30.

(1) tert-Butyl N-benzyl-N-[2-[4-[(4-hydroxyphenyl)thio]phenyl]ethyl]carbamate

NMR (CDCl₃, δ): 1.45 (9H, s), 2.6-2.85 (2H, m), 3.25-3.45 (2H, m), 4.3-4.45 (2H, m), 6.75-6.85 (2H, m), 6.9-7.4 (11H, m) (+)ESI-MS (m/z): 458 (M+Na)⁺

(2) tert-Butyl N-[(R)-2-(3-chlorophenyl)-2-hydroxyethyl]-N-[2-[4-[(4-cyanophenyl)thio]phenyl]ethyl]carbamate

(+)ESI-MS (m/z): 531, 533 (M+Na)⁺

(3) tert-Butyl N-benzyl-N-[2-[4-[(3-methoxyphenyl)thio]phenyl]ethyl]carbamate

(+)ESI-MS (m/z): 472 (M+Na)⁺

(4) tert-Butyl N-benzyl-N-[2-[4-[(3-hydroxyphenyl)thio]phenyl]ethyl]carbamate

NMR (CDCl₃, δ): 1.45 (9H, br s), 2.7-2.85 (2H, m), 3.3-3.5 (2H, m), 4.37 (2H, s), 6.55-6.7 (2H, m), 6.75-6.85 (1H, m), 7.05-7.4 (10H, m) (+)ESI-MS (m/z): 458 (M+Na)⁺

(5) tert-Butyl N-[(R)-2-(3-chlorophenyl)-2-hydroxyethyl]-N-[2-[4-[(3-cyanophenyl)thio]phenyl]ethyl]carbamate

NMR (CDCl₃, δ): 1.4-1.55 (9H, m), 2.65-2.9 (2H, m), 3.2-3.4 (4H, m), 4.8-4.95 (1H, m), 7.0-7.5 (12H, m) (+)ESI-MS (m/z): 531, 533 (M+Na)⁺

(6) tert-Butyl N-benzyl-N-[2-[4-[(2-methoxyphenyl)thio]phenyl]ethyl]carbamate

(+)ESI-MS (m/z): 472 (M+Na)⁺

(7) tert-Butyl N-benzyl-N-[2-[4-[(2-hydroxyphenyl)thio]phenyl]ethyl]carbamate

NMR (CDCl₃, δ): 1.43 (9H, br s), 2.6-2.85 (2H, m), 3.2-3.45 (2H, m), 4.25-4.45 (2H, m), 6.85-7.6 (13H, m) (+)ESI-MS (m/z): 458 (M+Na)⁺

(8) tert-Butyl N-benzyl-N-[2-[4-[(3,4-dimethoxyphenyl)thio]phenyl]ethyl]carbamate

NMR (CDCl₃, δ): 1.45 (9H, br s), 2.6-2.85 (2H, m), 3.2-3.5 (2H, m), 3.82 (3H, s), 3.88 (3H, s), 4.25-4.45 (2H, m), 6.83 (1H, d, J=8.3 Hz), 6.95-7.4 (11H, m) (+)ESI-MS (m/z): 502 (M+Na)⁺

Preparation 32

At room temperature, to a solution of N-benzyl-N-[2-[4-(4-methoxybenzenesulfonyl)phenyl]ethyl]carbamic acid tert-butyl ester (1.5 g) in ethyl acetate (10 ml) was added 4N hydrogen chloride in 1,4-dioxane (10 ml), and the mixture was stirred at the same temperature for 1 hour to give a precipitate. The precipitate was collected by filtration and washed with ethyl acetate followed by dissolution in a mixture of saturated aqueous sodium hydrogen carbonate and ethyl acetate. After separation, the organic layer was washed with brine, dried over anhydrous magnesium sulfate and evaporated under reduced pressure followed by dryness to give N-benzyl-N-[2-[4-(4-methoxybenzenesulfonyl)-phenyl]ethyl]amine (0.92 g).

NMR (CDCl₃, δ): 3.8-3.95 (4H, m), 3.80 (2H, s), 3.83 (3H, s), 6.9-7.0 (2H, m), 7.15-7.35 (7H, m), 7.75-7.9 (4H, m) (+)APCI-MS (m/z): 382 (M+H)⁺

Preparation 33

The following compounds were obtained according to a similar manner to that of Preparation 32.

(1) N-Benzyl-N-[2-[4-[[4-(4-fluorophenoxy)phenyl]-sulfonyl]phenyl]ethyl]amine

NMR (CDCl₃, δ): 2.8-2.95 (4H, m), 3.79 (2H, s), 6.9-7.4 (13H, m), 7.75-7.9 (4H, m) (+)APCI-MS (m/z): 462 (M+H)⁺

(2) N-Benzyl-N-[2-[4-[(3-methoxyphenyl)sulfonyl]-phenyl]ethyl]amine

NMR (CDCl₃, δ): 2.8-2.95 (4H, m), 3.78 (2H, s), 3.84 (3H, s), 7.05-7.1 (1H, m), 7.15-7.55 (10H, m), 7.85-7.9 (2H, m) (+)APCI-MS (m/z): 382 (M+H)⁺

(3) 3-[[4-[2-(Benzylamino)ethyl]phenyl]sulfonyl]phenol

NMR (CDCl₃, δ): 2.7-3.0 (4H, m), 3.81 (2H, s), 6.9-7.0 (1H, m), 7.1-7.5 (10H, m), 7.75-7.85 (2H, m) (−)APCI-MS (m/z): 366 (M−H)⁻

(4) N-Benzyl-N-[2-[4-[[3-(4-fluorophenoxy)phenyl]-sulfonyl]phenyl]ethyl]amine

NMR (CDCl₃, δ): 2.8-3.0 (4H, m), 3.79 (2H, s), 6.9-7.65 (15H, m), 7.75-7.9 (2H, m) (+)APCI-MS (m/z): 462 (M+H)⁺

(5) N-Benzyl-N-[2-[4-[(2-methoxyphenyl)sulfonyl]-phenyl]ethyl]amine

NMR (CDCl₃, δ): 2.8-3.0 (4H, m), 3.76 (3H, s), 3.79 (2H, s), 6.85-6.95 (1H, m), 7.05-7.35 (8H, m), 7.45-7.65 (1H, m), 7.85-7.95 (2H, m), 8.1-8.2 (1H, m) (+)APCI-MS (m/z): 382 (M+H)⁺

(6) 2-[[4-[2-(Benzylamino)ethyl]phenyl]sulfonyl]phenol

NMR (DMSO-d₆, δ): 2.65-2.9 (4H, m), 3.72 (2H, s), 6.8-7.05 (3H, m), 7.1-7.65 (7H, m), 7.7-7.9 (3H, m) (+)ESI-MS (m/z): 368 (M+H)⁺

(7) Ethyl [2-[[4-[2-(benzylamino)ethyl]phenyl]-sulfonyl]phenoxy]acetate

NMR (CDCl₃, δ): 1.23 (3H, t, J=7.1 Hz), 2.85-2.95 (4H, m), 3.79 (2H, s), 4.18 (2H, q, J=7.1 Hz), 4.60 (2H, s), 6.75-6.85 (1H, m), 7.15-7.35 (8H, m), 7.45-7.55 (1H, m), 7.95-8.05 (2H, m), 8.15-8.25 (1H, m) (+)APCI-MS (m/z): 454 (M+H)⁺

(8) N-Benzyl-N-[2-[4-[[2-(4-fluorophenoxy)phenyl]-sulfonyl]phenyl]ethyl]amine

NMR (CDCl₃, δ): 2.8-2.9 (4H, m), 3.80 (2H, s), 6.65-6.8 (3H, m), 6.85-7.0 (2H, m), 7.15-7.55 (9H, m), 7.85-7.95 (2H, m), 8.2-8.3 (1H, m) (+)APCI-MS (m/z): 462 (M+H)⁺

(9) N-Benzyl-N-[2-[4-[(3,4-dimethoxyphenyl)sulfonyl]-phenyl]ethyl]amine

NMR (CDCl₃, δ): 2.8-3.0 (4H, m), 3.78 (2H, s), 3.91 (6H, m), 6.92 (1H, d, J=8.5 Hz), 7.2-7.4 (8H, m), 7.5-7.6 (1H, m), 7.75-7.9 (2H, m) (+)ESI-MS (m/z): 412 (M+H)⁺

Preparation 34

Under nitrogen at 5° C., to a solution of N-benzyl-N-[2-[4-[(4-methoxyphenyl)sulfonyl]phenyl]ethyl]amine (400 mg) in dichloromethane (10 ml) was added 1M boron tribromide in dichloromethane (5.1 ml), and the mixture was stirred at the same temperature for 12 hours. The mixture was evaporated under reduced pressure. The residue was dissolved in a mixture of dichloromethane and saturated aqueous sodium hydrogen carbonate. After separation, the organic layer was dried over anhydrous magnesium sulfate and evaporated under reduced pressure followed by dryness to give 4-[[4-[2-(benzylamino)ethyl]phenyl]sulfonyl]phenol (400 mg).

NMR (DMSO-d₆, δ): 2.65-2.9 (4H, m), 3.68 (2H, s), 6.85-6.95 (2H, m), 7.1-7.45 (7H, m), 7.7-7.85 (4H, m) (+)APCI-MS (m/z): 368 (M+H)⁺

Preparation 35

The following compounds were obtained according to a similar manner to that of Preparation 34.

(1) 4-[[4-[2-(Benzylamino)ethyl]phenyl]thio]phenol

NMR (DMSO-d₆, δ): 2.65-2.75 (4H, m), 3.71 (2H, s), 6.75-6.85 (2H, m), 6.95-7.35 (11H, m) (+)APCI-MS (m/z): 336 (M+H)⁺

(2) 3-[[4-[2-(Benzylamino)ethyl]phenyl]thio]phenol

NMR (DMSO-d₆, δ): 2.7-2.85 (4H, m), 3.74 (2H, s), 7.55-7.75 (3H, m), 7.05-7.4 (10H, m) (+)APCI-MS (m/z): 336 (M+H)⁺

(3) 2-[[4-[2-(Benzylamino)ethyl]phenyl]thio]phenol

NMR (CDCl₃, δ): 2.65-2.95 (4H, m), 3.78 (2H, s), 6.8-7.6 (13H, m) (+)APCI-MS (m/z): 336 (M+H)⁺

Preparation 36

To a solution of tert-butyl N-benzyl-N-[2-[4-[(4-hydroxyphenyl)thio]phenyl]ethyl]carbamate (200 mg) in dichloromethane (6 ml) were added 4-fluorophenylboronic acid (130 mg), copper(II) acetate (83 mg), molecular sieves 4 Å (200 mg) and pyridine (0.19 ml) at room temperature, and the mixture was stirred at the same temperature of 7 days. The insoluble materials were removed by filtration with celite. The filtrate was poured into 0.1N hydrochloric acid and the aqueous mixture was extracted with ethyl acetate. The organic layer was washed successively with saturated aqueous sodium hydrogen carbonate and brine, dried over anhydrous magnesium sulfate and evaporated under reduced pressure. The residue was purified by column chromatography on silica gel (hexane:ethyl acetate=20:1 to 10:1) to give tert-butyl N-benzyl-N-[2-[4-[[4-(4-fluorophenoxy)phenyl]thio]phenyl]-ethyl]carbamate (95 mg).

NMR (CDCl₃, δ): 1.45 (9H, s), 2.65-2.9 (2H, m), 3.25-3.5 (2H, m), 4.3-4.45 (2H, m), 6.85-7.4 (17H, m) (+)ESI-MS (m/z): 552 (M+Na)⁺

Preparation 37

The following compounds were obtained according to a similar manner to that of Preparation 36.

(1) tert-Butyl N-benzyl-N-[2-[4-[[3-(4-fluorophenoxy)phenyl]thio]phenyl]ethyl]carbamate

NMR (CDCl₃, δ): 1.46 (9H, br s), 2.65-2.9 (2H, m), 3.25-3.5 (2H, m), 4.3-4.45 (2H, m), 6.7-7.4 (17H, m) (+)ESI-MS (m/z): 552 (M+Na)⁺

(2) tert-Butyl N-benzyl-N-[2-[4-[[2-(4-fluorophenoxy)-phenyl]thio]phenyl]ethyl]carbamate

NMR (CDCl₃, δ): 1.47 (9H, br s), 2.65-2.9 (2H, m), 3.25-3.5 (2H, m), 4.25-4.45 (2H, m), 6.8-7.4 (17H, m) (+)ESI-MS (m/z): 552 (M+Na)⁺

Preparation 38

Under nitrogen at 5° C., to a suspension of sodium hydride (60% in oil, 7.9 g) in N,N-dimethylformamide (100 ml) was added methyl 4-hydroxyphenylacetate (30 g) in N,N-dimethylformamide (55 ml), and the mixture was stirred at room temperature for 1 hour followed by cooling to 5° C. To this one was added dimethylthiocarbamoyl chloride (25 g) in N,N-dimethylformamide (55 ml), and the mixture was stirred at 45° C. for 2 hours. The resulting mixture was poured into water and the organic layer was extracted with a mixture of hexane and ethyl acetate (1:1). The organic layer was washed successively with water and brine, dried over anhydrous magnesium sulfate and evaporated under reduced pressure. The residue was purified by column chromatography on silica gel (toluene:ethyl acetate=20:1) to give methyl [4-[[(dimethylamino)thiocarbonyl]oxy]phenyl]acetate (34 g).

NMR (CDCl₃, δ): 3.33 (3H, s), 3.45 (3H, s), 3.63 (2H, s), 3.70 (3H, s), 6.95-7.05 (2H, m), 7.25-7.35 (2H, m) (+)ESI-MS (m/z): 276 (M+Na)⁺

Preparation 39

Under nitrogen, a mixture of methyl [4-[[(dimethylamino)thiocarbonyl]oxy]phenyl]acetate (34 g) in diphenyl ether (100 ml) was stirred at 240° C. for 29 hours. The resulting mixture was purified by column chromatography on silica gel (hexane:ethyl acetate=10:1 to 2:1) to give methyl [4-[[(dimethylamino)carbonyl]thio]phenyl]acetate (25 g).

NMR (CDCl₃, δ): 3.05 (6H, br s), 3.63 (2H, s), 3.68 (3H, s), 7.25-7.35 (2H, m), 7.4-7.5 (2H, m) (+)ESI-MS (m/z): 276 (M+Na)⁺

Preparation 40

Under nitrogen, to a solution of methyl [4-[[(dimethylamino)carbonyl]thio]phenyl]acetate (25 g) in ethanol (200 ml) was added potassium hydroxide (27 g) in water (100 ml), and the mixture was refluxed for 4 hours. The resulting mixture was cooled to 5° C., and to this one was added concentrated hydrochloric acid (40 ml) and water (20 ml). The mixture was stirred at the same temperature for 30 minutes to give a precipitate. The precipitate was collected by filtration and washed with water followed by dryness to give (4-mercaptophenyl)acetic acid (8.6 g).

(−)ESI-MS (m/z): 167 (M−H)⁻

Preparation 41

Under nitrogen at room temperature, to a suspension of (4-mercaptophenyl)acetic acid (1.0 g) in ethanol (20 ml) was added 4N hydrogen chloride in 1,4-dioxane (2 ml), and the mixture was stirred at the same temperature for 12 hours. The resulting mixture was evaporated under reducer pressure followed by dryness to give ethyl (4-mercaptophenyl)acetate (1.2 g).

NMR (CDCl₃, δ): 1.24 (3H, t, J=7.1 Hz), 3.55 (2H, s), 4.14 (2H, q, J=7.1 Hz), 7.1-7.3 (4H, m) (−)APCI-MS (m/z): 195 (M−H)⁻

Preparation 42

Under nitrogen at room temperature, to a solution of ethyl (4-meraptophenyl)acetate (540 mg) in N,N-dimethylformamide (10 ml) were added 4-fluorobenzonitrile (370 mg) and potassium carbonate (570 mg), and the mixture was stirred at 120° C. for 11 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=20:1 to 10:1) to give ethyl [4-[(4-cyanophenyl)thio]phenyl]acetate (590 mg).

NMR (CDCl₃, δ): 1.28 (3H, t, J=7.1 Hz), 3.66 (2H, s), 4.19 (2H, q, J=7.1 Hz), 7.15-7.2 (2H, m), 7.3-7.4 (2H, m), 7.4-7.55 (4H, m) (−)APCI-MS (m/z): 296 (M−H)⁻

Preparation 43

The following compound was obtained according to a similar manner to that of preparation 42.

Ethyl [4-[(3-cyanophenyl)thio]phenyl]acetate

NMR (CDCl₃, δ): 1.27 (3H, t, J=7.1 Hz), 3.64 (2H, s), 4.18 (2H, q, J=7.1 Hz), 7.25-7.5 (8H, m) (+)APCI-MS (m/z): 298 (M+H)⁺

Preparation 44

At 5° C., to a suspension of ethyl [4-[(4-cyanophenyl)thio]phenyl]acetate (570 mg) in a mixture of ethanol (8.5 ml) and tetrahydrofuran (2 ml) was added 1N sodium hydroxide (1.9 ml), and the mixture was stirred at room temperature for 3 hours. After cooling to 5° C., to this one was added 1N hydrochloric acid (1.9 ml), and the mixture was evaporated under reduced pressure followed by dryness to give [4-[(4-cyanophenyl)thio]phenyl]acetic acid (620 mg).

NMR (DMSO-d₆, δ): 3.66 (2H, s), 7.15-7.3 (2H, m), 7.35-7.55 (4H, m), 7.75-7.85 (2H, m) (+)ESI-MS (m/z): 292 (M+Na)⁺

Preparation 45

The following compound was obtained according to a similar manner to that of Preparation 44.

[4-[(3-Cyanophenyl)thio]phenyl]acetic acid

NMR (DMSO-d₆, δ): 3.61 (2H, s), 7.25-7.6 (6H, m), 7.65-7.8 (2H, m) (+)ESI-MS (m/z): 292 (M+Na)⁺

Preparation 46

Under nitrogen at 5° C., to a solution of [4-[(4-cyanophenyl)thio]phenyl]acetic acid (610 mg) in N,N-dimethylformamide (10 ml) were added (R)-2-amino-1-(3-chlorophenyl]ethanol hydrochloride (520 mg), 1-[3-(dimethylamino)propyl]-3-ethylcarbodiimide (380 mg) and 1-hydroxybenzotriazol (330 mg), and the mixture was stirred at room temperature for 12 hours. The resulting mixture was poured into 0.1N hydrochloric acid and the aqueous mixture was extracted with ethyl acetate. The organic layer was washed successively with water, 0.1N sodium hydroxide, water and brine, dried over anhydrous magnesium sulfate and evaporated under reduced pressure followed by dryness to give N-[(R)-2-(3-chlorophenyl)-2-hydroxyethyl]-2-[4-[(4-cyanophenyl)thio]phenyl]acetamide (800 mg).

NMR (CDCl₃, δ): 3.25-3.4 (1H, m), 3.58 (2H, s), 3.6-3.8 (1H, m), 4.8-4.9 (1H, m), 7.15-7.35 (8H, m), 7.4-7.55 (4H, m) (+)ESI-MS (m/z): 445, 447 (M+Na)⁺

Preparation 47

The following compound was obtained according to a similar manner to that of Preparation 46.

N-[(R)-2-(3-Chlorophenyl)-2-hydroxyethyl]-2-[4-[(3-cyanophenyl)thio]phenyl]acetamide

NMR (CDCl₃, δ): 3.25-3.4 (1H, m), 3.59 (2H, s), 3.6-3.8 (1H, m), 4.75-4.9 (1H, m), 7.1-7.55 (12H, m) (−)APCI-MS (m/z): 420, 422 (M−H)⁻

Preparation 48

Under nitrogen at 5° C., to a solution of N-[(R)-2-(3-chlorophenyl)-2-hydroxyethyl]-2-[4-[(4-cyanophenyl)thio]-phenyl]acetamide (760 mg) in tetrahydrofuran (15 ml) was added borane-methyl sulfide complex (0.51 ml), and the mixture was stirred at room temperature for 12 hours. To the resulting mixture was added methanol and the mixture was evaporated under reduced pressure. The residue was dissolved in acetic acid (5 ml) and stirred at 60° C. for 8 hours. The mixture was evaporated under reduced pressure. The residue was dissolved in a mixture of saturated aqueous sodium hydrogen carbonate and ethyl acetate. After separation, the organic layer was washed with brine, dried over anhydrous magnesium sulfate and evaporated under reduced pressure. The residue was purified by column chromatography on silica gel (chloroform:methanol=100:1 to 30:1) to give 4-[[4-[2-[[(R)-2-(3-chlorophenyl)-2-hydroxyethyl]amino]ethyl]phenyl]thio]benzonitrile (190 mg).

(+)APCI-MS (m/z): 409, 411 (M+H)⁺

Preparation 49

The following compound was obtained according to a similar manner to that of Preparation 48.

3-[[4-[2-[[(R)-2-(3-chlorophenyl)-2-hydroxyethyl]amino]ethyl]phenyl]thio]benzonitrile

NMR (CDCl₃, δ): 2.6-3.1 (6H, m), 4.6-4.8 (2H, m), 7.0-7.75 (12H, m) (+)APCI-MS (m/z): 409, 411 (M+H)⁺

Preparation 50

The following compound was obtained according to a similar manner to that of Example 21.

Ethyl [2-[[4-[2-[N-benzyl-N-(tert-butoxycarbonyl)-amino]ethyl]phenyl]thio]phenoxy]acetate

NMR (CDCl₃, δ): 1.29 (3H, t, J=7.2 Hz), 1.47 (9H, br s), 2.65-2.9 (2H, m), 3.25-3.5 (2H, m), 4.25 (2H, q, J=7.2 Hz), 4.25-4.55 (1H, m), 4.68 (2H, s), 6.7-7.4 (8H, m) (+)ESI-MS (m/z): 544 (M+Na)⁺

### EXAMPLE 1

Under nitrogen, to a solution of (S)-2-amino-3-[4-(phenylsulfonyl)phenyl]-1-propanol hydrochloride (63 mg) in ethanol (5 ml) were added N,N-diisopropylethylamine (50 μl) and (S)-2-[(4-fluorophenoxy)methyl]oxirane (58 mg) at room temperature, and the mixture was refluxed overnight. After removal of the solvent in vacuo, the residue was dissolved in a mixture of water and ethyl acetate. The organic layer was washed with brine, dried over anhydrous magnesium sulfate and evaporated under reduced pressure. The residue was purified by thin layer silica gel chromatography (chloroform:methanol=5:1) to give (2S)-2-[[(2S)-3-(4-fluorophenoxy)-2-hydroxypropyl]amino]-3-[4-(phenylsulfonyl)-phenyl]-1-propanol (38 mg).

NMR (DMSO-d₆, δ): 2.55-2.8 (5H, m), 3.7-3.9 (3H, m), 6.8-6.95 (2H, m), 7.05-7.2 (2H, m), 7.44 (2H, d, J=8.3 Hz), 7.5-7.7 (3H, m), 7.81 (2H, d, J=8.3 Hz), 7.9-8.0 (2H, m) (+)APCI-MS (m/z): 460 (M+H)⁺

### EXAMPLE 2

The following compounds were obtained according to a similar manner to that of Example 1.

(1) (2S)-2-[[(2S)-3-(9H-Carbazol-4-yloxy)-2-hydroxypropyl]-amino]-3-[4-(phenylsulfonyl)phenyl]-1-propanol

NMR (DMSO-d₆, δ): 2.6-3.0 (5H, m), 3.1-3.4 (2H, m), 3.9-4.2 (3H, m), 6.63 (1H, d, J=7.8 Hz), 7.05-7.15 (2H, m), 7.25-7.8 (10H, m), 7.85-7.95 (2H, m), 8.21 (1H, d, J=7.8 Hz) (+)APCI-MS (m/z): 531 (M+H)⁺

(2) (2S)-2-[[(2S)-3-(4-Fluorophenoxy)-2-hydroxypropyl]-amino]-3-[4-[(4-methoxyphenyl)sulfonyl]phenyl]-1-propanol

NMR (CD₃OD, δ): 2.65-3.0 (5H, m), 3.3-3.65 (2H, m), 3.75-4.1 (6H, m), 6.8-7.15 (6H, m), 7.43 (2H, d, J=8.0 Hz), 7.75-7.9 (4H, m) (+)ESI-MS (m/z): 490 (M+H)⁺

(3) (2S)-2-[[(2R)-2-(3-Chlorophenyl)-2-hydroxyethyl]amino]-3-[4-[(4-methoxyphenyl)sulfonyl]phenyl]-1-propanol

NMR (CD₃OD, δ): 2.7-3.0 (5H, m), 3.3-3.65 (2H, m), 3.84 (3H, s), 4.65-4.75 (1H, m), 7.0-7.45 (8H, m), 7.75-7.9 (4H, m) (+)ESI-MS (m/z): 476, 478 (M+H)⁺

(4) (2S)-2-[[(2S)-2-Hydroxy-3-phenoxypropyl]amino]-3-[4-(4-quinolinylsulfonyl)phenyl]-1-propanol

NMR (DMSO-d₆, δ): 2.5-2.8 (5H, m), 3.1-3.4 (2H, m), 3.7-3.9 (3H, m), 6.8-7.0 (3H, m), 7.2-7.35 (2H, m), 7.46 (2H, d, J=8.4 Hz), 7.7-7.95 (4H, m), 8.15-8.25 (2H, m), 8.5-8.6 (1H, m), 9.22 (1H, d, J=4.4 Hz) (+)APCI-MS (m/z): 493 (M+H)⁺

### EXAMPLE 3

To a solution of (2S)-2-[N-benzyl-N-[(2R)-2-[4-(benzyloxy)-3-nitrophenyl]-2-hydroxyethyl]amino]-3-[4-(phenylsulfonyl)phenyl]-1-propanol (100 mg) in a mixture of ethanol (6 ml) and water (2 ml) were added powdered iron (26 mg) and ammonium chloride (4 mg) at room temperature, and the mixture was refluxed for 1 hour. Insoluble materials were filtered off. The filtrate was evaporated under reduced pressure. The residue was dissolved in a mixture of saturated aqueous sodium hydrogen carbonate and ethyl acetate. After separation, the organic layer was washed with brine, dried over anhydrous magnesium sulfate and evaporated in reduced pressure. Under nitrogen at 5° C., to a solution of the residue in dichloromethane (5 ml) were added pyridine (19 μl) and methanesulfonyl chloride (13 μl) and the mixture was stirred at the same temperature for 5 hours. The resulting mixture was poured into saturated aqueous sodium hydrogen carbonate and the aqueous mixture was extracted with ethyl acetate. The organic layer was washed with brine, dried over anhydrous magnesium sulfate and evaporated under reduced pressure. The residue was purified by column chromatography on silica gel (chloroform:ethyl acetate=100:1) to give N-[5-[(1R)-2-[N-benzyl-N-[(1S)-2-hydroxy-1-[4-(phenylsulfonyl)benzyl]ethyl]amino]-1-hydroxyethyl]-2-(benzyloxy)phenyl]methanesulfonamide (60 mg).

NMR (CDCl₃, δ): 2.5-3.1 (8H, m), 3.3-3.55 (2H, m), 3.65-3.9 (2H, m), 4.5-4.6 (1H, m), 5.10 (2H, s), 6.95-7.6 (18H, m), 7.80 (2H, d, J=8.3 Hz), 7.9-8.0 (2H, m) (+)ESI-MS (m/z): 701 (M+H)⁺

### EXAMPLE 4

Under nitrogen, a mixture of (S)-2-(benzylamino)-3-[4-(phenylsulfonyl)phenyl]-1-propanol (100 mg) and (S)-2-(phenoxymethyl)oxirane (39 mg) in ethanol (10 ml) was refluxed for 24 hours. The resulting mixture was evaporated under reduced pressure and the residue was purified by column chromatography on silica gel (chloroform:ethyl acetate=20:1 to 5:1) to give (2S)-2-[N-benzyl-N-((2S)-2-hydroxy-3-phenoxypropyl) amino]-3-[4-(phenylsulfonyl) phenyl]-1-propanol (100 mg).

NMR (CDCl₃, δ): 2.5-3.25 (6H, m), 3.4-3.7 (3H, m), 3.75-4.0 (3H, m), 6.8-7.0 (3H, m), 7.1-7.35 (9H, m), 7.45-7.65 (3H, m), 7.75-8.0 (4H, m) (+)APCI-MS (m/z): 532 (M+H)⁺

### EXAMPLE 5

The following compound was obtained according to a similar manner to that of Example 4.

(2S)-2-[N-Benzyl-N-[(2R)-2-[4-(benzyloxy)-3-nitrophenyl]-2-hydroxyethyl]amino]-3-[4-(phenylsulfonyl)-phenyl]-1-propanol

NMR (CDCl₃, δ): 2.6-2.75 (2H, m), 2.8-2.95 (2H, m), 3.1-3.25 (1H, m), 3.5-3.9 (4H, m), 4.4-4.5 (1H, m), 5.22 (2H, s), 7.0-7.6 (17H, m), 7.7-8.0 (5H, m) (+)APCI-MS (m/z): 653 (M+H)⁺

### EXAMPLE 6

Under nitrogen, a mixture of N-benzyl-N-[2-[4-[(4-methoxyphenyl)sulfonyl]phenyl]ethyl]amine (150 mg) and (S)-2-[(4-fluorophenoxy)methyl]oxirane (79 mg) in ethanol (5 ml) was refluxed for 47 hours. The resulting mixture was evaporated under reduced pressure and the residue was purified by column chromatography on silica gel (hexane:ethyl acetate=3:1 to 1:1) to give (S)-1-[N-benzyl-N-[2-[4-[(4-methoxyphenyl)sulfonyl]phenyl]ethyl]amino]-3-(4-fluorophenoxy)-2-propanol (230 mg).

NMR (CDCl₃, δ): 2.65-2.9 (6H, m), 3.5-3.85 (7H, m), 3.9-4.05 (1H, m), 6.75-6.85 (2H, m), 6.9-7.05 (3H, m), 7.1-7.3 (8H, m), 7.75-7.9 (4H, m) (+)APCI-MS (m/z): 550 (M+H)⁺

### EXAMPLE 7

The following compounds were obtained according to a similar manner to that of Example 6.

(1) (S)-1-[N-Benzyl-N-[2-[4-[(4-methoxyphenyl)sulfonyl]-phenyl]ethyl]amino]-3-(1H-indol-4-yloxy)-2-propanol

NMR (CDCl₃, δ): 2.7-2.9 (6H, m), 3.55-3.85 (2H, m), 3.82 (3H, s), 4.05-4.2 (3H, m), 6.45-6.5 (1H, m), 6.55-6.6 (1H, m), 6.85-7.3 (10H, m), 7.7-7.9 (4H, m) (+)APCI-MS (m/z): 571 (M+H)⁺

(2) (S)-1-[N-Benzyl-N-[2-[4-[(4-methoxyphenyl)sulfonyl]-phenyl]ethyl]amino]-3-(9H-carbazol-4-yloxy)-2-propanol

NMR (CDCl₃, δ): 2.7-3.0 (6H, m), 3.55-3.9 (2H, m), 3.81 (3H, s), 4.15-4.3 (3H, m), 6.63 (1H, d, J=7.9 Hz), 6.85-7.45 (11H, m), 7.7-7.9 (4H, m), 8.09 (1H, br s), 8.23 (1H, d, J=7.7 Hz) (+)APCI-MS (m/z): 621 (M+H)⁺

(3) 4-[[4-[2-[N-Benzyl-N-[(R)-2-(3-chlorophenyl)-2-hydroxyethyl]amino]ethyl]phenyl]sulfonyl]phenol

NMR (CDCl₃, δ): 2.5-2.95 (6H, m), 3.5-3.95 (2H, m), 4.55-4.65 (1H, m), 6.85-6.95 (2H, m), 7.1-7.4 (11H, m), 7.75-7.9 (4H, m) (+)ESI-MS (m/z): 522, 524 (M+H)⁺

(4) (R)-2-[N-Benzyl-N-[2-[4-[[4-(4-fluorophenoxy)phenyl]-sulfonyl]phenyl]ethyl]amino]-1-(3-chlorophenyl)ethanol

NMR (CDCl₃, δ): 2.5-2.9 (6H, m), 3.5-3.9 (2H, m), 4.55-4.7 (1H, m), 6.95-7.35 (17H, m), 7.75-7.9 (4H, m) (+)ESI-MS (m/z): 616, 618 (M+H)⁺

(5) (S)-1-[N-Benzyl-N-[2-[4-[(3-methoxyphenyl)sulfonyl]-phenyl]ethyl]amino]-3-phenoxy-2-propanol

NMR (CDCl₃, δ): 2.65-2.9 (6H, m), 3.55-3.85 (2H, m), 3.84 (3H, s), 3.85-4.1 (3H, m), 6.85-7.55 (16H, m), 7.75-7.85 (2H, m) (+)APCI-MS (m/z): 532 (M+H)⁺

(6) (S)-1-[N-Benzyl-N-[2-[4-[(3-methoxyphenyl)sulfonyl]-phenyl]ethyl]amino]-3-(4-fluorophenoxy)-2-propanol

NMR (CDCl₃, δ): 2.65-2.9 (6H, m), 3.5-4.05 (8H, m), 6.75-6.85 (2H, m), 6.9-7.3 (10H, m), 7.35-7.55 (3H, m), 7.75-7.85 (2H, m) (+)APCI-MS (m/z): 550 (M+H)⁺

(7) (R)-2-[N-Benzyl-N-[2-[4-[(3-methoxyphenyl)sulfonyl]-phenyl]ethyl]amino]-1-(3-chlorophenyl)ethanol

NMR (CDCl₃, δ): 2.5-2.95 (6H, m), 3.45-3.95 (2H, m), 3.87 (3H, s), 4.55-4.65 (1H, m), 7.05-7.55 (15H, m), 7.8-7.85 (2H, m) (+)APCI-MS (m/z): 536, 538 (M+H)⁺

(8) 3-[[4-[2-[N-Benzyl-N-[(R)-2-(3-chlorophenyl)-2-hydroxyethyl]amino]ethyl]phenyl]sulfonyl]phenol

NMR (CDCl₃, δ): 2.45-3.0 (6H, m), 3.5-4.0 (2H, m), 4.45-4.55 (1H, m), 6.9-7.45 (14H, m), 7.5-7.55 (1H, m), 7.8-7.9 (2H, m) (+)APCI-MS (m/z): 522, 524 (M+H)⁺

(9) (R)-2-[N-Benzyl-N-[2-[4-[(2-methoxyphenyl)sulfonyl]-phenyl]ethyl]amino]-1-(3-chlorophenyl)ethanol

NMR (CDCl₃, δ): 2.5-3.0 (6H, m), 3.5-3.95 (2H, m), 3.75 (3H, s), 4.55-4.65 (1H, m), 6.85-6.9 (1H, m), 7.05-7.35 (12H, m), 7.45-7.6 (1H, m), 7.8-7.9 (2H, m), 8.1-8.2 (1H, m) (+)APCI-MS (m/z): 536, 538 (M+H)⁺

(10) 2-[[4-[2-[N-Benzyl-N-[(R)-2-(3-chlorophenyl)-2-hydroxyethyl]amino]ethyl]phenyl]sulfonyl]phenol

NMR (DMSO-d₆, δ): 2.55-2.8 (6H, m), 3.55-3.8 (2H, m), 4.6-4.75 (1H, m), 6.85-7.55 (14H, m), 7.7-7.8 (2H, m), 7.85-7.9 (1H, m) (+)APCI-MS (m/z): 522, 524 (M+H)⁺

(11) Ethyl [2-[[4-[2-[N-benzyl-N-[(R)-2-(3-chlorophenyl)-2-hydroxyethyl]amino]ethyl]phenyl]sulfonyl]phenoxy]-acetate

NMR (CDCl₃, δ): 1.26 (3H, t, J=7.1 Hz), 2.5-2.95 (6H, m), 3.5-3.95 (2H, m), 4.19 (2H, q, J=7.1 Hz), 4.5-4.65 (3H, m), 6.75-6.85 (1H, m), 7.1-7.35 (12H, m), 7.45-7.55 (1H, m), 7.9-8.0 (2H, m), 8.15-8.25 (1H, m) (+)APCI-MS (m/z): 608, 610 (M+H)⁺

(12) (R)-2-[N-Benzyl-N-[2-[4-[(3,4-dimethoxyphenyl)-sulfonyl]phenyl]ethyl]amino]-1-(3-chlorophenyl)ethanol

NMR (CDCl₃, δ): 2.5-2.95 (6H, m), 3.5-3.95 (2H, m), 3.90 (3H, s), 3.91 (3H, s), 4.55-4.65 (1H, m), 6.92 (1H, d, J=8.5 Hz), 7.1-7.4 (12H, m), 7.5-7.6 (1H, m), 7.75-7.85 (2H, m) (+)ESI-MS (m/z): 566, 568 (M+H)⁺

(13) (R)-2-[N-Benzyl-N-[2-[4-[[2-(4-fluorophenoxy)phenyl]-sulfonyl]phenyl]ethyl]amino]-1-(3-chlorophenyl)ethanol

NMR (CDCl₃, δ): 2.5-2.95 (6H, m), 3.5-3.95 (2H, m), 4.55-4.7 (1H, m), 6.65-6.8 (3H, m), 6.85-7.0 (2H, m), 7.1-7.35 (12H, m), 7.4-7.55 (1H, m), 7.8-7.9 (2H, m), 8.2-8.3 (1H, m) (+)ESI-MS (m/z): 616, 618 (M+H)⁺

(14) (S)-1-[N-Benzyl-N-[2-[4-[(3,4-dimethoxyphenyl)-sulfonyl]phenyl]ethyl]amino]-3-phenoxy-2-propanol

NMR (CDCl₃, δ): 2.65-2.9 (6H, m), 3.55-3.85 (2H, m), 3.85-4.1 (3H, m), 3.90 (3H, s), 3.91 (3H, s), 6.85-7.0 (3H, m), 7.1-7.4 (11H, m), 7.5-7.6 (1H, m), 7.75-7.85 (2H, m) (+)ESI-MS (m/z): 562 (M+H)⁺

### EXAMPLE 8

A mixture of (2S)-2-[N-benzyl-N-((2S)-2-hydroxy-3-phenoxypropyl)amino]-3-[4-(phenylsulfonyl)phenyl]-1-propanol (96 mg) and 10% palladium on activated carbon (50% wet, 30 mg) in methanol (5 ml) was stirred at room temperature in the presence of hydrogen at an atmospheric pressure for 7.5 hours. After filtration, the filtrate was evaporated under reduced pressure followed by trituration with hexane and dryness in vacuo to give (2S)-2-[((2S)-2-hydroxy-3-phenoxypropyl)amino]-3-[4-(phenylsulfonyl)phenyl]-1-propanol (42 mg).

NMR (DMSO-d₆, δ): 2.5-2.9 (5H, m), 3.15-3.55 (2H, m), 3.6-3.95 (3H, m), 6.8-7.0 (3H, m), 7.2-7.35 (2H, m), 7.44 (2H, d, J=8.3 Hz), 7.55-7.7 (3H, m), 7.80 (2H, d, J=8.3 Hz), 7.85-8.0 (2H, m) (+)APCI-MS (m/z): 442 (M+H)⁺

### EXAMPLE 9

A mixture of N-[5-[(1R)-2-[N-benzyl-N-[(1S)-2-hydroxy-1-[4-(phenylsulfonyl)benzyl]ethyl]amino]-1-hydroxyethyl]-2-(benzyloxy)phenyl]methanesulfonamide (57 mg) and 10% palladium on activated carbon (50% wet, 50 mg) in methanol (3 ml) was stirred at room temperature in the presence of hydrogen at an atmospheric pressure for 3 hours. After filtration, the filtrate was evaporated under reduced pressure. The residue was purified by thin layer silica gel chromatography (chloroform:methanol=3:1) to give N-[2-hydroxy-5-[(1R)-1-hydroxy-2-[[(1S)-2-hydroxy-1-[4-(phenylsulfonyl)benzyl]ethyl]amino]ethyl]phenyl]-methanesulfonamide (17 mg).

NMR (DMSO-d₆, δ): 2.4-2.8 (5H, m), 2.91 (3H, s), 3.05-3.6 (2H, m), 4.35-4.45 (1H, m), 6.80 (1H, d, J=8.2 Hz), 6.9-7.0 (1H, m), 7.16 (1H, m), 7.41 (2H, d, J=8.2 Hz), 7.55-7.75 (3H, m), 7.82 (2H, d, J=8.1 Hz), 7.9-8.0 (2H, m) (+)APCI-MS (m/z): 521 (M+H)⁺

### EXAMPLE 10

A mixture of (S)-1-[N-benzyl-N-[2-[4-[(4-methoxyphenyl)sulfonyl]phenyl]ethyl]amino]-3-(4-fluorophenoxy)-2-propanol (220 mg), 10% palladium on activated carbon (50% wet, 110 mg) and hydrogen chloride-methanol reagent 10 (Tokyo Kasei, 0.24 ml) in methanol (5 ml) was stirred at room temperature in the presence of hydrogen at an atmospheric pressure for 2 hours. After filtration, the filtrate was evaporated and dryness in vacuo gave (S)-1-(4-fluorophenoxy)-3-[[2-[4-[(4-methoxyphenyl)-sulfonyl]phenyl]ethyl]amino]-2-propanol hydrochloride (160 mg).

NMR (DMSO-d₆, δ): 2.85-3.25 (6H, m), 3.82 (3H, s), 3.85-3.95 (2H, m), 4.0-4.2 (1H, m), 6.85-7.0 (2H, m), 7.05-7.2 (4H, m), 7.49 (2H, d, J=8.4 Hz), 7.85-7.95 (4H, m) (+)APCI-MS (m/z): 460 (M-HCl+H)⁺

### EXAMPLE 11

The following compounds were obtained according to a similar manner to that of Example 10.

(1) (2S)-1-(9H-Carbazol-4-yloxy)-3-[[2-[4-[(4-methoxyphenyl)sulfonyl]phenyl]ethyl]amino]-2-propanol hydrochloride

NMR (DMSO-d₆, δ): 2.95-3.4 (6H, m), 3.82 (3H, s), 4.15-4.45 (3H, m), 6.69 (1H, d, J=7.8 Hz), 7.05-7.15 (4H, m), 7.25-7.55 (5H, m), 7.85-7.9 (4H, m), 8.21 (1H, d, J=7.7 Hz), 11.29 (1H, s) (+)APCI-MS (m/z): 531 (M-HCl+H)⁺

(2) (S)-1-[[2-[4-[(3-Methoxyphenyl)sulfonyl]phenyl]ethyl]-amino]-3-phenoxy-2-propanol hydrochloride

NMR (DMSO-d₆, δ): 2.95-3.3 (6H, m), 3.83 (3H, s), 3.9-4.0 (2H, m), 4.1-4.25 (1H, m), 6.85-7.0 (3H, m), 7.2-7.35 (3H, m), 7.4-7.6 (5H, m), 7.9-8.0 (2H, m) (+)APCI-MS (m/z): 442 (M-HCl+H)⁺

(3) (S)-1-(4-Fluorophenoxy)-3-[[2-[4-[(3-methoxyphenyl)-sulfonyl]phenyl]ethyl]amino]-2-propanol hydrochloride

NMR (DMSO-d₆, δ): 3.95-3.5 (6H, m), 3.83 (3H, s), 3.9-4.0 (2H, m), 4.1-4.25 (1H, m), 6.9-7.0 (2H, m), 7.05-7.3 (3H, m), 7.4-7.6 (5H, m), 7.9-8.0 (2H, m) (+)APCI-MS (m/z): 460 (M-HCl+H)⁺

### EXAMPLE 12

Under nitrogen, to a solution of (S)-2-amino-3-[4-(4-methoxybenzenesulfonyl)phenyl]propan-1-ol hydrochloride (70 mg) in ethanol (5 ml) was added sodium methoxide (28% in methanol, 41 μl) at 5° C. After being stirred at room temperature for 20 minutes, to this one was added (S)-2-(phenoxymethyl)oxirane (32.3 mg), and the solution was refluxed overnight. The mixture was diluted with chloroform and insoluble materials were removed by filtration. The filtrate was evaporated under reduced pressure and the residue was purified by column chromatography on silica gel (chloroform/methanol=20:1 to 10:1) to give (2S)-2-((2S)-2-hydroxy-3-phenoxypropylamino)-3-[4-(4-methoxybenzenesulfonyl)phenyl]propan-1-ol (36 mg).

### EXAMPLE 13

The following compounds were obtained according to a similar manner to that of Example 12.

(1) (2S)-2-[((2S)-2-Hydroxy-3-phenoxypropyl)amino]-3-[4-(2-thienylsulfonyl)phenyl]-1-propanol

NMR (DMSO-d₆, δ): 2.5-2.8 (5H, m), 3.1-3.4 (2H, m), 3.7-3.9 (3H, m), 6.85-7.0 (3H, m), 7.15-7.35 (3H, m), 7.46 (2H, d, J=8.4 Hz), 7.75-7.9 (3H, m), 8.05 (1H, dd, J=1.3, 4.9 Hz) (+)ESI-MS (m/z): 448 (M+H)⁺

(2) (2S)-3-[4-[(4-Fluorophenyl)sulfonyl]phenyl]-2-[((2S)-2-hydroxy-3-phenoxypropyl)amino]-1-propanol

NMR (DMSO-d₆, δ): 2.55-2.8 (5H, m), 3.1-3.4 (2H, m), 3.7-3.95 (3H, m), 6.85-7.0 (3H, m), 7.25-7.55 (6H, m), 7.81 (2H, d, J=8.3 Hz), 7.95-8.1 (2H, m) (+)APCI-MS (m/z): 460 (M+H)⁺

### EXAMPLE 14

Under nitrogen at room temperature, to a solution of [4-[(4-methoxyphenyl)sulfonyl]phenyl]acetaldehyde (190 mg) in 1,2-dichloroethane (5 ml) were added (S)-1-amino-3-phenoxy-2-propanol hydrochloride (150 mg), sodium triacetoxyborohydride (350 mg) and acetic acid (0.11 ml), and the mixture was stirred at the same temperature for 9 hours. The resulting mixture was poured into a mixture of 1N sodium hydroxide and ethyl acetate, and the mixture was stirred for 30 minutes. After separation, the organic layer was washed with brine, dried over anhydrous magnesium sulfate and evaporated under reduced pressure. The residue was purified by column chromatography on silica gel (chloroform:methanol=30:1 to 15:1) followed by treatment with 4N hydrogen chloride in 1,4-dioxane to give (S)-1-[[2-[4-[(4-methoxyphenyl)sulfonyl]phenyl]ethyl]amino]-3-phenoxy-2-propanol hydrochloride (57 mg).

NMR (DMSO-d₆, δ): 2.95-3.4 (6H, m), 3.82 (3H, s), 3.9-4.0 (2H, m), 4.1-4.2 (1H, m), 6.9-7.0 (3H, m), 7.1-7.2 (2H, m), 7.25-7.4 (2H, m), 7.45-7.6 (2H, m), 7.8-7.95 (4H, m) (+)APCI-MS (m/z): 442 (M-HCl+H)⁺

### EXAMPLE 15

The following compound was obtained according to a similar manner to that of Example 14.

(R)-1-(3-Chlorophenyl)-2-[[2-[4-[(4-methoxyphenyl)-sulfonyl]phenyl]ethyl]amino]ethanol hydrochloride

NMR (DMSO-d₆, δ): 2.9-3.4 (6H, m), 3.82 (3H, s), 4.9-5.05 (1H, m), 7.1-7.2 (2H, m), 7.3-7.55 (6H, m), 7.85-7.95 (4H, m) (+)APCI-MS (m/z): 446, 448 (M-HCl+H)⁺

### EXAMPLE 16

A mixture of (S)-1-[N-benzyl-N-[2-[4-[(4-methoxyphenyl)sulfonyl]phenyl]ethyl]amino]-3-(1H-indol-4-yloxy)-2-propanol (140 mg) and 10% palladium on activated carbon (50% wet, 70 mg) in methanol (3 ml) and chlorobenzene (3 ml) was stirred at room temperature in the presence of hydrogen at an atmospheric pressure for 1 hour. After filtration, the filtrate was evaporated under reduced pressure and triturated with hexane followed by dryness to give (S)-1-(1H-indol-4-yloxy)-3-[[2-[4-[(4-methoxyphenyl)-sulfonyl]phenyl]ethyl]amino]-2-propanol hydrochloride (120 mg).

NMR (DMSO-d₆, δ): 2.9-3.7 (6H m), 3.81 (3H, s), 3.95-4.45 (3H, m), 6.45-7.55 (9H, m), 7.8-7.95 (4H, m) (+)ESI-MS (m/z): 481 (M-HCl+H)⁺

### EXAMPLE 17

A mixture of 4-[[4-[2-[N-benzyl-N-[(R)-2-(3-chlorophenyl)-2-hydroxyethyl]amino]ethyl]phenyl]sulfonyl]-phenol (86 mg) and 10% palladium on activated carbon (50% wet, 43 mg) in a mixture of methanol (2 ml) and chlorobenzene (2 ml) was stirred at room temperature in the presence of hydrogen at an atmospheric pressure for 2 hours. After filtration, the filtrate was evaporated under reduced pressure. The residue was dissolved in a mixture of saturated aqueous sodium hydrogen carbonate and ethyl acetate. After separation, the organic layer was dried over magnesium sulfate and evaporated under reduced pressure. The residue was purified by column chromatography on silica gel (chloroform:methanol=20:1 to 8:1) followed by treatment with 4N hydrogen chloride in 1,4-dioxane and dryness to give 4-[[4-[2-[[(2R)-2-(3-chlorophenyl)-2-hydroxyethyl]amino]ethyl]phenyl]sulfonyl]phenol hydrochloride (35 mg).

NMR (DMSO-d₆, δ): 2.9-3.6 (6H, m), 4.85-5.0 (1H, m), 6.85-7.0 (2H, m), 7.2-7.55 (6H, m), 7.7-7.9 (4H, m) (+)ESI-MS (m/z): 432, 434 (M-HCl+H)⁺

### EXAMPLE 18

The following compounds were obtained according to a similar manner to that of Example 17.

(1) (R)-1-(3-Chlorophenyl)-2-[[2-[4-[(4-ethoxyphenyl)-sulfonyl]phenyl]ethyl]amino]ethanol hydrochloride

NMR (DMSO-d₆, δ): 1.32 (3H, t, J=6.9 Hz), 2.95-3.4 (6H, m), 4.10 (2H, q, J=6.9 Hz), 7.05-7.15 (2H, m), 7.3-7.55 (6H, m), 7.8-7.95 (4H, m) (+)APCI-MS (m/z): 460, 462 (M-HCl+H)⁺

(2) Methyl [4-[[4-[2-[[(R)-2-(3-chlorophenyl)-2-hydroxyethyl]amino]ethyl]phenyl]sulfonyl]phenoxy]-acetate

NMR (DMSO-d₆, δ): 2.6-2.8 (6H, m), 3.69 (3H, s), 4.5-4.65 (1H, m), 4.92 (2H, s), 7.05-7.15 (2H, m), 7.2-7.45 (6H, m), 7.75-7.9 (4H, m) (+)APCI-MS (m/z): 504, 506 (M+H)⁺

(3) Methyl [3-[[4-[2-[[(R)-2-(3-chlorophenyl)-2-hydroxyethyl]amino]ethyl]phenyl]sulfonyl]phenoxy]-acetate

NMR (DMSO-d₆, δ): 2.55-2.8 (6H, m), 3.69 (3H, s), 4.5-4.65 (1H, m), 4.93 (2H, s), 7.2-7.6 (10H, m), 7.8-7.9 (2H, m) (+)APCI-MS (m/z): 504, 506 (M+H)⁺

(4) (R)-1-(3-Chlorophenyl)-2-[[2-[4-[[4-(4-fluorophenoxy)-phenyl]sulfonyl]phenyl]ethyl]amino]ethanol hydrochloride

NMR (DMSO-d₆, δ): 2.9-3.45 (6H, m), 4.85-5.0 (1H, m), 7.05-7.6 (12H, m), 7.85-8.0 (4H, m) (+)APCI-MS (m/z): 526, 528 (M-HCl+H)⁺

(5) (R)-1-(3-Chlorophenyl)-2-[[2-[4-[(3-methoxyphenyl)-sulfonyl]phenyl]ethyl]amino]ethanol hydrochloride

NMR (DMSO-d₆, δ): 2.95-3.3 (6H, m), 3.83 (3H, s), 4.85-5.0 (1H, m), 7.2-7.6 (10H, m), 7.9-8.0 (2H, m) (+)APCI-MS (m/z): 446, 448 (M-HCl+H)⁺

(6) 3-[[4-[2-[[(R)-2-(3-Chlorophenyl)-2-hydroxyethyl]-amino]ethyl]phenyl]sulfonyl]phenol hydrochloride

NMR (DMSO-d₆, δ): 2.9-3.5 (6H, m), 4.85-5.0 (1H, m), 7.0-7.1 (1H, m), 7.2-7.6 (9H, m), 7.85-7.95 (2H, m) (+)APCI-MS (m/z): 432, 434 (M-HCl+H)⁺

(7) (R)-1-(3-Chlorophenyl)-2-[[2-[4-[(3-ethoxyphenyl)-sulfonyl]phenyl]ethyl]amino]ethanol hydrochloride

NMR (DMSO-d₆, δ): 1.33 (3H, t, J=6.9 Hz), 2.9-3.4 (6H, m), 4.10 (2H, q, J=6.9 Hz), 4.9-5.0 (1H, m), 7.15-7.6 (10H, m), 7.9-8.0 (2H, m) (+)ESI-MS (m/z): 460, 462 (M-HCl+H)⁺

(8) (R)-2-[N-Benzyl-N-[2-[4-[[3-(4-fluorophenoxy)phenyl]-sulfonyl]phenyl]ethyl]amino]-1-(3-chlorophenyl)ethanol

NMR (CDCl₃, δ): 2.55-2.95 (6H, m), 3.5-3.95 (2H, m), 4.55-4.65 (1H, m), 6.9-7.65 (19H, m), 7.75-7.85 (2H, m) (+)ESI-MS (m/z): 616, 618 (M+H)⁺

(9) (R)-1-(3-Chlorophenyl)-2-[[2-[4-[[3-(4-fluorophenoxy)-phenyl]sulfonyl]phenyl]ethyl]amino]ethanol hydrochloride

NMR (DMSO-d₆, δ): 2.95-3.5 (6H, m), 4.85-5.05 (1H, m), 7.1-7.75 (14H, m), 7.85-8.0 (2H, m) (+)ESI-MS (m/z): 526, 528 (M-HCl+H)⁺

(10) (R)-1-(3-Chlorophenyl)-2-[[2-[4-[(2-methoxyphenyl)-sulfonyl]phenyl]ethyl]amino]ethanol hydrochloride

NMR (DMSO-d₆, δ): 3.0-3.45 (6H, m), 3.74 (3H, s), 4.9-5.0 (1H, m), 7.1-7.25 (2H, m), 7.3-7.55 (6H, m), 7.6-7.75 (1H, m), 7.8-7.9 (2H, m), 7.95-8.05 (1H, m) (+)APCI-MS (m/z): 446, 448 (M-HCl+H)⁺

(11) 2-[[4-[2-[[(R)-2-(3-Chlorophenyl)-2-hydroxyethyl]-amino]ethyl]phenyl]sulfonyl]phenol hydrochloride

NMR (DMSO-d₆, δ): 2.8-3.55 (6H, m), 4.85-5.05 (1H, m), 6.85-7.1 (2H, m), 7.2-7.6 (7H, m), 7.8-8.0 (3H, m) (+)ESI-MS (m/z): 432, 434 (M-HCl+H)⁺

(12) Ethyl [2-[[4-[2-[[(R)-2-(3-chlorophenyl)-2-hydroxyethyl]amino]ethyl]phenyl]sulfonyl]phenoxy]-acetate

NMR (CDCl₃, δ): 1.16 (3H, t, J=7.1 Hz), 2.55-2.9 (6H, m), 4.12 (2H, q, J=7.1 Hz), 4.55-4.65 (1H, m), 4.81 (2H, s), 7.05-7.45 (8H, m), 7.55-7.7 (1H, m), 7.85-7.95 (2H, m), 7.95-8.05 (1H, m) (+)APCI-MS (m/z): 518, 520 (M+H)⁺

(13) (R)-1-(3-Chlorophenyl)-2-[[2-[4-[[2-(4-fluorophenoxy)-phenyl]sulfonyl]phenyl]ethyl]amino]ethanol hydrochloride

NMR (DMSO-d₆, δ): 2.95-3.7 (6H, m), 4.85-5.05 (1H, m), 6.3-6.4 (1H, m), 6.65-6.8 (2H, m), 6.8-6.95 (1H, m), 7.1-7.25 (2H, m), 7.25-7.55 (6H, m), 7.6-7.75 (1H, m), 7.8-7.9 (2H, m), 8.2-8.3 (1H, m) (+)APCI-MS (m/z): 526, 528 (M-HCl+H)⁺

(14) (R)-1-(3-Chlorophenyl)-2-[[2-[4-[(3,4-dimethoxyphenyl)-sulfonyl]phenyl]ethyl]amino]ethanol hydrochloride

NMR (DMSO-d₆, δ): 2.95-3.45 (6H, m), 3.82 (3H, s), 3.83 (3H, s), 4.85-5.0 (1H, m), 7.15 (1H, d, J=8.6 Hz), 7.3-7.6 (8H, m), 7.85-8.0 (2H, m) (+)ESI-MS (m/z): 476, 478 (M-HCl+H)⁺

### EXAMPLE 19

Under nitrogen at 5° C., to a suspension of sodium hydride (60% in oil, 8.4 mg) in N,N-dimethylformamide (3 ml) was added 4-[[4-(2-[N-benzyl-N-[(R)-2-(3-chlorophenyl)-2-hydroxyethyl]amino]ethyl]phenyl]sulfonyl]phenol (100 mg), and the mixture was stirred at the same temperature for 30 minutes. To this one was added ethyl iodide (17 μl), and the mixture was stirred at room temperature for 1.5 days. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=5:1 to 3:1) to give (R)-2-[N-benzyl-N-[2-[4-[(4-ethoxyphenyl)sulfonyl]phenyl]-ethyl]amino]-1-(3-chlorophenyl)ethanol (81 mg).

NMR (CDCl₃, δ): 1.41 (3H, t, J=7.0 Hz), 2.5-2.95 (6H, m), 3.5-3.95 (2H, m), 4.05 (2H, q, J=7.0 Hz), 4.55-4.7 (1H, m), 6.9-7.0 (2H, m), 7.1-7.4 (11H, m), 7.75-7.9 (4H, m) (+)APCI-MS (m/z): 550, 552 (M+H)⁺

### EXAMPLE 20

The following compound was obtained according to a similar manner to that of Example 19.

(R)-2-[N-Benzyl-N-[2-[4-[(3-ethoxyphenyl)sulfonyl]-phenyl]ethyl]amino]-1-(3-chlorophenyl)ethanol

NMR (CDCl₃, δ): 1.41 (3H, t, J=7.0 Hz), 2.55-2.95 (6H, m), 3.5-3.95 (2H, m), 4.06 (2H, q, J=7.0 Hz), 4.55-4.7 (1H, m), 7.0-7.55 (15H, m), 7.75-7.9 (2H, m) (+)APCI-MS (m/z): 550, 552 (M+H)⁺

### EXAMPLE 21

Under nitrogen at 5° C., to a suspension of sodium hydride (60% in oil, 17 mg) in N,N-dimethylformamide (3 ml) was added 4-[[4-[2-[N-benzyl-N-[(R)-2-(3-chlorophenyl)-2-hydroxyethyl]amino]ethyl]phenyl]sulfonyl]phenol (200 mg), and the mixture was stirred at the same temperature for 30 minutes. To this one was added ethyl bromoacetate (47 μl), and the mixture was stirred at 5° C. for 5 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=5:1 to 2:1) to give ethyl [4-[[4-[2-[N-benzyl-N-[(R)-2-(3-chlorophenyl)-2-hydroxyethyl]amino]ethyl]phenyl]sulfonyl]phenoxy]acetate (200 mg).

NMR (CDCl₃, δ): 1.28 (3H, t, J=7.2 Hz), 2.55-2.9 (6H, m), 3.5-3.95 (2H, m), 4.26 (2H, q, J=7.2 Hz), 4.55-4.65 (1H, m), 4.63 (2H, s), 6.9-7.0 (2H, m), 7.1-7.35 (11H, m), 7.75-7.9 (4H, m) (+)APCI-MS (m/z): 608, 610 (M+H)⁺

### EXAMPLE 22

The following compound was obtained according to a similar manner to that of Example 21.

Ethyl [3-[[4-[2-[N-benzyl-N-[(R)-2-(3-chlorophenyl)-2-hydroxyethyl]amino]ethyl]phenyl]sulfonyl]phenoxy]acetate

NMR (CDCl_{3, δ):} 1.28 (3H, t, J=7.1 Hz), 2.5-2.95 (6H, m), 3.5-3.95 (2H, m), 4.26 (2H, q, J=7.1 Hz), 4.55-4.65 (1H, m), 4.64 (2H, s), 7.05-7.6 (15H, m), 7.75-7.85 (2H, m) (+)APCI-MS (m/z): 608, 610 (M+H)⁺

### EXAMPLE 23

At room temperature, to a solution of methyl [4-[[4-[2-[[(R)-2-(3-chlorophenyl)-2-hydroxyethyl]amino]ethyl]phenyl]-sulfonyl]phenoxy]acetate (32 mg) in ethanol (2 ml) was added 1N sodium hydroxide (62 μl), and the mixture was stirred at the same temperature for 4.5 hours. The resulting mixture was evaporated under reduced pressure and dried to give sodium [4-[[4-[2-[[(R)-2-(3-chlorophenyl)-2-hydroxyethyl]-amino]ethyl]phenyl]sulfonyl]phenoxy]acetate (34 mg).

NMR (DMSO-d₆, δ): 2.55-2.85 (6H, m), 4.15 (2H, s), 4.55-4.65 (1H, m), 6.85-6.95 (2H, m), 7.2-7.45 (6H, m), 7.7-7.9 (4H, m) (+)ESI-MS (m/z): 512, 514 (M+H)⁺

### EXAMPLE 24

The following compounds were obtained according to a similar manner to that of Example 23.

(1) Sodium [3-[[4-[2-[[(R)-2-(3-chlorophenyl)-2-hydroxyethyl]amino]ethyl]phenyl]sulfonyl]phenoxy]-acetate

NMR (DMSO-d₆, δ): 2.55-2.85 (6H, m), 4.14 (2H, s), 4.55-4.7 (1H, m), 7.0-7.1 (1H, m), 7.2-7.5 (9H, m), 7.75-7.9 (2H, m) (+)ESI-MS (m/z): 512, 514 (M+H)⁺

(2) Sodium [2-[[4-[2-[[(R)-2-(3-chlorophenyl)-2-hydroxyethyl]amino]ethyl]phenyl]sulfonyl]phenoxy]-acetate

NMR (DMSO-d₆, δ): 2.45-2.9 (6H, m), 4.03 (2H, s), 4.5-4.65 (1H, m), 6.85-7.6 (9H, m), 7.85-8.0 (3H, m) (+)ESI-MS (m/z): 512, 514 (M+H)⁺

### EXAMPLE 25

At room temperature, to a solution of methyl [4-[[4-[2-[[(R)-2-(3-chlorophenyl)-2-hydroxyethyl]amino]ethyl]phenyl]-sulfonyl]phenoxy]acetate (28 mg) in ethanol (2 ml) was added 3.95N hydrogen chloride in ethanol (1 ml), and the mixture was allowed to stand at the same temperature for 2 hours. The resulting mixture was evaporated under reduced pressure and dried to give ethyl [4-[[4-[2-[[(2R)-2-(3-chlorophenyl)-2-hydroxyethyl]amino]ethyl]phenyl]sulfonyl]phenoxy]acetate hydrochloride (31 mg).

NMR (DMSO-d₆, δ): 1.20 (3H, t, J=7.1 Hz), 2.95-3.3 (6H, m), 4.16 (2H, q, J=7.1 Hz), 4.85-5.0 (1H, m), 4.91 (2H, s), 7.1-7.2 (2H, m), 7.3-7.55 (6H, m), 7.8-7.95 (4H, m) (+)ESI-MS (m/z): 518, 520 (M-HCl+H)⁺

### EXAMPLE 26

The following compounds were obtained according to a similar manner to that of Example 25.

(1) Ethyl [3-[[4-[2-[[(R)-2-(3-chlorophenyl)-2-hydroxyethyl]amino]ethyl]phenyl]sulfonyl]phenoxy]-acetate hydrochloride

NMR (DMSO-d₆, δ): 1.19 (3H, t, J=7.1 Hz), 2.9-3.6 (6H, m), 4.16 (2H, q, J=7.1 Hz), 4.85-5.0 (3H, m), 7.2-7.6 (10H, m), 7.9-8.0 (2H, m) (+)ESI-MS (m/z): 518, 520 (M-HCl+H)⁺

(2) Ethyl [2-[[4-[2-[[(R)-2-(3-chlorophenyl)-2-hydroxyethyl]amino]ethyl]phenyl]sulfonyl]phenoxy]-acetate hydrochloride

NMR (DMSO-d₆, δ): 1.17 (3H, t, J=7.1 Hz), 2.95-3.5 (6H, m), 4.13 (2H, q, J=7.1 Hz), 4.83 (2H, s), 4.85-5.0 (1H, m), 7.05-7.5 (8H, m), 7.55-7.7 (1H, m), 7.9-8.1 (3H, m) (+)APCI-MS (m/z): 518, 520 (M-HCl+H)⁺

### EXAMPLE 27

Under nitrogen at 5° C., to a solution of tert-butyl N-[(R)-2-(3-chlorophenyl)-2-hydroxyethyl]-N-[2-[4-[(4-cyanophenyl)thio]phenyl]ethyl]carbamate (240 mg) in dichloromethane (10 ml) was added m-chloroperbenzoic acid (320 mg), and the mixture was stirred at room temperature for 7 hours. The resulting mixture was poured into aqueous sodium thiosulfate, and the aqueous mixture was extracted with ethyl acetate. The organic layer was washed successively with saturated aqueous sodium hydrogen carbonate and brine, dried over anhydrous magnesium sulfate and evaporated under reduced pressure. The residue was purified by column chromatography on silica gel (hexane:ethyl acetate=3:1 to 1:1) to give tert-butyl N-[(R)-2-(3-chlorophenyl)-2-hydroxyethyl]-N-[2-[4-[(4-cyanophenyl)sulfonyl]phenyl]ethyl]carbamate (91 mg).

(+)ESI-MS (m/z): 563, 565 (M+Na)⁺

### EXAMPLE 28

The following compound was obtained according to a similar manner to that of Example 27.

tert-Butyl N-[(R)-2-(3-chlorophenyl)-2-hydroxyethyl]-N-[2-[4-[(3-cyanophenyl)sulfonyl]phenyl]ethyl]carbamate

NMR (CDCl₃, δ): 1.36 (9H, br s), 2.7-3.0 (2H, m), 3.2-3.5 (4H, m), 4.75-4.9 (1H, m), 7.15-7.4 (6H, m), 7.55-7.7 (1H, m), 7.75-7.9 (3H, m), 8.1-8.25 (2H, m) (+)ESI-MS (m/z): 563, 565 (M+Na)⁺

### EXAMPLE 29

Under nitrogen at room temperature, to a solution of tert-butyl N-[(R)-2-(3-chlorophenyl)-2-hydroxyethyl]-N-[2-[4-[(4-cyanophenyl)sulfonyl]phenyl]ethyl]carbamate (86 mg) in ethanol (5 ml) were added hydroxylamine hydrochloride (12 mg) and potassium carbonate (27 mg), and the mixture was refluxed for 7 hours. The resulting mixture was cooled to room temperature and diluted with dichloromethane. The mixture was filtrated through a bed of silica gel and the silica gel was washed with a mixture of dichloromethane and methanol (10:1). The filtrate was evaporated under reduced pressure and dried to give tert-butyl N-[2-[4-[[4-[amino(hydroxyimino)methyl]phenyl]sulfonyl]phenyl]ethyl]-N-[(R)-2-(3-chlorophenyl)-2-hydroxyethyl]carbamate (86 mg).

(+)ESI-MS (m/z): 596, 598 (M+Na)⁺

### EXAMPLE 30

The following compound was obtained according to a similar manner to that of Example 29.

tert-Butyl N-[2-[4-[[3-[amino(hydroxyimino)methyl]-phenyl]sulfonyl]phenyl]ethyl]-N-[(R)-2-(3-chlorophenyl)-2-hydroxyethyl]carbamate

NMR (CDCl₃, δ): 1.36 (9H, br s), 2.7-2.95 (2H, m), 3.1-3.5 (4H, m), 4.7-4.85 (1H, m), 7.1-8.15 (12H, m) (+)ESI-MS (m/z): 596, 598 (M+Na)⁺

### EXAMPLE 31

Under nitrogen at 5° C., to a solution of tert-butyl N-[2-[4-[[4-[amino(hydroxyimino)methyl]phenyl]sulfonyl]-phenyl]ethyl]-N-[(R)-2-(3-chlorophenyl)-2-hydroxyethyl]carbamate (83 mg) in pyridine (2 ml) was added dropwise acetyl chloride (11 μl), and the mixture was stirred at room temperature for 1.5 hours followed by being refluxed for 3 hours. The resulting mixture was evaporated under reduced pressure. The residue was dissolved in a mixture of water and ethyl acetate. After separation, the organic layer was washed with brine, dried over anhydrous magnesium sulfate and evaporated under reduced pressure. The residue was purified by column chromatography on silica gel (hexane:ethyl acetate=2:1 to 1:1) to give tert-butyl N-[(R)-2-(3-chlorophenyl)-2-hydroxyethyl]-N-[2-[4-[[4-(5-methyl-1,2,4-oxadiazol-3-yl)phenyl]sulfonyl]phenyl]ethyl]-carbamate (33 mg).

NMR (CDCl₃, δ): 1.34 (9H, br s), 2.67 (3H, s), 2.65-2.95 (2H, m), 3.1-3.45 (4H, m), 4.75-4.9 (1H, m), 7.15-7.35 (6H, m), 7.85-7.9 (2H, m), 7.95-8.05 (2H, m), 8.15-8.2 (2H, m) (+)ESI-MS (m/z): 620, 622 (M+Na)⁺

### EXAMPLE 32

The following compound was obtained according to a similar manner to that of Example 31.

tert-Butyl N-[(R)-2-(3-chlorophenyl)-2-hydroxyethyl]-N-[2-[4-[[3-(5-methyl-1,2,4-oxadiazol-3-yl)phenyl]sulfonyl]-phenyl]ethyl]carbamate

NMR (CDCl₃, δ): 1.32 (9H, br s), 2.65-2.95 (2H, m), 2.67 (3H, s), 3.15-4.45 (4H, m), 4.8-4.9 (1H, m), 7.1-7.4 (6H, m), 7.55-7.65 (1H, m), 7.85-7.95 (2H, m), 8.0-8.1 (1H, m), 8.2-8.3 (1H, m), 8.6-8.65 (1H, m) (+)ESI-MS (m/z): 620, 622 (M+Na)⁺

### EXAMPLE 33

At room temperature, to a solution of tert-butyl N-[(R)-2-(3-chlorophenyl)-2-hydroxyethyl]-N-[2-[4-[[4-(5-methyl-1,2,4-oxadiazol-3-yl)phenyl]sulfonyl]phenyl]ethyl]-carbamate (29 mg) in ethyl acetate (1 ml) was added 4N hydrogen chloride in 1,4-dioxane (1 ml), and the mixture was stirred at the same temperature for 3.5 hours to give a precipitate. The precipitate was collected by filtration and dried to give (R)-1-(3-chlorophenyl)-2-[[2-[4-[[4-(5-methyl-1,2,4-oxadiazol-3-yl)phenyl]sulfonyl]phenyl]ethyl]-amino]ethanol dihydrochloride (14 mg).

NMR (DMSO-d₆, δ): 2.68 (3H, s), 2.9-3.4 (6H, m), 4.85-5.0 (1H, m), 7.3-7.45 (4H, m), 7.54 (2H, d, J=8.4 Hz), 7.98 (2H, d, J=8.4 Hz), 8.1-8.25 (4H, m) (+)ESI-MS (m/z): 489, 500 (M-2HCl+H)⁺

### EXAMPLE 34

The following compound was synthesized according to a similar manner to that of Example 33.

(R)-1-(3-Chlorophenyl)-2-[[2-[4-[[3-(5-methyl-1,2,4-oxadiazol-3-yl)phenyl]sulfonyl]phenyl]ethyl]amino]ethanol dihydrochloride

NMR (DMSO-d₆, δ): 2.70 (3H, s), 2.9-3.4 (6H, m), 4.85-5.0 (1H, m), 7.3-7.6 (6H, m), 7.75-7.9 (1H, m), 7.95-8.05 (2H, m), 8.15-8.35 (2H, m), 8.4-8.45 (1H, m) (+)ESI-MS (m/z): 498, 500 (M-2HCl+H)⁺

### EXAMPLE 35

A mixture of (S)-1-[N-benzyl-N-[2-[4-[(3,4-dimethoxyphenyl)sulfonyl]phenyl]ethyl]amino]-3-phenoxy-2-propanol (250 mg) and 10% palladium on activated carbon (50% wet, 130 mg) in methanol (5 ml) was stirred room temperature in the presence of hydrogen at an atmospheric pressure for 2 hours. After filtration, the filtrate was evaporated under reduced pressure. The residue was purified by column chromatography on silica gel (chloroform:methanol=50:1 to 20:1) followed by treatment with hydrogen chloride-methanol reagent 10 (Tokyo Kasei) and dryness to give (S)-1-[[2-[4-[(3,4-dimethoxyphenyl)sulfonyl]phenyl]ethyl]amino]-3-phenoxy-2-propanol hydrochloride (180 mg).

NMR (DMSO-d₆, δ): 2.9-3.5 (6H, m), 3.82 (3H, s), 3.83 (3H, s), 3.9-4.0 (2H, m), 4.05-4.25 (1H, m), 6.85-7.0 (3H, m), 7.15 (1H, d, J=8.5 Hz), 7.25-7.6 (6H, m), 7.85-8.0 (2H, m) (+)ESI-MS (m/z): 472 (M-HCl+H)⁺

Preparation 51

The following compounds were obtained according to a similar manner to that of Preparation 2.

(1) 3-[[4-[2-[N-Benzyl-N-(tert-butoxycarbonyl)amino]ethyl]-phenyl]thio]phenyl trifluoromethanesulfonate

NMR (CDCl₃, δ): 1.47 (9H, s), 2.80 (2H, br s), 3.39 (2H, br s), 4.38 (2H, br s), 6.95-7.45 (13H, m) (+)APCI-MS (m/z): 590 (M+Na)⁺

(2) 4-[[4-[2-[N-Benzyl-N-(tert-butoxycarbonyl)amino]ethyl]-phenyl]thio]phenyl trifluoromethanesulfonate

NMR (CDCl₃, δ): 1.47 (9H, s), 2.78 (2H, m), 3.39 (2H, m), 4.38 (2H, m), 7.05-7.40 (13H, m) (+)APCI-MS (m/z): 590 (M+Na)⁺

(3) 4-[[4-[(2R)-2-[(2,2,2-Trifluoroacetyl)amino]propyl]-phenyl]sulfonyl]phenyl trifluoromethanesulfonate

NMR (CDCl₃, δ): 1.23 (3H, d, J=7 Hz), 2.87 (1H, dd, J=14, 7 Hz), 2.98 (1H, dd, J=14, 6 Hz), 4.28 (1H, m), 6.08 (1H, br d, J=7 Hz), 7.36 (2H, d, J=7 Hz), 7.42 (2H, d, J=7 Hz), 7.90 (2H, d, J=7 Hz), 8.03 (2H, d, J=7 Hz) (+)APCI-MS (m/z): 542 (M+Na)⁺

Preparation 52

A mixture of 3-[[4-[2-[N-benzyl-N-(tert-butoxycarbonyl)amino]ethyl]phenyl]thio]phenyl trifluoromethanesulfonate (521 mg), palladium(II) acetate (22 mg), 1,3-bis(diphenylphosphino)propane (46 mg), ethanol (2.1 ml), and triethylamine (0.4 ml) in N,N-dimethylformamide (4.2 ml) was heated to 60° C. under carbon monoxide (1 atm) atmosphere for 5.5 hours. After being allowed to cool to room temperature, the mixture was partitioned between hexane/ethyl acetate (1/3) and water. The organic layer was separated, washed successively with water, brine, dried over magnesium sulfate, and filtered. The filtrate was concentrated and the residue was purified by column chromatography (silica gel, hexane/ethyl acetate) to give ethyl 3-[[4-[2-[N-benzyl-N-(tert-butoxycarbonyl)-amino]ethyl]phenyl]thio]benzoate (389 mg) as a colorless oil.

NMR (CDCl₃, δ): 1.36 (3H, t, J=7 Hz), 1.46 (9H, s), 2.76 (2H, br s), 3.36 (2H, br s), 4.34 (2H, q, J=7 Hz), 4.36 (2H, br s), 6.95-7.50 (11H, m), 7.87 (1H, d, J=7 Hz), 7.98 (1H, s) (+)APCI-MS (m/z): 514 (M+Na)⁺

Preparation 53

The following compounds were obtained according to a similar manner to that of Preparation 19.

(1) Ethyl 3-[[4-[2-[N-benzyl-N-(tert-butoxycarbonyl)amino]-ethyl]phenyl]sulfonyl]benzoate

NMR (CDCl₃, δ): 1.38 (9H, s), 1.40 (3H, t, J=7 Hz), 2.81 (2H, br s), 3.35 (2H, br s), 4.38 (2H, br s), 4.40 (2H, q, J=7 Hz), 7.00-7.45 (7H, m), 7.58 (1H, t, J=8 Hz), 7.86 (2H, d, J=8 Hz), 8.10 (1H, d, J=8 Hz), 8.22 (1H, d, J=8 Hz), 8.57 (1H, s) (+)APCI-MS (m/z): 546 (M+Na)⁺

(2) Ethyl 4-[[4-[2-[N-benzyl-N-(tert-butoxycarbonyl)-amino]ethyl]phenyl]sulfonyl]benzoate

NMR (CDCl₃, δ): 1.38 (3H, t, J=7 Hz), 1.39 (9H, s), 2.80 (2H, br s), 3.35 (2H, br s), 4.36 (2H, br s), 4.39 (2H, q, J=7 Hz), 7.00-7.45 (7H, m), 7.84 (2H, d, J=8 Hz), 7.98 (2H, d, J=8 Hz), 8.14 (2H, d, J=8 Hz) (+)APCI-MS (m/z): 546 (M+Na)⁺

Preparation 54

The following compounds were obtained according to a similar manner to that of Preparation 32.

(1) Ethyl 3-[[4-[2-(benzylamino)ethyl]phenyl]sulfonyl]-benzoate

NMR (CDCl₃, δ): 1.40 (3H, t, J=7 Hz), 2.86 (4H, m), 3.78 (2H, s), 4.40 (2H, q, J=7 Hz), 7.10-7.43 (7H, m), 7.58 (1H, t, J=8 Hz), 7.87 (2H, d, J=8 Hz), 8.11 (1H, d, J=8 Hz), 8.22 (1H, d, J=8 Hz), 8.58 (1H, s) (+)APCI-MS (m/z): 424 (M+H)⁺

(2) Ethyl 4-[[4-[2-(benzylamino)ethyl)phenyl]sulfonyl]-benzoate

NMR (CDCl₃, δ): 1.38 (3H, t, J=7 Hz), 2.87 (4H, m), 3.74 (2H, s), 4.39 (2H, q, J=7 Hz), 7.10-7.45 (7H, m), 7.86 (2H, d, J=8 Hz), 7.99 (2H, d, J=8 Hz), 8.15 (2H, d, J=8 Hz) (+)APCI-MS (m/z): 424 (M+H)⁺

Preparation 55

The following compounds were obtained according to a similar manner to that of Preparation 52.

(1) Ethyl 4-[[4-[2-[N-benzyl-N-(tert-butoxycarbonyl)amino]-ethyl]phenyl]thio]benzoate

NMR (CDCl₃, δ): 1.36 (3H, t, J=7 Hz), 1.47 (9H, s), 2.80 (2H, br s), 3.39 (2H, br s), 4.34 (2H, q, J=7 Hz), 4.36 (2H, br s), 7.00-7.50 (12H, m), 7.88 (1H, d, J=7 Hz) (+)APCI-MS (m/z): 514 (M+Na)⁺

(2) Ethyl 4-[[4-[(2R)-2-[(trifluoroacetyl)amino]propyl]-phenyl]sulfonyl]benzoate

NMR (CDCl₃, δ): 1.21 (3H, d, J=7 Hz), 1.39 (3H, t, J=7 Hz), 2.84 (1H, dd, J=14, 7 Hz), 2.98 (1H, dd, J=14, 6 Hz), 4.26 (1H, m), 4.39 (2H, q, J=7 Hz) 6.08 (1H, br d, J=7 Hz), 7.33 (2H, d, J=8 Hz), 7.89 (2H, d, J=7 Hz), 7.99 (2H, d, J=7 Hz), 8.16 (2H, d, J=8 Hz) (+)APCI-MS (m/z): 466 (M+Na)⁺

Preparation 56

To a solution of ethyl 4-[[4-[(2R)-2-[(trifluoroacetyl)amino]propyl]phenyl]sulfonyl]benzoate (1.58 g) in ethanol (16 ml) was added 1N sodium hydroxide solution (8.6 ml), and the mixture was heated to 50° C. for 3 hours. After the solvent was evaporated, and the residue was dissolved in 4 M hydrogen chloride/ethanol (16 ml) and kept at room temperature for 7 days. The solvent was evaporated, and the residue was partitioned between ethyl acetate and sodium hydrogen carbonate solution. The organic layer was washed with brine, dried over magnesium sulfate. Filtration followed by evaporation gave ethyl 4-[[4-[(2R)-2-aminopropyl]phenyl]sulfonyl]benzoate (1.09 g) as an off-white powder.

NMR (DMSO-d₆, δ): 1.11 (3H, d, J=6 Hz), 1.32 (3H, t, J=7 Hz), 2.81 (1H, dd, J=13, 8 Hz), 3.07 (1H, dd, J=13, 6 Hz), 3.28-3.58 (1H, m), 4.34 (2H, q, J=7 Hz), 7.54 (2H, d, J=8 Hz), 7.80-8.40 (8H, m) (+)APCI-MS (m/z): 348 (M+H)⁺

Preparation 57

The following compounds were obtained according to a similar manner to that of Preparation 68.

(1) N-[2-[4-[(3-Chloro-4-methoxyphenyl)sulfonyl]-phenyl]ethyl]-2,2,2-trifluoroacetamide

(+)APCI-MS (m/z): 444 (M+Na)⁺

(2) 2,2,2-Trifluoro-N-[(1R)-2-[4-[(4-methoxyphenyl)-sulfonyl]phenyl]-1-methylethyl]acetamide

(+)APCI-MS (m/z): 424 (M+Na)⁺

Preparation 58

The following compounds were obtained according to a similar manner to that of Preparation 34.

(1) N-[2-[4-[(3-Chloro-4-hydroxyphenyl)sulfonyl]-phenyl]ethyl]-2,2,2-trifluoroacetamide

NMR (CDCl₃, δ): 2.95 (2H, t, J=7.1 Hz), 3.61 (2H, q-like, J=6.8 Hz), 6.16 (1H, br s), 6.39 (1H, br s), 7.11 (1H, d, J=8.6 Hz), 7.34 (2H, d, J=8.3 Hz), 7.75 (1H, dd, J=8.6, 2.3 Hz), 7.87 (2H, d, J=8.3 Hz), 7.93 (1H, d, J=2.3 Hz) (+)APCI-MS (m/z): 430 (M+Na)⁺

(2) 2,2,2-Trifluoro-N-[(1R)-2-(4-[(4-hydroxyphenyl)-sulfonyl]phenyl]-1-methylethyl]acetamide

(+)APCI-MS (m/z): 410 (M+Na)⁺

(3) 2,2,2-Trifluoro-N-[2-[4-[(4-hydroxyphenyl)sulfonyl]-phenyl]-1,1-dimethylethyl]acetamide

MS (m/z): 402 (M+H)

(4) N-[(1R)-2-[4-[(3-Chloro-4-hydroxyphenyl)sulfonyl]-phenyl]-1-methylethyl]-2,2,2-trifluoroacetamide

NMR (CDCl₃, δ): 1.20 (3H, d, J=3 Hz), 2.80-3.00 (2H, m), 4.20-4.40 (1H, m), 7.00-7.10 (2H, m), 7.20-7.35 (2H, m), 7.80-7.95 (4H, m)

(5) 2,2,2-Trifluoro-N-[2-[4-[(4-hydroxy-3-methylphenyl) sulfonyl]phenyl]ethyl]acetamide

MS (m/z): 388 (M+H)

(6) 2,2,2-Trifluoro-N-[2-[4-[(3-fluoro-4-hydroxyphenyl) sulfonyl]phenyl]ethyl]acetamide

MS (m/z): 389 (M−H)

(7) 2,2,2-Trifluoro-N-[(1R)-2-[4-[(3-hydroxyphenyl)-sulfonyl]phenyl]-1-methylethyl]acetamide

MS (m/z): 376 (M+H)

Preparation 59

To a solution of N-[2-[4-[(3-chloro-4-hydroxyphenyl)-sulfonyl]phenyl]ethyl]-2,2,2-trifluoroacetamide (687 mg) in N,N-dimethylformamide (7.0 ml) was added potassium carbonate (279 mg) at room temperature and the resulting suspension was stirred at the same temperature for 40 minutes. To the mixture was added chloroacetic acid tert-butyl ester (290 μl) and the mixture was stirred at room temperature for 23 hours. The mixture was quenched by the addition of water (20 ml) and extracted with ethyl acetate (20 ml×1, 5 ml×1). The combined extracts were washed with water (20 ml×2) and brine (20 ml×1) and dried over magnesium sulfate. Filtration followed by evaporation gave brown foam (716 mg). The crude product was chromatographed on silica gel (eluent: hexane/ethyl acetate) to give tert-butyl [2-chloro-4-[[4-[2-[(trifluoroacetyl)amino]ethyl]phenyl]sulfonyl]phenoxy]-acetate (502 mg) as white foam.

(+)APCI-MS (m/z): 544 (M+Na)⁺

Preparation 60

To a suspension of tert-butyl [2-chloro-4-[[4-[2-[(trifluoroacetyl)amino]ethyl]phenyl]sulfonyl]phenoxy]-acetate (496 mg) in methanol (10.0 ml) was added 1N sodium hydroxide solution (2.85 ml) at room temperature and the mixture was stirred at the same temperature for 5 hours. The mixture was quenched by the addition of 1N hydrochloric acid (1.9 ml) and the solvent was removed by evaporation. The residual solid was suspended in 4N hydrogen chloride in ethanol (10 ml) and the suspension was stirred at room temperature overnight. The solvent was removed by evaporation and the residual white solid was suspended in ethyl acetate (10 ml). To the suspension were added a saturated aqueous sodium hydrogen carbonate solution (5 ml) and water (5 ml) and the whole was stirred vigorously. The organic layer was separated and the aqueous layer was extracted with ethyl acetate (10 ml×2). The combined extracts were washed with brine (5 ml) and dried over magnesium sulfate. Filtration followed by evaporation gave ethyl [4-[[4-(2-aminoethyl)phenyl]sulfonyl]-2-chlorophenoxy]acetate (398 mg) as a pale yellow paste.

(+)APCI-MS (m/z): 398 (M+H)⁺

Preparation 61

To a solution of N-[2-[4-[(3-chloro-4-hydroxyphenyl)-sulfonyl]phenyl]ethyl]-2,2,2-trifluoroacetamide (874 mg) in methanol (8.0 ml) was added 1N sodium hydroxide solution (6.43 ml) and the solution was stirred at room temperature for 1 hour. To the solution was added 1N hydrochloric acid (4.29 ml) and the mixture was stirred at room temperature for 1 hour. The precipitates were collected by filtration, washed with a small portion of methanol, and dried under reduced pressure to give 4-[[4-(2-aminoethyl)phenyl]-sulfonyl]-2-chlorophenol (595 mg) as a white powder.

(+)APCI-MS (m/z): 312 (M+H)⁺

Preparation 62

To a solution of 2,2,2-trifluoro-N-[(1R)-1-methyl-2-phenylethyl]acetamide (10.0 g) in chloroform (100 ml) was added chlorosulfonic acid (50 ml) dropwise under 5° C. over 90 minutes. The solution was stirred at the same temperature for 1 hour and at room temperature overnight. The reaction mixture was carefully added dropwise to a stirred mixture of water (150 ml) and chloroform (50 ml) under ice-water cooling. The organic layer was separated and washed with water (200 ml×1) and dried over magnesium sulfate. Filtration followed by evaporation gave a white solid (14.2 g). The solid was chromatographed on silica gel (eluent: hexane/ethyl acetate) to give 4-[(2R)-2-[(trifluoroacetyl)-amino]propyl]benzenesulfonyl chloride (11.5 g) as a white solid.

NMR (CDCl₃, δ): 1.27 (3H, d, J=6.7 Hz), 2.92 (1H, dd, J=7.3, 13.6 Hz), 3.07 (1H, dd, J=6.1, 13.6 Hz), 4.32 (1H, h, J=7.0 Hz), 6.19 (1H, br), 7.44 (2H, d, J=8.5 Hz), 8.00 (2H, d, J=8.5 Hz)

Preparation 63

To a solution of 2,2,2-trifluoro-N-[(1R)-2-[4-[(4-methoxyphenyl)sulfonyl]phenyl]-1-methylethyl]acetamide (500 mg) in a mixed solvent of methanol (5.0 ml) and water (1.5 ml) was added potassium carbonate (344 mg) and the mixture was stirred at room temperature for 30 minutes. The mixture was warmed to 50° C. and stirred for 6 hours. After cooling to room temperature, the solvent was removed by evaporation. The residue was dissolved in ethyl acetate (20 ml) and washed with brine (5 ml×1). The aqueous washing was extracted with ethyl acetate (5 ml×2). The organic layers were combined and dried over magnesium sulfate. Filtration followed by evaporation gave (2R)-1-[4-[(4-methoxyphenyl)-sulfonyl]phenyl]-2-propanamine (342 mg) as a white solid.

(+)APCI-MS (m/z): 306 (M+H)⁺

Preparation 64

The following compound was obtained according to a similar manner to that of Preparation 59.

tert-Butyl [4-[[4-[(2R)-2-[(trifluoroacetyl)amino]-propyl]phenyl]sulfonyl]phenoxy]acetate

(+)APCI-MS (m/z): 524 (M+Na)⁺

Preparation 65

To a solution of tert-butyl [4-[[4-[(2R)-2-[(trifluoroacetyl)amino]propyl]phenyl]sulfonyl]phenoxy]-acetate (1.46 g) in a mixed solvent of methanol (15 ml) and water (5 ml) was added potassium carbonate (805 mg) and the solution was stirred at 50° C. for 2 hours. To the solution was added 1N sodium hydroxide (2.91 ml) and the mixture was stirred at the same temperature for 6 hours. After cooling to room temperature, the solvent was removed by evaporation. The residue was dissolved in 4N hydrogen chloride in ethanol (20 ml) and the mixture was stirred at room temperature overnight. The mixture was diluted with ethyl acetate (50 ml) and basified with a saturated aqueous sodium hydrogen carbonate solution (50 ml). The organic layer was separated and the aqueous layer was extracted with ethyl acetate (25 ml×1). The combined organic layers were washed with brine (75 ml×1) and dried over magnesium sulfate. Filtration followed by evaporation gave ethyl [4-[[4-[(2R)-2-aminopropyl]phenyl]sulfonyl]phenoxy]acetate (1.08 g) as a pale yellow crystalline solid.

(+)APCI-MS (m/z): 378 (M+H)⁺

Preparation 66

To a solution of 1,1-dimethyl-2-phenylethylamine (10 g) and triethylamine (12.1 ml) in tetrahydrofuran (5 ml) was added trifluoroacetic anhydride (10.4 ml) under ice-cooling and the mixture was stirred at the same temperature for 2 hours. The resulting mixture was poured into saturated aqueous sodium hydrogen carbonate solution, and extracted with ethyl acetate. The organic layer was washed with brine, dried over magnesium sulfate, and evaporated in vacuo, and the residue was triturated with diisopropyl ether to give N-(1,1-dimethyl-2-phenylethyl)-2,2,2-trifluoroacetamide (16.3 g) as a colorless powder.

MS (m/z): 268 (M+Na)

Preparation 67

To a solution of N-(1,1-dimethyl-2-phenylethyl)-2,2,2-trifluoroacetamide (15.46 g) in chloroform (100 ml) was added dropwise chlorosulfonic acid (68.3 ml) under ice-cooling and the mixture was stirred at the same temperature for 2 hours. To the resulting mixture was added dropwise water under ice-cooling, and extracted with chloroform. The organic layer was washed with brine, dried over magnesium sulfate, and evaporated in vacuo, and the residue was triturated with diisopropyl ether to give 4-[2-methyl-2-[(trifluoroacetyl)amino]propyl]benzenesulfonyl chloride (15 g) as a colorless powder.

NMR (CDCl₃, δ): 1.43 (6H, s), 3.26 (2H, s), 7.30-7.40 (2H, m), 7.90-8.05 (2H, m)

Preparation 68

To a solution of 4-[2-methyl-2-[(trifluoroacetyl)-amino]propyl]benzenesulfonyl chloride (8.16 g) and methoxybenzene (3.1 ml) in 1,2-dichloroethane (90 ml) was added trichloroaluminium (4.11 g) at room temperature and the mixture was stirred at 90° C. for 20 hours. The resulting mixture was poured into water and extracted with ethyl acetate. The organic layer was washed with brine, dried over magnesium sulfate, and evaporated in vacuo. The residue was purified by column chromatography on silica gel (hexane:ethyl acetate=1:1) to give 2,2,2-trifluoro-N-[2-[4-[(4-methoxyphenyl)sulfonyl]phenyl]-1,1-dimethylethyl]-acetamide (2.15 g) as a colorless powder.

MS (m/z): 438 (M+Na)

Preparation 69

To a solution of 2,2,2-trifluoro-N-[2-[4-[(4-methoxyphenyl)sulfonyl]phenyl]-1,1-dimethylethyl]acetamide (510 mg) in ethanol (5 ml) was added 1N sodium hydroxide solution (2.0 ml) at room temperature and the mixture was stirred at 80° C. for 4 hours. The resulting mixture was poured into water and extracted with chloroform. The organic layer was washed with brine, dried over magnesium sulfate, and evaporated in vacuo. The residue was purified by a column chromatography on silica gel (hexane:ethyl acetate=1:1) to give 1-[4-[(4-methoxyphenyl)sulfonyl]-phenyl]-2-methyl-2-propanamine (310 mg) as a colorless powder.

NMR (CDCl₃, δ): 1.10 (6H, s), 2.69 (2H, s), 3.86 (3H, s), 6.92-7.00 (2H, m), 7.20-7.35 (2H, m), 7.80-7.95 (4H, m)

Preparation 70

To a solution of 2,2,2-trifluoro-N-[2-[4-[(4-hydroxyphenyl)sulfonyl]phenyl]-1,1-dimethylethyl]acetamide (950 mg) and potassium carbonate (360 mg) in N,N-dimethylformamide (5 ml) was added ethyl bromoacetate (0.289 ml) at room temperature and the mixture was stirred at room temperature for 18 hours. The resulting mixture was poured into sautrated aqueous sodium hydrogen carbonate solution, and extracted with ethyl acetate. The organic layer was washed with brine, dried over magnesium sulfate, and evaporated in vacuo. The residue was purified by column chromatography on silica gel (hexane:ethyl acetate=1:1) to give ethyl [4-[[4-[2-methyl-2-[(trifluoroacetyl)amino]-propyl]phenyl]sulfonyl]phenoxy]acetate (870 mg) as a colorless powder.

MS (m/z): 486 (M−H)

Preparation 71

To a solution of ethyl [4-[[4-[2-methyl-2-[(trifluoroacetyl)amino]propyl]phenyl]sulfonyl]phenoxy]-acetate (950 mg) in ethanol (5 ml) was added 1N sodium hydroxide solution (2.0 ml) at room temperature and the mixture was stirred at 80° C. for 4 hours. The resulting mixture was evaporated in vacuo. To the residue was added 4N hydrogen chloride in ethanol (5.0 ml) at room temperature and the mixture was stirred at the same temperature for 18 hours. The reaction mixture was evaporated in vacuo. The residue was poured into saturated aqueous sodium hydrogen carbonate solution, and extracted with chloroform. The organic layer was washed with brine, dried over magnesium sulfate, and evaporated in vacuo to give ethyl [4-[[4-(2-amino-2-methylpropyl)phenyl]sulfonyl]phenoxy]acetate (710 mg) as a colorless oil.

MS (m/z): 392 (M+H)

Preparation 72

The following compounds were obtained according to a similar manner to that of Preparation 68.

(1) N-[(1R)-2-[4-[(3-Chloro-4-methoxyphenyl)sulfonyl]-phenyl]-1-methylethyl]-2,2,2-trifluoroacetamide

NMR (CDCl₃, δ): 1.20 (3H, d, J=3 Hz), 2.80-3.00 (2H, m), 3.95 (3H, s), 4.20-4.40 (1H, m), 6.92-7.00 (2H, m), 7.20-7.35 (2H, m), 7.80-7.95 (4H, m)

(2) 2,2,2-Trifluoro-N-[2-[4-[(4-methoxy-3-methylphenyl)sulfonyl]phenyl]ethyl]acetamide

MS (m/z): 438 (M+H)

(3) 2,2,2-Trifluoro-N-[2-[4-[(3-fluoro-4-methoxyphenyl)sulfonyl]phenyl]ethyl]acetamide

MS (m/z): 406 (M+H)

Preparation 73

The following compounds were obtained according to a similar manner to that of Preparation 70.

(1) Ethyl [2-methyl-4-[[4-[2-[(trifluoroacetyl)amino]-ethyl]phenyl]sulfonyl]phenoxy]acetate

MS (m/z): 474 (M+H)

(2) Ethyl [2-methyl-4-[[4-[2-[(trifluoroacetyl)amino]-ethyl]phenyl]sulfonyl]phenoxy]acetate

MS (m/z): 474 (M+H)

(3) Ethyl [2-chloro-4-[[4-[(2R)-2-[(trifluoroacetyl)amino]-propyl]phenyl]sulfonyl]phenoxy]acetate

MS (m/z): 508 (M+H)

(4) Ethyl [3-[[4-[(2R)-2-[(trifluoroacetyl)amino]propyl]-phenyl]sulfonyl]phenoxy]acetate

MS (m/z): 474 (M+H)

Preparation 74

The following compounds were obtained according to a similar manner to that of Preparation 71.

(1) Ethyl [4-[[4-(2-aminoethyl)phenyl]sulfonyl]-2-methylphenoxy]acetate

MS (m/z): 378 (M+H)

(2) Ethyl [4-[[4-[(2R)-2-aminopropyl]phenyl]sulfonyl]-2-chlorophenoxy]acetate

MS (m/z): 412 (M+H)

(3) Ethyl [4-[[4-(2-aminoethyl)phenyl]sulfonyl]-2-fluorophenoxy]acetate

MS (m/z): 382 (M+H)

(4) Ethyl [3-[[4-[(2R)-2-aminopropyl]phenyl]sulfonyl]-phenoxy]acetate

MS (m/z): 378 (M+H)

Preparation 75

The following compound was obtained according to a similar manner to that of Preparation 66.

2,2,2-Trifluoro-N-(2-phenylethyl)acetamide

NMR (CDCl₃, δ): 2.89 (2H, t, J=7 Hz), 3.64 (2H, q, J=7 Hz), 7.20-7.40 (5H, m)

Preparation 76

The following compound was obtained according to a similar manner to that of Preparation 67.

4-[2-[(Trifluoroacetyl)amino]ethyl]benzenesulfonyl chloride

NMR (DMSO-d₆, δ): 2.83 (2H, t, J=7 Hz), 3.40 (2H, q, J=7 Hz), 7.10-7.20 (2H, m), 7.40-7.60 (2H, m)

Preparation 77

To a solution of 2,2,2-trifluoro-N-[(1R)-1-methyl-2-phenylethyl]acetamide (485 mg) and 3-methoxybenzenesulfonyl chloride (390 mg) in 1,2-dichloroethane (7.0 ml) was added copper(II) trifluoromethanesulfonate (152 mg) and trichloroaluminium (475 mg) at room temperature and the mixture was refluxed for 7 hours. The resulting mixture was evaporated and partitioned between ethyl acetate and water. The organic layer was separated, washed with water and brine, dried over magnesium sulfate and evaporated. The residue was purified by column chromatography on silica gel (hexane/ethyl acetate=2/1) to give 2,2,2-trifluoro-N-((1R)-2-[4-[(3-methoxyphenyl)sulfonyl]phenyl]-1-methylethyl)acetamide (205 mg) as a colorless oil.

MS (m/z): 402 (M+H)

### EXAMPLE 36

A mixture of (R)-4-[[4-[2-[N-benzyl-N-[2-(3-chlorophenyl)-2-hydroxyethyl]amino]ethyl]phenyl]sulfonyl]-phenol (1.31 g), triethylamine (3.3 ml) and 10% pallasium on activated carbon (50% wet, 0.65 g) in a mixture of methanol (13 ml) and chlorobenzene (13 ml) was stirred at room temperature in the presence of hydrogen at an atmospheric pressure for 5 hours. After filtration, the filtrate was evaporated under reduced pressure. The residue was dissolved into a mixture of ethyl acetate and saturated aqueous sodium hydrogen carbonate. After separation, the organic layer was washed with brine, dried over anhydrous magnesium sulfate and evaporated under reduced pressure. The residue was purified by column chromatography on silica gel (chloroform:methanol=20:1 to 8:1) to give (R)-4-[[4-[2-[[2-(3-chlorophenyl)-2-hydroxyethyl]amino]ethyl]phenyl]-sulfonyl]phenol (789 mg).

NMR (DMSO-d₆, δ): 2.55-2.85 (6H, m), 4.55-4.6 (1H, m), 6.9-6.95 (2H, m), 7.2-7.8 (4H, m) (+)ESI-MS (m/z): 432, 434 (M+H)⁺

### EXAMPLE 37

Under nitrogen at room temperature, to a solution of (R)-4-[[4-[2-[[2-(3-chlorophenyl)-2-hydroxyethyl]amino]-ethyl]phenyl]sulfonyl]phenol (1.0 g) in tetrahydrofuran (8 ml) was added di-tert-butyl dicarbonate (0.56 g) in tetrahydrofuran (2 ml), and the mixture was stirred at the same temperature for 12 hours. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (hexane:ethyl acetate=2:1 to 1:1) to give tert-butyl (R)-N-[2-(3-chlorophenyl)-2-hydroxyethyl]-N-[2-[4-[(4-hydroxyphenyl)sulfonyl]phenyl]-ethyl]carbamate (1.1 g).

NMR (CDCl₃, δ): 1.2-1.5 (9H, m), 2.6-2.95 (2H, m), 3.15-3.6 (4H, m), 4.8-4.95 (1H, m), 6.8-6.95 (2H, m), 7.15-7.45 (6H, m), 7.7-7.9 (2H, m) (+)ESI-MS (m/z): 554, 556 (M+Na)⁺

### EXAMPLE 38

Under nitrogen at 5° C., to a solution of tert-butyl (R)-N-[2-(3-chlorophenyl)-2-hydroxyethyl]-N-[2-[4-[(4-hydroxyphenyl)sulfonyl]phenyl]ethyl]carbamate (100 mg) in N,N-dimethylformamide (2 ml) was added sodium hydride (60% in oil, 8.3 mg), and the mixture was stirred at the same temperature for 1 hour. To this one was added isopropyl bromoacetate (0.027 ml) and the mixture was stirred at the same temperature for 2 hours. The resulting mixture was poured into saturated aqueous sodium hydrogen carbonate and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=2:1 to 1:1) to give isopropyl (R)-[4-[[4-[2-[N-(tert-butoxycarbonyl)-N-[2-(3-chlorophenyl)-2-hydroxyethyl]-amino]ethyl]phenyl]sulfonyl]phenoxy]acetate (106 mg).

NMR (CDCl₃, δ): 1.2-1.45 (15H, m), 2.65-2.9 (2H, m), 3.2-3.45 (4H, m), 4.61 (2H, s), 4.8-4.9 (1H, m), 5.05-5.2 (1H, m), 6.9-6.95 (2H, m), 7.15-7.4 (6H, m), 7.75-7.9 (4H, m) (+)ESI-MS (m/z): 654, 656 (M+Na)⁺

### EXAMPLE 39

At room temperature, to a solution of isopropyl (R)-[4-[[4-[2-[N-(tert-butoxycarbonyl)-N-[2-(3-chlorophenyl)-2-hydroxyethyl]amino]ethyl]phenyl]sulfonyl]phenoxy]acetate (103 mg) in ethyl acetate (1 ml) was added 4N hydrogen chloride in 1,4-dioxane (1 ml), and the mixture was stirred at the same temperature for 1.5 hours to give a precipitate. The precipitate was collected by filtration and washed with ethyl acetate, followed by dryness to give isopropyl (R)-[4-[[4-[2-[[2-(3-chlorophenyl)-2-hydroxyethyl]amino]ethyl]-phenyl]sulfonyl]phenoxy]acetate hydrochloride (66 mg).

NMR (DMSO-d₆, δ): 1.21 (6H, d, J=6.4 Hz), 2.95-3.5 (6H, m), 4.87 (2H, s), 4.85-5.05 (2H, m), 7.05-7.15 (2H, m), 7.3-7.55 (6H, m), 7.85-7.95 (4H, m) (+)ESI-MS (m/z): 532, 534 (M-HCl+H)⁺

### EXAMPLE 40

The following compounds were obtained according to a similar manner to that of Example 38.

(1) Propyl (R)-[4-[[4-[2-[N-(tert-butoxycarbonyl)-N-[2-(3-chlorophenyl)-2-hydroxyethyl]amino]ethyl]phenyl]-sulfonyl]phenoxy]acetate

NMR (CDCl₃, δ): 0.91 (3H, t, J=7.4 Hz), 1.2-1.5 (9H, m), 1.55-1.8 (2H, m), 2.7-2.9 (2H, m), 3.2-3.45 (4H, m), 4.1-4.2 (2H, m), 4.65 (2H, s), 4.8-4.9 (1H, m), 6.9-7.0 (2H, m), 7.15-7.4 (6H, m), 7.8-7.9 (4H, m) (+)ESI-MS (m/z): 654, 656 (M+Na)⁺

(2) tert-Butyl (R)-[4-[[4-[2-[N-(tert-butoxycarbonyl)-N-[2-(3-chlorophenyl)-2-hydroxyethyl]amino]ethyl]phenyl]-sulfonyl]phenoxy]acetate

NMR (CDCl₃, δ): 1.25-1.45 (9H, m), 1.48 (9H, s), 2.65-2.9 (2H, m), 3.2-3.45 (4H, m), 4.54 (2H, s), 4.8-4.9 (1H, m), 6.9-7.0 (2H, m), 7.15-7.4 (6H, m), 7.8-7.9 (4H, m) (+)ESI-MS (m/z): 668, 670 (M+Na)⁺

(3) Cyclohexyl (R)-[4-[[4-[2-[N-(tert-butoxycarbonyl)-N-[2-(3-chlorophenyl)-2-hydroxyethyl]amino]ethyl]phenyl]-sulfonyl]phenoxy]acetate

NMR (CDCl₃, δ): 1.2-1.95 (19H, m), 2.65-2.9 (2H, m), 3.15-3.45 (4H, m), 4.63 (2H, s), 4.8-4.95 (2H, m), 6.9-7.0 (2H, m), 7.1-7.4 (6H, m), 7.75-7.9 (4H, m) (+)ESI-MS (m/z): 694, 696 (M+Na)⁺

### EXAMPLE 41

The following compounds were obtained according to a similar manner to that of Example 39.

(1) Propyl (R)-[4-[[4-[2-[[2-(3-chlorophenyl)-2-hydroxyethyl]amino]ethyl]phenyl]sulfonyl]phenoxy]-acetate hydrochloride

NMR (DMSO-d₆, δ): 0.82 (3H, t, J=7.4 Hz), 1.5-1.7 (2H, m), 2.9-3.5 (6H, m), 4.06 (2H, t, J=6.6 Hz), 4.85-5.0 (3H, m), 7.05-7.2 (2H, m), 7.3-7.55 (6H, m), 7.8-7.95 (4H, m) (+)ESI-MS (m/z): 532, 534 (M-HCl+H)⁺

(2) tert-Butyl (R)-[4-[[4-[2-[[2-(3-chlorophenyl)-2-hydroxyethyl]amino]ethyl]phenyl]sulfonyl]phenoxy]-acetate hydrochloride

NMR (DMSO-d₆, δ): 1.41 (9H, s), 2.95-3.3 (6H, m), 4.78 (2H, s), 4.9-5.0 (1H, m), 7.05-7.15 (2H, m), 7.3-7.55 (6H, m), 7.85-7.95 (4H, m) (+)ESI-MS (m/z): 546, 548 (M+HCl+H)⁺

(3) Cyclohexyl (R)-[4-[[4-[2-[[2-(3-chlorophenyl)-2-hydroxyethyl]amino]ethyl]phenyl]sulfonyl]phenoxy]-acetate hydrochloride

NMR (DMSO-d₆, δ): 1.05-1.85 (10H, m), 2.8-3.4 (6H, m), 4.65-5.0 (4H, m), 7.05-7.2 (2H, m), 7.25-7.55 (6H, m), 7.8-7.95 (4H, m) (+)ESI-MS (m/z): 572, 574 (M+HCl+H)⁺

### EXAMPLE 42

Under nitrogen at room temperature, to a solution of tert-butyl (R)-N-[2-[3-chlorophenyl]-2-hydroxyethyl]-N-[2-[4-[(4-hydroxyphenyl)sulfonyl]phenyl]ethyl]carbamate (900 mg) in N,N-dimethylformamide (10 ml) was added powdered potassium carbonate (257 mg) and ethyl bromoacetate (0.21 ml), and the mixture was stirred at 60° C. for 1.5 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=2:1 to 1:2) to give ethyl (R)-[4-[[4-[2-[N-(tert-butoxycarbonyl)-N-[2-(3-chlorophenyl)-2-hydroxyethyl]amino]ethyl]phenyl]sulfonyl]-phenoxy]acetate (1.0 g).

NMR (CDCl₃, δ): 1.25-1.5 (12H, m), 2.65-2.95 (2H, m), 3.15-3.5 (4H, m), 4.2-4.3 (2H, m)., 4.64 (2H, s), 5.85-5.95 (1H, m), 6.9-6.95 (2H, m), 7.15-7.4 (6H, m), 7.8-7.9 (4H, m) (+)ESI-MS (m/z): 640, 642 (M+H)⁺

### EXAMPLE 43

At room temperature, to a solution of ethyl (R)-[4-[[4-[2-[N-(tert-butoxycarbonyl)-N-[2-(3-chlorophenyl)-2-hydroxyethyl]amino]ethyl]phenyl]sulfonyl]phenoxy]acetate (374 mg) in ethanol (10 ml) was added aqueous 1N sodium hydroxide (0.61 ml), and the mixture was stirred at the same temperature for 1.5 hours. The resulting mixture was evaporated under reduced pressure and dried to give sodium (R)-[4-[[4-[2-[N-(tert-butoxycarbonyl)-N-[2-(3-chlorophenyl)-2-hydroxyethyl]amino]ethyl]phenyl]sulfonyl]-phenoxy]acetate (372 mg).

NMR (DMSO-d₆, δ): 1.05-1.35 (9H, m), 2.7-2.9 (2H, m), 3.1-3.5 (4H, m), 4.18 (2H, s), 4.65-4.8 (1H, m), 6.9-6.95 (2H, m), 7.15-7.45 (6H, m), 7.75-7.85 (4H, m) (+)ESI-MS (m/z): 588, 590 (M−Na−N)⁻

### EXAMPLE 44

Under nitrogen at room temperature, to a solution of sodium (R)-[4-[[4-[2-[N-(tert-butoxycarbonyl)-N-[2-(3-chlorophenyl)-2-hydroxyethyl]amino]ethyl]phenyl]sulfonyl]-phenoxy]acetate (60 mg) in N,N-dimethylformamide (2 ml) were added sodium iodide (22 mg) and 2-bromoethanol (0.010 ml), and the mixture was stirred at 60° C. for 1 hour. The resulting mixture was poured into saturated aqueous sodium hydrogen carbonate and the aqueous mixture was extracted with ethyl acetate. The organic layer was dried over anhydrous magnesium sulfate and evaporated under reduced pressure, followed by dryness to give 2-hydroxyethyl (R)-[4-[[4-[2-[N-(tert-butoxycarbonyl)-N-[2-(3-chlorophenyl)-2-hydroxyethyl]amino]ethyl]phenyl]sulfonyl]phenoxy]acetate (56 mg).

NMR (CDCl₃, δ): 1.25-1.5 (9H, m), 2.65-3.0 (2H, m), 3.1-3.6 (4H, m), 3.85-3.9 (2H, m), 4.3-4.35 (2H, m), 4.71 (2H, s), 4.85-4.9 (1H, m), 6.9-7.0 (2H, m), 7.1-7.4 (6H, m), 7.75-7.9 (4H, m) (+)ESI-MS (m/z): 656, 658 (M+Na)⁺

### EXAMPLE 45

Under nitrogen at room temperature, to a solution of 2-hydroxyethyl (R)-[4-[[4-[2-[N-(tert-butoxycarbonyl)-N-[2-(3-chlorophenyl)-2-hydroxyethyl]amino]ethyl]phenyl]sulfonyl]-phenoxy]acetate (53 mg) in dichloromethane (3 ml) was added trifluoroacetic acid (0.5 ml), and the mixture was stirred at the same temperature for 45 minutes. The resulting mixture was poured into saturated aqueous sodium hydrogen carbonate and the aqueous mixture was extracted with ethyl acetate. The organic layer was washed successively with water and brine, dried over anhydrous magnesium sulfate and evaporated under reduced pressure. The residue was purified by column chromatography on silica gel (chloroform:methanol=20:1 to 10:1), followed by treatment with 4N hydrogen chlororide in 1,4-dioxane and dryness to give 2-hydroxyethyl (R)-[4-[[4-[2-[[2-(3-chlorophenyl)-2-hydroxyethyl]amino]-ethyl]phenyl]sulfonyl]phenoxy]acetate hydrochloride (24 mg).

NMR (DMSO-d₆, δ): 2.9-3.4 (6H, m), 3.5-3.7 (2H, m), 4.05-4.2 (2H, m), 4.8-5.0 (3H, m), 7.05-7.2 (2H, m), 7.3-7.6 (6H, m), 7.8-8.0 (4H, m) (+)ESI-MS (m/z): 534, 536 (M-HCl+H)⁺

### EXAMPLE 46

The following compound was obtained according to a similar manner to that of Example 42.

2-Ethoxy-1-(ethoxymethyl)ethyl (R)-[4-[[4-[2-[N-(tert-butoxycarbonyl)-N-[2-(3-chlorophenyl)-2-hydroxyethyl]-amino]ethyl]phenyl]sulfonyl]phenoxy]acetate

NMR (CDCl₃, δ): 1.15-1.2 (6H, m), 1.3-1.4 (9H, m), 2.65-2.95 (2H, m), 3.2-3.6 (12H, m), 4.70 (2H, s), 4.85-4.9 (1H, s), 5.25-5.3 (1H,.m), 6.9-6.95 (2H, m), 7.1-7.4 (6H, m), 7.8-7.9 (4H, m) (+)ESI-MS (m/z): 742, 744 (M+Na)⁺

### EXAMPLE 47

The following compound was obtained according to a similar manner to that of Example 44.

2-Pyridylmethyl (R)-[4-[[4-[2-[N-(tert-butoxycarbonyl)-N-[2-(3-chlorophenyl)-2-hydroxyethyl]amino]-ethyl]phenyl]sulfonyl]phenoxy]acetate

NMR (CDCl₃, δ): 1.25-1.5 (9H, m), 2.65-2.95 (2H, m), 3.1-3.6 (4H, m), 4.77 (2H, s), 4.8-4.9 (1H, m), 5.33 (2H, s), 6.95-7.0 (2H, m), 7.1-7.4 (8H, m), 7.65-7.75 (1H, m), 7.8-7.9 (4H, m), 8.6-8.65 (1H, m) (+)ESI-MS (m/z): 703, 705 (M+Na)⁺

### EXAMPLE 48

The following compounds were obtained according to a similar manner to that of Example 45.

(1) 2-Ethoxy-1-(ethoxymethyl)ethyl (R)-[4-[[4-[2-[[2-(3-chlorophenyl)-2-hydroxyethyl]amino]ethyl]phenyl]-sulfonyl]phenoxy]acetate hydrochloride

NMR (DMSO-d₆, δ): 1.04 (6H, t, J=7.0 Hz), 2.9-3.6 (14H, m), 4.85-5.2 (4H, m), 7.05-7.2 (2H, m), 7.3-7.6 (6H, m), 7.8-8.0 (4H, m) (+)ESI-MS (m/z): 620, 622 (M-HCl+H)⁺

(2) 2-Pyridylmethyl (R)-[4-[[4-[2-[[2-(3-chlorophenyl)-2-hydroxyethyl]amino]ethyl]phenyl]sulfonyl]phenoxy]-acetate dihydrochloride

NMR (DMSO-d₆, δ): 2.95-3.4 (6H, m), 4.9-5.0 (1H, m), 5.06 (2H, s), 5.27 (2H, s), 7.1-7.25 (2H, m), 7.3-7.55 (8H, m), 7.8-7.95 (5H, m), 8.55-8.6 (1H, m) (+)ESI-MS (m/z): 581, 583 (M-2HCl+H)⁺

### EXAMPLE 49

Under nitrogen at 5° C., to a solution of ethyl (R)-[4-[[4-[2-[N-(tert-butoxycarbonyl)-N-[2-(3-chlorophenyl)-2-hydroxyethyl]amino]ethyl]phenyl]sulfonyl]phenoxy]acetate (92 mg) in tetrahydrofuran (4 ml) was added sodium borohydride (19 mg), followed by methanol (2 ml) dropwise. The mixture was stirred at room temperature for 12 hours. The resulting mixture was poured into saturated aqueous sodium hydrogen carbonate and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate 1:1 to 1:2) to give tert-butyl (R)-N-[2-(3-chlorophenyl)-2-hydroxyethyl]-N-[2-[4-[[4-(2-hydroxyethyl)phenyl]-sulfonyl]phenyl]ethyl]carbamate (71 mg).

NMR (CDCl_{3, δ):} 1.2-1.5 (9H, m), 2.65-2.9 (2H, m), 3.1-3.5 (4H, m), 3.9-4.0 (2H, m), 4.05-4.15 (2H, m), 4.8-4.9 (1H, m), 6.9-7.0 (2H, m), 7.1-7.4 (6H, m) (+)ESI-MS (m/z): 598, 600 (M+Na)⁺

### EXAMPLE 50

At room temperature, to a solution of tert-butyl (R)-N-[2-(3-chlorophenyl)-2-hydroxyethyl]-N-[2-[4-[[4-(2-hydroxyethoxy)phenyl]sulfonyl]phenyl]ethyl]carbamate (67 mg) in ethyl acetate (1 ml) was added 4N hydrogen chloride in 1,4-dioxane (1 ml), and the mixture was stirred at the same temperature for 1.5 hours. The resulting mixture was poured into saturated aqueous sodium hydrogen carbonate and the aqueous mixture was extracted with ethyl acetate. The organic layer was dried over anhydrous magnesium sulfate and evaporated under reduced pressure. The residue was purified by column chromatography on silica gel (chloroform:methanol=20:1 to 10:1), followed by treatment with 4N hydrogen chlororide in 1,4-dioxane and dryness to give (R)-1-(3-chlorophenyl)-2-[[2-[4-[[4-(2-hydroxyethoxy)phenyl]-sulfonyl]phenyl]ethyl]amino]ethanol hydrochloride (36 mg).

NMR (DMSO-d₆, δ): 2.9-3.5 (6H, m), 3.65-3.8 (2H, m), 4.0-4.15 (2H, m), 4.85-5.0 (1H, m), 7.05-7.2 (2H, m), 7.3-7.6 (6H, m), 7.8-7.95 (4H, m) (+)ESI-MS (m/z): 476, 478 (M-HCl+H)⁺

### EXAMPLE 51

The following compounds were obtained according to a similar manner to that of Example 6.

(1) Ethyl 3-[[4-[2-[N-benzyl-N-[(2R)-2-(3-chlorophenyl)-2-hydroxyethyl]amino]ethyl]phenyl]sulfonyl]benzoate

NMR (CDCl₃, δ): 1.40 (3H, t, J=7 Hz), 2.45-3.00 (6H, m), 3.54 (1H, d, J=13 Hz), 3.62 (1H, br s), 3.89 (1H, d, J=13 Hz), 4.40 (2H, q, J=7 Hz), 4.60 (1H, dd, J=10, 4 Hz), 7.00-7.40 (11H, m), 7.58 (1H, t, J=8 Hz), 7.84 (2H, d, J=8 Hz), 8.10 (1H, d, J=8 Hz), 8.22 (1H, d, J=8 Hz), 8.59 (1H, s) (+)APCI-MS (m/z): 578 (M+H)⁺

(2) Ethyl 4-[[4-[2-[N-benzyl-N-[(2R)-2-(3-chlorophenyl)-2-hydroxyethyl]amino]ethyl]phenyl]sulfonyl]benzoate

NMR (CDCl₃, δ): 1.38 (3H, t, J=7 Hz), 2.45-3.00 (6H, m), 3.54 (1H, d, J=13 Hz), 3.60 (1H, br s), 3.90 (1H, d, J=13 Hz), 4.38 (2H, q, J=7 Hz), 4.59 (1H, dd, J=10, 4 Hz), 7.05-7.45 (11H, m), 7.83 (2H, d, J=8 Hz), 7.98 (2H, d, J=8 Hz), 8.14 (2H, d, J=8 Hz) (+)APCI-MS (m/z): 578 (M+H)⁺

### EXAMPLE 52

To a solution of ethyl 3-[[4-[2-[N-benzyl-N-[(2R)-2-(3-chlorophenyl)-2-hydroxyethyl]amino]ethyl]phenyl]sulfonyl]-benzoate (182 mg) in chlorobenzene (1.8 ml)—methanol (1.8 ml) was added triethylamine (0.36 ml), and the solution was hydrogenated (1 atm) over 10% palladium on carbon (43 mg) at room temperaturefor 3 hours. After the catalyst was filtered off, the filtrate was concentrated and purified by column chromatography (silica gel, chloroform/methanol) to give ethyl 3-[[4-[2-[[(2R)-2-(3-chlorophenyl)-2-hydroxyethyl)amino]ethyl]phenyl]sulfonyl]benzoate (107 mg) as an oil.

NMR (CDCl₃, δ): 1.41 (3H, t, J=7 Hz), 2.68 (1H, dd, J=12, 9 Hz), 2.75-3.05 (5H, m), 4.40 (2H, q, J=7 Hz), 4.65 (1H, dd, J=9, 4 Hz), 7.15-7.30 (3H, m), 7.30-7.40 (3H, m), 7.59 (1H, t, J=7.8 Hz), 7.89 (2H, d, J=8 Hz), 8.12 (1H, ddd, J=7.8, 1.8, 1.3 Hz), 8.23 (1H, dt, J=7.8, 1.3 Hz), 8.58 (1H, t, J=1.3 Hz) (+)APCI-MS (m/z): 488 (M+H)⁺

### EXAMPLE 53

Ethyl 3-[[4-[2-[[(2R)-2-(3-chlorophenyl)-2-hydroxyethyl]amino]ethyl]phenyl]sulfonyl]benzoate (28 mg) was dissolved in 4N hydrogen chloride/ethanol (0.6 ml), and the solution was evaporated to give ethyl 3-[[4-[2-[[(2R)-2-(3-chlorophenyl)-2-hydroxyethyl)amino]ethyl]phenyl]-sulfonyl]benzoate hydrochloride (19 mg) as a white powder.

NMR (DMSO-d₆, δ): 1.34 (3H, t, J=7 Hz), 2.85-3.40 (6H, m), 4.36 (2H, q, J=7 Hz), 4.96 (1H, m), 6.31 (1H, d, J=4 Hz, OH), 7.25-7.50 (4H, m), 7.54 (2H, d, J=8 Hz), 7.80 (1H, t, J=8 Hz), 7.98 (2H, d, J=8 Hz), 8.23 (2H, d, J=8 Hz), 8.40 (1H, s), 8.92 (2H, br s) (+)APCI-MS (m/z): 488 (M+H)⁺

### EXAMPLE 54

To a solution of ethyl 3-[[4-[2-[[(2R)-2-(3-chlorophenyl)-2-hydroxyethyl]amino]ethyl]phenyl]sulfonyl]-benzoate (56 mg) in ethanol (0.57 ml) was added 1N sodium hydroxide solution (0.35 ml), and the mixture was stirred at room temperature for 2 hours. After the solvent was evaporated, 1N hydrochloric acid (1 ml) was added to the residue, and the mixture was triturated with acetonitrile to give 3-[[4-[2-[[(2R)-2-(3-chlorophenyl)-2-hydroxyethyl]-amino]ethyl]phenyl]sulfonyl]benzoic acid hydrochloride (31 mg) as a white powder.

NMR (DMSO-d₆, δ): 2.85-3.50 (6H, m), 4.98 (1H, m), 6.32 (1H, d, J=4 Hz, OH), 7.25-7.50 (4H, m), 7.54 (2H, d, J=8 Hz), 7.77 (1H, t, J=8 Hz), 7.98 (2H, d, J=8 Hz), 8.21 (2H, d, J=8 Hz), 8.38 (1H, s), 8.94 (2H, br s), 13.60 (1H, br s) (+)APCI-MS (m/z): 458 (M−H)⁻

### EXAMPLE 55

The following compounds were obtained according to a similar manner to that of Preparation 30.

(1) Ethyl 3-[[4-[2-[N-(tert-butoxycarbonyl)-N-[(2R)-2-(3-chlorophenyl)-2-hydroxyethyl]amino]ethyl]phenyl]-sulfonyl]benzoate

NMR (CDCl₃, δ): 1.33 (9H, s), 1.41 (3H, t, J=7 Hz), 2.55-3.00 (2H, m), 3.10-3.60 (4H, m), 4.24 (1H, br s, OH), 4.41 (2H, q, J=7 Hz), 4.85 (1H, m), 7.10-7.40 (6H, m), 7.57 (1H, t, J=8 Hz), 7.88 (2H, d, J=8 Hz), 8.09 (1H, d, J=8 Hz), 8.21 (1H, d, J=8 Hz), 8.57 (1H, s) (+)APCI-MS (m/z): 610 (M+Na)⁺

(2) Ethyl 4-[[4-[2-[N-(tert-butoxycarbonyl)-N-[(2R)-2-(3-chlorophenyl)-2-hydroxyethyl]amino]ethyl]phenyl]-sulfonyl]benzoate

NMR (CDCl₃, δ): 1.35 (9H, s), 1.39 (3H, t, J=7 Hz), 2.55-3.00 (2H, m), 3.10-3.60 (4H, m), 4.24 (1H, br s, OH), 4.39 (2H, q, J=7 Hz), 4.84 (1H, m), 7.00-7.35 (6H, m), 7.86 (2H, d, J=8 Hz), 7.98 (2H, d, J=8 Hz), 8.13 (2H, d, J=8 Hz) (+)APCI-MS (m/z): 610 (M+Na)⁺

(3) Ethyl 4-[[4-[(2R)-2-[N-(tert-butoxycarbonyl)-N-[(2R)-2-(3-chlorophenyl)-2-hydroxyethyl]amino]propyl]phenyl]-sulfonyl]benzoate

NMR (CDCl₃, δ): 1.23 (9H, s), 1.25 (3H, d, J=6 Hz), 1.39 (3H, t, J=7 Hz), 2.50-3.70 (4H, m), 4.00-4.25 (1H, m), 4.39 (2H, q, J=7 Hz), 4.67 (1H, m), 5.21 (1H, br s), 7.05-7.45 (6H, m), 7.86 (2H, d, J=8 Hz), 7.97 (2H, d, J=8 Hz), 8.11 (2H, d, J=8 Hz) (+)APCI-MS (m/z): 624 (M+Na)⁺

### EXAMPLE 56

To a solution of ethyl 3-[[4-[2-[N-(tert-butoxycarbonyl)-N-[(2R)-2-(3-chlorophenyl)-2-hydroxyethyl]-amino]ethyl]phenyl]sulfonyl]benzoate (3.06 g) in 1,4-dioxane (31 ml) was added 1N sodium hydroxide solution (6.8 ml), and the mixture was stirred at room temperature for 2.5 hours. After the solution was neutralized with 10% citric acid, the mixture was extracted with ethyl acetate. The organic layer was washed with brine, dried over magnesium sulfate, and filtered. The filtrate was concentrated to give 3-[[4-[2-[N-(tert-butoxycarbonyl)-N-[(2R)-2-(3-chlorophenyl)-2-hydroxyethyl]amino]ethyl]phenyl]sulfonyl]benzoic acid (3.05 g) as a white solid.

NMR (DMSO-d₆, δ): 1.02, 1.18 (total 9H, a pair of s), 2.60-3.00 (2H, m), 3.00-3.70 (4H, m), 4.73 (1H, m), 5.58 (1H, br s), 7.05-7.60 (6H, m), 7.75 (1H, t, J=8 Hz), 7.90 (2H, d, J=8 Hz), 8.19 (2H, d, J=8 Hz), 8.37 (1H, s), 13.41 (1H, br s) (−)APCI-MS (m/z): 558 (M−H)⁻

### EXAMPLE 57

To a solution of 3-[[4-[2-[N-(tert-butoxycarbonyl)-N-[(2R)-2-(3-chlorophenyl)-2-hydroxyethyl]amino]ethyl]phenyl]-sulfonyl]benzoic acid (84 mg) and 1-hydroxybenzotriazole (24 mg) in N,N-dimethylformamide (0.84 ml) was added 1-(3-dimethylaminopropyl)-3-ethylcarbodiimide hydrochloride (37 mg), and the mixture was stirred at room temperature for 1 hour. Ammonia solution (28%, 0.84 ml) was added to the mixture and the mixture was stirred at the same temperature for 2 hours. The mixture was partitioned between hexane/ethyl acetate (1/3) and water. The organic layer was separated, washed successively with sodium hydrogen carbonate solution and brine, dried over magnesium sulfate, and filtered. The filtrate was concentrated, and the residue was purified by column chromatography (silica gel, hexane/ethyl acetate) to give tert-butyl N-[2-[4-[[3-(aminocarbonyl)phenyl]sulfonyl]phenyl]ethyl]-N-[(2R)-2-(3-chlorophenyl)-2-hydroxyethyl]carbamate (80 mg) as a white amorphous powder.

NMR (CDCl₃, δ): 1.36 (9H, s), 2.60-3.60 (6H, m), 4.36 (1H, br s), 4.62 (1H, m), 5.77 (1H, br s), 6.35 (1H, br s), 7.05-7.40 (6H, m), 7.57 (1H, t, J=8 Hz), 7.89 (2H, d, J=8 Hz), 7.98 (1H, d, J=8 Hz), 8.07 (1H, d, J=8 Hz), 8.29 (1H, s) (+)APCI-MS (m/z): 581 (M+Na)⁺

### EXAMPLE 58

The following compounds were obtained according to a similar manner to that of Example 33.

(1) 3-[[4-[2-[[(2R)-2-(3-Chlorophenyl)-2-hydroxyethyl]-amino]ethyl]phenyl]sulfonyl]benzamide hydrochloride

NMR (DMSO-d₆, δ): 2.90-3.35 (6H, m), 5.00 (1H, m), 7.30-7.50 (4H, m), 7.54 (2H, d, J=8 Hz), 7.65 (1H, br s), 7.72 (1H, t, J=8 Hz), 7.97 (2H, d, J=8 Hz), 8.10 (1H, d, J=8 Hz), 8.16 (1H, d, J=8 Hz), 8.31 (1H, br s), 8.42 (1H, s), 8.96 (1H, br s), 9.29 (1H, br s) (+)APCI-MS (m/z): 459 (M+H)⁺

(2) 4-[[4-[2-[[(2R)-2-(3-Chlorophenyl)-2-hydroxyethyl]-amino]ethyl]phenyl]sulfonyl]benzamide hydrochloride.

NMR (DMSO-d₆, δ): 2.85-3.35 (6H, m), 5.00 (1H, dd, J=8, 2 Hz), 7.25-7.50 (4H, m), 7.53 (2H, d, J=8 Hz), 7.63 (1H, br s), 7.96 (2H, d, J=8 Hz), 7.96-8.12 (4H, m), 8.20 (1H, br s), 8.96 (1H, br s), 9.26 (1H, br s) (+)APCI-MS (m/z): 459 (M+H)⁺

(3) 4-[[4-[(2R)-2-[[(2R)-2-(3-Chlorophenyl)-2-hydroxyethyl]amino]propyl]phenyl]sulfonyl]benzamide hydrochloride

NMR (DMSO-d₆, δ): 1.09 (3H, d, J=6 Hz), 2.65-3.70 (5H, m), 5.02 (1H, m), 6.35 (1H, br s), 7.30-7.60 (6H, m), 7.64 (1H, br s), 7.94-8.12 (4H, m), 7.97 (2H, d, J=8 Hz), 8.19 (1H, br s), 8.83 (1H, br s), 9.27 (1H, br s) (+)APCI-MS (m/z): 473 (M+H)⁺

(4) 4-[[4-[(2R)-2-[[(2R)-2-(3-Chlorophenyl)-2-hydroxyethyl]amino]propyl]phenyl]sulfonyl]benzoic acid hydrochloride

NMR (DMSO-d₆, δ): 1.09 (3H, d, J=6 Hz), 2.60-3.70 (5H, m), 5.03 (1H, m), 6.36 (1H, br d, J=3 Hz), 7.25-7.65 (6H, m), 7.97 (2H, d, J=8 Hz), 8.00-8.21 (4H, m), 8.84 (1H, br s), 9.31 (1H, br s), 13.52 (1H, br s) (−)APCI-MS (m/z): 472 (M−H)⁻

### EXAMPLE 59

To a solution of ethyl 3-[[4-[2-[N-benzyl-N-[(2R)-2-(3-chlorophenyl)-2-hydroxyethyl]amino]ethyl]phenyl]-sulfonyl]benzoate (8.25 g) in ethyl acetate (41 ml) was added 4N hydrogen chloride/ethyl acetate (10.7 ml). After the solvent was evaporated, the residue was dissolved in chlorobenzene (58 ml)-ethanol (25 ml), and the solution was hydrogenated (1 atm) over 10% palladium on carbon (409 mg) at room temperature for 1 hour. After the catalyst was filtered off, the filtrate was concentrated to give ethyl 3-[[4-[2-[[(2R)-2-(3-chlorophenyl)-2-hydroxyethyl]amino]-ethyl]phenyl]sulfonyl]benzoate hydrochloride (6.87 g) as a white solid.

NMR (DMSO-d₆, δ): 1.34 (3H, t, J=7 Hz), 2.85-3.40 (6H, m), 4.36 (2H, q, J=7 Hz), 4.98 (1H, m), 6.32 (1H, d, J=4 Hz, OH), 7.25-7.50 (4H, m), 7.54 (2H, d, J=8 Hz), 7.80 (1H, t, J=8 Hz), 7.98 (2H, d, J=8 Hz), 8.23 (2H, d, J=8 Hz), 8.40 (1H, s), 8.99 (2H, br s) (+)APCI-MS (m/z): 488 (M+H)⁺

### EXAMPLE 60

To a suspension of ethyl 3-[[4-[2-[[(2R)-2-(3-chlorophenyl)-2-hydroxyethyl]amino]ethyl]phenyl]sulfonyl]-benzoate hydrochloride (6.86 g) in tetrahydrofuran (34 ml) were added 1N sodium hydroxide solution (13.5 ml) and di-tert-butyl dicarbonate (3.18 g), and the mixture was stirred at room temperature for 1 hour. The mixture was partitioned between ethyl acetate and water. The organic layer was separated, washed successively with water, brine, dried over magnesium sulfate, and filtered. The filtrate was concentrated and the residue was purified by column chromatography (silica gel, hexane/ethyl acetate) to give ethyl 3-[[4-[2-[N-(tert-butoxycarbonyl)-N-[(2R)-2-(3-chlorophenyl)-2-hydroxyethyl]amino]ethyl]phenyl]sulfonyl]-benzoate (7.75 g) as a colorless oil.

NMR (CDCl₃, δ): 1.33 (9H, s), 1.41 (3H, t, J=7 Hz), 2.55-3.00 (2H, m), 3.10-3.60 (4H, m), 4.26 (1H, br s, OH), 4.41 (2H, q, J=7 Hz), 4.85 (1H, m), 7.05-7.40 (6H, m), 7.57 (1H, t, J=8 Hz), 7.88 (2H, d, J=8 Hz), 8.10 (1H, d, J=8 Hz), 8.21 (1H, d, J=8 Hz), 8.57 (1H, s) (+)APCI-MS (m/z): 610 (M+Na)⁺

### EXAMPLE 61

The following compound was obtained according to a similar manner to that of Example 52.

Ethyl 4-[[4-[2-[[(2R)-2-(3-chlorophenyl)-2-hydroxyethyl]amino]ethyl]phenyl]sulfonyl]benzoate

NMR (CDCl₃, δ): 1.39 (3H, t, J=7 Hz), 2.66 (1H, dd, J=12, 9 Hz), 2.70-3.10 (5H, m), 4.39 (2H, q, J=7 Hz), 4.63 (1H, dd, J=9, 4 Hz), 7.10-7.45 (6H, m), 7.87 (2H, d, J=8 Hz), 8.00 (2H, d, J=8 Hz), 8.15 (2H, d, J=8 Hz) (+)APCI-MS (m/z): 488 (M+H)⁺

### EXAMPLE 62

The following compound was obtained according to a similar manner to that of Example 53.

Ethyl 4-[[4-[2-[[(2R)-2-(3-chlorophenyl)-2-hydroxyethyl]amino]ethyl]phenyl]sulfonyl]benzoate hydrochloride

NMR (DMSO-d₆, δ): 1.31 (3H, t, J=7 Hz), 2.9-3.35 (6H, m), 4.34 (2H, q, J=7 Hz), 4.95 (1H, m), 6.32 (1H, d, J=4 Hz, OH), 7.25-7.50 (4H, m), 7.54 (2H, d, J=8 Hz), 7.96 (2H, d, J=8 Hz), 8.03-8.21 (4H, m), 8.91 (2H, br s) (+)APCI-MS (m/z): 488 (M+H)⁺

### EXAMPLE 63

The following compounds were obtained according to a similar manner to that of Example 54.

(1) 4-[[4-[2-[[(2R)-2-(3-Chlorophenyl)-2-hydroxyethyl]-amino]ethyl]phenyl]sulfonyl]benzoic acid hydrochloride

NMR (DMSO-d₆, δ): 2.90-3.35 (6H, m), 4.93 (1H, m), 6.27 (1H, br s, OH), 7.30-7.50 (4H, m), 7.53 (2H, d, J=8 Hz), 7.96 (2H, d, J=8 Hz), 8.00-8.20 (4H, m), 8.75 (2H, br s) (−)APCI-MS (m/z): 458 (M−H)⁻

(2) 4-[[4-[(2R)-2-[[(2R)-2-(3-Chlorophenyl)-2-hydroxyethyl]amino]propyl]phenyl]sulfonyl]benzoic acid hydrochloride

NMR (DMSO-d₆, δ): 1.02 (3H, d, J=6 Hz), 2.55-3.45 (5H, m), 4.92 (1H, m), 7.20-7.55 (6H, m), 7.87 (2H, d, J=8 Hz), 7.97 (2H, d, J=8 Hz), 8.09 (2H, d, J=8 Hz) (−)APCI-MS (m/z): 472 (M−H)⁻

### EXAMPLE 64

The following compounds were obtained according to a similar manner to that of Example 56.

(1) 4-[[4-[2-[N-(tert-Butoxycarbonyl)-N-[(2R)-2-(3-chlorophenyl)-2-hydroxyethyl]amino]ethyl]phenyl]-sulfonyl]benzoic acid

NMR (DMSO-d₆, δ): 1.07, 1.19 (total 9H, a pair of s), 2.65-3.00 (2H, m), 3.00-3.60 (4H, m), 4.72 (1H, m), 5.58 (1H, br s), 7.10-7.60 (6H, m), 7.89 (2H, d, J=8 Hz), 7.96-8.20 (4H, m), 13.55 (1H, br s) (−)APCI-MS (m/z): 558 (M−H)⁻

(2) 4-[[4-[(2R)-2-[N-(tert-Butoxycarbonyl)-N-[(2R)-2-(3-chlorophenyl)-2-hydroxyethyl]amino]propyl]phenyl]-sulfonyl]benzoic acid

NMR (CDCl₃, δ): 1.23 (9H, s), 1.25 (3H, d, J=6 Hz), 2.10-3.70 (5H, m), 4.00-4.25 (1H, m), 4.66 (1H, m), 7.05-7.50 (6H, m), 7.88 (2H, d, J=8 Hz), 8.01 (2H, d, J=8 Hz), 8.16 (2H, d, J=8 Hz) (−)APCI-MS (m/z): 572 (M−H)⁻

### EXAMPLE 65

The following compound was obtained according to a similar manner to that of Example 57.

(1) tert-Butyl N-[2-[4-[[4-(aminocarbonyl)phenyl]sulfonyl]-phenyl]ethyl]-N-[(2R)-2-(3-chlorophenyl)-2-hydroxyethyl]carbamate

NMR (DMSO-d₆, δ): 1.02, 1.19 (total 9H, a pair of s), 2.65-3.60 (6H, m), 4.73 (1H, m), 5.58 (1H, br s), 7.10-7.50 (6H, m), 7.62 (1H, br s), 7.89 (2H, d, J=8 Hz), 7.92-8.12 (4H, m), 8.16 (1H, br s) (+)APCI-MS (m/z): 581 (M+Na)⁺

(2) tert-Butyl N-[(1R)-2-[4-[[4-(aminocarbonyl)phenyl]-sulfonyl]phenyl]-1-methylethyl]-N-[(2R)-2-(3-chlorophenyl)-2-hydroxyethyl]carbamate

NMR (CDCl₃, δ): 1.24 (3H, d, J=6 Hz), 1.26 (9H, s), 2.50-3.70 (4H, m), 3.95-4.25 (1H, m), 4.62 (1H, m), 5.20 (1H, br s), 5.79 (1H, br s), 6.10 (1H, br s), 7.10-7.45 (6H, m), 7.86 (2H, d, J=8 Hz), 7.86 (2H, d, J=8 Hz), 7.98 (2H, d, J=8 Hz) (+)APCI-MS (m/z): 595 (M+Na)⁺

### EXAMPLE 66

To a solution of ethyl 4-[[4-[(2R)-2-aminopropyl]-phenyl]sulfonyl]benzoate (1.06 g) in dimethyl sulfoxide (8.5 ml) was added N,O-bis(trimethylsilyl)acetamide (0.46 ml) at room temperature. After 15 minutes, (R)-2-(3-chlorophenyl)-oxirane (621 mg) was added to the mixture, and the mixture was heated to 80° C. for 44.5 hours before allowed to cool to room temperature. To the solution was added 1 M tetrabutylammonium fluoride in tetrahydrofuran (1.3 ml) and the mixture was stirred at room temperature for 1.5 hours. The mixture was partitioned between hexane/ethyl acetate (1/3) and water. The organic layer was separated, washed successively with water, brine, dried over magnesium sulfate, and filtered. The filtrate was concentrated and the residue was purified by column chromatography (silica gel, chloroform/methanol) to give ethyl 4-[[4-[(2R)-2-[[(2R)-2-(3-chlorophenyl)-2-hydroxyethyl]amino]propyl]phenyl]-sulfonyl]benzoate (409 mg) as a pale yellow solid.

NMR (CDCl₃, δ): 1.05 (3H, d, J=6 Hz), 1.38 (3H, t, J=7 Hz), 2.50-3.10 (5H, m), 4.39 (2H, q, J=7 Hz), 4.53 (1H, dd, J=9, 4 Hz), 7.05-7.40 (6H, m), 7.87 (2H, d, J=8 Hz), 8.00 (2H, d, J=8 Hz), 8.15 (2H, d, J=8 Hz) (+)APCI-MS (m/z): 502 (M+H)⁺

### EXAMPLE 67

To a solution of ethyl [4-[[4-(2-aminoethyl)phenyl]-sulfonyl]-2-chlorophenoxy]acetate (388 mg) in ethanol (8.0 ml) was added (2R)-2-(3-chlorophenyl)oxirane (166 mg) and the solution was refluxed for 13 hours. After cooling to room temperature, the solvent was removed by evaporation. The residue was chromatographed on silica gel (eluent: chloroform/methanol) to give ethyl [2-chloro-4-[[4-[2-[[(2R)-2-(3-chlorophenyl)-2-hydroxyethyl]amino]ethyl]-phenyl]sulfonyl]phenoxy]acetate (115 mg) as a white foam.

(+)APCI-MS (m/z): 552 (M+H)⁺

### EXAMPLE 68

Ethyl [2-chloro-4-[[4-[2-[[(2R)-2-(3-chlorophenyl)-2-hydroxyethyl)amino]ethyl]phenyl]sulfonyl]phenoxy]acetate (64.0 mg) was suspended in 4N hydrogen chloride in ethanol (500 μl) and the mixture was stirred at room temperature for 1 hour. The precipitates were collected by filtration, washed with ethanol, and dried under reduced pressure to give ethyl [2-chloro-4-[[4-[2-[[(2R)-2-(3-chlorophenyl)-2-hydroxyethyl]amino]ethyl]phenyl]sulfonyl]phenoxy]acetate hydrochloride (54.0 mg) as a white solid.

NMR (CDCl₃, δ): 1.20 (3H, t, J=7.1 Hz), 2.84-3.42 (6H, m), 4.16 (2H, q, J=7.1 Hz), 4.94-4.99 (1H, m), 5.05 (2H, s), 6.30 (1H, d, J=4.0 Hz), 7.26-7.53 (7H, m), 7.84-8.03 (4H, m), 8.89 (2H, br s) (+)APCI-MS (m/z): 552 (M+H)⁺

### EXAMPLE 69

To a solution of ethyl [2-chloro-4-[[4-[2-[[(2R)-2-(3-chlorophenyl)-2-hydroxyethyl]amino]ethyl)phenyl]sulfonyl]-phenoxy]acetate (50.0 mg) in ethanol (1.5 ml) was added 1N sodium hydroxide solution (181 μl) at room temperature and the solution was stirred for 3.5 hours. To the solution was added 1N hydrochloric acid (362 μl) and the solution was stirred for 5 minutes. The solvent was removed by evaporation to give a white solid. The solid was applied on a solid phase extraction cartridge (BOND ELUT C18, 20 ml VARIAN) and eluted with water (20 ml). Further elution with methanol/1N hydrochloric acid (90/10) gave [2-chloro-4-[[4-[2-[[(2R)-2-(3-chlorophenyl)-2-hydroxyethyl]amino]ethyl]-phenyl]sulfonyl]phenoxy]acetic acid hydrochloride (52.8 mg) as a white solid.

NMR (CDCl₃, δ): 2.94-3.18 (6H, m), 4.85 (2H, s), 4.96-5.05 (1H, m), 7.19-7.51 (7H, m), 7.83-8.03 (4H, m) (−)APCI-MS (m/z): 522 (M−H)⁻

### EXAMPLE 70

To a suspension of 4-[[4-(2-aminoethyl)phenyl]-sulfonyl]-2-chlorophenol (579 mg) in dimethyl sulfoxide (2.9 ml) was added (2R)-2-(3-chlorophenyl)oxirane (287 mg) and the mixture was stirred at 80° C. for 48 hours. After cooling to room temperature, the mixture was diluted with ethyl acetate (30 ml) and washed with water (30 ml×1). The aqueous layer was extracted with ethyl acetate (15 ml×2). The combined organic layers were dried over magnesium sulfate, filtered, and concentrated in vacuo to give brown foam (827 mg). The crude product was chromatographed on silica gel (eluent: chloroform/methanol) to give a white solid (209 mg). The solid was suspended in 4N hydrogen chloride in ethyl acetate (1 ml) and stirred for 5 minutes. The solvent was removed by evaporation to give 2-chloro-4-[[4-[2-[[(2R)-2-(3-chlorophenyl)-2-hydroxyethyl]amino]ethyl]phenyl]-sulfonyl]phenol hydrochloride (208 mg) as a white solid.

NMR (CDCl₃, δ): 2.97-3.18 (6H, m), 4.98-5.03 (1H, m), 6.34 (1H, d, J=3.9 Hz), 7.20 (1H, d, J=8.6 Hz), 7.33-7.46 (4H, m), 7.50 (2H, d, J=8.1 Hz), 7.74 (1H, dd, J=2.3, 8.6 Hz), 7.89 (1H, s), 7.92 (2H, d, J=8.1 Hz), 8.96 (1H, br s), 9.23 (1H, br s), 11.7 (1H, br s) (+)APCI-MS (m/z): 466 (M+H)⁺

### EXAMPLE 71

The following compounds were obtained according to a similar manner to that of Example 67.

(1) (1R)-1-(3-Chlorophenyl)-2-[[(1R)-2-[4-[(4-methoxyphenyl)sulfonyl]phenyl]-1-methylethyl]amino]-ethanol

(+)APCI-MS (m/z): 460 (M+H)⁺

(2) [4-[[4-[(2R)-2-[[(2R)-2-(3-Chlorophenyl)-2-hydroxyethyl]amino]propyl]phenyl]sulfonyl]phenoxy]-acetate

(+)APCI-MS (m/z): 532 (M+H)⁺

(3) Ethyl [4-[[4-[2-[[(2R)-2-(3-chlorophenyl)-2-hydroxyethyl]amino]-2-methylpropyl]phenyl]sulfonyl]-phenoxy]acetate

MS (m/z): 547 (M+H)

### EXAMPLE 72

The following compounds were obtained according to a similar manner to that of Example 68.

(1) (1R)-1-(3-Chlorophenyl)-2-[[(1R)-2-[4-[(4-methoxyphenyl)sulfonyl]phenyl]-1-methylethyl]amino]-ethanol hydrochloride

NMR (CDCl₃, δ): 1.09 (3H, d, J=6.3 Hz), 2.78 (1H, dd, J=10.7, 12.7 Hz) 3.11-3.49 (3H, m), 3.83 (3H, s), 5.02-5.07 (1H, m), 6.36 (1H, d, J=4.0 Hz), 7.13 (2H, d, J=8.9 Hz), 7.37-7.51 (6H, m), 7.85-7.91 (4H, m), 8.84 (1H, br s), 9.36 (1H, br s) (+)APCI-MS (m/z): 460 (M+H)⁺

(2) [4-[[4-[(2R)-2-[[(2R)-2-(3-Chlorophenyl)-2-hydroxyethyl]amino]propyl]phenyl]sulfonyl]phenoxy]-acetate hydrochloride

NMR (CDCl₃, δ): 1.09 (3H, d, J=6.4 Hz), 1.19 (3H, t, J=7.1 Hz), 2.79 (1H, dd, J=10.7, 12.8 Hz), 3.06-3.21 (2H, m), 3.30-3.51 (2H, m), 4.16 (2H, q, J=7.1 Hz), 4.91 (2H, s), 5.05-5.08 (1H, m), 6.36 (1H, d, J=4.0 Hz), 7.13 (2H, d, J=8.9 Hz), 7.38-7.51 (6H, m), 7.87-7.91 (4H, m), 8.87 (1H, br s), 9.44 (1H, br s) (+)APCI-MS (m/z): 532 (M+H)⁺

### EXAMPLE 73

To a solution of ethyl [4-[[4-[(2R)-2-[[(2R)-2-(3-chlorophenyl)-2-hydroxyethyl]amino]propyl]phenyl]sulfonyl]-phenoxy]acetate (176 mg) in ethanol (1.8 ml) was added 1N sodium hydroxide solution (0.331 ml) at room temperature and the mixture was stirred overnight. The precipitates were collected by filtration, washed with ethanol, and dried under reduced pressure to give sodium [4-[[4-[(2R)-2-[[(2R)-2-(3-chlorophenyl)-2-hydroxyethyl]amino]propyl]phenyl]-sulfonyl]phenoxy]acetate (140 mg) as a white crystalline solid.

NMR (CDCl₃, δ): 0.88 (3H, d, J=6.2 Hz), 2.53-2.84 (5H, m), 4.18 (2H, s), 4.55 (1H, dd, J=5.7, 10.0 Hz), 5.40 (1H, d, J=4.2 Hz), 6.93 (2H, d, J=8.9 Hz), 7.23-7.31 (3H, m), 7.34-7.36 (3H, m), 7.76 (2H, d, J=8.4 Hz), 7.77 (2H, d, J=8.9 Hz) (−)APCI-MS (m/z): 552 (M−Na)⁻

### EXAMPLE 74

A solution of 1-[4-[(4-methoxyphenyl)sulfonyl]phenyl]-2-methyl-2-propanamide (310 mg), (2R)-2-(3-chlorophenyl)-oxirane (150 mg) in ethanol (10 ml) was refluxed for 20 hours. The mixture was evaporated in vacuo. A mixture of residue was chromatographed (chloroform-methanol) over silica gel and triturated with 4N hydrochloride in 1,4-dioxane to give (1R)-1-(3-chlorophenyl)-2-[[2-[4-[(4-methoxyphenyl)sulfonyl]phenyl]-1,1-dimethylethyl]amino]-ethanol hydrochloride (95 mg) as a colorless powder.

NMR (CD₃OD, δ): 1.3 (6H, s), 3.10-3.40 (4H, m), 3.85 (3H, s), 4.90-5.00 (1H, m), 7.00-7.10 (2H, m), 7.30-7.50 (6H, m), 7.80-7.95 (4H, m)

MS (m/z): 474 (M+H)

### EXAMPLE 75

The following compounds were obtained according to a similar manner to that of Example 23.

(1) Sodium [4-[[4-[2-[[(2R)-2-(3-chlorophenyl)-2-hydroxyethyl]amino]-2-methylpropyl]phenyl]sulfonyl]-phenoxy]acetate

NMR (DMSO-d₆, δ): 0.89 (3H, s), 0.91 (3H, s), 2.60-2.80 (4H, m), 4.24 (2H, s), 4.50-4.60 (1H, m), 6.90-7.00 (2H, m), 7.10-7.40 (6H, m), 7.70-7.90 (4H, m)

MS (m/z): 516 (M−H)

(2) Sodium [2-chloro-4-[[4-[(2R)-2-[[(2R)-2-(3-chlorophenyl)-2-hydroxyethyl]amino]propyl]phenyl]-sulfonyl]phenoxy]acetate

MS (m/z): 536 (M−H)

(3) Sodium (4-[[4-[2-[[(2R)-2-(3-chlorophenyl)-2-hydroxyethyl]amino]ethyl]phenyl]sulfonyl]-2-methylphenoxy]acetate

NMR (DMSO-d₆, δ): 2.17 (3H, s), 2.70-2.90 (6H, m), 4.20 (2H, s), 4.60-4.70 (1H, m), 6.80-6.90 (1H, m), 7.20-7.40 (6H, m), 7.7-7.90 (4H, m)

MS (m/z): 502 (M−H)

(4) Sodium [4-[[4-[2-[[(2R)-2-(3-chlorophenyl)-2-hydroxyethyl]amino]ethyl]phenyl]sulfonyl]-2-fluorophenoxy]acetate

NMR (DMSO-d₆, δ): 2.70-2.90 (6H, m), 4.27 (2H, s), 4.60-4.70 (1H, m), 6.80-7.90 (11H, m)

MS (m/z): 506 (M−H)

(5) Sodium [3-[[4-[(2R)-2-[[(2R)-2-(3-chlorophenyl)-2-hydroxyethyl]amino]propyl]phenyl]sulfonyl]phenoxy]-acetate

NMR (DMSO-d₆, δ): 0.89 (2H, d, J=6.2 Hz), 2.62-2.65 (3H, m), 2.80-2.85 (2H, m), 4.15 (2H, s), 4.55 (1H, t, J=6.2 Hz), 7.03-7.08 (1H, m), 7.27-7.48 (9H, m), 7.78-7.82 (2H, d, J=8.3 Hz)

MS (m/z): 502 (M−H)

### EXAMPLE 76

Ethyl [4-[[4-[(2R)-2-aminopropyl]phenyl]sulfonyl]-2-chlorophenoxy]acetate (107 mg), (2R)-2-(3-chlorophenyl)-oxirane (48.2 mg) and bis(trimethylsilyl)acetamide (0.032 ml) in dimethyl sulfoxide (5 ml) was refluxed for 20 hours. To the reaction mixture were added acetic acid (0.5 ml) and water (0.5 ml) and stirred for 1 hour. The resulting mixture was poured into water and extracted with ethyl acetate. The organic layer was washed with brine, dried over magnesium sulfate, and evaporated in vacuo. The residue was chromatographed (chloroform-methanol) over silica gel to give ethyl [2-chloro-4-[[4-[(2R)-2-[[(2R)-2-(3-chlorophenyl)-2-hydroxyethyl]amino]propyl]phenyl]-sulfonyl]phenoxy]acetate (100 mg) as a colorless powder.

MS (m/z): 566 (M+H)

### EXAMPLE 77

The following compounds were obtained according to a similar manner to that of Example 76.

(1) Ethyl [4-[[4-[2-[[(2R)-2-(3-chlorophenyl)-2-hydroxyethyl]amino]ethyl]phenyl]sulfonyl]-2-methylphenoxy]acetate

MS (m/z): 532 (M+H)

(2) Ethyl [4-[[4-[2-[[(2R)-2-(3-chlorophenyl)-2-hydroxyethyl]amino]ethyl]phenyl]sulfonyl]-2-fluorophenoxy]acetate

MS (m/z): 532 (M+H)

### EXAMPLE 78

Ethyl [3-[[4-[(2R)-2-aminopropyl]phenyl]sulfonyl]-phenoxy]acetate (145 mg) and (2R)-2-(3-chlorophenyl)oxirane (65 mg) in ethanol (2.5 ml) was refluxed for 6 hours. The mixture was evaporated. The residue was purified by column chromatography on silica gel (chloroform/methanol=100/3) to give ethyl [3-[[4-[(2R)-2-[[(2R)-2-(3-chlorophenyl)-2-hydroxyethyl]amino]propyl]phenyl]sulfonyl]phenoxy]acetate (90 mg) as a colorless oil.

NMR (CDCl₃, δ): 1.06 (2H, d, J=6.2 Hz), 1.28 (3H, t, J=7.0 Hz), 2.60-2.74 (2H, m), 2.77-2.99 (3H, m), 4.24 (2H, q, J=7.0 Hz), 4.54 (1H, m) 4.64 (2H, s), 7.11-7.55 (10H, m), 7.85 (2H, d, J=8.3 Hz)

MS (m/z): 533 (M+H)

### EXAMPLE 79

Ethyl [3-[[4-[(2R)-2-[[(2R)-2-(3-chlorophenyl)-2-hydroxyethyl]amino]propyl]phenyl]sulfonyl]phenoxy]acetate (50 mg) was triturated with 4N hydrochloride in ethyl acetate (1.0 ml) to give ethyl [3-[[4-[(2R)-2-[[(2R)-2-(3-chlorophenyl)-2-hydroxyethyl]amino]propyl]phenyl]sulfonyl]-phenoxy]acetate hydrochloride (50 mg) as a colorless powder.

MS (m/z): 533 (M+H)

### EXAMPLE 80

To a solution of ethyl (R)-[4-[[4-[2-[N-(tert-butoxycarbonyl)-N-[2-(3-chlorophenyl)-2-hydroxyethyl]-amino]ethyl]phenyl]sulfonyl]phenoxy]acetate (277 mg) in methanol (3 mL) was added ammonia (2 M in methanol, 1 mL) at room temperature, and the mixture was sealed at the same temperature for 4.5 days. The resulting mixture was evaporated under reduced pressure. The residue was dissolved into a mixture of water and ethyl acetate. After seperation, the organic layer was washed with brine, dried over anhydrous magnesium sulfate and evaporated under reduced pressure. The residue was dried in vacuo to give (R)-[4-[[4-[2-[N-(tert-butoxycarbonyl)-N-[2-(3-chlorophenyl)-2-hydroxyethyl]amino]ethyl]phenyl]sulfonyl]-phenoxy]acetamide (248 mg).

NMR (DMSO-d₆, δ): 1.05-1.25 (9H, m), 2.75-2.9 (2H, m), 3.1-3.5 (4H, m), 4.35 (2H, s), 4.65-4.8 (1H, m), 7.05-7.1 (2H, m), 7.15-7.45 (6H, m), 7.75-7.9 (4H, m) (+)ESI-MS (m/z): 611, 613 (M+Na)⁺

### EXAMPLE 81

The following compound was obtained according to a similar manner to that of Example 39.

(R)-2-[4-[[4-[2-[[2-(3-Chlorophenyl)-2-hydroxyethyl]-amino]ethyl]phenyl]sulfonyl]phenoxy]acetamide hydrochloride

NMR (DMSO-d₆, δ): 2.9-3.5 (6H, m), 4.55 (2H, s), 4.85-5.0 (1H, m), 7.11 (2H, d, J=8.9 Hz), 7.3-7.65 (6H, m), 7.8-7.95 (4H, m) (+)ESI-MS (m/z): 489, 491 (M-HCl+H)⁺

Preparation 78

Under nitrogen at 5° C., to a solution of tert-butyl N-[(R)-2-(3-chlorophenyl)-2-hydroxyethyl]-N-[2-[4-[(4-hydroxyphenyl)sulfonyl]phenyl]ethyl]carbamate (241 mg) in N,N-dimethylformamide (5 ml) was added sodium hydride (60% in oil, 40 mg), and the mixture was stirred at the same temperature for 50 minutes. To this one was added ethyl 2-bromo-2-methylpropionate (0.146 ml) and the mixture was stirred at room temperature for 12 hours. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (hexane:ethyl acetate=2:1 to 1:2) to give ethyl (R)-2-[4-[[4-[2-[5-(3-chlorophenyl)-2-oxo-1,3-oxazolidin-3-yl]ethyl]phenyl]sulfonyl]phenoxy]-2-methylpropanoate (43 mg).

NMR (CDCl₃, δ): 1.20 (3H, t, J=7.1 Hz), 1.62 (6H, s), 2.85-4.05 (6H, m), 4.21 (2H, q, J=7.1 Hz), 5.3-5.7 (1H, m), 6.8-6.9 (2H, m), 7.05-7.4 (6H, m), 7.75-7.85 (4H, m) (+)ESI-MS (m/z): 594, 596 (M+Na)⁺

Preparation 79

The following compound was obtained according to a similar manner to that of Preparation 78.

Ethyl 2-[3-[[4-[2-[N-benzyl-N-(tert-butoxycarbonyl)-amino]ethyl]phenyl]thio]phenoxy]-2-methylpropanoate

(+)APCI-MS (m/z): 450 (M-Boc+H)⁺

Preparation 80

The following compounds were obtained according to a similar manner to that of Preparation 18.

(1) Ethyl 2-[3-[[4-[2-[N-benzyl-N-(tert-butoxycarbonyl)-amino]ethyl]phenyl]sulfonyl]phenoxy]-2-methylpropanoate

(+)ESI-MS (m/z): 604 (M+Na)⁺

(2) tert-Butyl N-benzyl-N-[2-[2-[(3-hydroxyphenyl)sulfonyl]phenyl]ethyl]carbamate

MS (m/z): 468 (M+H)

(3) tert-Butyl [2-[6-[(4-methoxyphenyl)sulfonyl]-3-pyridyl]ethyl]carbamate

(+)ESI-MS (m/z): 415 (M+Na)⁺

(4) tert-Butyl [2-[6-[(4-hydroxyphenyl)sulfonyl]-3-pyridyl]ethyl]carbamate

(+)ESI-MS (m/z): 401 (M+Na)⁺

(5) tert-Butyl N-benzyl-N-[2-[3-[(4-methoxyphenyl)sulfonyl]phenyl]ethyl]carbamate

MS (m/z): 504 (M+Na)

(6) tert-Butyl N-benzyl-N-[2-[3-[(3-hydroxyphenyl)sulfonyl]phenyl]ethyl]carbamate

MS (m/z): 468 (M+H)

(7) tert-Butyl N-benzyl-N-[(1S)-2-hydroxy-1-[4-[(4-hydroxyphenyl)sulfonyl]benzyl]ethyl]carbamate

NMR (CDCl₃, δ): 1.42 (9H, s), 3.01 (2H, m), 3.63 (3H, m), 3.90-4.20 (2H, m), 4.25 (1H, br d, J=14 Hz), 6.87 (2H, d, J=9 Hz), 6.90-7.40 (8H, m), 7.75 (2H, d, J=8 Hz), 7.77 (2H, d, J=9 Hz) (+)ESI-MS (m/z): 520 (M+H)⁺

(8) 2,2,2-Trifluoro-N-[2-[4-[(4-hydroxyphenyl)sulfonyl]-phenyl]-1,1-dimethylethyl]acetamide

NMR (CDCl₃, δ): 1.38 (6H, s), 3.15 (2H, s), 5.82 (1H, br s), 6.91 (2H, d, J=9 Hz), 7.22 (2H, d, J=8 Hz), 7.82 (2H, d, J=9 Hz), 7.83 (2H, d, J=8 Hz) (+)ESI-MS (m/z): 424 (M+H)⁺

(9) 2,2,2-Trifluoro-N-[(1R)-2-[4-[(3-methoxyphenyl)-sulfonyl]phenyl]-1-methylethyl]acetamide

NMR (CDCl₃, δ): 1.21 (3H, d, J=7 Hz), 2.83 (1H, dd, J=14 and 7 Hz), 2.97 (1H, dd, J=14 and 6 Hz), 3.85 (3H, s), 4.27 (1H, m), 6.09 (1H, br d, J=7 Hz), 7.02-7.18 (1H, m), 7.20-7.68 (5H, m), 7.89 (2H, d, J=8 Hz) (+)ESI-MS (m/z): 424 (M+H)⁺

(10) 2,2,2-Trifluoro-N-[(1S)-2-[4-[(4-hydroxyphenyl)-sulfonyl]phenyl]-1-methylethyl]acetamide

NMR (DMSO-d₆, δ): 1.14 (3H, d, J=7 Hz), 2.70-2.97 (2H, m), 4.08 (1H, m), 6.90 (2H, d, J=9 Hz), 7.40 (2H, d, J=8 Hz), 7.73 (2H, d, J=9 Hz), 7.79 (2H, d, J=8 Hz), 9.30 (1H, br d, J=8 Hz), 10.64 (1H, br s) (+)ESI-MS (m/z): 410 (M+H)⁺

(11) Ethyl 4-[[4-[[N-benzyl-N-(tert-butoxycarbonyl)amino]-methyl]phenyl]sulfonyl]benzoate

NMR (CDCl₃, δ): 1.39 (3H, t, J=7 Hz), 1.47 (9H, s), 4.36 (4H, br s), 4.40 (2H, q, J=7 Hz), 7.03-7.45 (7H, m), 7.84 (2H, d, J=8 Hz), 8.00 (2H, d, J=8 Hz), 8.16 (2H, d, J=8 Hz) (+)ESI-MS (m/z): 532 (M+Na)⁺

(12) tert-Butyl N-benzyl-N-[4-[(4-hydroxyphenyl)sulfonyl]-benzyl]carbamate

NMR (CDCl₃, δ): 1.48 (9H, s), 4.36 (2H, br s), 4.40 (2H, br s), 6.89 (2H, d, J=9 Hz), 7.05-7.45 (7H, m), 7.76 (2H, d, J=8 Hz), 7.83 (2H, d, J=8 Hz) (+)ESI-MS (m/z): 476 (M+Na)⁺

Preparation 81

The following compounds were obtained according to a similar manner to that of Preparation 32.

(1) Ethyl 2-[3-[[4-[2-(benzylamino)ethyl]phenyl]-sulfonyl]phenoxy]-2-methylpropanoate

(+)APCI-MS (m/z): 482 (M+H)⁺

(2) 3-[[2-[2-(Benzylamino)ethyl]phenyl]sulfonyl]phenol

MS (m/z): 368 (M+H)

(3) N-Benzyl-2-[3-[(4-methoxyphenyl)sulfonyl]phenyl]-ethanamine

MS (m/z): 382 (M+H)

(4) 3-[[3-[2-(Benzylamino)ethyl]phenyl]sulfonyl]phenol

MS (m/z): 368 (M+H)

(5) Ethyl [4-[[4-[2-(benzylamino)ethyl]phenyl]sulfonyl]-phenoxy]acetate

MS (m/z): 454 (M+H)

(6) 4-[[4-[(2S)-2-(Benzylamino)-3-hydroxypropyl]phenyl]-sulfonyl]phenol

NMR (DMSO-d₆, δ): 2.58-2.86 (2H, m), 3.15-3.45 (3H, m), 3.57 (2H, s), 6.92 (2H, d, J=9 Hz), 7.15 (5H, m), 7.39 (2H, d, J=8 Hz), 7.76 (2H, d, J=9 Hz), 7.77 (2H, d, J=8 Hz) (+)ESI-MS (m/z): 398 (M+H)⁺

(7) Ethyl 4-[[4-[(2R)-2-(benzylamino)propyl]phenyl]-sulfonyl]benzoate

NMR (DMSO-d₆, δ): 0.92 (3H, d, J=6 Hz), 1.31 (3H, t, J=7 Hz), 2.40-3.00 (3H, m), 3.67 (1H, d, J=13 Hz), 3.71 (1H, d, J=13 Hz), 4.34 (2H, q, J=7 Hz), 7.17 (5H, m), 7.43 (2H, d, J=8 Hz), 7.87 (2H, d, J=8 Hz), 8.10 (2H, d, J=8 Hz), 8.13 (2H, d, J=8 Hz) (+)ESI-MS (m/z): 438 (M+H)⁺

(8) Ethyl 3-[[3-[2-(benzylamino)ethyl]phenyl]sulfonyl]-benzoate

NMR (CDCl₃, δ): 1.40 (3H, t, J=7 Hz), 2.90 (4H, s), 3.80 (2H, s), 4.40 (2H, q, J=7 Hz), 7.12-7.53 (7H, m), 7.57 (1H, t, J=8 Hz), 7.70-7.90 (2H, m), 8.10 (1H, d, J=8 Hz), 8.22 (1H, d, J=8 Hz), 8.59 (1H, s) (+)ESI-MS (m/z): 424 (M+H)⁺

(9) Ethyl 4-[[3-[2-(benzylamino)ethyl]phenyl]sulfonyl]-benzoate

NMR (CDCl₃, δ): 1.39 (3H, t, J=7 Hz), 2.90 (4H, s), 3.80 (2H, s), 4.39 (2H, q, J=7 Hz), 7.13-7.55 (7H, m), 7.70-7.88 (2H, m), 7.99 (2H, d, J=8 Hz), 8.14 (2H, d, J=8 Hz) (+)ESI-MS (m/z): 424 (M+H)⁺

(10) Ethyl 4-[[4-[(benzylamino)methyl]phenyl]sulfonyl]-benzoate

NMR (CDCl₃, δ): 1.38 (3H, t, J=7 Hz), 3.78 (2H, s), 3.85 (2H, s), 4.39 (2H, q, J=7 Hz), 7.15-7.45 (5H, m), 7.52 (2H, d, J=8 Hz), 7.90 (2H, d, J=8 Hz), 7.99 (2H, d, J=8 Hz), 8.15 (2H, d, J=8 Hz) (+)ESI-MS (m/z): 410 (M+H)⁺

(11) Ethyl 4-[[4-[3-(benzylamino)propyl]phenyl]sulfonyl]-benzoate

NMR (DMSO-d₆, δ): 1.31 (3H, t, J=7 Hz), 1.70 (2H, quintet, J=7 Hz), 2.32 (1H, br s), 2.44 (2H, t, J=7 Hz), 2.69 (2H, t, J=7 Hz), 3.64 (2H, s), 4.34 (2H, q, J=7 Hz), 7.10-7.38 (5H, m), 7.45 (2H, d, J=8 Hz), 7.86 (2H, d, J=8 Hz), 8.09 (2H, d, J=8 Hz), 8.13 (2H, d, J=8 Hz) (+)ESI-MS (m/z): 438 (M+H)⁺

(12) 4-[[4-[(Benzylamino)methyl]phenyl]sulfonyl]phenol

NMR (DMSO-d₆, δ): 3.66 (2H, s), 3.73 (2H, s), 6.92 (2H, d, J=9 Hz), 7.10-7.45 (5H, m), 7.55 (2H, d, J=8 Hz), 7.76 (2H, d, J=9 Hz), 7.83 (2H, d, J=8 Hz), 10.50 (1H, br s) (+)ESI-MS (m/z): 354 (M+H)⁺

(13) 2-[6-[(4-Methoxyphenyl)sulfonyl]-3-pyridyl]ethanamine (+)ESI-MS (m/z): 293 (M+H)⁺

Preparation 82

Under nitrogen at room temperature, to a solution of 2,2,2-trifluoro-N-[2-(4-mercaptophenyl)ethyl]acetamide (1.1 g) in N,N-dimethylformamide (23 ml) were added 6-chloronicotinic acid (765 mg) and potassium carbonate (1.34 g), and the mixture was stirred at 100° C. for 27 hours. The resulting mixture was poured into 0.1N hydrochloric acid and the aqueous mixture was extracted with ethyl acetate. The organic layer was washed with 0.1N hydrochloric acid two times, dried over anhydrous magnesium sulfate and evaporated under reduced pressure. The residue was dissolved into 7N hydrogen chlorode in ethanol (40 ml), and the mixture was refluxed for 11 hours. The resulting mixture was evaporated under reduced pressure. The residue was dissolved into a mixture of saturated aqueous sodium bicarbonate and ethyl acetate. After seperation, the organic layer was dried over anhydrous magnesium sulfate and evaporated under reduced pressure. To a mixture of the residue in a mixture of tetrahydrofuran (30 ml) and water (30 ml) was added di-tert-butyl dicarbonate (4.62 g) in tetrahydrofuran (5 ml) with being adjusted to about pH 8.5 by 1N sodium hydroxide at room temperature, and the mixture was stirred at the same temperature for 12 hours. The resulting mixture was diluted with ethyl acetate, and seperated. The organic layer was dried over anhydrous magnesium sulfate and evaporated under reduced pressure. The residue was purified by column chromatography on silica gel (hexane:ethyl acetate=5:1 to 2:1) to give ethyl 6-[[4-[2-[(tert-butoxycarbonyl)-amino]ethyl]phenyl]thio]nicotinate (1.0 g).

NMR (CDCl₃, δ): 1.37 (3H, t, J=7.1 Hz), 1.4-1.55 (9H, m), 2.86 (2H, t, J=7.1 Hz), 3.35-3.5 (2H, m), 4.37 (2H, q, J=7.1 Hz), 6.85-6.9 (1H, m), 7.25-7.35 (2H, m), 7.5-7.6 (2H, m), 8.02 (1H, dd, J=2.4, 8.5 Hz), 9.00 (1H, d, J=1.7 Hz) (+)ESI-MS (m/z): 425 (M+H)⁺

Preparation 83

Under nitrogen at 5° C., to a solution of ethyl 6-[[4-[2-[(tert-butoxycarbonyl)amino]ethyl]phenyl]thiol]nicotinate (960 mg) in dichloromethane (20 ml) was added m-chloroperbenzoic acid (1.23 g), and the mixture was stirred at room temperature for 2 hours. The resulting mixture was poured into aqueous sodium hydrogensulfite and the aqueous mixture was extracted with ethyl acetate. The organic layer was washed successively with saturated aqueous sodium bicarbonate two times and brine, dried over anhydrous magnesium sulfate and evaporated under reduced pressure. The residue was purified by column chromatography on silica gel (hexane:ethyl acetate=2:1 to 1:1) to give ethyl 6-[[4-[2-[(tert-butoxycarbonyl)amino]ethyl]phenyl]sulfonyl]-nicotinate (786 mg).

NMR (CDCl₃, δ): 1.3-1.5 (12H, m), 2.87 (2H, t, J=6.9 Hz), 3.3-3.5 (2H, m), 4.43 (2H, q, J=7.1 Hz), 7.37 (2H, d, J=8.3 Hz), 8.00 (2H, d, J=8.3 Hz), 8.27 (1H, d, J=7.9 Hz), 8.52 (1H, dd, J=2.0, 8.1 Hz), 9.22 (1H, m) (+)ESI-MS (m/z): 457 (M+Na)⁺

Preparation 84

To a solution of ethyl 6-[[4-[2-[(tert-butoxycarbonyl)-amino]ethyl]phenyl]sulfonyl]nicotinate (754 mg) in ethyl acetate (5 ml) was added 4N hydrogen chloride in ethyl acetate (5 ml) at room temperature, and the mixture was stirred at the same temperature for 2 hours. The resulting mixture was evaporated under reduced pressure. The residue was dissolved into a mixture of saturated aqueous sodium bicarbonate and chloroform. After seperation, the organic layer was dried over anhydrous magnesium sulfate, evaporated under reduced pressure and dried in vacuo to give ethyl 6-[[4-(2-aminoethyl)phenyl]sulfonyl]nicotinate (656 mg).

NMR (DMSO-d₆, δ): 1.32 (3H, t, J=7.1 Hz), 2.6-3.0 (4H, m), 4.37 (2H, q, J=7.1 Hz), 7.45-7.5 (2H, m), 7.85-7.95 (2H, m), 8.3-8.35 (1H, m), 8.55-8.6 (1H, m), 9.1 (1H, m) (+)ESI-MS (m/z): 335 (M+H)⁺

Preparation 85

Under nitrogen at room temperature, to a solution of ethyl 6-[[4-(2-aminoethyl)phenyl]sulfonyl]nicotinate (646 mg) in chloroform (10 ml) was added benzaldehyde (0.206 ml), and the mixture was stirred at the same temperature for 20 minutes. The resulting mixture was evaporated under reduced pressure. Under nitrogen at 5° C., to a solution of the residue in tetrahydrofuran (6 ml) was added sodium borohydride (80 mg), followed by ethanol (6 ml) dropwise and the mixture was stirred at room temperature for 12 hours. The resulting mixture was poured into saturated aqueous bicarbonate and the aqueous mixture was extracted with ethyl acetate. The organic layer was washed successively with water and brine, dried over anhydrous magnesium sulfate and evaporated under reduced pressure. The residue was purified by column chromatography on silica gel (chloroform:methanol=50:1 to 10:1) to give ethyl 6-[[4-[2-(benzylamino)ethyl]phenyl]sulfonyl]nicotinate (135 mg).

NMR (CDCl₃, δ): 1.39 (3H, t, J=7.1 Hz), 2.8-3.1 (4H, m), 3.78 (2H, s), 4.42 (2H, q, J=7.1 Hz), 7.15-8.6 (11H, m), 9.21 (1H, m) (+)ESI-MS (m/z): 425 (M+H)⁺

Preparation 86

To a solution of ethyl [4-[[5-[2-[(tert-butoxycarbonyl)amino]ethyl]-2-pyridyl)sulfonyl]phenoxy]-acetate (260 mg) in tetrahydrofuran (1.5 ml) was added 3.95N hydrogen chloride in ethanol (1.5 ml), and the mixture was stirred at room temperature for 12 hours. The mixture was evaporated under reduced pressure. The residue was dissolved in dichloromethane (40 ml) and methanol (5 ml), and washed with saturated aqueous sodium bicarbonate. The aqueous layer was extracted with dichloromethane (20 ml) and methanol (2 ml). The combined organic layers were dried over magnesium sulfate and evaporated under reduced pressure to give ethyl [4-[[5-(2-aminoethyl)-2-pyridinyl]sulfonyl]-phenoxy]acetate (215 mg) a colorless oil.

(+)ESI-MS (m/z): 365 (M+H)⁺

Preparation 87

Under nitrogen at room temperature, to a suspension of sodium borohydride (9.75 g) in tetrahydrofuran (300 ml) was added 4-iodo-L-phenylalanine (30 g, J. Org. Chem. 59(15), 4206(1994)). The mixture was cooled to 5° C., and concentrated sulfuric acid (7.2 ml) in diethyl ether (10 ml) was added dropwise. The mixture was stirred at room temperature for 24 hours. To the resulting mixture was added methanol (10 ml) carefully, followed by 5N sodium hydroxide (300 ml). After removal of tetrahydrofuran by evaporation, the residual aqueous solution was refluxed for 3 hours. To the resulting mixture were added dichloromethane, tetrahydrofuran and water. After separation, the aqueous layer was extracted with dichloromethane three times. The combined organic layer was dried over anhydrous magnesium sulfate, evaporated under reduced pressure and dried in vacuo to give (S)-2-amino-3-(4-iodophenyl)-1-propanol (22.3 g).

NMR (CDCl₃, δ): 2.4-2.55 (1H, m), 2.6-2.8 (1H, m), 3.0-3.15 (1H, m), 3.3-3.45 (1H, m), 3.55-3.7 (1H, m), 6.95 (2H, d, J=8.2 Hz), 7.63 (2H, d, J=8.2 Hz) (+)ESI-MS (m/z): 278 (M+H)⁺

Preparation 88

Under nitrogen at room temperature, to a solution of (S)-2-amino-3-(4-iodophenyl)-1-propanol (1.0 g) in dichloromethane (20 ml) was added benzaldehyde (0.385 ml), and the mixture was stirred at the same temperature for 1 hour. The resulting mixture was evaporated under reduced pressure. Under nitrogen at room temperature, to a solution of the residue in a mixture of dichloromethane (10 ml) and ethanol (20 ml) was added sodium borohydride (150 mg) carefully and the mixture was stirred at room temperature for 2 hours. The resulting mixture was concentrated to about 5 ml under reduced pressure. The residue was poured into water and the aqueous mixture was extracted with ethyl acetate. The organic layer was washed with brine, dried over anhydrous magnesium sulfate, evaporated under reduced pressure and dried in vacuo to give (S)-2-(benzylamino)-3-(4-iodophenyl)-1-propanol (1.17 g).

NMR (CDCl₃, δ): 2.6-3.1 (3H, m), 3.25-3.4 (1H, m), 3.55-3.7 (1H, m), 3.77 (2H, s), 6.85-6.95 (2H, m), 7.1-7.4 (5H, m), 7.55-7.7 (2H, m) (+)ESI-MS (m/z): 367 (M+H)⁺

Preparation 89

To a solution of 3-(trifluoromethyl)benzaldehyde (5 g) in tetrahydrofuran (50 ml) was added potassium tert-butoxide (3.87 g) on ice-cooling and the mixture was stirred at the same temperature for 1 hour. To the mixture was added methyltriphenylphosphonium bromide (12.3 g) and the mixture was stirred at room temperature for 18 hours. The resulting mixture was poured into saturated aqueous sodium bicarbonate solution, and extracted with ethyl acetate. The organic layer was washed with brine, dried over magnesium sulfate, and evaporated in vacuo to give 1-(trifluoromethyl)-3-vinylbenzene (2.18 g) as colorless oil.

MS (m/z): 173 (M+H)

Preparation 90

To a solution of AD mix-beta (17.78 g) (J. Org. Chem. Vol. 57, No 10, 1992 2768-2771) in tert-butanol (60 ml) and water (60 ml) was added 1-(trifluoromethyl)-3-vinylbenzene (2.18 g) on ice-cooling and the mixture was stirred at the same temperature for 4 hours. To the mixture was added sodium sulfite (19 g). The resulting mixture was poured into saturated aqueous sodium bicarbonate solution, and extracted with ethyl acetate. The organic layer was washed with brine, dried over magnesium sulfate, and evaporated in vacuo to give (1R)-1-[3-(trifluoromethyl)phenyl]-1,2-ethanediol (2.5 g) as colorless oil.

NMR (CDCl₃, δ): 3.63 (1H, dd, J=8, 11 Hz), 3.80 (1H, dd, J=3.5, 11 Hz), 4.9 (1H, dd, J=3.5, 8), 7.40-7.70 (4H, m)

Preparation 91

The following compound was obtained according to a similar manner to that of Preparation 89.

3-Vinylbenzonitrile

NMR (DMSO-d₆, δ): 5.40 (1H, d, J=11 Hz), 6.00 (1H, d, J=17 Hz), 6.70 (1H, dd, J=11, 17 Hz), 7.30-8.00 (4H, m)

Preparation 92

The following compounds were obtained according to a similar manner to that of Preparation 90.

(1) 3-[(1R)-1,2-Dihydroxyethyl]benzonitrile

NMR (DMSO-d₆, δ): δ): 3.40-3.55(2H, m), 6.70 (1H, t, J=5 Hz), 7.50-2.70 (4H, m)

(2) (1R)-1-(4-Chlorophenyl)-1,2-ethanediol

NMR (CDCl₃, δ): 3.50-3.80 (2H, m), 4.70-4.85 (1H, m), 7.20-7.40 (4H, m)

Preparation 93

Trimethylsilyl chloride (0.369 ml) was added to the solution of (1R)-1-[3-(trifluoromethyl)phenyl]-1,2-ethanediol (500 mg) and trimethyl orthoacetate (0.367 ml) in dichloromethane (10 ml) on ice-cooling. The solution was stirred for 1 hour and evaporated. The crude product was dissolved in dry methanol and potassium carbonate (825 mg) was. added. The suspension was stirred vigorously for 100 minutes, then filtered and the residue was washed with dichloromethane. The filtrate was washed with brine, dried over magnesium sulfate, and evaporated in vacuo to give (2R)-2-[3-(trifluoromethyl)phenyl]oxirane (320 mg) as a colorless oil.

NMR (CDCl₃, δ): 2.80-2.84 (1H, m), 3.10-3.20 (1H, m), 3.90-3.95 (1H, m), 7.40-7.70 (4H, m)

Preparation 94

The following compounds were obtained according to a similar manner to that of Preparation 28.

(1) Ethyl 4-[[4-[(2R)-2-(benzylamino)propyl]phenyl]-sulfonyl]benzoate

MS (m/z): 438 (M+H)

(2) N-Benzyl-2-[2-[(3-methoxyphenyl)thio]phenyl]ethanamine

MS (m/z): 350 (M+H)

(3) A mixture of N-benzyl-2-[4-[(4-methoxy-3,5-dimethylphenyl)sulfonyl]phenyl]ethanamine and N-benzyl-2-[4-[(3-methoxy-2,4-dimethylphenyl)sulfonyl]-phenyl]ethanamine

MS (m/z): 410 (M+H)

(4) Methyl 4-[[4-[2-(benzylamino)ethyl]phenyl]sulfonyl]-benzoate

MS (m/z): 410 (M+H)

(5) N-Benzyl-2-[3-[(4-methoxyphenyl)thio]phenyl]ethanamine

MS (m/z): 350 (M+H)

(6) N-Benzyl-2-[3-[(3-methoxyphenyl)sulfonyl]phenyl]-ethanamine

MS (m/z): 350 (M+H)

(7) (2R)-N-Benzyl-1-[4-[(4-methoxyphenyl)sulfonyl]phenyl]-2-propanamine

NMR (CDCl₃, δ): 1.06 (3H, d, J=6 Hz), 2.50-3.05 (3H, m), 3.73 (1H, d, J=13 Hz), 3.82 (1H, d, J=13 Hz), 3.84 (3H, s), 6.96 (2H, d, J=9 Hz), 7.10-7.40 (7H, m), 7.81 (2H, d, J=8 Hz), 7.87 (2H, d, J=9 Hz) (+)ESI-MS (m/z): 396 (M+H)⁺

(8) N-Benzyl-3-[4-[(4-methoxyphenyl)sulfonyl]phenyl]-1-propanamine

NMR (CDCl₃, δ): 1.79 (2H, quintet, J=7 Hz), 2.64 (2H, t, J=7 Hz), 2.70 (2H, t, J=7 Hz), 3.76 (2H, s), 3.84 (3H, s), 6.96 (2H, d, J=9 Hz), 7.15-7.45 (7H, m), 7.80 (2H, d, J=8 Hz), 7.87 (2H, d, J=9 Hz) (+)ESI-MS (m/z): 396 (M+H)⁺

(9) Ethyl 4-[[4-[2-(benzylamino)ethyl]phenyl]sulfonyl]-2-fluorobenzoate

(+)APCI-MS (m/z): 442 (M+H)⁺

(10) Methyl 4-[[4-[2-(benzylamino)ethyl]phenyl]sulfonyl]-2-chlorobenzoate

(+)APCI-MS (m/z): 444 (M+H)⁺

(11) Ethyl 4-[[4-[2-(benzylamino)ethyl]phenyl]sulfonyl]-2-methylbenzoate

(+)APCI-MS (m/z): 438 (M+H)⁺

(12) Ethyl 4′-[[4-[2-(benzylamino)ethyl]phenyl]sulfonyl]-2′-chloro-1,1′-biphenyl-4-carboxylate

NMR (CDCl₃, δ): 1.41 (3H, t, J=7.1 Hz), 1.52 (1H, br), 2.83-2.94 (4H, m), 3.79 (2H, s), 4.41 (2H, q, J=7.1 Hz), 7.25-7.48 (10H, m), 7.86-7.92 (3H, m), 8.05-8.14 (3H, m) (+)APCI-MS (m/z): 534 (M+H)⁺

(13) Ethyl 4′-[[4-[2-(benzylamino)ethyl]phenyl]sulfonyl]-2′-chloro-1,1′-biphenyl-3-carboxylate

NMR (CDCl₃, δ): 1.39 (3H, t, J=7.1 Hz), 1.51 (1H, br), 2.83-2.94 (4H, m), 3.80 (2H, m), 4.39 (2H, q, J=7.1 Hz), 7.25-7.58 (10H, m), 7.86-7.92 (3H, m), 8.05-8.12 (3H, m) (+)APCI-MS (m/z): 534 (M+H)⁺

Preparation 95

The following compounds were obtained according to a similar manner to that of Preparation 93.

(1) 3-[(2R)-2-Oxiranyl]benzonitrile

NMR (CDCl₃, δ): 2.70-2.80 (1H, m), 3.10-3.20 (1H, m), 3.90-4.10 (1H, m), 7.40-7.70 (4H, m)

(2) (2R)-2-(4-Chlorophenyl)oxirane

NMR (CDCl₃, δ): 2.75 (1H, dd, J=2.5, 5.5 Hz), 3.14 (1H, dd, J=4.0, 5.5 Hz), 3.80-3.86 (1H, m), 7.18-7.40 (4H, m)

Preparation 96

The following compounds were obtained according to a similar manner to that of Preparation 68.

(1) 2,2,2-Trifluoro-N-[(1R)-2-[4-[(4-methoxy-3-methylphenyl)sulfonyl]phenyl]-1-methylethyl]-acetamide

MS (m/z): 416 (M+H)

(2) 2,2,2-Trifluoro-N-[(1R)-2-[4-[(3-fluoro-4-methoxyphenyl)sulfonyl]phenyl]-1-methylethyl]-acetamide

MS (m/z): 442 (M+Na)

(3) A mixture of 2,2,2-trifluoro-N-[2-[4-[(4-methoxy-3,5-dimethylphenyl)sulfonyl]phenyl]ethyl]acetamide and 2,2,2-trifluoro-N-[2-[4-[(3-methoxy-2,4-dimethylphenyl)sulfonyl]phenyl]ethyl]acetamide

MS (m/z): 416 (M+H)

(4) 2,2,2-Trifluoro-N-[3-[4-[(4-methoxyphenyl)sulfonyl]-phenyl]propyl]acetamide

NMR (CDCl₃, δ): 1.91 (2H, quintet, J=7 Hz), 2.70 (2H, t, J=7 Hz), 3.37 (2H, q, J=7 Hz), 3.84 (3H, s), 6.41 (1H, br s), 6.96 (2H, d, J=9 Hz), 7.28 (2H, d, J=8 Hz), 7.83 (2H, d, J=8 Hz), 7.86 (2H, d, J=9 Hz) (+)ESI-MS (m/z): 424 (M+Na)⁺

By-product: 2,2,2-Trifluoro-N-[3-[4-[(2-methoxyphenyl)-sulfonyl]phenyl]propyl]acetamide

NMR (CDCl₃, δ): 1.93 (2H, quintet, J=7 Hz), 2.73 (2H, t, J=7 Hz), 3.39 (2H, q, J=7 Hz), 3.77 (3H, s), 6.40 (1H, br s), 6.91 (1H, d, J=8 Hz), 7.10 (1H, dd, J=8 and 7 Hz), 7.28 (2H, d, J=8 Hz), 7.54 (1H, ddd, J=8, 7 and 2 Hz), 7.89 (2H, d, J=8 Hz), 8.14 (1H, dd, J=8 and 2 Hz) (+)ESI-MS (m/z): 424 (M+Na)⁺

Preparation 97

The following compounds were obtained according to a similar manner to that of Preparation 34.

(1) 2,2,2-Trifluoro-N-[(1R)-2-[4-[(4-hydroxy-3-methylphenyl)sulfonyl]phenyl]-1-methylethyl]-acetamide

MS (m/z): 399 (M−H)

(2) 2,2,2-Trifluoro-N-[(1R)-2-[4-[(3-fluoro-4-hydroxyphenyl)sulfonyl]phenyl]-1-methylethyl]-acetamide

MS (m/z): 403 (M−H)

(3) 3-[[2-[2-(Benzylamino)ethyl]phenyl]thio]phenol

MS (m/z): 336 (M+H)

(4) A mixture of 4-[[4-[2-(benzylamino)ethyl]phenyl]-sulfonyl]-2,6-dimethylphenol and 3-[[4-[2-benzylamino)-ethyl]phenyl]sulfonyl]-2,6-dimethylphenol

MS (m/z): 396 (M+H)

(5) 3-[[3-[2-(Benzylamino)ethyl]phenyl]thio]phenol

MS (m/z): 336 (M+H)

(6) 4-[[3-[2-(Benzylamino)ethyl]phenyl]sulfonyl]phenol

MS (m/z): 368 (M+H)

(7) 2,2,2-Trifluoro-N-[(1R)-2-[4-[(3-hydroxyphenyl)-sulfonyl]phenyl]-1-methylethyl]acetamide

NMR (CDCl₃, δ): 1.24 (3H, d, J=7 Hz), 2.73-3.07 (2H, m), 4.27 (1H, m), 6.18 (1H, br s), 6.22 (1H, br s), 6.95-7.12 (1H, m), 7.20-7.65 (5H, m), 7.87 (2H, d, J=8 Hz) (−)ESI-MS (m/z): 386 (M−H)⁻

(8) 4-[4-[(2R)-2-[(Benzylamino)propyl]phenyl]sulfonyl]-phenol

NMR (DMSO-d₆, δ): 0.93 (3H, d, J=6 Hz), 2.40-3.00 (3H, m), 3.72 (1H, d, J=14 Hz), 3.76 (1H, d, J=14 Hz), 6.92 (2H, d, J=9 Hz), 7.06-7.36 (5H, m), 7.38 (2H, d, J=8 Hz), 7.76 (2H, d, J=9 Hz), 7.78 (2H, d, J=8 Hz) (+)ESI-MS (m/z): 382 (M+H)⁺

(9) 4-[[4-[(Benzylamino)methyl]phenyl]thio]phenol

NMR (DMSO-d₆, δ): 4.08 (2H, s), 4.12 (2H, s), 6.87 (2H, d, J=9 Hz), 7.10 (2H, d, J=8 Hz), 7.20-7.60 (9H, m), 9.46 (1H, br s), 10.00 (1H, br s) (−)ESI-MS (m/z): 320 (M−H)⁻

(10) 4-[[4-[3-(Benzylamino)propyl]phenyl]sulfonyl]phenol

NMR (CDCl₃, δ): 1.81 (2H, quintet, J=7 Hz), 2.67 (2H, t, J=7 Hz), 2.69 (2H, t, J=7 Hz), 3.79 (2H, s), 6.78 (2H, d, J=9 Hz), 7.15-7.40 (7H, m), 7.76 (2H, d, J=9 Hz), 7.77 (2H, d, J=8 Hz) (+)ESI-MS (m/z): 382 (M+H)⁺

Preparation 98

The following compounds were obtained according to a similar manner to that of Preparation 71.

(1) Ethyl [4-[[4-[(2R)-2-aminopropyl]phenyl]sulfonyl]-2-fluorophenoxy]acetate

MS (m/z): 396 (M+H)

(2) Ethyl [4-[[4-[(2R)-2-aminopropyl]phenyl]sulfonyl]-2-methylphenoxy]acetate

MS (m/z): 392 (M+H)

(3) A mixture of 2-[4-[(4-methoxy-3,5-dimethylphenyl)-sulfonyl]phenyl]ethanamine and 2-[4-[(3-methoxy-2,4-dimethylphenyl)sulfonyl]phenyl]ethanamine

MS (m/z): 320 (M+H)

Preparation 99

The following compounds were obtained according to a similar manner to that of Preparation 16.

(1) 2-[(3-Methoxyphenyl)thio]benzaldehyde

MS (m/z): 267 (M+Na)

(2) 3-[(3-Methoxyphenyl)sulfonyl]benzaldehyde

MS (m/z): 267 (M+Na)

(3) 3-[(4-Methoxyphenyl)thio]benzaldehyde

MS (m/z): 267 (M+Na)

Preparation 100

The following compounds were obtained according to a similar manner to that of Preparation 24.

(1) 1-[(3-Methoxyphenyl)thio]-2-(2-nitroethenyl)benzene

MS (m/z): 310 (M+Na)

(2) Methyl 3-[[3-(2-nitroethenyl)phenyl]thio]phenyl ether

NMR (CDCl₃, δ): 3.80 (3H, s), 6.90-7.00 (3H, m), 7.20-7.50 (7H, m), 7.90 (1H, d, J=13 Hz)

(3) Methyl 4-[[3-(2-nitroethenyl)phenyl]thio]phenyl ether

NMR (CDCl₃, δ): 3.80 (3H, s), 6.90-7.00 (3H, m), 7.20-7.50 (7H, m), 7.90 (1H, d, J=13 Hz)

Preparation 101

The following compounds were obtained according to a similar manner to that of Preparation 26.

(1) 2-[2-[(3-Methoxyphenyl)thio]phenyl]ethanamine

MS (m/z): 260 (M+H)

(2) 2-[3-[(3-Methoxyphenyl)sulfonyl]phenyl]ethanamine

MS (m/z): 260 (M+H)

(3) 2-[3-[(4-Methoxyphenyl)thio]phenyl]ethanamine

MS (m/z): 260 (M+H)

Preparation 102

The following compounds were obtained according to a similar manner to that of Preparation 30.

(1) tert-Butyl N-benzyl-N-[2-[2-[(3-hydroxyphenyl)thio]-phenyl]ethyl]carbamate

MS (m/z): 436 (M+H)

(2) tert-Butyl N-benzyl-N-[2-[3-[(4-methoxyphenyl)thio]-phenyl]ethyl]carbamate

NMR (CDCl₃, δ): 1.55 (9H, s), 2.50-2.70 (2H, m), 3.20-3.40 (2H, m), 3.80 (3H, s), 4.30-4.40 (2H, m), 6.90-7.50 (8H, m)

MS (m/z): 472 (M+Na)

(3) tert-Butyl N-benzyl-N-[2-[3-[(3-hydroxyphenyl)thio]-phenyl]ethyl]carbamate

MS (m/z): 436 (M+H)

(4) tert-Butyl N-benzyl-N-[(1S)-2-hydroxy-1-(4-iodobenzyl)ethyl]carbamate

NMR (CDCl₃, δ): 1.45 (9H, s), 2.60-3.10 (2H, m), 3.45-3.80 (4H, m), 4.00 (1H, m), 4.30 (1H, br d, J=15 Hz), 6.85 (2H, d, J=8 Hz), 7.05-7.40 (5H, m), 7.56 (2H, d, J=8 Hz) (+)ESI-MS (m/z): 490 (M+Na)⁺

(5) tert-Butyl N-benzyl-N-[(1R)-2-[4-[(4-hydroxyphenyl)-sulfonyl]phenyl]-1-methylethyl]carbamate

NMR (CDCl₃, δ): 1.13 (3H, d, J=7 Hz), 1.28 (9H, s), 2.55-3.10 (2H, m), 4.11 (1H, br m), 4.25 (2H, br s), 6.86 (2H, d, J=9 Hz), 6.86 (1H, br s), 7.00-7.40 (7H, m), 7.76 (2H, d, J=9 Hz), 7.76 (2H, d, J=8 Hz) (+)ESI-MS (m/z): 504 (M+Na)⁺

Preparation 103

[2-[[(2R)-2-(3-Chlorophenyl)-2-hydroxyethyl]amino]-ethyl]benzene (30 g) and carbonyldiimidazole (26.5 g) in tetrahydrofuran (300 ml) was refluxed for 4 hours. The resulting mixture was poured into water and extracted with ethyl acetate. The organic layer was washed with brine, dried over magnesium sulfate, and evaporated in vacuo to give (5R)-5-(3-chlorophenyl)-3-(2-phenylethyl)-1,3-oxazolidin-2-one (28 g) as a colorless oil.

MS (m/z): 324 (M+Na)

Preparation 104

The following compounds were obtained according to a similar manner to that of Preparation 67.

(1) 4-[2-[(5R)-5-(3-Chlorophenyl)-2-oxo-1,3-oxazolidin-3-yl]ethyl]benzenesulfonyl chloride

NMR (CDCl₃, δ): 3.00 (2H, t, J=6 Hz), 3.25 (1H, dd, J=6, 8 Hz), 3.50-3.90 (3H, m), 5.30-5.45 (1H, m), 7.10-7.40 (6H, m), 7.90-8.00 (2H, m)

(2) 4-[3-[(Trifluoroacetyl)amino]propyl]benzenesulfonyl chloride

NMR (CDCl₃, δ): 1.99 (2H, quintet, J=7 Hz), 2.81 (2H, t, J=7 Hz), 3.44 (2H, q, J=7 Hz), 6.36 (1H, br s), 7.44 (2H, d, J=8 Hz), 7.97 (2H, d, J=8 Hz)

Preparation 105

To a stirred suspension of zinc powder (1.14 g) and dichlorodimethylsilane (2.12 ml) in 1,2-dichloroethane (20 ml) was successively added the mixed solution of 4-[2-[(5R)-5-(3-chlorophenyl)-2-oxo-1,3-oxazolidin-3-yl]ethyl]-benzenesulfonyl chloride (2.0 g) and dimethylacetamide (1.9 ml) in 1,2-dichloroethane (10 ml). The mixture was stirred for 1 hour at room temprature. After the solution was filtered and evaporated, methanol (10 ml) was added to the residue and then evaporated. The residue was purified by column chromatography (silica gel, hexane/ethyl acetate) to give (5R)-5-(3-chlorophenyl)-3-[2-(4-mercaptophenyl)ethyl]-1,3-oxazolidin-2-one (800 mg) as a colorless oil.

MS (m/z): 356 (M+Na)

Preparation 106

To a solution of (5R)-5-(3-chlorophenyl)-3-[2-(4-mercaptophenyl)ethyl]-1,3-oxazolidin-2-one (200 mg) in ethanol (3 ml) was added 3N sodium hydroxide (3.0 ml) at room temperature and the mixture was stirred at 80° C. for 4 hours. The resulting mixture was evaporated in vacuo. To the residue was added 3N hydrogen chloride (3.0 ml) and di-tert-butyl dicarbonate (131 mg) at room temperature and the mixture was stirred at the same temperature for 18 hours. The reaction mixture was evaporated in vacuo. The residue was poured into saturated aqueous sodium bicarbonate solution, and extracted with chloroform. The organic layer was washed with brine, dried over magnesium sulfate, and evaporated in vacuo to give tert-butyl N-[(2R)-2-(3-chlorophenyl)-2-hydroxyethyl]-N-[2-(4-mercaptophenyl)ethyl]-carbamate (276 mg) as a colorless oil.

MS (m/z): 408 (M+H)

Preparation 107

To a solution of (1R)-2-chloro-1-(6-chloro-3-pyridyl)ethanol (2.3 g) (WO99/32475) in 1N sodium hydroxide (24 ml), water (24 ml) and diethyl ether (24 ml) was stirred at room temperature for 1 hour. The resulting mixture was poured into saturated aqueous sodium bicarbonate solution, and extracted with ethyl acetate. The organic layer was washed with brine, dried over magnesium sulfate, and evaporated in vacuo to give 2-chloro-5-[(2R)-2-oxiranyl]pyridine (2.12 g) as a colorless oil.

NMR (DMSO-d₆, δ): 2.80 (1H, dd, J=2, 5 Hz), 3.20 (1H, dd, J=4, 5 Hz), 3.80-3.90 (1H, m), 7.30-7.50 (2H, m), 8.30 (1H, d, J=2 Hz)

Preparation 108

The following compound was obtained according to a similar manner to that of Preparation 105.

2,2,2-Trifluoro-N-[2-(4-mercaptophenyl)ethyl]acetamide

NMR (DMSO-d₆, δ): 2.70-2.90 (2H, m), 3.30-3.40 (2H, m), 5.31 (1H, s), 7.00-7.40 (6H, m)

MS (m/z): 372 (M+Na)

Preparation 109

Under nitrogen atmosphere, tris(dibenzylideneacetone)dipalladium(0) (910 mg) and bis(2-diphenylphosphinophenyl)ether (1.11 g) were dissolved in toluene (93 ml) at room temperature. After 5 minutes, to the solution were added tert-butyl N-benzyl-N-[(1S)-2-hydroxy-1-(4-iodobenzyl)ethyl]carbamate (9.31 g), 4-mercaptophenol (2.82 g), and potassium tert-butoxide (2.48 g), and the mixture was heated to 100° C. for 2 hours. After being allowed to cool to room temperature, the mixture was filtered to remove the insoluble matter, concentrated, and the residue was purified by column chromatography (silica gel, hexane/ethyl acetate) to give tert-butyl N-benzyl-N-[(1S)-2-hydroxy-1-[4-[(4-hydroxyphenyl)thio]benzyl]ethyl]-carbamate (6.35 g) as a viscous oil.

NMR (CDCl₃, δ): 1.44 (9H, s), 2.60-3.10 (2H, m), 3.40-4.20 (5H, m), 4.36 (1H, br d, J=15 Hz), 6.09 (1H, br s), 6.79 (2H, d, J=9 Hz), 6.90-7.45 (11H, m) (+)ESI-MS (m/z): 488 (M+Na)⁺

Preparation 110

To a solution of 2,2,2-trifluoro-N-(2-phenyl-1,1-dimethylethyl)acetamide (19.85 g) in acetic acid (135 ml)—water (27 ml)—sulfuric acid (4.1 ml) were added iodine (8.26 g) and periodic acid dihydrate (3.71 g) at room temperature, and the mixture was heated to 60° C. for 10 hours. After being allowed to cool to room temperature, the mixture was partitioned between hexane/ethyl acetate and water. The organic layer was separated, washed successively with water, sodium sulfite solution, water, and brine, dried over magnesium sulfate, and filtered. The filtrate was concentrated and the residue was recrystallized from diisopropyl ether (26 ml)—hexane (78 ml) to give 2,2,2-trifluoro-N-[2-(4-iodophenyl)-1,1-dimethylethyl]acetamide (16.42 g) as a white powder.

NMR (CDCl₃, δ): 1.40 (6H, s), 3.02 (2H, s), 5.79 (1H, br s), 6.86 (2H, d, J=8 Hz), 7.63 (2H, d, J=8 Hz) (+)ESI-MS (m/z): 394 (M+Na)⁺

Preparation 111

The following compounds were obtained according to a similar manner to that of Preparation 109.

(1) 2,2,2-Trifluoro-N-[2-[4-[(4-hydroxyphenyl)thio]phenyl]-1,1-dimethylethyl]acetamide

NMR (CDCl₃, δ): 1.39 (6H, s), 2.99 (2H, s), 5.24 (1H, s), 5.84 (1H, br s), 6.84 (2H, d, J=9 Hz), 6.98 (2H, d, J=8 Hz), 7.10 (2H, d, J=8 Hz), 7.37 (2H, d, J=9 Hz) (+)ESI-MS (m/z): 392 (M+Na)⁺

(2) 3-[[4-[2-Methyl-2-[(trifluoroacetyl)amino]propyl]-phenyl]thio]benzoic acid

NMR (CDCl₃, δ): 1.42 (6H, s), 3.08 (2H, s), 5.86 (1H, br s), 7.10 (2H, d, J=8 Hz), 7.26-7.60 (4H, m), 7.84-8.03 (2H, m) (−)ESI-MS (m/z): 396 (M−H)⁻

(3) 2,2,2-Trifluoro-N-[(1R)-2-[4-[(3-methoxyphenyl)thio]-phenyl]-1-methylethyl]acetamide

NMR (CDCl₃, δ): 1.22 (3H, d, J=7 Hz), 2.68-2.98 (2H, m), 3.76 (3H, s), 4.24 (1H, m), 6.08 (1H, br d, J=6 Hz), 6.70-6.98 (3H, m), 7.11 (2H, d, J=8 Hz), 7.21 (1H, t, J=8 Hz), 7.32 (2H, d, J=8 Hz) (+)ESI-MS (m/z): 392 (M+Na)⁺

(4) 2,2,2-Trifluoro-N-[(1S)-2-[4-[(4-hydroxyphenyl)thio]-phenyl]-1-methylethyl]acetamide

NMR (CDCl₃, δ): 1.20 (3H, d, J=7 Hz), 2.60-2.92 (2H, m), 4.25 (1H, m), 5.16 (1H, s), 6.06 (1H, br d, J=7 Hz), 6.83 (2H, d, J=9 Hz), 7.03 (2H, d, J=8 Hz), 7.12 (2H, d, J=8 Hz), 7.36(2H, d, J=9 Hz) (+)ESI-MS (m/z): 378 (M+Na)⁺

(5) tert-Butyl N-benzyl-N-[2-[4-[(4-hydroxyphenyl)-thio]phenyl]ethyl]carbamate

NMR (CDCl₃, δ): 1.45 (9H, s), 2.71 (2H, br s), 3.35 (2H, br s), 4.36 (2H, br s), 5.62 (1H, br s), 6.81 (2H, d, J=8 Hz), 6.90-7.40 (11H, m) (+)ESI-MS (m/z): 458 (M+Na)⁺

Preparation 112

The following compounds were obtained according to a similar manner to that of Preparation 2.

(1) 4-[[4-[2-Methyl-2-[(trifluoroacetyl)amino]propyl]-phenyl]sulfonyl]phenyl trifluoromethanesulfonate

NMR (CDCl₃, δ): 1.40 (6H, s), 3.18 (2H, s), 5.78 (1H, br s), 7.28 (2H, d, J=8 Hz), 7.42 (2H, d, J=9 Hz), 7.88 (2H, d, J=8 Hz), 8.06 (2H, d, J=9 Hz) (−)APCI-MS (m/z): 532 (M−H)⁻

(2) 3-[[4-[(2R)-2-[(2,2,2-Trifluoroacetyl)amino]propyl]-phenyl]sulfonyl]phenyl trifluoromethanesulfonate

NMR (CDCl₃, δ): 1.22 (3H, d, J=7 Hz), 2.86 (1H, dd, J=14 and 7 Hz), 2.98 (1H, dd, J=14 and 6 Hz), 4.27 (1H, m), 6.08 (1H, br d, J=7 Hz), 7.36 (2H, d, J=8 Hz), 7.40-7.60 (1H, m), 7.63 (1H, t, J=8 Hz), 7.78-8.05 (4H, m) (+)ESI-MS (m/z): 542 (M+Na)⁺

(3) 4-[[4-[(2S)-2-[(2,2,2-Trifluoroacetyl)amino]propyl]-phenyl]sulfonyl]phenyl trifluoromethanesulfonate

NMR (CDCl₃, δ): 1.23 (3H, d, J=7 Hz), 2.86 (1H, dd, J=13 and 7 Hz), 2.99 (1H, dd, J=13 and 6 Hz), 4.28 (1H, m), 6.08 (1H, br d, J=7 Hz), 7.36 (2H, d, J=8 Hz), 7.41 (2H, d, J=9 Hz), 7.90 (2H, d, J=8 Hz), 8.03 (2H, d, J=9 Hz) (+)ESI-MS (m/z): 542 (M+Na)⁺

(4) 4-[[4-[(2R)-2-[N-Benzyl-N-(tert-butoxycarbonyl)amino]-propyl]phenyl]sulfonyl]phenyl trifluoromethanesulfonate

NMR (CDCl₃, δ): 1.14 (3H, d, J=7 Hz), 1.30 (9H, s), 2.50-3.15 (2H, m), 4.03 (1H, br m), 4.23 (2H, br s), 7.00-7.40 (7H, m), 7.40 (2H, d, J=9 Hz), 7.80 (2H, d, J=8 Hz), 8.04 (2H, d, J=9 Hz) (+)ESI-MS (m/z): 636 (M+Na)⁺

(5) 3-[[3-[2-[N-Benzyl-N-(tert-butoxycarbonyl)amino]ethyl]-phenyl]sulfonyl]phenyl trifluoromethanesulfonate

NMR (CDCl₃, δ): 1.42 (9H, s), 2.84 (2H, br s), 3.38 (2H, br s), 4.35 (2H, br s), 7.05-8.00 (13H, m) (+)ESI-MS (m/z): 621 (M+Na)⁺

(6) 4-[[3-[2-[N-Benzyl-N-(tert-butoxycarbonyl)amino]-ethyl]phenyl]sulfonyl]phenyl trifluoromethanesulfonate

NMR (CDCl₃, δ): 1.42 (9H, s), 2.85 (2H, m), 3.40 (2H, m), 4.35 (2H, br s), 7.05-7.52 (9H, m), 7.60-7.90 (2H, m), 8.03 (2H, d, J=9 Hz) (+)ESI-MS (m/z): 622 (M+Na)⁺

(7) 4-[[4-[[N-Benzyl-N-(tert-butoxycarbonyl)amino]-methyl]phenyl]thio]phenyl trifluoromethanesulfonate

NMR (CDCl₃, δ): 1.49 (9H, s), 4.11 (2H, br s), 4.14 (2H, br s), 7.05-7.48 (13H, m) (+)ESI-MS (m/z): 576 (M+Na)⁺

(8) 4-[[4-[3-[N-Benzyl-N-(tert-butoxycarbonyl)amino]-propyl]phenyl]sulfonyl]phenyl trifluoromethanesulfonate

NMR (CDCl₃, δ): 1.43 (9H, s), 1.78 (2H, quintet, J=7 Hz), 2.60 (2H, t, J=7 Hz), 3.22 (2H, br s), 4.40 (2H, s), 7.10-7.40 (7H, m), 7.40 (2H, d, J=9 Hz), 7.83 (2H, d, J=8 Hz), 8.03(2H, d, J=9 Hz) (+)ESI-MS (m/z): 636 (M+Na)⁺

(9) 2-Fluoro-4-[[4-[2-[(trifluoroacetyl)amino]ethyl]-phenyl]sulfonyl]phenyl trifluoromethanesulfonate

(+)APCI-MS (m/z): 546 (M+Na)⁺

(10) 2-Chloro-4-[[4-[2-[(trifluoroacetyl)amino]ethyl]-phenyl]sulfonyl]phenyl trifluoromethanesulfonate

(+)APCI-MS (m/z): 562 (M+Na)⁺

(11) 2-Methyl-4-[[4-[2-[(trifluoroacetyl)amino]ethyl]-phenyl]sulfonyl]phenyl trifluoromethanesulfonate

(+)APCI-MS (m/z): 542 (M+Na)⁺

(12) 2-Fluoro-4-[[4-[(2R)-2-[(trifluoroacetyl)amino]-propyl]phenyl]sulfonyl]phenyl trifluoromethanesulfonate

(+)APCI-MS (m/z): 560 (M+Na)⁺

(13) 2-Chloro-4-[[4-[(2R)-2-[(trifluoroacetyl)amino]-propyl]phenyl]sulfonyl]phenyl trifluoromethanesulfonate

NMR (CDCl₃, δ): 1.24 (3H, d, J=6.8 Hz), 2.87 (1H, dd, J=7.3, 13.5 Hz), 3.00 (1H, dd, J=6.2, 13.5 Hz), 4.28 (1H, heptuplet, J=7.0 Hz), 6.13 (1H, d, J=7.6 Hz), 7.38 (2H, d, J=8.4 Hz), 7.49 (1H, d, J=8.7 Hz), 7.87-7.92 (3H, m), 8.09 (1H, d, J=2.2 Hz) (+)APCI-MS (m/z): 576 (M+Na)⁺

(14) 2-Methyl-4-[[4-[(2R)-2-[(trifluoroacetyl)amino]-propyl]phenyl]sulfonyl]phenyl trifluoromethanesulfonate

NMR (CDCl₃, δ): 1.23 (3H, d, J=6.8 Hz), 2.43 (3H, s), 2.89 (1H, dd, J=7.2, 13.5 Hz), 2.98 (1H, dd, J=6.3, 13.5 Hz), 4.28 (1H, heptuplet, J=7.0 Hz), 6.20 (1H, d, J=7.8 Hz), 7.33-7.40 (3H, m), 7.80-7.92 (4H, m) (+)APCI-MS (m/z): 556 (M+Na)⁺

(15) 2-Methoxy-5-[[4-[2-[(trifluoroacetyl)amino]ethyl]-phenyl]sulfonyl]phenyl trifluoromethanesulfonate

(−)APCI-MS (m/z): 534 (M−Na)⁺

(16) 2-Methoxy-4-[[4-[2-[(trifluoroacetyl)amino]ethyl]-phenyl]sulfonyl]phenyl trifluoromethanesulfonate

(+)APCI-MS (m/z): 558 (M+Na)⁺

Preparation 113

The following compounds were obtained according to a similar manner to that of Preparation 52.

(1) Ethyl 4-[[4-[2-methyl-2-[(trifluoroacetyl)amino]-propyl]phenyl]sulfonyl]benzoate

NMR (CDCl₃, δ): 1.39 (6H, s), 1.39 (3H, t, J=7 Hz), 3.17 (2H, s), 4.39 (2H, q, J=7 Hz), 5.78 (1H, br s), 7.26 (2H, d, J=8 Hz), 7.88 (2H, d, J=8 Hz), 8.01 (2H, d, J=9 Hz), 8.16 (2H, d, J=9 Hz) (+)ESI-MS (m/z): 480 (M+H)⁺

(2) Ethyl 3-[[4-[(2R)-2-[(trifluoroacetyl)amino]propyl]-phenyl]sulfonyl]benzoate

NMR (CDCl₃, δ): 1.21 (3H, d, J=7 Hz), 1.41 (3H, t, J=7 Hz), 2.84 (1H, dd, J=14 and 7 Hz), 2.98 (1H, dd, J=14 and 6 Hz), 4.27 (1H, m), 4.41 (2H, q, J=7 Hz), 6.11 (1H, br d, J=8 Hz), 7.33 (2H, d, J=8 Hz), 7.60 (1H, t, J=8 Hz), 7.92 (2H, d, J=8 Hz), 8.11 (1H, d, J=8 Hz), 8.24 (1H, d, J=8 Hz), 8.58 (1H, s) (+)ESI-MS (m/z): 466 (M+Na)⁺

(3) Ethyl 4-[[4-[(2S)-2-[(trifluoroacetyl)amino]propyl]-phenyl]sulfonyl]benzoate

NMR (CDCl₃, δ): 1.21 (3H, d, J=7 Hz), 1.39 (3H, t, J=7 Hz), 2.83 (1H, dd, J=14 and 7 Hz), 2.98 (1H, dd, J=14 and 6 Hz), 4.26 (1H, m), 4.39 (2H, q, J=7 Hz), 6.09 (1H, br d, J=7 Hz), 7.33 (2H, d, J=8 Hz), 7.90 (2H, d, J=8 Hz), 7.99 (2H, d, J=8 Hz), 8.16 (2H, d, J=8 Hz) (+)ESI-MS (m/z): 466 (M+Na)⁺

(4) Ethyl 4-[[4-[(2R)-2-[N-benzyl-N-(tert-butoxycarbonyl)-amino]propyl]phenyl]sulfonyl]benzoate

NMR (CDCl₃, δ): 1.13 (3H, d, J=7 Hz), 1.31 (9H, s), 1.38 (3H, t, J=7 Hz), 2.55-3.15 (2H, m), 4.00 (1H, br m), 4.21 (2H, br s), 4.39 (2H, q, J=7 Hz), 6.95-7.40 (7H, m), 7.80 (2H, d, J=8 Hz), 7.99 (2H, d, J=8 Hz), 8.14 (2H, d, J=8 Hz) (+)ESI-MS (m/z): 560 (M+Na)⁺

(5) Ethyl 3-[[3-[2-[N-benzyl-N-(tert-butoxycarbonyl)amino]-ethyl]phenyl]sulfonyl]benzoate

NMR (CDCl₃, δ): 1.40 (3H, t, J=7 Hz), 1.42 (9H, s), 2.82 (2H, br s), 3.37 (2H, br s), 4.33 (2H, br s), 4.40 (2H, q, J=7 Hz), 7.08-7.50 (7H, m), 7.50-7.90 (3H, m), 8.09 (1H, d, J=8 Hz), 8.23 (1H, d, J=8 Hz), 8.58 (1H, s) (+)ESI-MS (m/z): 546 (M+Na)⁺

(6) Ethyl 4-[[4-[[N-benzyl-N-(tert-butoxycarbonyl)amino]-methyl]phenyl]thio]benzoate

NMR (CDCl₃, δ): 1.37 (3H, t, J=7 Hz), 1.50 (9H, s), 4.36 (2H, q, J=7 Hz), 4.40 (4H, br s), 7.10-7.40 (9H, m), 7.43 (2H, d, J=8 Hz), 7.91 (2H, d, J=8 Hz) (+)ESI-MS (m/z): 500 (M+Na)⁺

(7) Ethyl 4-[[4-[3-[N-benzyl-N-(tert-butoxycarbonyl)amino]-propyl]phenyl]sulfonyl]benzoate

NMR (CDCl₃, δ): 1.39 (3H, t, J=7 Hz), 1.43 (9H, s), 1.77 (2H, quintet, J=7 Hz), 2.59 (2H, t, J=7 Hz), 3.21 (2H, br s), 4.39 (2H, q, J=7 Hz), 4.40 (2H, s), 7.10-7.40 (7H, m), 7.83 (2H, d, J=8 Hz), 7.98 (2H, d, J=8 Hz), 8.14 (2H, d, J=8 Hz) (+)ESI-MS (m/z): 560 (M+Na)⁺

(8) Ethyl 2-fluoro-4-[[4-[2-[(trifluoroacetyl)amino]-ethyl]phenyl]sulfonyl]benzoate

(+)APCI-MS (m/z): 470 (M+Na)⁺

(9) Ethyl 2-chloro-4-[[4-[2-[(trifluoroacetyl)amino]-ethyl]phenyl]sulfonyl]benzoate

(+)APCI-MS (m/z): 486 (M+Na)⁺

(10) Ethyl 2-methyl-4-[[4-[2-[(trifluoroacetyl)amino]-ethyl]phenyl]sulfonyl]benzoate

(+)APCI-MS (m/z): 466 (M+Na)⁺

(11) Ethyl 2-fluoro-4-[[4-[(2R)-2-[(trifluoroacetyl)amino]-propyl]phenyl]sulfonyl]benzoate

(+)APCI-MS (m/z): 484 (M+Na)⁺

(12) Ethyl 2-chloro-4-[[4-[(2R)-2-[(trifluoroacetyl)amino]-propyl]phenyl]sulfonyl]benzoate

(+)APCI-MS (m/z): 500 (M+Na)⁺

(13) Ethyl 2-methyl-4-[[4-[(2R)-2-[(trifluoroacetyl)amino]-propyl]phenyl]sulfonyl]benzoate

(+)APCI-MS (m/z): 480 (M+Na)⁺

(14) Ethyl 2-methoxy-5-[[4-[2-[(trifluoroacetyl)amino]-ethyl]phenyl]sulfonyl]benzoate

NMR (CDCl₃, δ): 1.39 (3H, t, J=7.1 Hz), 2.95 (3H, t, J=7.1 Hz), 3.56-3.66 (2H, m), 3.95 (3H, s), 4.37 (2H, q, J=7.1 Hz), 6.36 (1H, br), 7.06 (1H, d, J=8.9 Hz), 7.33 (2H, d, J=8.3 Hz), 7.88 (2H, d, J=8.3 Hz), 8.02 (1H, dd, J=2.5, 8.8 Hz), 8.31 (1H, d, J=2.5 Hz) (+)APCI-MS (m/z): 482 (M+Na)⁺

(15) Ethyl 2-methoxy-4-[[4-[2-[(trifluoroacetyl)amino]-ethyl]phenyl]sulfonyl]benzoate

NMR (CDCl₃, δ): 1.36 (3H, t, J=7.1 Hz), 2.96 (2H, t, J=7.1 Hz), 3.56-3.67 (2H, m), 3.95 (3H, s), 4.36 (2H, q, J=7.1 Hz), 6.37 (1H, br), 7.35 (2H, d, J=8.3 Hz), 7.47-7.52 (2H, m), 7.81 (1H, d, J=8.2 Hz), 7.90 (2H, d, J=8.3 Hz) (+)APCI-MS (m/z): 482 (M+Na)⁺

(16) Ethyl 4-[[3-[2-[N-benzyl-N-(tert-butoxycarbonyl)amino]-ethyl]phenyl]sulfonyl]benzoate

NMR (CDCl₃, δ): 1.38 (3H, t, J=7 Hz), 1.43 (9H, s), 2.83 (2H, m), 3.37 (2H, m), 4.30 (2H, br s), 4.39 (2H, q, J=7 Hz), 7.05-7.50 (7H, m), 7.60-7.85 (2H, m), 7.98 (2H, d, J=8 Hz), 8.15 (2H, d, J=8 Hz). (+)ESI-MS (m/z): 546 (M+Na)⁺

Preparation 114

To a suspension of ethyl 4-[[4-[2-methyl-2-[(trifluoroacetyl)amino]propyl]phenyl]sulfonyl]benzoate (970 mg) in ethanol (9.7 ml) was added 1N sodium hydroxide solution (5.1 ml), and the mixture was heated to reflux for 4 hours. After the mixture was allowed to cool to room temperature, the solvent was evaporated and the residual solid was dried in vacuo. To the solid was added 4M hydrogen chloride/ethanol (9.7 ml), and the mixture was stirred at room temperature for 8 days. The solvent was evaporated, and the residue was partitioned between ethyl acetate/methanol and sodium bicarbonate solution. The organic layer was separated, washed with brine, and dried over magnesium sulfate. Filtration followed by evaporation gave ethyl 4-[[4-(2-amino-2-ethylpropyl)phenyl]sulfonyl]-benzoate (579 mg) as a pale yellow solid.

NMR (DMSO-d₆, δ): 1.06 (6H, s), 1.31 (3H, t, J=7 Hz), 2.77 (2H, s), 4.34 (2H, q, J=7 Hz), 4.84 (2H, br s), 7.34 (2H, d, J=8 Hz), 7.92 (2H, d, J=8 Hz), 8.00-8.25 (4H, m) (+)ESI-MS (m/z): 362 (M+H)⁺

Preparation 115

To a solution of 3-[[4-[2-methyl-2-[(trifluoroacetyl)-amino]propyl]phenyl]thio]benzoic acid (789 mg) in ethyl acetate (16 ml)—water (12 ml) were added tetrabutylammonium hydrogensulfate (134 mg) and OXONE (2.57 g), and the mixture was heated to 70° C. for 5 hours. After being allowed to cool to room temperature, the mixture was partitioned between ethyl acetate and water. The organic layer was separated, washed successively with water, sodium hydrogensulfite solution and brine, dried over magnesium sulfate, and filtered. The filtrate was concentrated to give 3-[[4-[2-methyl-2-[(trifluoroacetyl)amino]propyl]-phenyl]sulfonyl]benzoic acid (833 mg) as a pale yellow solid.

NMR (DMSO-d₆, δ): 1.28 (6H, s), 3.10 (2H, s), 7.34 (2H, d, J=8 Hz), 7.77 (1H, t, J=8 Hz), 7.94 (2H, d, J=8 Hz), 8.12-8.30 (2H, m), 8.39 (1H, s), 8.67 (1H, br s), 13.60 (1H, br s) (−)ESI-MS (m/z): 428 (M−H)⁻

Preparation 116

To a solution of 3-[[4-[2-methyl-2-[(trifluoroacetyl)-amino]propyl]phenyl]sulfonyl]benzoic acid (818 mg) in ethanol (4.2 ml) was added 1N sodium hydroxide solution (4.1 ml), and the mixture was heated to reflux for 9.5 hours. After being allowed to cool to room temperature, the mixture was concentrated and the residue was neutralized with 1N hydrochloric acid. The precipitate formed was collected by filtration to give 3-[[4-(2-amino-2-methylpropyl)phenyl]-sulfonyl]benzoic acid (632 mg) as a pale yellow powder.

NMR (DMSO-d₆+NaOD, δ): 0.95 (6H, s), 2.63 (2H, s), 7.39 (2H, d, J=8 Hz), 7.49 (1H, t, J=8 Hz), 7.84 (2H, d, J=8 Hz), 7.88 (1H, d, J=8 Hz), 8.09 (1H, d, J=8 Hz), 8.34 (1H, s) (−)ESI-MS (m/z): 332 (M−H)⁻

Preparation 117

Thionyl chloride (0.20 ml) was added dropwise to ethanol (3.1 ml) at 0° C. To the solution was added 3-[[4-(2-amino-2-methylpropyl)phenyl]sulfonyl]benzoic acid (622 mg), and the mixture was stirred at room temperature for 41.5 hours. The solvent was evaporated, and the residue was partitioned between ethyl acetate/methanol and sodium bicarbonate solution. The organic layer was separated, washed with brine, dried over magnesium sulfate. Filtration followed by evaporation gave ethyl 3-[[4-(2-amino-2-methylpropyl)phenyl]sulfonyl]benzoate (551 mg) as a brown oil.

NMR (DMSO-d₆, δ): 0.97 (6H, s), 1.34 (3H, t, J=7 Hz), 2.66 (2H, s), 4.36 (2H, q, J=7 Hz), 7.46 (2H, d, J=8 Hz), 7.79 (1H, t, J=8 Hz), 7.91 (2H, d, J=8 Hz), 8.15-8.31 (2H, m), 8.39 (1H, s) (+)APCI-MS (m/z): 362 (M+H)⁺

Preparation 118

The following compounds were obtained according to a similar manner to that of Preparation 110.

(1) 2,2,2-Trifluoro-N-[(1R)-2-(4-iodophenyl)-1-methylethyl]acetamide

NMR (CDCl₃, δ): 1.21 (3H, d, J=7 Hz), 2.74 (1H, dd, J=14 and 7 Hz), 2.85 (1H, dd, J=14 and 6 Hz), 4.26 (1H, m), 6.04 (1H, br s), 6.92 (2H, d, J=8 Hz), 7.65 (2H, d, J=8 Hz) (+)ESI-MS (m/z): 380 (M+Na)⁺

(2) 2,2,2-Trifluoro-N-[(1S)-2-(4-iodophenyl)-1-methylethyl]acetamide

NMR (CDCl₃, δ): 1.21 (3H, d, J=7 Hz), 2.73 (1H, dd, J=14 and 7 Hz), 2.85 (1H, dd, J=14 and 6 Hz), 4.25 (1H, m), 6.04 (1H, br s), 6.92 (2H, d, J=8 Hz), 7.65 (2H, d, J=8 Hz) (−)ESI-MS (m/z): 356 (M−H)⁻

(3) 2,2,2-Trifluoro-N-[2-(4-iodophenyl)ethyl]acetamide

NMR (CDCl₃, δ): 2.84 (2H, t, J=7 Hz), 3.59 (2H, q, J=7 Hz), 6.33 (1H, br s), 6.94 (2H, d, J=8 Hz), 7.66 (2H, d, J=8 Hz) (+)ESI-MS (m/z): 366 (M+Na)⁺

Preparation 119

The following compound was obtained according to a similar manner to that of Preparation 114.

Ethyl 3-[[4-[(2R)-2-aminopropyl]phenyl]sulfonyl]-benzoate

NMR (DMSO-d₆, δ): 0.93 (3H, d, J=7 Hz), 1.34 (3H, t, J=7 Hz), 2.50-2.72 (2H, m), 2.90-3.13 (1H, m), 3.33 (2H, br s), 4.36 (2H, q, J=7 Hz), 7.45 (2H, d, J=8 Hz), 7.79 (1H, t, J=8 Hz), 7.90 (2H, d, J=8 Hz), 8.13-8.33 (2H, m), 8.39 (1H, s) (+)ESI-MS (m/z): 348 (M+H)⁺

Preparation 120

The following compound was obtained according to a similar manner to that of Preparation 116.

4-[[4-[(2S)-2-Aminopropyl]phenyl]sulfonyl]benzoic acid

NMR (DMSO-d₆+NaOD, δ): 0.91 (3H, d, J=7 Hz), 2.47-2.69 (2H, m), 2.97 (1H, m), 7.42 (2H, d, J=8 Hz), 7.83 (2H, d, J=8 Hz), 7.84 (2H, d, J=8 Hz), 7.99 (2H, d, J=8 Hz) (+)ESI-MS (m/z): 342 (M+Na)⁺

Preparation 121

The following compound was obtained according to a similar manner to that of Preparation 117.

Ethyl 4-[[4-[(2S)-2-aminopropyl]phenyl]sulfonyl]-benzoate

NMR (DMSO-d₆, δ): 0.93 (3H, d, J=7 Hz), 1.31 (3H, t, J=7 Hz), 1.99 (2H, br s), 2.50-2.72 (2H, m), 3.02 (1H, m), 4.34 (2H, q, J=7 Hz), 7.46 (2H, d, J=8 Hz), 7.89 (2H, d, J=8 Hz), 8.00-8.22 (4H, m) (+)ESI-MS (m/z): 348 (M+H)⁺

Preparation 122

The following compounds were obtained according to a similar manner to that of Example 60.

(1) tert-Butyl N-benzyl-N-[2-[3-[(4-hydroxyphenyl)-sulfonyl]phenyl]ethyl]carbamate

NMR (CDCl₃, δ): 1.43 (9H, s), 2.82 (2H, m), 3.36 (2H, m), 4.30 (2H, s), 6.75-7.50 (10H, m), 7.55-7.90 (4H, m) (+)ESI-MS (m/z): 490 (M+Na)⁺

(2) tert-Butyl N-benzyl-N-[4-[(4-hydroxyphenyl)thio]-benzyl]carbamate

NMR (CDCl₃, δ): 1.48 (9H, s), 4.28 (2H, br s), 4.35 (2H, br s), 5.78 (1H, br s), 6.82 (2H, d, J=8 Hz), 6.95-7.45 (11H, m) (+)ESI-MS (m/z): 444 (M+Na)⁺

(3) tert-Butyl N-benzyl-N-[3-[4-[(4-hydroxyphenyl)-sulfonyl]phenyl]propyl]carbamate

NMR (CDCl₃, δ): 1.43 (9H, s), 1.80 (2H, quintet, J=7 Hz), 2.54 (2H, t, J=7 Hz), 3.19 (2H, br s), 4.39 (2H, s), 6.90 (2H, d, J=9 Hz), 7.05-7.45 (7H, m), 7.77 (2H, d, J=9 Hz), 7.77 (2H, d, J=8 Hz) (+)ESI-MS (m/z): 504 (M+Na)⁺

(4) tert-Butyl [2-(4-iodophenyl)ethyl]carbamate

NMR (CDCl₃, δ): 1.43 (9H, s), 2.74 (2H, t, J=7 Hz), 3.34 (2H, q, J=7 Hz), 4.51 (1H, br s), 6.94 (2H, d, J=8 Hz), 7.62 (2H, d, J=8 Hz) (+)ESI-MS (m/z): 370 (M+Na)⁺

Preparation 123

A solution of 4-[(4-methoxyphenyl)thio]benzaldehyde (4.88 g) and benzylamine (2.4 ml) in dichloromethane (49 ml) was stirred at room temperature for 2 hours. The mixture was evaporated, and the residual solid was suspended in ethanol (49 ml)—tetrahydrofuran (12 ml). Sodium borohydride (750 mg) was slowly added to the suspension, and the mixture was stirred at room temperature for 1 hour. The mixture was poured onto water and partitioned between hexane/ethyl acetate and water. The organic layer was separated, washed with brine, dried over magnesium sulfate, and filtered. The filtrate was concentrated and the residue was purified by column chromatography (silica gel, ethyl acetate) to give N-benzyl-N-[4-[(4-methoxyphenyl)thio]-benzyl]amine (6.26 g) as a colorless oil.

NMR (CDCl₃, δ): 3.75 (2H, s), 3.79 (2H, s), 3.82 (3H, s), 6.88 (2H, d, J=9 Hz), 7.08-7.50 (11H, m) (+)ESI-MS (m/z): 336 (M+H)⁺

Preparation 124

The following compound was obtained according to a similar manner to that of Preparation 69.

3-[4-[(4-Methoxyphenyl)sulfonyl]phenyl]-1-propanamine

NMR (CDCl₃, δ): 1.75 (2H, quintet, J=7 Hz), 2.68 (2H, t, J=7 Hz), 2.72 (2H, t, J=7 Hz), 3.84 (3H, s), 6.96 (2H, d, J=9 Hz), 7.28 (2H, d, J=8 Hz), 7.81 (2H, d, J=8 Hz), 7.87 (2H, d, J=9 Hz) (+)ESI-MS (m/z): 306 (M+H)⁺

Preparation 125

To an ice-cooled suspension of sodium hydride (60% in mineral oil, 441 mg) in N,N-dimethylformamide (17 ml) was added tert-butyl [2-(4-iodophenyl)ethyl]carbamate (3.47 g), and the mixture was heated to 40° C. for 20 minutes. After the mixture was cooled with ice again, benzyl bromide (1.3 ml) was added, and the resulting suspension was stirred at room temperature for 2.5 hours and partitioned between hexane/ethyl acetate and water. The organic layer was separated, washed successively with water and brine, dried over magnesium sulfate, and filtered. The filtrate was concentrated and the residue was purified by column chromatography (silica gel, hexane/ethyl acetate) to give tert-butyl N-benzyl-N-[2-(4-iodophenyl)ethyl]carbamate (3.14 g) as a viscous oil.

NMR (CDCl₃, δ): 1.45 (9H, s), 2.71 (2H, br s), 3.35 (2H, br s), 4.38 (2H, br s), 6.88 (2H, br s), 7.10-7.40 (5H, m), 7.58 2H, d, J=8 Hz) (+)ESI-MS (m/z): 460 (M+Na)⁺

Preparation 126

The following compounds were obtained according to a similar manner to that of Preparation 70.

(1) Ethyl [4-[[4-[2-[N-benzyl-N-(tert-butoxycarbonyl)-amino]ethyl]phenyl]sulfonyl]phenoxy]acetate

MS (m/z): 576 (M+Na)

(2) Ethyl [2-methyl-4-[[4-[(2R)-2-[(trifluoroacetyl)amino]-propyl]phenyl]sulfonyl]phenoxy]acetate

MS (m/z): 488 (M+H)

(3) Ethyl [2-fluoro-4-f[4-[(2R)-2-[(trifluoroacetyl)amino]-propyl]phenyl]sulfonyl]phenoxy]acetate

MS (m/z): 492 (M+H)

(4) Ethyl 4-[[4-[2-[(5R)-5-(3-chlorophenyl)-2-oxo-1,3-oxazolidin-3-yl]ethyl]phenyl]thio]butanoate

MS (m/z): 448 (M+H)

(5) Methyl 4-[[[4-[2-[N-(tert-butoxycarbonyl)-N-[(2R)-2-(3-chlorophenyl)-2-hydroxyethyl]amino]ethyl]phenyl]-thio]methyl]benzoate

MS (m/z): 556 (M+H)

Preparation 127

To a suspension of ethyl 2-fluoro-4-[[4-[2-[(trifluoroacetyl)amino]ethyl]phenyl]sulfonyl]benzoate (2.95 g) in ethanol (30 ml) was added 1N sodium hydroxide solution (16.5 ml) and the resulting solution was stirred at room temperature for 24 hours. To the solution was added 1N hydrochloric acid (16.5 ml) and the solvent was removed by evaporation. To the residue was added 7N hydrogen chloride in ethanol (30 ml) and the resulting suspension was refluxed for 24 hours. After cooling to room temperature, the solvent was removed by evaporation and the residual solid was partitioned between ethyl acetate (30 ml) and water (30 ml). The mixture was basified with a saturated aqueous sodium bicarbonate solution and the organic layer was separated. The extract was washed with water (30 ml), dried over magnesium sulfate, and concentrated in vacuo to give ethyl 4-[[4-(2-aminoethyl)phenyl]sulfonyl]-2-fluorobenzoate (1.44 g) as a yellow paste.

(+)APCI-MS (m/z): 352 (M+H)⁺

Preparation 128

To a solution of ethyl 2-chloro-4-[[4-[2-[(trifluoroacetyl)amino]ethyl]phenyl]sulfonyl]benzoate (2.93 g) in ethanol (30 ml) was added 1N sodium hydroxide solution (15.8 ml) and the solution was stirred at room temperature for 19 hours. To the solution was added 1N hydrochloric acid (15.8 ml) and the solvent was removed by evaporation. To the residual yellow solid was added 7N hydrogen chloride in ethanol (30 ml) and the suspension was refluxed for 13 hours. After cooling to room temperature, the solvent was removed by evaporation and the residual solid was dissolved in water (30 ml). The solution was basified with saturated aqueous sodium bicarbonate (30 ml) and extracted with chloroform (60 ml). The extract was dried over magnesium sulfate, filtered, and concentrated in vacuo to give ethyl 4-[[4-(2-aminoethyl)phenyl]sulfonyl]-2-chlorobenzoate (2.18 g) as an orange paste.

(+)APCI-MS (m/z): 368 (M+H)⁺

Preparation 129

The following compounds were obtained according to a similar manner to that of Preparation 128.

(1) Ethyl 4-[[4-(2-aminoethyl)phenyl]sulfonyl]-2-methylbenzoate

(+)APCI-MS (m/z): 348 (M+H)⁺

(2) Ethyl 4-[[4-[(2R)-2-aminopropyl]phenyl]sulfonyl]-2-fluorobenzoate

(+)APCI-MS (m/z): 366 (M+H)⁺

(3) Ethyl 4-[[4-[(2R)-2-aminopropyl]phenyl]sulfonyl]-2-chlorobenzoate

(+)APCI-MS (m/z): 382 (M+H)⁺

(4) Ethyl 4-[[4-[(2R)-2-aminopropyl]phenyl]sulfonyl]-2-methylbenzoate

(+)APCI-MS (m/z): 362 (M+H)⁺

(5) Ethyl 4′-[[4-(2-aminoethyl)phenyl]sulfonyl]-2′-chloro-1,1′-biphenyl-4-carboxylate

(+)APCI-MS (m/z): 444 (M+H)⁺

(6) Ethyl 4′-[[4-(2-aminoethyl)phenyl]sulfonyl]-2′-chloro-1,1′-biphenyl-3-carboxylate

(+)APCI-MS (m/z): 444 (M+H)⁺

Preparation 130

To a solution of 2-chloro-4-[[4-[2-[(trifluoroacetyl)-amino]ethyl]phenyl]sulfonyl]phenyl trifluoromethanesulfonate (1.00 g) and (4-methoxycarbonylphenyl)boronic acid (433 mg) in 1,2-dimethoxyethane (10 ml) were added successively tetrakis(triphenylphosphine)palladium (107 mg) and 2N aqueous sodium carbonate solution (1.95 ml). The mixture was stirred at 80° C. for 4 hours. After cooling to room temperature, the mixture was diluted with ethyl acetate (20 ml) and washed with water (20 ml) and brine (20 ml), and dried over magnesium sulfate. Filtration followed by evaporation gave a crude product, which was chromatographed on silica gel (eluent: hexane/ethyl acetate=2/1) to give methyl 2′-chloro-4′-[[4-[2-[(trifluoroacetyl)amino]ethyl]-phenyl]sulfonyl]-1,1′-biphenyl-4-carboxylate (727 mg).

NMR (CDCl₃, δ): 2.98 (2H, t, J=7.1 Hz), 3.58-3.68 (2H, m), 3.95 (3H, m), 6.50 (1H, br), 7.37-7.50 (5H, m), 7.85-7.96 (3H, m), 8.04-8.13 (3H, m) (+)APCI-MS (m/z): 548 (M+Na)⁺

Preparation 131

The following compound was obtained according to a similar manner to that of Preparation 130.

Methyl 2′-chloro-4′-[[4-[2-[(trifluoroacetyl)amino]-ethyl]phenyl]sulfonyl]-1,1′-biphenyl-3-carboxylate

NMR (CDCl₃, δ): 2.98 (2H, t, J=7.1 Hz), 3.58-3.68 (2H, m), 3.92 (3H, s), 6.49 (1H, br), 7.37-7.60 (5H, m), 7.85-7.96 (3H, m), 8.05-8.11 (3H, m) (+)APCI-MS (m/z): 548 (M+Na)⁺

Preparation 132

To a suspension of 4-[2-[(trifluoroacetyl)amino]ethyl]-benzenesulfonyl chloride (10.0 g) in 1,2-dichloroethane (50 ml) were added successively 1,2-dimethoxybenzene (5.22 ml) and aluminum trichloride (6.34 g) and the mixture was refluxed for 18 hours. An additional portion of aluminum trichloride (8.45 g) was added and the mixture was refluxed for 7 hours. The mixture was quenched by addition of water (200 ml) and extracted with ethyl acetate (200 ml, 100 ml). The combined extracts were washed with brine (300 ml) and dried over magnesium sulfate. Filtration followed by evaporation gave a dark purple paste, which was chromatographed on silica gel (eluent: hexane/ethyl acetate) to give the coupling products. The products were dissolved in dichloromethane (100 ml). To the solution was added 1.0 M solution of boron tribromide (83 ml) at 0° C. and the mixture was warmed to room temperature. After stirring for 12 hours, the solvent was removed by evaporation. The residue was suspended in ethyl acetate (100 ml) and carefully basified with saturated aqueous sodium bicarbonate (150 ml) under cooling at 0° C. The aqueous layer was separated and extracted with ethyl acetate (50 ml). The combined organic layers were dried over magnesium sulfate, filtered, and evaporated to give a brown solid, which was chromatographed on silica gel (eluent: hexane/ethyl acetate) to give N-[2-[4-[(3,4-dihydroxyphenyl)sulfonyl]phenyl]ethyl]-2,2,2-trifluoroacetamide (5.29 g) as a light brown solid.

(+)APCI-MS (m/z): 412 (M+Na)⁺

Preparation 133

To a solution of N-[2-[4-[(3,4-dihydroxyphenyl)-sulfonyl]phenyl]ethyl]-2,2,2-trifluoroacetamide (1.00 g) in N,N-dimethylformamide (10 ml) was added potassium carbonate (powder, 390 mg) and the mixture was cooled to 0° C. To the mixture was added iodomethane (208 μl) and the whole was stirred at 0° C. for 20 minutes. The mixture was warmed to room temperature and stirred for 2 hours. The mixture was quenched by addition of water (20 ml) and extracted with ethyl acetate (20 ml and 5 ml). The combined extracts were washed with water (25 ml×2) and brine (25 ml×1), and dried over magnesium sulfate. Filtration followed by evaporation gave a crude oil, which was chromatographed on silica gel (eluent: hexane/ethyl acetate) to give 2,2,2-trifluoro-N-[2-[4-((3-hydroxy-4-methoxyphenyl)sulfonyl]-phenyl]ethyl]acetamide (185 mg) as a pale yellow solid.

(+)APCI-MS (m/z): 426 (M+Na)⁺

Preparation 134

A solution of ethyl 2-methoxy-5-[[4-[2-[(trifluoroacetyl)amino)ethyl]phenyl]sulfonyl]benzoate (113 mg) in 7N hydrogen chloride in ethanol (2.0 ml) was refluxed for 14 hours. After cooling to room temperature, the solvent was removed by evaporation and the residue was dissolved in water (2.0 ml). The solution was basified with saturated aqueous sodium bicarbonate (5 ml) and extracted with chloroform (5 ml×3). The extracts were dried over magnesium sulfate, filtered, and concentrated in vacuo to give ethyl 5-[[4-(2-aminoethyl)phenyl]sulfonyl]-2-methoxybenzoate (84.6 mg) as a white crystalline solid.

NMR (CDCl₃, δ): 1.38 (3H, t, J=7.1 Hz), 1.50 (2H, br), 2.80 (2H, t, J=6.6 Hz), 2.98 (2H, t, J=6.6 Hz), 3.94 (3H, s), 4.37 (2H, q, J=7.1 Hz), 7.05 (1H, d, J=8.8 Hz), 7.34 (2H, d, J=8.4 Hz), 7.86 (2H, d, J=8.4 Hz), 8.03 (1H, dd, J=2.4, 8.8 Hz), 8.32 (1H, d, J=2.4 Hz) (+)APCI-MS (m/z): 364 (M+H)⁺

Preparation 135

The following compounds were obtained according to a similar manner to that of Preparation 133.

(1) N-[2-[4-[[4-(Benzyloxy)-3-hydroxyphenyl]sulfonyl]-phenyl]ethyl]-2,2,2-trifluoroacetamide

(+)APCI-MS (m/z): 502 (M+Na)⁺

(2) N-[2-[4-[[4-(Benzyloxy)-3-methoxyphenyl]sulfonyl]-phenyl]ethyl]-2,2,2-trifluoroacetamide

(+)APCI-MS (m/z): 494 (M+H)⁺

Preparation 136

To a solution of N-[2-[4-[[4-(benzyloxy)-3-methoxyphenyl]sulfonyl]phenyl]ethyl]-2,2,2-trifluoroacetamide (505 mg) in methanol (10 ml) was added 10% palladium on activated carbon (50% wet, 50 mg) and the mixture was hydrogenated (1 atm) for 1 hour. The catalyst was removed by filtration and the filtrate was concentrated in vacuo to give 2,2,2-trifluoro-N-[2-[4-[(4-hydroxy-3-methoxyphenyl)sulfonyl]phenyl]ethyl]acetamide (436 mg) as white foam.

(−)APCI-MS (m/z): 402 (M−H)⁻

Preparation 137

The following compound was obtained according to a similar manner to that of Preparation 134.

Ethyl 5-[[4-(2-aminoethyl)phenyl]sulfonyl]-2-methoxybenzoate

NMR (CDCl₃, δ): 1.36 (3H, t, J=7.1 Hz), 1.43 (2H, br), 2.77-2.85 (2H, m), 2.95-3.02 (2H, m), 3.95 (3H, s), 4.36 (2H, q, J=7.1 Hz), 7.35 (2H, d, J=8.3 Hz), 7.47-7.54 (2H, m), 7.80 (1H, d, J=7.9 Hz), 7.86 (2H, d, J=8.3 Hz) (+)APCI-MS (m/z): 364 (M+H)⁺

Preparation 138

The following compounds were obtained according to a similar manner to that of Example 76.

(1) (1R)-1-(3-Chlorophenyl)-2-[[2-[3-[(4-methoxyphenyl)thio]phenyl]ethyl]amino]ethanol

NMR (MeOD-d₄, δ): 2.50-2.90 (6H, m), 3.80 (3H, s), 4.60-4.80 (1H, m), 6.80-7.50 (12H, m).”

MS (m/z): 414 (M+H)

(2) 2-[[(2R)-2-(3-Chlorophenyl)-2-hydroxyethyl]amino]-ethylbenzene

NMR (DMSO-d₆, δ): 2.95-3.30 (6H, m), 5.00-5.10 (1H, m), 7.20-7.60 (9H, m)

Preparation 139

The following compound was obtained according to a similar manner to that of Example 50.

4-[[4-[2-[[(2R)-2-(3-Chlorophenyl)-2-hydroxyethyl]-amino]ethyl]phenyl]thio]butanoic acid hydrochloride

NMR (DMSO-d₆, δ): 1.60-1.80 (2H, m), 2.30-2.40 (2H, m), 2.80-3.30 (8H, m), 4.90-5.00 (1H, m), 7.10-7.45 (8H, m)

MS (m/z): 394 (M+H)

Preparation 140

The following compound was obtained according to a similar manner to that of Preparation 106.

4-[[4-[2-[N-(tert-Butoxycarbonyl)-N-[(2R)-2-(3-chlorophenyl)-2-hydroxyethyl]amino]ethyl]phenyl]-thio]butanoic acid

MS (m/z): 494 (M+H)

Preparation 141

To a solution of N-[2-(2-chlorophenyl)ethyl]-2,2,2-trifluoroacetamide (1.5 g) and 3-nitrobenzenesulfonyl chloride (1.19 g) in 1,2-dichloroethane (12 ml) was added trichloroaluminium (1.8 g) at room temprature and the mixture was refluxed for 24 hours. The resulting mixture was evaporated and partitioned between ethyl acetate and water. The organic layer was seperated, washed with water and brine, dried over magnesium sulfate and evaporated under reduced pressure. The residue was purified by column chromatography on silica gel (hexane/ethyl acetate=2/1) to give N-[2-[2-chloro-4-[(3-nitrophenyl)sulfonyl]phenyl]-ethyl]-2,2,2-trifluoroacetamide (530 mg) as a yellow solid.

(+)ESI-MS m/z: 459 (M+Na)⁺

Preparation 142

The following compound was obtained according to a similar manner to that of Preparation 67.

3-Chloro-4-[2-[(trifluoroacetyl)amino]ethyl]-benzenesulfonyl chloride

NMR (CDCl₃, δ): 3.15 (2H, t, J=7.0 Hz), 3.66-3.76 (2H, m), 6.47 (1H, br), 7.63-7.67 (1H, m), 7.86-7.92 (2H, m)

Preparation 143

The following compound was obtained according to a similar manner to that of Preparation 68.

N-[2-[2-Chloro-4-[(4-methoxyphenyl)sulfonyl]phenyl]-ethyl]-2,2,2-trifluoroacetamide

(+)ESI-MS m/z: 444 (M+Na)⁺

Preparation 144

A suspension of N-[2-[2-chloro-4-[(3-nitrophenyl)-sulfonyl]phenyl]ethyl]-2,2,2-trifluoroacetamide (520 mg) in methanol (5 ml) and tetrahydrofuran (5 ml) was hydrogenated over palladium on carbon (10% w/w, 50% wet, 220 mg) under hydrogen atmosphere for 2.5 hours. The catalyst was filtered off, and the filtrate was evaporated under reduced pressure to give N-[2-[4-[(3-aminophenyl)sulfonyl]phenyl]ethyl]-2,2,2-trifluoroacetamide (490 mg) as a yellow oil.

(−)ESI-MS (m/z): 371(M−H)⁻

Preparation 145

To a solution of N-[2-[4-[(3-aminophenyl)sulfonyl]-phenyl)ethyl]-2,2,2-trifluoroacetamide (200 mg), 4-dimethylaminopyridine (33 mg) and N,N-diisopropylethylamine (0.2 ml) in dichloromethane (3.0 ml) was added acetic anhydride (0.25 ml), and the mixture was stirred at room temperature for 2 hours. The mixture was diluted with dichloromethane, washed with saturated aqueous sodium bicarbonate and brine, dried over magnesium sulfate and evaporated under reduced pressure. The residue was purified by column chromatography on silica gel (hexane/ethyl acetate=1/2) to give N-[2-[4-[[3-(acetylamino)phenyl]sulfonyl]-phenyl)ethyl]-2,2,2-trifluoroacetamide (127 mg) as a colorless oil.

(+)ESI-MS (m/z): 437 (M+Na)⁺

Preparation 146

To a solution of 3-chloro-4-[2-[(trifluoroacetyl)-amino]ethyl]benzenesulfonyl chloride (600 mg) and ethyl phenoxyacetate (500 mg) in 1,2-dichloroethane (5.0 ml) was added trichloroaluminium (1.4 g) at room temprature and the mixture was refluxed for 8 hours. The resulting mixture was partitioned between ethyl acetate and water. The organic layer was seperated, washed with water and brine, dried over magnesium sulfate and evaporated under reduced pressure. The residue was suspended in 3.95N hydrogen chloride in ethanol (2.5 ml) and stirred for 3 hours. The solvent was removed by evaporation. The residue was purified by column chromatography on silica gel (hexane/ethyl acetate=2/1) to give ethyl [4-[[3-chloro-4-[2-[(trifluoroacetyl)amino]-ethyl]phenyl]sulfonyl]phenoxy]acetate (205 mg) as a white solid.

(+)ESI-MS (m/z): 516 (M+H)⁺

Preparation 147

The following compound was obtained according to a similar manner to that of Preparation 71.

Ethyl [4-[[4-(2-aminoethyl)-3-chlorophenyl]-sulfonyl]phenoxy]acetate

(+)ESI-MS (m/z): 398 (M+H)⁺

Preparation 148

A suspension of N-[2-[2-chloro-4-[(3-nitrophenyl)sulfonyl]phenyl]ethyl]-2,2,2-trifluoroacetamide (360 mg) and formaldehyde (37% w/w solution in water,180 μl ) in methanol (3.5 ml) and tetrahydrofuran (1.5 ml) was hydrogenated over palladium on carbon (10% w/w, 50% wet, 220 mg) under hydrogen atmosphere at 50° C. for 6 hours. The catalyst was filtered off, and the filtrate was evaporated under reduced pressure. The residue was purified by column chromatography on silica gel (hexane/ethyl acetate=2/1) to give N-[2-[4-[[3-(dimethylamino)phenyl]sulfonyl]phenyl]-ethyl]-2,2,2-trifluoroacetamide (146 mg) as a colorless oil.

(+)ESI-MS (m/z): 423 (M+Na)⁺

Preparation 149

To a solution of tert-butyl 2-(1-oxido-3-pyridyl)-ethylcarbamate (480 mg) in toluene (5.0 ml) was added diethylcarbamoyl chloride under 5° C. The mixture was stirred at the same temperature for 5 minutes. A solution of triethylamine (0.55 ml) and 3-methoxybenzenethiol (424 mg) in toluene (1.0 ml) was added to the mixture. The reaction mixture was refluxed for 4 hours. The resulting mixture was partitioned between ethyl acetate and water. The organic layer was seperated, washed with an aqueous solution of sodium hydroxide (1N) and brine, dried over magnesium sulfate and evaporated under reduced pressure. The residue was purified by column chromatography on silica gel (hexane/ethyl acetate=4/1) to give tert-butyl 2-[6-[(4-methoxyphenyl)thio]-3-pyridyl]ethylcarbamate (149 mg) as a white solid.

(+)ESI-MS (m/z): 361 (M+H)⁺

Preparation 150

The following compound was obtained according to a similar manner to that of Preparation 149.

tert-Butyl 2-[6-[(4-hydroxyphenyl)thio]-3-pyridyl]ethylcarbamate

(+)ESI-MS (m/z): 347 (M+H)⁺

Preparation 151

The following compound was obtained according to a similar manner to that of Preparation 70.

Ethyl [4-[[5-[2-[(tert-butoxycarbonyl)amino]ethyl]-2-pyridyl]sulfonyl)phenoxy]acetate

(+)ESI-MS (m/z): 465 (M+H)⁺

Preparation 152

The following compounds were obtained according to a similar manner to that of Preparation 63.

(1) N-[3-[[4-(2-Aminoethyl)phenyl]sulfonyl]phenyl]-N,N-dimethylamine

(+)ESI-MS (m/z): 305 (M+H)⁺

(2) N-[3-[[4-(2-Aminoethyl)phenyl]sulfonyl]phenyl]acetamide

(+)ESI-MS (m/z): 319 (M+H)⁺

(3) 2-[2-Chloro-4-[(4-methoxyphenyl)sulfonyl]phenyl]-ethanamine

(+)ESI-MS (m/z): 326 (M+H)⁺

### EXAMPLE 82

To a solution of ethyl (R)-[4-[[4-[2-[N-(tert-butoxycarbonyl)-N-[2-(3-chlorophenyl)-2-hydroxy-ethyl]amino]ethyl]phenyl]sulfonyl]phenoxy]acetate (231 mg) in methanol (3 ml) was added 40% methylamine in methanol (0.5 ml) at room temperature, and the mixture was sealed at the same temperature for 12 hours. The resulting mixture was evaporated under reduced pressure. The residue was dissolved into a mixture of water and ethyl acetate. After seperation, the organic layer was washed with brine, dried over anhydrous magnesium sulfate and evaporated under reduced pressure. The residue was dried in vacuo to give tert-butyl N-[(R)-2-(3-chlorophenyl)-2-hydroxyethyl]-N-[2-[4-[[4-[2-(methylamino)-2-oxoethoxy]phenyl]sulfonyl]-phenyl]ethyl]carbamate (198 mg).

NMR (CDCl₃, δ): 1.25-1.45 (9H, m), 2.7-2.9 (2H, m), 2.92 (3H, d, J=2.5 Hz), 3.1-3.55 (4H, m), 4.50 (2H, s), 4.8-4.85 (1H, m), 6.97 (2H, d, J=4.5 Hz), 7.1-7.4 (6H, m), 7.8-7.9 (4H, m) (+)ESI-MS (m/z): 625, 627 (M+Na)⁺

### EXAMPLE 83

At room temperature, to a solution of tert-butyl N-[(R)-2-(3-chlorophenyl)-2-hydroxyethyl]-N-[2-[4-[[4-[2-(methylamino)-2-oxoethoxy]phenyl]sulfonyl]phenyl]ethyl]-carbamate (195 mg) in ethyl acetate (2 ml) was added 4N hydrogen chloride in 1,4-dioxane (2 ml), and the mixture was stirred at the same temperature for 2 hours to give a precipitate. The precipitate was collected by filtration and washed with ethyl acetate, followed by dryness to give (R)-2-[4-[[4-[2-[[2-(3-chlorophenyl)-2-hydroxyethyl]amino]-ethyl]phenyl]sulfonyl]phenoxy]-N-methylacetamide hydrochloride (170 mg).

NMR (DMSO-d₆, δ): 2.63 (3H, d, J=4.6 Hz), 2.9-3.3 (6H, m), 4.58 (2H, s), 4.9-5.05 (1H, m), 7.05-7.2 (2H, m), 7.3-7.55 (6H, m), 7.8-7.95 (4H, m) (+)ESI-MS (m/z): 503, 505 (M-HCl+H)⁺

### EXAMPLE 84

Under nitrogen at 5° C., to a solution of sodium [4-[[4-[2-[N-(tert-butoxycarbonyl)-N-[(2R)-2-(3-chlorophenyl)-2-hydroxyethyl]amino]ethyl]phenyl]sulfonyl]phenoxy]acetate (65 mg) in N,N-dimethylformamide (2 ml) were added dimethylamine hydrochloride. (9.6 mg), 1-(3-dimethylaminopropyl)-3-ethylcarbodiimide hydrochloride (23 mg) and 1-hydroxybenzotriazole (16 mg), and the mixture was stirred at room temperature overnight. The resulting mixture was poured into saturated aqueous sodium bicarbonate and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=1:2 to 1:10) to give tert-butyl N-[(R)-2-(3-chlorophenyl)-2-hydroxyethyl]-N-[2-[4-[[4-[2-(dimethylamino)-2-oxoethoxy]phenyl]sulfonyl]phenyl]ethyl]carbamate (55 mg).

NMR (CDCl₃, δ): 1.2-1.5 (9H, m), 2.65-2.9 (2H, m), 2.97 (3H, s), 3.06 (3H, s), 3.1-3.45 (4H, m), 4.73 (2H, s), 4.8-4.95 (1H, m), 6.9-7.0 (2H, m), 7.15-7.4 (6H, m), 7.75-7.9 (4H, m) (+)ESI-MS (m/z): 639, 641 (M+Na)⁺

### EXAMPLE 85

The following compound was obtained according to a similar manner to that of Example 83.

(R)-2-[4-[[4-[2-[[2-(3-Chlorophenyl)-2-hydroxyethyl]amino]ethyl]phenyl]sulfonyl]phenoxy]-N,N-dimethylacetamide hydrochloride

NMR (DMSO-d₆, δ): 2.81 (3H, s), 2.9-3.45 (9H, m), 4.85-5.0 (3H, m), 7.0-7.15 (2H, m), 7.3-7.55 (6H, m), 7.8-7.95 (4H, m) (+)ESI-MS (m/z): 517, 519 (M-HCl+H)⁺

### EXAMPLE 86

To a solution of ethyl (R)-2-[4-[[4-[2-[5-(3-chlorophenyl)-2-oxo-1,3-oxazolidin-3-yl]ethyl]phenyl]-sulfonyl]phenoxy]-2-methylpropanoate (41 mg) was added 3N sodium hydoxide (3 ml) at room temperature, and the mixture was refluxed for 7 hours. The resulting mixture was cooled to 5° C., and to this one was added concentrated hydrochloric acid (0.75 ml). Ethanol was removed by evaporation under reduced pressure. To the residue was added 1N hydrochloric acid to give a precipitate. The precipitate was collected and washed with water, followed by dryness in vacuo to give (R)-2-[4-[[4-[2-[[2-(3-chlorophenyl)-2-hydroxyethyl]amino]-ethyl]phenyl]sulfonyl]phenoxy]-2-methylpropanoic acid hydrochloride (46 mg).

NMR (DMSO-d₆, δ): 1.55 (6H, s), 2.8-3.5 (6H, m), 4.85-4.95 (1H, m), 6.85-7.0 (2H, m), 7.3-7.55 (6H, m), 7.8-7.95 (4H, m) (−)ESI-MS (m/z): 516, 518 (M-HCl−H)⁻

### EXAMPLE 87

A mixture of (R)-2-[4-[[4-[2-[[2-(3-chlorophenyl)-2-hydroxyethyl]amino]ethyl]phenyl]sulfonyl]phenoxy]-2-methylpropanoic acid hydrochloride (29 mg), and 4N hydrogen chloride in ethanol (5 ml) was stirred at room temperature for 6 days. The mixture was evaporated under reduced pressure and dried to give ethyl (R)-2-[4-[[4-[2-[[2-(3-chlorophenyl)-2-hydroxyethyl]amino]ethyl]phenyl]sulfonyl]-phenoxy]-2-methylpropanoate hydrochloride (22 mg).

NMR (DMSO-d₆, δ): 1.11 (3H, t, J=7.1 Hz), 1.57 (6H, s), 2.8-3.55 (6H, m), 4.15 (2H, q, J=7.1 Hz), 4.85-5.0 (1H, m), 6.85-7.0 (2H, m), 7.3-7.55 (6H, m), 7.8-7.95 (4H, m) (+)ESI-MS (m/z): 546, 548 (M-HCl+H)⁺

### EXAMPLE 88

Under nitrogen at 5° C., to a solution of (R)-4-[[4-[2-[N-benzyl-N-[2-(3-chlorophenyl)-2-hydroxyethyl]amino]ethyl]-phenyl]sulfonyl]phenol (400 mg) in N,N-dimethylformamide (10 ml) was added sodium hydride (60% in oil, 34 mg), and the mixture was stirred at the same temperature for 1.5 hours. To this one was added bromoacetonitrile (0.059 ml) and the mixture was stirred at room temperature for 12 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane:ethyl acetate=2:1 to 1:1) to give (R)-[4-[[4-[2-]N-benzyl-N-[2-(3-chlorophenyl)-2-hydroxyethyl]amino]ethyl]phenyl]sulfonyl]phenoxy]-acetonitrile (322 mg).

NMR (CDCl₃, δ): 2.5-2.9 (6H, m), 3.5-3.95 (2H, m), 4.55-4.65 (1H, m), 4.80 (2H, s), 7.0-7.35 (11H, m), 7.75-7.9 (2H, m), 7.9-8.0 (2H, m) (+)ESI-MS (m/z): 561, 563 (M+H)⁺

### EXAMPLE 89

Under nitrogen at room temperature, to a solution of (R)-[4-[[4-[2-[N-benzyl-N-[2-(3-chlorophenyl)-2-hydroxyethyl)amino]ethyl]phenyl]sulfonyl]phenoxy]-acetonitrile (320 mg) in N,N-dimethylformamide (5 ml) were added ammonium chloride (153 mg) and sodium azide (185 mg), and the mixture was stirred at 100° C. for 4 hours. To the resulting mixture was added water at room temperature and the mixture was stirred for 30 minutes to give a precipitate. The precipitate was collected and washed with water, followed by dryness in vacuo to give (R)-2-[N-benzyl-N-[2-[4-[[4-(1H-tetrazol-5-ylmethoxy)phenyl]sulfonyl]phenyl]-ethyl]amino]-1-(3-chlorophenyl)ethanol (311 mg).

NMR (DMSO-d₆, δ): 2.65-2.9 (6H, m), 3.7-3.9 (2H, m), 4.65-4.8 (1H, m), 5.54 (2H, s), 7.05-7.4 (13H, m), 7.7-8.0 (4H, m) (−)ESI-MS (m/z): 602, 604 (M−H)⁻

### EXAMPLE 90

A mixture of (R)-2-(N-benzyl-N-(2-[4-[[4-(1H-tetrazol-5-ylmethoxy)phenyl]sulfonyl]phenyl]ethyl]amino]-1-(3-chlorophenyl)ethanol (302 mg), triethylamine (2 ml) and 10% palladium on activated carbon (50% wet, 100 mg) in a mixture of methanol (8 ml) and chlorobenzene (8 ml) was stirred at room temperature in the presence of hydrogen at an atmospheric pressure for 6 hours. After filtration, the filtrate was evaporated under reduced pressure. The residue was dissolved into methanol and added 10% hydrogen chloride in methanol. The mixture was evaporated under reduced pressure. The residue was purified by reversed phase chromatography (water:methanol=9:1 to 0:1), followed by treatment with 10% hydrogen chloride in methanol, evaporation under reduced pressure and dryness in vacuo to give (R)-1-(3-chlorophenyl)-2-[[2-[4-[[4-(1H-tetrazol-5-ylmethoxy)phenyl]sulfonyl]phenyl]ethyl]amino]ethanol hydrochloride (83 mg).

NMR (DMSO-d₆, δ): 3.0-3.3 (6H, m), 4.95-5.0 (1H, m), 5.60 (2H, s), 7.25-7.3 (2H, m), 7.35-7.55 (6H, m), 7.9-7.95 (4H, m) (−)ESI-MS (m/z): 512, 514 (M-HCl−H)⁻

### EXAMPLE 91

Under nitrogen at 5° C., to a solution of tert-butyl N-[(R)-2-(3-chlorophenyl)-2-hydroxyethyl]-N-[2-[4-[(4-hydroxyphenyl)sulfonyl]phenyl]ethyl]carbamate (236 mg) in N,N-dimethylformamide (5 ml) was added sodium hydride (60% in oil, 20 mg), and the mixture was stirred at the same temperature for 50 minutes. To this one was added ethyl bromodifluoroacetate (0.063 ml) and the mixture was stirred at room temperature for 6 days. The resulting mixture was poured into water and the aqueous mixture was extracted with ethyl acetate. The organic layer was washed with water, dried over anhydrous magnesium sulfate and evaporated under reduced pressure. The residue was purified by column chromatography on silica gel (hexane:ethyl acetate=3:1 to 2:1) to give tert-butyl N-[(R)-2-(3-chlorophenyl)-2-hydroxyethyl]-N-[2-[4-[[4-(difluoromethoxy)phenyl]sulfonyl]-phenyl]ethyl]carbamate (109 mg).

NMR (DMSO-d₆, δ): 1.2-1.5 (9H, m), 2.7-3.6 (6H, m), 4.8-4.95 (1H, m), 6.55 (1H, t, J=72.5 Hz), 7.15-7.45 (8H, m), 7.8-8.0 (4H, m) (+)ESI-MS (m/z): 604, 606 (M+Na)⁺

### EXAMPLE 92

To a solution of tert-butyl N-[(R)-2-(3-chlorophenyl)-2-hydroxyethyl]-N-[2-[4-[[4-(difluoromethoxy)-phenyl]sulfonyl]phenyl]ethyl]carbamate (106 mg) in ethyl acetate (1 ml) was added 4N hydrogen chloride in 1,4-dioxane (1 ml) at room temperature, and the mixture was stirred at the same temperature for 1.5 hours. The resulting mixture was evaporated under reduced pressure. The residue was dissolved into a mixture of saturated aqueous sodium bicarbonate and ethyl acetate. After seperation, the organic layer was washed successively with water and brine, dried over anhydrous magnesium sulfate and evaporated under reduced pressure. The residue was purified by column chromatography on silica gel (chloroform:methnol=20:1 to 15:1) to give (R)-1-(3-chlorophenyl)-2-[(2-[4-[[4-(difluoromethoxy)phenyl]sulfonyl]phenyl]ethyl]amino]ethanol (68 mg)

NMR (DMSO-d₆, δ): 2.55-2.9 (6H, m), 4.5-4.65 (1H, m), 7.0-7.75 (9H, m), 7.8-7.9 (2H, m), 7.95-8.05 (2H, m) (+)ESI-MS (m/z): 482, 484 (M+H)⁺

### EXAMPLE 93

To a solution of (R)-1-(3-chlorophenyl)-2-[[2-[4-[[4-(difluoromethoxy)phenyl]sulfonyl]phenyl]ethyl]amino]ethanol (34 mg) in ethanol (2 ml) was added 4N hydrogen chloride in ethanol (0.5 ml), and the mixture was evaporated under reduced pressure, followed by dryness in vacuo to give (R)-1-(3-chlorophenyl)-2-[[2-[4-[[4-(difluoromethoxy)phenyl]-sulfonyl]phenyl]ethyl]amino]ethanol hydrochloride (36 mg).

NMR (DMSO-d₆, δ): 2.9-3.4 (6H, m), 4.9-5.0 (1H, m), 7.0-7.75 (9H, m), 7.9-8.1 (4H, m) (+)ESI-MS (m/z): 482, 484 (M-HCl+H)⁺

### EXAMPLE 94

The following compound was obtained according to a similar manner to that of Example 93.

Ethyl (R)-2-[3-[[4-[2-[[2-(3-chlorophenyl)-2-hydroxyethyl]amino]ethyl]phenyl]sulfonyl]phenoxy]-2-methylpropanoate hydrochloride

NMR (DMSO-d₆, δ): 1.11 (3H, t, J=7.1 Hz), 2.95-3.3 (6H, m), 4.13 (2H, q, J=7.1 Hz), 4.9-5.0 (1H, m), 7.05-7.15 (1H, m), 7.2-7.6 (9H, m), 7.85-7.95 (2H, m) (+)ESI-MS (m/z): 546, 548 (M-HCl+H)⁺

### EXAMPLE 95

The following compounds were obtained according to a similar manner to that of Example 6.

(1) Ethyl (R)-2-[3-[[4-[2-[N-benzyl-N-[2-(3-chlorophenyl)-2-hydroxyethyl]amino]ethyl]phenyl]sulfonyl]phenoxy]-2-methylpropanoate

NMR (CDCl₃, δ): 1.21 (3H, t, J=7.1 Hz), 1.59 (6H, s), 2.5-2.9 (6H, s), 3.5-3.95 (2H, s), 4.20 (2H, q, J=7.1 Hz), 4.55-4.65 (1H, m), 6.95-7.0 (1H, m), 7.1-7.45 (13H, m), 7.5-7.55 (1H, m), 7.75-7.85 (2H, m) (+)APCI-MS (m/z): 636, 638 (M+H)⁺

(2) (S)-1-[N-Benzyl-N-[2-[4-[(3,4-dimethoxyphenyl)-sulfonyl]phenyl]ethyl]amino]-3-(4-fluorophenoxy)-2-propanol

NMR (CDCl₃, δ): 2.65-2.9 (6H, m), 3.5-4.0 (1H, m), 6.75-7.0 (5H, m), 7.1-7.3 (7H, m), 7.35 (1H, m), 7.5-7.55 (1H, m), 7.75-7.8 (2H, m) (+)ESI-MS (m/z): 580 (M+H)⁺

(3) (S)-1-[N-Benzyl-N-[2-[4-[(3,4-dimethoxyphenyl)-sulfonyl]phenyl]ethyl]amino]-3-(1H-indol-4-yloxy)-2-propanol

NMR (CDCl₃, δ): 2.65-2.9 (6H, m), 3.55-3.85 (2H, m), 3.89 (3H, s), 3.90 (3H, s), 4.05-4.2 (3H, m), 6.45-6.65 (2H, m), 6.85-7.55 (13H, m), 7.7-7.8 (2H, m) (+)ESI-MS (m/z): 601 (M+H)⁺

(4) Ethyl (R)-6-[[4-[2-[N-benzyl-N-[2-(3-chlorophenyl)-2-hydroxyethyl]amino]ethyl]phenyl]sulfonyl]-nicotinate

NMR (CDCl₃, δ): 1.39 (3H, t, J=7.1 Hz), 2.5-2.95 (6H, m), 3.5-3.95 (2H, m), 4.42 (2H, q, J=7.1 Hz), 6.55-6.65 (1H, m), 7.1-7.4 (1H, m), 7.95 (1H, d, J=8.3 Hz), 8.25 (1H, d, J=7.8 Hz), 8.50 (1H, dd, J=2.2, 8.3 Hz), 9.2 (1H, m) (+)ESI-MS (m/z): 579, 581 (M+H)⁺

(5) Methyl 4-[[4-[(2R)-2-[N-benzyl-N-[(2R)-2-(6-chloro-3-pyridyl)-2-hydroxyethyl]amino]propyl]phenyl]-sulfonyl]benzoate

MS (m/z): 579 (M+H)

(6) Methyl 4-[[4-[2-[N-benzyl-N-[(2R)-2-(6-chloro-3-pyridyl)-2-hydroxyethyl]amino]ethyl]phenyl]sulfonyl]-benzoate

MS (m/z): 565 (M+H)

(7) 4-[[4-[2-[N-Benzyl-N-[(2R)-2-(3-chlorophenyl)-2-hydroxyethyl]amino]ethyl]phenyl]sulfonyl]-2,6-dimethylphenol

MS (m/z): 550 (M+H)

(8) 3-[[2-[2-[N-Benzyl-N-[(2R)-2-(3-chlorophenyl)-2-hydroxyethyl]amino]ethyl]phenyl]sulfonyl]phenol

MS (m/z): 522 (M+H)

(9) (2S)-1-[N-Benzyl-N-[2-[3-[(4-methoxyphenyl)sulfonyl]-phenyl]ethyl]amino]-3-phenoxy-2-propanol

MS (m/z): 532 (M+H)

(10) 4-[[3-[2-[N-Benzyl-N-[(2R)-2-(3-chlorophenyl)-2-hydroxyethyl]amino]ethyl]phenyl]sulfonyl]phenol

MS (m/z): 522 (M+)

(11) 3-[[3-[2-[N-Benzyl-N-[(2R)-2-(3-chlorophenyl)-2-hydroxyethyl]amino]ethyl]phenyl]sulfonyl]phenol

MS (m/z): 522 (M+H)

(12) Ethyl [4-[[4-[2-[N-benzyl-N-[(2R)-2-(6-chloro-3-pyridyl)-2-hydroxyethyl]amino]ethyl]phenyl]sulfonyl]-phenoxy]acetate

MS (m/z): 609 (M+H)

(13) 4-[[4-[(2S)-2-[N-Benzyl-N-[(2R)-2-(3-chlorophenyl)-2-hydroxyethyl]amino]-3-hydroxypropyl]phenyl]sulfonyl]-phenol

NMR (DMSO-d₆, δ): 2.60-2.90 (5H, m), 3.18-3.35 (1H, m), 3.35-3.55 (1H, m), 3.67 (1H, d, J=14 Hz), 3.75 (1H, d, J=14 Hz), 4.37 (1H, br s, OH), 4.47 (1H, m), 5.02 (1H, br s, OH), 6.83-7.40 (13H, m), 7.71 (2H, d, J=8 Hz), 7.77 (2H, d, J=8 Hz), 10.62 (1H, br s) (+)ESI-MS (m/z): 552 (M+H)⁺

(14) Ethyl 4-[[4-[2-[[(2R)-2-(3-chlorophenyl)-2-hydroxyethyl]amino]-2-methylpropyl]phenyl]sulfonyl]-benzoate

NMR (CDCl₃, δ): 1.04 (3H, s), 1.06 (3H, s), 1.39 (3H, t, J=7 Hz), 2.50-3.05 (4H, m), 4.40 (2H, q, J=7 Hz), 4.58 (1H, dd, J=8 and 4 Hz), 7.10-7.45 (6H, m), 7.85 (2H, d, J=8 Hz), 8.01 (2H, d, J=8 Hz), 8.17 (2H, d, J=8 Hz) (+)ESI-MS (m/z): 516 (M+H)⁺

(15) Ethyl 3-[[4-[2-[[(2R)-2-(3-chlorophenyl)-2-hydroxyethyl]amino]-2-methylpropyl]phenyl]sulfonyl]-benzoate

NMR (CDCl₃, δ): 1.04 (3H, s), 1.06 (3H, s), 1.41 (3H, t, J=7 Hz), 2.62-2.82 (2H, m), 2.63 (1H, dd, J=12 and 8 Hz), 2.94 (1H, dd, J=12 and 4 Hz), 4.42 (2H, q, J=7 Hz), 4.58 (1H, dd, J=8 and 4 Hz), 7.10-7.45 (6H, m), 7.61 (1H, t, J=8 Hz), 7.87 (2H, d, J=8 Hz), 8.13 (1H, d, J=8 Hz), 8.24 (1H, d, J=8 Hz), 8.60 (1H, s) (+)ESI-MS (m/z): 516 (M+H)⁺

(16) Ethyl 3-[[4-[(2R)-2-[[(2R)-2-(3-chlorophenyl)-2-hydroxyethyl]amino]propyl]phenyl]sulfonyl]benzoate

NMR (CDCl₃, δ): 1.05 (3H, d, J=6 Hz), 1.41 (3H, t, J=7 Hz), 2.52-3.02 (5H, m), 4.40 (2H, q, J=7 Hz), 4.54 (1H, dd, J=8 and 4 Hz), 7.06-7.42 (6H, m), 7.59 (1H, t, J=8 Hz), 7.89 (2H, d, J=8 Hz), 8.12 (1H, d, J=8 Hz), 8.23 (1H, d, J=8 Hz), 8.59 (1H, s) (+)ESI-MS (m/z): 502 (M+H)⁺

(17) Ethyl 4-[[4-[(2S)-2-[[(2R)-2-(3-chlorophenyl)-2-hydroxyethyl]amino]propyl)phenyl]sulfonyl]benzoate

NMR (CDCl₃, δ): 1.04 (3H, d, J=6 Hz), 1.38 (3H, t, J=7 Hz), 2.45-3.06 (5H, m), 4.39 (2H, q, J=7 Hz), 4.59 (1H, dd, J=8 and 4 Hz), 7.07-7.42 (6H, m), 7.86 (2H, d, J=8 Hz), 8.00 (2H, d, J=8 Hz), 8.16 (2H, d, J=8 Hz) (+)ESI-MS (m/z): 502 (M+H)⁺

(18) Ethyl 4-[[4-[(2R)-2-[N-benzyl-N-[(2R)-2-(3-chlorophenyl)-2-hydroxyethyl]amino]propyl]phenyl]-sulfonyl]benzoate

NMR (CDCl₃, δ): 1.02 (3H, d, J=7 Hz), 1.38 (3H, t, J=7 Hz), 2.40-2.95 (4H, m), 3.00-3.26 (1H, m), 3.49 (1H, d, J=13 Hz), 3.50 (1H, br s), 3.80 (1H, d, J=13 Hz), 4.39 (2H, q, J=7 Hz), 4.55 (1H, dd, J=10 and 4 Hz), 6.90-7.40 (11H, m), 7.78 (2H, d, J=8 Hz), 8.01 (2H, d, J=8 Hz), 8.17 (2H, d, J=8 Hz) (+)ESI-MS (m/z): 592 (M+H)⁺

(19) Ethyl 3-[[3-[2-[N-benzyl-N-[(2R)-2-(3-chlorophenyl)-2-hydroxyethyl]amino]ethyl]phenyl]sulfonyl]benzoate

NMR (CDCl₃, δ): 1.39 (3H, t, J=7 Hz), 2.40-3.00 (6H, m), 3.57 (1H, d, J=13 Hz), 3.91 (1H, d, J=13 Hz), 4.38 (2H, q, J=7 Hz), 4.52 (1H, dd, J=8 and 4 Hz), 7.00-7.39 (10H, m), 7.43 (1H, t, J=8 Hz), 7.55 (1H, t, J=8 Hz), 7.68-7.88 (2H, m), 8.09 (1H, d, J=8 Hz), 8.20 (1H, d, J=8 Hz), 8.59 (1H, s) (+)ESI-MS (m/z): 578 (M+H)⁺

(20) Ethyl 4-[[3-[2-[N-benzyl-N-[(2R)-2-(3-chlorophenyl)-2-hydroxyethyl]amino]ethyl]phenyl]sulfonyl]benzoate

NMR (CDCl₃, δ): 1.38 (3H, t, J=7 Hz), 2.40-3.00 (6H, m), 3.53 (1H, br s, OH), 3.57 (1H, d, J=13 Hz), 3.92 (1H, d, J=13 Hz), 4.38 (2H, q, J=7 Hz), 4.52 (1H, dd, J=10 and 4 Hz), 7.02-7.50 (11H, m), 7.65-7.88 (2H, m), 7.98 (2H, d, J=8 Hz), 8.11 (2H, d, J=8 Hz) (+)ESI-MS (m/z): 578 (M+H)⁺

(21) Ethyl 4-[[4-[[N-benzyl-N-[(2R)-2-(3-chlorophenyl)-2-hydroxyethyl]amino]methyl]phenyl]sulfonyl]benzoate

NMR (CDCl₃, δ): 1.38 (3H, t, J=7 Hz), 2.58 (1H, dd, J=13 and 9 Hz), 2.64 (1H, dd, J=13 and 4 Hz), 3.45 (1H, br s, OH), 3.50 (1H, d, J=13 Hz), 3.55 (1H, d, J=14 Hz), 3.84 (1H, d, J=13 Hz), 3.89 (1H, d, J=14 Hz), 4.39 (2H, q, J=7 Hz), 4.68 (1H, dd, J=9 and 4 Hz), 6.95-7.45 (9H, m), 7.44 (2H, d, J=8 Hz), 7.91(2H, d, J=8 Hz), 8.00 (2H; d, J=8 Hz), 8.16 (2H, d, J=8 Hz) (+)ESI-MS (m/z): 564 (M+H)⁺

(22) Ethyl 4-[[4-[3-[N-benzyl-N-[(2R)-2-(3-chlorophenyl)-2-hydroxyethyl]amino]propyl]phenyl]sulfonyl]benzoate

NMR (CDCl₃, δ): 1.39 (3H, t, J=7 Hz), 1.80 (2H, quintet, J=7 Hz), 2.35-2.80 (6H, m), 3.48 (1H, d, J=13 Hz), 3.87 (1H, d, J=13 Hz), 3.90 (1H, br s), 4.39 (2H, q, J=7 Hz), 4.60 (1H, dd, J=10 and 4 Hz), 7.05-7.42 (11H, m), 7.82 (2H, d, J=8 Hz), 7.99 (2H, d, J=8 Hz), 8.15 (2H, d, J=8 Hz) (+)ESI-MS (m/z): 592 (M+H)⁺

(23) Ethyl 4-[[4-[2-[N-benzyl-N-[(2R)-2-(3-chlorophenyl)-2-hydroxyethyl]amino]ethyl]phenyl]sulfonyl]-2-fluorobenzoate

(+)APCI-MS (m/z): 596 (M+H)⁺

(24) Methyl 4-[[4-[2-[N-benzyl-N-[(2R)-2-(3-chlorophenyl)-2-hydroxyethyl]amino]ethyl]phenyl]sulfonyl]-2-chlorobenzoate

(+)APCI-MS (m/z): 598 (M+H)⁺

(25) Ethyl 4-[[4-[2-[N-benzyl-N-[(2R)-2-(3-chlorophenyl)-2-hydroxyethyl]amino]ethyl]phenyl]sulfonyl]-2-methylbenzoate

(+)APCI-MS (m/z): 592 (M+H)⁺

(26) Ethyl 4-[[4-[(2R)-2-[[(2R)-2-(3-chlorophenyl)-2-hydroxyethyl]amino]propyl]phenyl]sulfonyl]-2-fluorobenzoate

(+)APCI-MS (m/z): 520 (M+H)⁺

(27) Ethyl 2-chloro-4-[[4-1(2R)-2-[[(2R)-2-(3-chlorophenyl)-2-hydroxyethyl]amino]propyl]phenyl]sulfonyl]benzoate

(+)APCI-MS (m/z): 536 (M+H)⁺

(28) Ethyl 4-[[4-[(2R)-2-[[(2R)-2-(3-chlorophenyl)-2-hydroxyethyl]amino]propyl]phenyl]sulfonyl]-2-methylbenzoate

(+)APCI-MS (m/z): 516 (M+H)⁺

(29) Ethyl 4′-[[4-[2-[N-benzyl-N-[(2R)-2-(3-chlorophenyl)-2-hydroxyethyl]amino]ethyl]phenyl]sulfonyl]-2′-chloro-1,1′-biphenyl-4-carboxylate

(+)APCI-MS (m/z): 688 (M+H)⁺

(30) Ethyl 4′-[[4-[2-[N-benzyl-N-[(2R)-2-(3-chlorophenyl)-2-hydroxyethyl]amino]ethyl]phenyl]sulfonyl]-2′-chloro-1,1′-biphenyl-3-carboxylate

(+)APCI-MS (m/z): 688 (M+H)⁺

(31) 4-[[4-[[N-Benzyl-N-[(2R)-2-(3-chlorophenyl)-2-hydroxyethyl]amino]methyl]phenyl]sulfonyl]phenol

NMR (CDCl₃, δ): 2.58 (1H, dd, J=13 and 9 Hz), 2.65 (1H, dd, J=13 and 4 Hz), 3.50 (1H, d, J=13 Hz), 3.54 (1H, d, J=14 Hz), 3.84 (1H, d, J=13 Hz), 3.88 (1H, d, J=14 Hz), 4.66 (1H, dd, J=9 and 4 Hz), 6.88 (2H, d, J=9 Hz), 6.93-7.55 (1H, m), 7.81 (2H, d, J=9 Hz), 7.86 (2H, d, J=8 Hz) (+)ESI-MS (m/z): 508 (M+H)⁺

(32) 4-[[4-[3-[N-Benzyl-N-[(2R)-2-(3-chlorophenyl)-2-hydroxyethyl]amino]propyl]phenyl]sulfonyl]phenol

NMR (CDCl₃, δ): 1.80 (2H, quintet, J=7 Hz), 2.30-2.80 (6H, m), 3.48 (1H, d, J=13 Hz), 3.87 (1H, d, J=13 Hz), 4.59 (1H, dd, J=10 and 4 Hz), 6.88 (2H, d, J=9 Hz), 7.05-7.48 (11H, m), 7.78 (2H, d, J=8 Hz), 7.79 (2H, d, J=9 Hz) (+)ESI-MS (m/z): 536 (M+H)⁺

(33) 3-[[4-[2-[N-Benzyl-N-[(2R)-2-(3-chlorophenyl)-2-hydroxyethyl]amino]ethyl]phenyl]sulfonyl]-2,6-dimethylphenol

MS (m/z): 550 (M+H)

### EXAMPLE 96

A mixture of ethyl (R)-2-[3-[[4-[2-[N-benzyl-N-[2-(3-chlorophenyl)-2-hydroxyethyl]amino]ethyl]phenyl]sulfonyl]-phenoxy]-2-methylpropanoate (613 mg) and 10% palladium on activated carbon (50% wet, 300 mg) in a mixture of ethanol (6 ml) and chlorobenzene (6 ml) was stirred at room temperature in the presence of hydrogen at an atmospheric pressure for 2 hours. After filtration, the filtrate was evaporated under reduced pressure. The residue was dissolved into a mixture of saturated aqueous sodium bicarbonate and ethyl acetate. After seperation, the organic layer was dried over anhydrous magnesium sulfate and evaporated under reduced pressure. The residue was purified by column chromatography on silica gel (chloroform methanol=30:1 to 20:1) to give ethyl (R)-2-[3-[[4-[2-[[2-(3-chlorophenyl)-2-hydroxyethyl]amino]ethyl]phenyl]-sulfonyl]phenoxy]-2-methylpropanoate (275 mg).

NMR (DMSO-d₆, δ): 1.11 (3H, t, J=7.1 Hz), 1.54 (6H, s), 2.55-2.85 (6H, m), 4.11 (2H, q, J=7.1 Hz), 4.5-4.65 (1H, m), 7.05-7.6 (10H, m), 7.75-7.85 (2H, m) (+)ESI-MS (m/z): 546, 548 (M+H)⁺

### EXAMPLE 97

To a solution of ethyl (R)-2-[3-[[4-[2-[[2-(3-chlorophenyl)-2-hydroxyethyl]amino]ethyl]phenyl]sulfonyl]-phenoxy]-2-methylpropanoate (138 mg) in ethanol (5 ml) was added 1N sodium hydroxide (0.25 ml) at room temperature, and the mixture was stirred at 60° C. for 5 hours. The resulting mixture was evaporated under reduced pressure and dried in vacuo to give sodium 2-[3-[[4-[2-[[(2R)-2-(3-chlorophenyl)-2-hydroxyethyl]amino]ethyl]phenyl]sulfonyl]phenoxy]-2-methylpropanoate (128 mg).

NMR (DMSO-d₆, δ): 1.36 (6H, s), 2.5-2.85 (6H, m), 4.5-4.65 (1H, m), 7.0-7.05 (1H, m), 7.15-7.45 (9H, m), 7.75-7.85 (2H, m) (+)ESI-MS (m/z): 540, 542 (M+H)⁺

### EXAMPLE 98

The following compounds were obtained according to a similar manner to that of Example 35.

(1) (S)-1-[[2-[4-[(3,4-Dimethoxyphenyl)sulfonyl]phenyl]-ethyl]amino]-3-(4-fluorophenoxy)-2-propanol hydrochloride

NMR (DMSO-d₆, δ): 2.9-3.3 (6H, m), 3.82 (3H, s), 3.83 (3H, s), 3.9-3.95 (2H, m), 4.1-4.2 (1H, m), 6.9-7.2 (5H, m), 7.35-7.6 (4H, m), 7.9-7.95 (2H, m) (+)ESI-MS (m/z): 490 (M-HCl+H)⁺

(2) (S)-1-[[2-[4-[(3,4-Dimethoxyphenyl)sulfonyl]phenyl]-ethyl]amino]-3-(1H-indol-4-yloxy)-2-propanol hydrochloride

NMR (DMSO-d₆, δ): 3.0-3.5 (6H, m), 3.81 (3H, s), 3.82 (3H, s), 3.9-4.5 (3H, m), 6.45-6.8 (2H, m), 6.95-7.25 (4H, m), 7.35-7.55 (4H, m), 7.85-7.95 (2H, m) (+)ESI-MS (m/z): 511 (M+H)⁺

### EXAMPLE 99

To a solution of ethyl (R)-6-[[4-[2-[N-benzyl-N-[2-(3-chlorophenyl)-2-hydroxyethyl]amino]ethyl]phenyl]sulfonyl]-nicotinate (93 mg) in ethyl acetate (3 ml) was added 4N hydrogen chloride in ethyl acetate (0.12 ml) at room temperature, and the mixture was evaporated under reduced pressure. A mixture of the residue and 10% palladium on activated carbon (50% wet, 185 mg) in a mixture of ethanol (0.9 ml) and chlorobenzene (2.1 ml) was stirred at room temperature in the presence of hydrogen at an atmospheric pressure for 37 hours. After the catalyst was filtered off, the filtrate was evaporated under reduced pressure. The residue was dissolved into a mixture of saturated aqueous sodium bicarbonate and ethyl acetate. After seperation, the organic layer was washed with brine, dried over anhydrous magnesium sulfate and evaporated under reduced pressure. The residue was purified by column chromatography on silica gel (chloroform:methanol=20:1 to 15:1) to give ethyl (R)-6-[[4-[2-[[2-(3-chlorophenyl)-2-hydroxyethyl]amino]-ethyl]phenyl]sulfonyl]nicotinate (33 mg).

NMR (CDCl₃, δ): 1.40 (3H, t, J=7.1 Hz), 2.67 (1H, dd, J=8.9, 12.3 Hz), 2.8-3.05 (5H, m), 4.43 (2H, q, J=7.1 Hz), 4.63 (1H, dd, J=3.6, 8.8 Hz), 7.15-7.3 (6H, m), 7.95-8.05 (2H, m), 8.25-8.3 (1H, m), 8.52 (1H, dd, J=2.0, 8.1 Hz), 9.2 (1H, m) (+)ESI-MS (m/z): 488, 490 (M+H)⁺

### EXAMPLE 100

At room temperature, to a solution of ethyl (R)-[4-[[4-[2-[N-benzyl-N-[2-(3-chlorophenyl)-2-hydroxyethyl]amino]-ethyl]phenyl]sulfonyl]phenoxy]acetate (5.55 g) in ethyl acetate (56 ml) was added 4N hydrogen chloride in ethyl acetate (3.4 ml), and the mixture was evaporated under reduced pressure and dried in vacuo. A mixture of the residue and 10% palladium on activated carbon (50% wet, 0.28 g) in a mixture of ethanol (17 ml) and chlorobenzene (39 ml) was stirred at room temperature in the presence of hydrogen at an atmospheric pressure for 1.2 hours to give precipitates. To the reaction mixture was added ethanol to dissolve the precipitates. After removal of 10% palladium on activated carbon by filtration, the filtrate was evaporated under reduced pressure. The residue was dissolved into a mixture of saturated aqueous sodium bicarbonate and ethyl acetate. After separation, the organic layer was washed with brine, dried over magnesium sulfate and evaporated under reduced pressure. The residue was purified by column chromatography on silica gel (chloroform:methanol=30:1 to 20:1) to give ethyl (R)-[4-[[4-[2-[[2-(3-chlorophenyl)-2-hydroxyethyl]amino]ethyl]phenyl]sulfonyl]phenoxy]acetate (4.25 g).

NMR (CDCl₃, δ): 1.29 (3H, t, J=7.2 Hz), 2.6-3.0 (6H, m), 4.26 (2H, q, J=7.2 Hz), 4.6-4.7 (3H, m), 6.9-7.0 (2H, m), 7.15-7.4 (6H, m), 7.8-7.9 (4H, m) (+)ESI-MS (m/z): 518, 520 (M+H)⁺

### EXAMPLE 101

To a solution of sodium (R)-[4-[[4-[2-[[2-(3-chlorophenyl)-2-hydroxyethyl]amino]ethyl]phenyl]sulfonyl]-phenoxy]acetate (1.38 g) in methanol (10 ml) was added 1N hydrochloric acid (2.7 ml) at room temperature, and the mixture was stirred at the same temperature for 1 hour to give a precipitate. The precipitate was collected and washed with methanol, followed by dryness in vacuo to give (R)-[4-[[4-[2-[[2-(3-chlorophenyl)-2-hydroxyethyl]amino]-ethyl]phenyl]sulfonyl]phenoxy]acetic acid (1.23 g).

NMR (DMSO-d₆, δ): 2.7-3.1 (6H, m), 4.52 (1H, s), 4.75-4.85 (1H, m), 6.9-7.1 (2H, m), 7.25-7.5 (6H, m), 7.75-7.9 (4H, m) (−)ESI-MS (m/z): 488, 490 (M−H)⁻

### EXAMPLE 102

The following compounds were obtained according to a similar manner to that of Example 4.

(1) Ethyl 4-[[4-[(2R)-2-[N-benzyl-N-[(2R)-2-hydroxy-2-[3-(trifluoromethyl)phenyl]ethyl]amino]propyl]phenyl]-sulfonyl]benzoate

MS (m/z): 626 (M+H)

(2) Ethyl 4-[[4-[(2R)-2-[N-benzyl-N-[(2R)-2-(3-cyanophenyl)-2-hydroxyethyl]amino]propyl]phenyl]-sulfonyl]benzoate

MS (m/z): 583 (M+H)

(3) Methyl 4-[[4-[(2R)-2-[N-benzyl-N-[(2R)-2-(4-chlorophenyl)-2-hydroxyethyl]amino]propyl]phenyl]-sulfonyl]benzoate

MS (m/z): 579 (M+H)

(4) Methyl 4-[[4-[2-[N-benzyl-N-[(2R)-2-hydroxy-2-[3-(trifluoromethyl)phenyl]ethyl]amino]ethyl]phenyl]-sulfonyl]benzoate

MS (m/z): 598 (M+H)

(5) Methyl 4-[[4-[2-[N-benzyl-N-[(2R)-2-(3-cyanophenyl)-2-hydroxyethyl]amino]ethyl]phenyl]sulfonyl]benzoate

MS (m/z): 555 (M+H)

(6) Methyl 4-[[4-[2-[N-benzyl-N-[(2R)-2-(4-chlorophenyl)-2-hydroxyethyl]amino]ethyl]phenyl]sulfonyl]benzoate

MS (m/z): 565 (M+H)

### EXAMPLE 103

The following compounds were obtained according to a similar manner to that of Example 17.

(1) Methyl 4-[[4-[2-[[(2R)-2-(4-chlorophenyl)-2-hydroxyethyl]amino]ethyl]phenyl]sulfonyl]benzoate

MS (m/z): 474 (M+H)

(2) Methyl 4-[[4-[(2R)-2-[[(2R)-2-(4-chlorophenyl)-2-hydroxyethyl]amino]propyl]phenyl]sulfonyl]benzoate

MS (m/z): 489 (M+H)

(3) Methyl 4-[[4-[2-[[(2R)-2-(3-cyanophenyl)-2-hydroxyethyl]amino]ethyl]phenyl]sulfonyl]benzoate

MS (m/z): 465 (M+H)

(4) Methyl 4-[[4-[2-[[(2R)-2-hydroxy-2-[3-(trifluoromethyl)phenyl]ethyl]amino]ethyl]phenyl]-sulfonyl]benzoate

MS (m/z): 508 (M+H)

(5) Ethyl [3-[[2-[2-[[(2R)-2-(3-chlorophenyl)-2-hydroxyethyl]amino]ethyl]phenyl]sulfonyl]phenoxy]-acetate

MS (m/z): 519 (M+H)

(6) Ethyl 4-[[4-[(2R)-2-[[(2R)-2-hydroxy-2-[3-(trifluoromethyl)phenyl]ethyl]amino]propyl]phenyl]-sulfonyl]benzoate

MS (m/z): 536 (M+H)

(7) Ethyl 4-[[4-[(2R)-2-[[(2R)-2-(3-cyanophenyl)-2-hydroxyethyl]amino]propyl]phenyl]sulfonyl]benzoate

MS (m/z): 493 (M+H)

(8) Ethyl (2S)-2-[4-[[4-[2-[[(2R)-2-(3-chlorophenyl)-2-hydroxyethyl]amino]ethyl]phenyl]sulfonyl]phenoxy]-propanoate

MS (m/z): 532 (M+H)

(9) Ethyl (2S)-2-[4-[[4-[2-[[(2R)-2-(3-chlorophenyl)-2-hydroxyethyl]amino]ethyl]phenyl]sulfonyl]phenoxy]-propanoate

MS (m/z): 532 (M+H)

(10) Ethyl [4-[[4-[2-[[(2R)-2-(3-chlorophenyl)-2-hydroxyethyl]amino]ethyl]phenyl]sulfonyl]-2,6-dimethylphenoxy]acetate

MS (m/z): 546 (M+H)

(11) Ethyl [3-[[4-[2-[[(2R)-2-(3-chlorophenyl)-2-hydroxyethyl]amino]ethyl]phenyl]sulfonyl]-2,6-dimethylphenoxy]acetate

MS (m/z): 546 (M+H)

(12) Ethyl [4-[[3-[2-[[(2R)-2-(3-chlorophenyl)-2-hydroxyethyl]amino]ethyl]phenyl]sulfonyl]-phenoxy]acetate

MS (m/z): 518 (M+H)

(13) Ethyl [3-[[3-[2-[[(2R)-2-(3-chlorophenyl)-2-hydroxyethyl]amino]ethyl]phenyl]sulfonyl]phenoxy]-acetate

MS (m/z): 518 (M+H)

(14) (2S)-1-[[2-[3-[(4-Methoxyphenyl)sulfonyl]phenyl]-ethyl]amino]-3-phenoxy-2-propanol hydrochloride

NMR (MeOD-d₄, δ): 3.05-3.40 (6H, m), 3.85 (3H, s), 4.00-4.10 (2H, m), 4.20-4.40 (1H, m), 7.00-7.30 (7H, m), 7.60-7.90 (4H, m)

MS (m/z): 442 (M+H)

(15) (1R)-1-(3-Chlorophenyl)-2-[[2-[3-[(3-methoxyphenyl)-sulfonyl]phenyl]ethyl]amino]ethanol hydrochloride

NMR (MeOD-d₄, δ): 3.10-3.40 (6H, m), 3.85 (3H, s), 4.90-5.00 (1H, m), 7.00-7.90 (12H, m)

MS (m/z): 446 (M+H)

(16) Ethyl [4-[[4-(2-aminoethyl)phenyl]sulfonyl]phenoxy]-acetate

MS (m/z): 364 (M+H)

(17) (1R)-1-(3-Chlorophenyl)-2-[[2-[4-[[3-(2-hydroxyethoxy)phenyl]sulfonyl]phenyl]ethyl]amino]-ethanol hydrochloride

NMR (DMSO-d₆, δ): 3.0-3.4 (6H, m), 3.67-3.75 (2H, m), 4.01-4.09 (2H, m), 4.87-4.98 (1H, m), 6.3-6.33 (1H, br), 7.22-7.26 (1H, m), 7.35-7.53 (9H, m), 7.93-7.97 (2H, m), 8.96-9.26 (1H, br) (+)ESI-MS (m/z): 476(M-HCl+H)⁺

(18) Ethyl 3-[3-[[4-[2-[[(2R)-2-(3-chlorophenyl)-2-hydroxyethyl]amino]ethyl]phenyl]sulfonyl]phenoxy]-propanoate hydrochloride

NMR (DMSO-d₆, δ): 1.18 (3H, t, J=7.0 Hz), 2.79 (2H, t, J=5.9 Hz), 2.96-3.31 (6H, m), 4.10 (2H, q, J=7.0 Hz), 4.26 (2H, t, J=5.9 Hz), 4.89-4.95 (1H, m), 6.26-6.28 (1H, m), 7.22-7.54 (10H, m), 7.96(2H, d, J=8.2 Hz), 8.73 (1H, br) (+)ESI-MS (m/z): 532 (M-HCl+H)⁺

### EXAMPLE 104

The following compounds were obtained according to a similar manner to that of Example 23.

(1) Sodium 4-[[4-[(2R)-2-[[(2R)-2-(4-chlorophenyl)-2-hydroxyethyl]amino]propyl]phenyl]sulfonyl]benzoate

NMR (DMSO-d₆, δ): 2.50-2.90 (6H, m), 4.50-4.60 (1H, m), 7.30-7.50 (6H, m), 7.80 (4H, d, J=8 Hz), 8.00 (2H, d, J=8 Hz)

MS (m/z): 258 (M−H)

(2) Sodium 4-[[4-[2-[[(2R)-2-hydroxy-2-(3-pyridyl)ethyl]-amino]ethyl]phenyl]sulfonyl]benzoate

NMR (DMSO-d₆, δ): 2.50-2.80 (6H, m), 4.60-4.70 (1H, m), 7.20-8.50 (12H, m)

MS (m/z): 472 (M−H)

(3) Sodium 4-[[4-[(2R)-2-[[(2R)-2-hydroxy-2-(3-pyridyl)-ethyl]amino]propyl]phenyl]sulfonyl]benzoate

NMR (DMSO-d₆, δ): 0.90 (3H, d, J=5 Hz), 2.50-3.00 (5H, m), 4.60-4.70 (1H, m), 7.20-7.40 (3H, m), 7.79-8.10 (7H, m), 8.40-8.60 (2H, m)

MS (m/z): 439 (M−H)

(4) Sodium 4-[[4-[2-[[(2R)-2-(4-chlorophenyl)-2-hydroxyethyl]amino]propyl]phenyl]sulfonyl]benzoate

NMR (DMSO-d₆, δ): 0.86 (3H, d, J=6 Hz), 2.50-2.90 (5H, m), 4.50-4.65 (1H, m), 7.30-7.50 (6H, m), 7.80 (4H, d, J=8 Hz), 7.90 (2H, d, J=8 Hz)

MS (m/z): 472 (M−H)

(5) Sodium 4-[[4-[2-[[(2R)-2-(3-cyanophenyl)-2-5 hydroxyethyl]amino]ethyl]phenyl]sulfonyl]benzoate

NMR (DMSO-d₆, δ): 2.60-2.80 (6H, m), 4.60-4.80 (1H, m), 7.40-8.10 (12H, m)

MS (m/z): 451 (M+H)

(6) Sodium 4-[[4-[2-[[(2R)-2-hydroxy-2-[3-(trifluoromethyl)phenyl]ethyl]amino]ethyl]phenyl]-sulfonyl]benzoate

NMR (DMSO-d₆, δ): 2.50-2.80 (6H, m), 4.60-4.70 (1H, m), 7.30-8.05 (12H, m)

MS (m/z): 494 (M+H)

(7) Sodium 4-[[4-[(2R)-2-[[(2R)-2-hydroxy-2-[3-(trifluoromethyl)phenyl]ethyl]amino]propyl]phenyl]-sulfonyl]benzoate

NMR (DMSO-d₆, δ): 0.87 (3H, d, J=6 Hz), 2.60-2.80 (5H, m), 4.60-4.70 (1H, m), 7.10-8.00 (12H, m)

MS (m/z): 506 (M−H)

(8) Sodium [3-[[2-[2-[[(2R)-2-(3-chlorophenyl)-2-hydroxyethyl]amino]ethyl]phenyl]sulfonyl]phenoxy]-acetate

NMR (DMSO-d₆, δ): 2.50-2.70 (2H, m), 2.80-3.00 (4H, m), 4.50-4.60 (1H, m), 7.10-7.80 (1H, m), 8.00-8.08 (1H, m)

MS (m/z): 490 (M+H)

(9) Sodium 4-[[4-[(2R)-2-[[(2R)-2-(3-cyanophenyl)-2-hydroxyethyl]amino]propyl]phenyl]sulfonyl]benzoate

NMR (DMSO-d₆, δ): 0.88 (3H, d, J=3 Hz), 2.50-2.80 (5H, m), 4.60-4.70 (1H, m), 7.30-8.00 (12H, m)

MS (m/z): 463 (M−H)

(10) Sodium (2S)-2-[4-[[4-[2-[[(2R)-2-(3-chlorophenyl)-2-hydroxyethyl]amino]ethyl]phenyl]sulfonyl]-phenoxy]propanoate

NMR (DMSO-d₆, δ): 1.35 (3H, d, J=6 Hz), 2.50-2.80 (6H, m), 4.30 (1H, q, J=6 Hz), 6.90 (1H, d, J=8 Hz), 7.20-7.40 (6H, m), 7.70-7.80 (4H, m)

MS (m/z): 502 (M−H)

(11) Sodium (2R)-2-[4-[[4-[2-[[(2R)-2-(3-chlorophenyl)-2-hydroxyethyl]amino]ethyl]phenyl]sulfonyl]phenoxy]-propanoate

NMR (DMSO-d₆, δ): 1.37 (3H, d, J=6 Hz), 2.50-2.80 (6H, m), 4.30 (1H, q, J=6 Hz), 6.90 (1H, d, J=8 Hz), 7.20-7.40 (6H, m), 7.70-7.80 (4H, m)

MS (m/z): 502 (M−H)

(12) Sodium [4-[[4-[(2R)-2-[[(2R)-2-(3-chlorophenyl)-2-hydroxyethyl]amino]propyl]phenyl]sulfonyl]-2-methylphenoxy]acetate

NMR (DMSO-d₆, δ): 0.85 (3H, d, J=6 Hz), 2.18 (3H, s), 2.50-2.90 (5H, m), 4.23 (2H, s), 4.40-4.60 (1H, m), 6.70-6.80 (1H, m), 7.20-7.40 (6H, m), 7.70-7.80 (4H, m)

MS (m/z): 516 (M−H)

(13) Sodium [4-[[4-[(2R)-2-[[(2R)-2-(3-chlorophenyl)-2-hydroxyethyl]amino]propyl]phenyl]sulfonyl]-2-fluorophenoxy]acetate

NMR (DMSO-d₆, δ): 0.90 (3H, d, J=6H), 2.50-2.90 (5H, m), 4.25 (2H, s), 4.50-4.60 (1H, m), 6.90-8.00 (11H, m)

MS (m/z): 520 (M−H)

(14) Sodium [4-[[4-[(2R)-2-[[(2R)-2-(3-chlorophenyl)-2-hydroxyethyl]amino]ethyl]phenyl]sulfonyl]-2,6-dimethylphenoxy]acetate

NMR (DMSO-d₆, δ): 2.26 (6H, s), 2.50-2.90 (6H, m), 3.90 (2H, s), 4.50-4.60 (1H, m), 7.10-7.90 (10H, m)

MS (m/z): 518 (M+H)

(15) Sodium [3-[[4-[(2R)-2-[[(2R)-2-(3-chlorophenyl)-2-hydroxyethyl]amino]ethyl]phenyl]sulfonyl]-2,6-dimethylphenoxy]acetate

NMR (DMSO-d₆, δ): 2.25 (3H, s), 2.27 (3H, s), 2.50-2.90 (6H, m), 3.80 (2H, s), 4.50-4.60 (1H, m), 7.10-7.90 (10H, m)

MS (m/z): 518 (M+H)

(16) Sodium [4-[[3-[2-[[(2R)-2-(3-chlorophenyl)-2-hydroxyethyl]amino]ethyl]phenyl]sulfonyl]phenoxy]-acetate

NMR (MeOD-d₄, δ): 2.70-3.10 (6H, m), 4.40 (2H, s), 4.70-4.80 (1H, m), 7.00-7.10 (2H, m), 7.20-7.60 (6H, m), 7.80-7.90 (4H, m)

MS (m/z): 488 (M−H)

(17) Sodium [3-[[3-[2-[[(2R)-2-(3-chlorophenyl)-2-hydroxyethyl]amino]ethyl]phenyl]sulfonyl]phenoxy]-acetate

NMR (MeOD-d₄, δ): 2.70-3.10 (6H, m), 4.40 (2H, s), 4.70-4.80 (1H, m), 7.00-7.60 (10H, m), 7.80-7.90 (2H, m)

MS (m/z): 488 (M−H)

(18) Sodium [4-[[4-[2-[[(2R)-2-hydroxy-2-(3-pyridyl)-ethyl]amino]ethyl]phenyl]sulfonyl]phenoxy]acetate

NMR (DMSO-d₆, δ): 2.50-2.80 (6H, m), 4.19 (2H, s), 4.60-4.70 (1H, m), 6.90-7.00 (2H, m), 7.10-7.90 (9H, m), 8.40-8.60 (1H, m)

MS (m/z): 478 (M+Na)

(19) Sodium [4-[[4-[2-[[(2R)-2-(6-chloro-3-pyridyl)-2-hydroxyethyl]amino]ethyl]phenyl]sulfonyl]phenoxy]-acetate

NMR (DMSO-d₆, δ): 2.70-2.-90 (6H, m), 4.20 (2H, s), 4.60-4.70 (1H, m), 6.90-7.00 (2H, m), 7.10-7.50 (3H, m), 7.70-7.90 (5H, m), 8.30-8.40 (1H, m)

MS (m/z): 512 (M+Na)

(20) Sodium [4-[[4-[[[(2R)-2-(3-chlorophenyl)-2-hydroxyethyl]amino]methyl]phenyl]sulfonyl]phenoxy]-acetate

NMR (DMSO-d₆, δ): 2.60 (2H, d, AB of ABX), 3.76 (2H, s), 4.09 (2H, s), 4.65 (1H, t, X of ABX), 5.50 (1H, br s, OH), 6.93 (2H, d, J=9 Hz), 7.10-7.50 (4H, m), 7.49 (2H, d, J=8 Hz), 7.77 (2H, d, J=9 Hz), 7.82 (2H, d, J=8 Hz) (+)ESI-MS (m/z): 476 (free, M+H)⁺

(21) Sodium [4-[[4-[3-[[(2R)-2-(3-chlorophenyl)-2-hydroxyethyl]amino]propyl]phenyl]sulfonyl]phenoxy]-acetate

NMR (DMSO-d₆, δ): 1.66 (2H, quintet, J=7 Hz), 2.37-2.77 (6H, m), 4.18 (2H, s), 4.60 (1H, m), 5.44 (1H, br s, OH), 6.92 (2H, d, J=9 Hz), 7.12-7.50 (6H, m), 7.77 (2H, d, J=9 Hz), 7.78 (2H, d, J=8 Hz) (+)ESI-MS (m/z): 504 (free, M+H)⁺

(22) Sodium [4-[[3-chloro-4-[2-[[(2R)-2-(3-chlorophenyl)-2-hydroxyethyl]amino]ethyl]phenyl]sulfonyl]phenoxy]-acetate

NMR (DMSO-d₆, δ): 3.02-3.35 (6H, m),4.01 (2H, s), 4.52-4.62 (1H, m), 5.62 (1H, br), 6.89-6.94 (2H, m), 7.19-7.36 (4H, m), 7.55-7.90 (5H, m) (−)ESI-MS (m/z): 522, 524 (M−Na−H)⁻

(23) Sodium [4-[[5-[2-[[(2R)-2-(3-chlorophenyl)-2-hydroxyethyl]amino]ethyl]-2-pyridyl]sulfonyl]-phenoxy]acetate

NMR (DMSO-d₆, δ): 2.60-2.76 (6H, m), 4.18 (2H, s), 4.56-4.59 (1H, m), 5.46 (1H, br), 6.92-6.95 (2H, m), 7.24-7.36 (4H, m), 7.75-7.79 (2H, m), 7.88-7.91 (1H, m), 8.01-8.03 (1H, m), 8.53 (1H, s) (−)ESI-MS (m/z): 489 (M−Na−H)⁻

### EXAMPLE 105

Methyl 4-[[4-[(2R)-2-[N-benzyl-N-[(2R)-2-(6-chloro-3-pyridyl)-2-hydroxyethyl]amino]propyl]phenyl]sulfonyl]-benzoate (480 mg), ammonium formate (200 mg) and palladium on carbon powder (120 mg) in methanol (5 ml) was refluxed for 30 minutes. The reaction mixture was filtrated and poured into water and extracted with ethyl acetate. The organic layer was washed with brine, dried over magnesium sulfate, and evaporated in vacuo. A mixture of the residue was chromatographed (chloroform-methanol) over silica gel to give methyl 4-[[4-[(2R)-2-[[(2R)-2-hydroxy-2-(3-pyridyl)ethyl]amino]propyl]phenyl]sulfonyl]benzoate (150 mg) as a colorless foam.

MS (m/z): 455 (M+H)

### EXAMPLE 106

The following compound was obtained according to a similar manner to that of Example 105.

Methyl 4-[[4-[2-[[2R)-2-hydroxy-2-(3-pyridyl)ethyl]-amino]ethyl]phenyl]sulfonyl]benzoate

MS (m/z): 441 (M+H)

### EXAMPLE 107

The following compounds were obtained according to a similar manner to that of Preparation 70.

(1) Ethyl [3-[[2-[2-[N-benzyl-N-[(2R)-2-(3-chlorophenyl)-2-hydroxyethyl]amino]ethyl]phenyl]sulfonyl]phenoxy]-acetate

MS (m/z): 609 (M+H)

(2) Ethyl (2R)-2-[4-[[4-[2-[N-benzyl-N-[(2R)-2-(3-chlorophenyl)-2-hydroxyethyl]amino]ethyl]phenyl]-sulfonyl]phenoxy]propanoate

MS (m/z): 622 (M+H)

(3) (1R)-2-[N-Benzyl-N-[2-[3-[(3-methoxyphenyl)sulfonyl]-phenyl]ethyl]amino]-1-(3-chlorophenyl)ethanol

MS (m/z): 536 (M+H)

(4) Ethyl [4-[[4-[(2S)-2-[N-benzyl-N-[(2R)-2-(3-chlorophenyl)-2-hydroxyethyl]amino]-3-hydroxypropyl]phenyl]sulfonyl]phenoxy]acetate

NMR (CDCl₃, δ): 1.28 (3H, t, J=7 Hz), 2.53-3.23 (5H, m), 3.40-3.70 (2H, m), 3.70 (1H, d, J=13 Hz), 3.85 (1H, d, J=13 Hz), 4.26 (2H, q, J=7 Hz), 4.49 (1H, dd, J=8 and 4 Hz), 4.65 (2H, s), 6.96 (2H, d, J=9 Hz), 7.00-7.40 (11H, m), 7.78 (2H, d, J=8 Hz), 7.87 (2H, d, J=9 Hz) (+)ESI-MS (m/z): 638 (M+H)⁺

(5) Ethyl [4-[[4-[2-[N-benzyl-N-[(2R)-2-(3-chlorophenyl)-2-hydroxyethyl]amino]ethyl]phenyl]sulfonyl]-2,6-dimethylphenoxy]acetate

MS (m/z): 636 (M+H)

(6) Ethyl [3-[[4-[2-[N-benzyl-N-[(2R)-2-(3-chlorophenyl)-2-hydroxyethyl]amino]ethyl]phenyl]sulfonyl]-2,6-dimethylphenoxy)acetate

MS (m/z): 636 (M+H)

(7) Ethyl (2S)-2-[4-[[4-[2-[N-benzyl-N-[(2R)-2-(3-chlorophenyl)-2-hydroxyethyl]amino]ethyl]phenyl]-sulfonyl]phenoxy]propanoate

MS (m/z): 622 (M+H)

(8) Ethyl [3-[[3-[2-[N-benzyl-N-[(2R)-2-(3-chlorophenyl)-2-hydroxyethyl]amino]ethyl]phenyl]sulfonyl]phenoxy]-acetate

MS (m/z): 608 (M+H)

(9) Ethyl [4-[[3-(2-[N-benzyl-N-[(2R)-2-(3-chlorophenyl)-2-hydroxyethyl]amino]ethyl]phenyl]sulfonyl]phenoxy]-acetate

MS (m/z): 608 (M+H)

### EXAMPLE 108

The following compounds were obtained according to a similar manner to that of Example 76.

(1) Ethyl [4-[[4-[(2R)-2-[[(2R)-2-(3-chlorophenyl)-2-hydroxyethyl]amino]propyl]phenyl]sulfonyl]-2-methylphenoxy]acetate

MS (m/z): 546 (M+H)

(2) Ethyl [4-[[4-[(2R)-2-[[(2R)-2-(3-chlorophenyl)-2-hydroxyethyl]amino]propyl]phenyl]sulfonyl]-2-fluorophenoxy]acetate

MS (m/z): 550 (M+H)

(3) Ethyl [4-[[4-[2-[[(2R)-2-[6-chloro-3-pyridyl)-2-hydroxyethyl]amino]ethyl]phenyl]sulfonyl]phenoxy]-acetate

MS (m/z): 519 (M+H)

### EXAMPLE 109

[4-[[4-[2-[[(2R)-2-(3-Chlorophenyl)-2-hydroxyethyl]-amino]-2-methylpropyl]phenyl]sulfonyl]phenoxy]acetate (50 mg) was triturated with 4N hydrogenchloride in 1,4-dioxane (1.0 ml) to give [4-[[4-[2-[[(2R)-2-(3-chlorophenyl)-2-hydroxyethyl]amino]-2-methylpropyl]phenyl]sulfonyl]phenoxy]-acetate hydrochloride (50 mg) as a colorless powder.

NMR (CDCl₃, δ): 1.04 (3H, s), 1.07 (3H, s), 1.30 (3H, t, J=7 Hz), 2.50-3.10 (4H, m), 3.85 (3H, s), 4.30 (2H, q, J=7 Hz), 4.40-4.55 (1H, m), 4.66 (2H, s), 6.90-7.00 (2H, m), 7.10-7.40 (6H, m), 7.70-7.90 (4H, m)

MS (m/z): 547 (M+H)

### EXAMPLE 110

The following compounds were obtained according to a similar manner to that of Example 109.

(1) Ethyl [2-chloro-4-[[4-[(2R)-2-[[(2R)-2-(3-chlorophenyl)-2-hydroxyethyl]amino]propyl]phenyl]-sulfonyl]phenoxy]acetate hydrochloride

NMR (DMSO-d₆, δ): 1.10 (3H, d, J=3 Hz), 1.17 (3H, t, J=3 Hz), 2.70-2.80 (1H, m), 3.00-3.40 (4H, m), 4.10 (2H, q, J=3 Hz), 4.90-5.10 (3H, m), 7.10-7.40 (7H m), 7.70-7.90 (4H, m)

MS (m/z): 566 (M+H)

(2) Ethyl [4-[[4-[2-[[(2R)-2-(3-chlorophenyl)-2-hydroxyethyl]amino]ethyl]phenyl]sulfonyl]-2-methylphenoxy]acetate hydrochloride

NMR (DMSO-d₆, δ): 1.10 (3H, t, J=3 Hz), 2.23 (3H, s), 3.00-3.40 (6H, m), 4.10 (2H, q, J=3 Hz), 4.90-5.10 (3H, m), 7.00-7.40 (7H, m), 7.70-7.90 (4H, m)

MS (m/z): 532 (M+H)

(3) Ethyl [4-[[4-[2-[[(2R)-2-(3-chlorophenyl)-2-hydroxyethyl]amino]ethyl]phenyl]sulfonyl]-2-fluorophenoxy]acetate hydrochloride

MS (m/z): 536 (M+H)

(4) Ethyl (2R)-2-[4-[[4-[2-[[(2R)-2-(3-chlorophenyl)-2-hydroxyethyl]amino]ethyl]phenyl]sulfonyl]phenoxy]-propanoate hydrochloride

NMR (DMSO-d₆, δ): 1.20 (3H, t, J=7 Hz), 1.50 (3H, d, J=7 Hz), 2.90-3.20 (6H, m), 4.23 (2H, q, J=7 Hz), 4.90-5.00 (1H, m), 5.10 (1H, q, J=7 Hz), 6.35 (1H, d, J=4 Hz), 7.05 (1H, d, J=8 Hz), 7.30-7.50 (6H, m), 7.80-7.90 (4H, m)

MS (m/z): 532 (M+H)

(5) Ethyl [4-[[4-[(2R)-2-[[(2R)-2-(3-chlorophenyl)-2-hydroxyethyl]amino]propyl]phenyl]sulfonyl]-2-fluorophenoxy]acetate hydrochloride

NMR (DMSO-d₆, δ): 1.10-1.20 (6H, m), 2.80-3.50 (5H, m), 4.20 (2H, q, J=7 Hz), 5.00 (2H, s), 5.10-5.20 (1H, m), 7.20-8.00 (11H, m)

MS (m/z): 550 (M+H)

(6) Ethyl [4-[[4-[(2R)-2-[[(2R)-2-(3-chlorophenyl)-2-hydroxyethyl]amino]propyl]phenyl]sulfonyl]-2-methylphenoxy]acetate hydrochloride

NMR (DMSO-d₆, δ): 1.10 (3H, d, J=7 Hz), 1.20 (3H, t, J=7 Hz), 2.23 (3H, s), 2.60-3.20 (5H, m), 4.23 (2H, q, J=7 Hz), 4.94 (2H, s), 5.10-5.20 (1H, m), 7.05 (1H, d, J=8 Hz), 7.30-7.50 (6H, m), 7.80-7.90 (4H, m)

MS (m/z): 546 (M+H)

(7) Ethyl [4-[[4-[(2R)-2-[[(2R)-2-(3-chlorophenyl)-2-hydroxyethyl]amino]ethyl]phenyl]sulfonyl]-2,6-dimethylphenoxy]acetate hydrochloride

NMR (DMSO-d₆, δ): 1.20 (3H, t, J=7 Hz), 2.29 (6H, s), 2.90-3.30 (6H, m), 4.20 (2H, q, J=7 Hz), 4.50 (2H, s), 5.00-5.10 (1H, m), 7.20-7.90 (10H, m)

MS (m/z): 546 (M+H)

(8) Ethyl [3-[[4-[(2R)-2-[[(2R)-2-(3-chlorophenyl)-2-hydroxyethyl]amino]ethyl]phenyl]sulfonyl]-2,6-dimethylphenoxy]acetate hydrochloride

NMR (DMSO-d₆, δ): 1.20 (3H, t, J=7 Hz), 2.28 (3H, s), 2.30 (3H, s), 2.90-3.30 (6H, m), 4.20 (2H, q, J=7 Hz), 4.50 (2H, s), 5.00-5.10 (1H, m), 7.30-7.90 (10H, m)

MS (m/z): 546 (M+H)

(9) Ethyl [4-[[3-[2-[[(2R)-2-(3-chlorophenyl)-2-hydroxyethyl]amino]ethyl]phenyl]sulfonyl]phenoxy]-acetate hydrochloride

NMR (DMSO-d₆, δ): 1.20 (3H, t, J=7 Hz), 3.00-3.40 (6H, m), 4.20 (2H, q, J=7 Hz), 3.80 (3H, s), 4.90 (2H, s), 4.90-5.05 (1H, m), 7.00-7.10 (2H, m), 7.30-7.60 (6H, m), 7.80-7.90 (4H, m)

MS (m/z): 518 (M+H)

(10) Ethyl [3-[[3-[2-[[(2R)-2-(3-chlorophenyl)-2-hydroxyethyl]amino]ethyl]phenyl]sulfonyl]phenoxy]-acetate hydrochloride

NMR (DMSO-d₆, δ): 1.25 (3H, t, J=7 Hz), 3.00-3.50 (6H, m), 4.20 (2H, q, J=7 Hz), 3.80 (3H, s), 4.90 (2H, s), 4.90-5.05 (1H, m), 7.00-7.10 (2H, m), 7.30-7.60 (6H, m), 7.80-7.90 (4H, m)

MS (m/z): 518 (M+H)

### EXAMPLE 111

The following compound was obtained according to a similar manner to that of Example 43.

4-[[[4-[2-[N-(tert-Butoxycarbonyl)-N-[(2R)-2-(3-chlorophenyl)-2-hydroxyethyl]amino]ethyl]phenyl]sulfonyl]-methyl]benzoic acid

MS (m/z): 574 (M+H)

### EXAMPLE 112

The following compounds were obtained according to a similar manner to that of Example 50.

(1) 4-[[[4-[2-[[(2R)-2-(3-Chlorophenyl)-2-hydroxyethyl]-amino]ethyl]phenyl]sulfonyl]methyl]benzoic acid hydrochloride

NMR (DMSO-d₆, δ): 2.80-3.50 (6H, m), 4.90 (2H, s), 5.00-5.10 (1H, m), 7.20-8.20 (13H, m)

MS (m/z): 474 (M+H)

(2) 4-[[4-[2-[[(2R)-2-(3-Chlorophenyl)-2-hydroxyethyl]-amino]ethyl]phenyl]sulfonyl]butanoic acid hydrochloride

NMR (DMSO-d₆, δ): 1.60-1.80 (2H, m), 2.30-2.40 (2H, m), 3.00-3.40 (8H, m), 5.00-5.10 (1H, m), 7.30-7.70 (6H, m), 7.90 (2H, d, J=8 Hz)

MS (m/z): 426 (M+H)

### EXAMPLE 113

A solution of N-benzyl-2-[3-[(4-methoxyphenyl)-sulfonyl]phenyl]ethanamine (141 mg), (2R)-2-(3-chlorophenyl)oxirane (57.1 mg) in ethanol (5 ml) was refluxed for 20 hours and evaporated in vacuo. To the residue were added 10% palladium on activated carbon (50% wet, 20 mg), methanol (3.0 ml) and chlorobenzene (3.0 ml) and then stirred at room temperature in the presence of hydrogen at an atmospheric pressure for 1 hour. After filtration, the filtrate was evaporated in vacuo. To the residue was added 4N hydrogen chloride in 1,4-dioxane (1.0 ml), and the mixture was stirred at room temperature for 1 hour and evaporated in vacuo to give (1R)-1-(3-chlorophenyl)-2-[[2-[3-[(4-methoxyphenyl)sulfonyl]phenyl]-ethyl]amino]ethanol hydrochloride (50 mg) as a colorless foam.

NMR (MeOD-d₄, δ): 3.00-3.50 (6H, m), 3.80 (3H, s), 4.90-5.00 (1H, m), 7.00-7.10 (2H, m), 7.30-7.60 (6H, m), 7.80-8.00 (4H, m)

MS (m/z): 446 (M+H)

### EXAMPLE 114

The following compound was obtained according to a similar manner to that of Example 8.

Ethyl [4-[[4-[2-[[(2R)-2-hydroxy-2-(3-pyridyl)ethyl]-amino]ethyl]phenyl]sulfonyl]phenoxy]acetate

NMR (CDCl₃, δ): 1.25 (3H, t, J=9 Hz), 2.60-3.00 (6H, m), 4.25 (2H, q, J=9 Hz), 4.60-4.70(3H, m), 6.90-7.00 (2H, m), 7.20-7.30 (3H, m), 7.70-7.90 (5H, m), 8.50-8.60 (1H, m)

### EXAMPLE 115

4-[[4-[2-[N-Benzyl-N-[(2R)-2-(3-chlorophenyl)-2-hydroxyethyl]amino]ethyl]phenyl]sulfonyl]phenol (180 mg), N-(2-bromoethyl)phthalimide (96 mg) and potassium carbonate (57.2 mg) in N,N-dimethylformamide (5 ml) was stirred for 20 hours. The resulting mixture was poured into water and extracted with ethyl acetate. The organic layer was washed with brine, dried over magnesium sulfate, and evaporated in vacuo. To the residue was added hydrazine (20.7 mg), methanol (3 ml) and tetrahydrofuran (3 ml) and refluxed for 4 hours. The resulting mixture was poured into water and extracted with ethyl acetate. The organic layer was washed with brine, dried over magnesium sulfate, and evaporated in vacuo. The residue, 10% palladium on carbon (50% wet, 30 mg) and 4N hydrogen chloride in 1,4-dioxane (1 ml) in methanol was stirred at room temperature in the presence of hydrogen at an atmosperic pressure for 2 hours. Afer filtration, the filtrate was evaporated in vacuo to give (1R)-2-[[2-[4-[[4-(2-aminoethoxy)phenyl]sulfonyl]phenyl]-ethyl]amino)-1-(3-chlorophenyl)ethanol dihydrochloride (50 mg) as a colorless powder.

NMR (DMSO-d₆, δ): 2.90-3.30 (8H, m), 4.20-4.40 (2H, m), 4.90-5.00 (1H, m), 7.10-7.50 (8H, m), 7.70-7.90 (4H, m)

MS (m/z): 475 (M+H)

### EXAMPLE 116

To a mixture of 3-[[4-[2-[N-(tert-butoxycarbonyl)-N-[(2R)-2-(3-chlorophenyl)-2-hydroxyethyl]amino]ethyl]phenyl]-sulfonyl]benzoic acid (1.60 g), N,O-dimethylhydroxylamine hydrochloride (321 mg), and 1-hydroxybenzotriazole (391 mg) in dichloromethane (16 ml)-N,N-dimethylformamide (0.8 ml) was added 1-[3-(dimethylamino)propyl]-3-ethylcarbodiimide (490 mg), and the mixture was stirred at room temperature for 17.5 hours. The mixture was partitioned between ethyl acetate and water. The organic layer was separated, washed successively with sodium bicarbonate solution and brine, dried over magnesium sulfate, and filtered. The filtrate was concentrated and the residue was purified by column chromatography (silica gel, hexane/ethyl acetate) to give tert-butyl N-[(2R)-2-(3-chlorophenyl)-2-hydroxyethyl]-N-[2-[4-[[3-[[methoxy(methyl]amino]carbonyl]phenyl]sulfonyl]-phenyl]ethyl]carbamate (1.13 g) as a white amorphous powder.

NMR (CDCl₃, δ): 1.36 (9H, s), 2.50-3.60 (6H, m), 3.36 (3H, s), 3.52 (3H, s), 4.25 (1H, br s, OH), 4.87 (1H, m), 7.05-7.42 (6H, m), 7.53 (1H, t, J=8 Hz), 7.86 (2H, d, J=8 Hz), 7.86 (1H, d, J=8 Hz), 8.01 (1H, d, J=8 Hz), 8.25 (1H, s) (+)ESI-MS (m/z): 625 (M+Na)⁺

### EXAMPLE 117

To an ice-cooled solution of tert-butyl N-[(2R)-2-(3-chlorophenyl)-2-hydroxyethyl]-N-[2-[4-[[3-[[methoxy(methyl)-amino]carbonyl]phenyl]sulfonyl]phenyl]ethyl]carbamate (1.12 g) in tetrahydrofuran (9 ml) was added lithium aluminum hydride (72 mg), and the mixture was stirred at room temperature for 6.5 hours. After the mixture was diluted with ether (9 ml) and cooled with ice, sodium fluoride (320 mg) was added. Water (0.36 ml) was added to the mixture with vigorous stirring, and the precipitate formed was removed by filtration. The filtrate was concentrated and the residue was purified by column chromatography (silica gel, hexane/ethyl acetate) to give tert-butyl N-[(2R)-2-(3-chlorophenyl)-2-hydroxyethyl]-N-[2-[4-[(3-formylphenyl)-sulfonyl]phenyl]ethyl]carbamate (779 mg) as a viscous oil.

NMR (CDCl₃, δ): 1.34 (9H, s), 2.50-3.70 (6H, m), 4.21 (1H, br s, OH), 4.82 (1H, m), 7.00-7.50 (6H, m), 7.67 (1H, t, J=8 Hz), 7.89 (2H, d, J=8 Hz), 8.05 (1H, d, J=8 Hz), 8.17 (1H, d, J=8 Hz), 8.40 (1H, s), 10.04 (1H, s) (+)ESI-MS (m/z): 566 (M+Na)⁺

### EXAMPLE 118

To a mixture of tert-butyl N-[(2R)-2-(3-chlorophenyl)-2-hydroxyethyl]-N-[2-[4-[(3-formylphenyl)sulfonyl]phenyl]-ethyl]carbamate (243 mg) and methyl (triphenylphosphoranylidene)acetate (227 mg) in tetrahydrofuran (1.9 ml) was heated to 60° C. for 1.5 hours. After being cooled to room temperature, the mixture was concentrated and the residue was purified by column chromatography (silica gel, hexane/ethyl acetate) to give methyl (2E)-3-[3-[[4-[2-[N-(tert-butoxycarbonyl)-N-[(2R)-2-(3-chlorophenyl)-2-hydroxyethyl]amino]ethyl]phenyl]-sulfonyl]phenyl]-2-propenoate (211 mg) as a white amorphous powder.

NMR (CDCl₃, δ): 1.33 (9H, s), 2.60-3.60 (6H, m), 3.82 (3H, s), 4.23 (1H, br s, OH), 4.85 (1H, m), 6.50 (1H, d, J=16 Hz), 7.08-7.42 (6H, m), 7.50 (1H, t, J=8 Hz), 7.66 (1H, d, J=16 Hz), 7.66 (1H, d, J=8 Hz), 7.87 (2H, d, J=8 Hz), 7.91 (1H, d, J=8 Hz), 8.05 (1H, s) (+)ESI-MS (m/z): 622 (M+Na)⁺

### EXAMPLE 119

The following compounds were obtained according to a similar manner to that of Example 56.

(1) (2E)-3-[3-[[4-[2-[N-(tert-Butoxycarbonyl)-N-[(2R)-2-(3-chlorophenyl)-2-hydroxyethyl]amino]ethyl]phenyl]-sulfonyl]phenyl]-2-propenoic acid

NMR (CDCl₃, δ): 1.34 (9H, s), 2.60-3.60 (6H, m), 4.85 (1H, m), 6.51 (1H, d, J=16 Hz), 7.05-7.45 (6H, m), 7.52 (1H, t, J=8 Hz), 7.69 (1H, d, J=8 Hz), 7.74 (1H, d, J=16 Hz), 7.88 (2H, d, J=8 Hz), 7.94 (1H, d, J=8 Hz), 8.07 (1H, s) (+)ESI-MS (m/z): 608 (M+Na)⁺

(2) [[3-[[4-[2-[N-(tert-Butoxycarbonyl)-N-[(2R)-2-(3-chlorophenyl)-2-hydroxyethyl]amino]ethyl]phenyl]-sulfonyl]benzoyl]amino]acetic acid

NMR (DMSO-d₆, δ): 1.02, 1.19 (total 9H, a pair of s), 2.70-2.95 (2H, m), 3.08-3.60 (4H, m), 3.95 (2H, d, J=6 Hz), 4.75 (1H, m), 5.59 (1H, br s, OH), 7.15-7.50 (6H, m), 7.73 (1H, t, J=8 Hz), 7.90 (2H, d, J=8 Hz), 8.10 (1H, d, J=8 Hz), 8.15 (1H, d, J=8 Hz), 8.43 (1H, s), 9.19 (1H, br t, J=6 Hz), 12.70 (1H, br s) (−)ESI-MS (m/z): 615 (M−H)⁻

(3) [[4-[[4-[2-[N-(tert-Butoxycarbonyl)-N-[(2R)-2-(3-chlorophenyl)-2-hydroxyethyl]amino]ethyl]phenyl]-sulfonyl]benzoyl]amino]acetic acid

NMR (DMSO-d₆, δ): 1.06, 1.19 (total 9H, a pair of s), 2.70-2.95 (2H, m), 3.10-3.60 (4H, m), 3.93 (2H, d, J=6 Hz), 4.73 (1H, m), 5.59 (1H, br s, OH), 7.15-7.49 (6H, m), 7.89 (2H, m), 8.04 (4H, m), 9.07 (1H, t, J=6 Hz), 12.50 (1H, br s) (−)ESI-MS (m/z): 615 (M−H)⁻

(4) [[4-[[4-[(2R)-2-[N-(tert-Butoxycarbonyl)-N-[(2R)-2-(3-chlorophenyl)-2-hydroxyethyl]amino]propyl]phenyl]-sulfonyl]benzoyl]amino]acetic acid

NMR (CDCl₃, δ): 1.22 (3H, d, J=6 Hz), 1.26 (9H, s), 2.50-3.60 (4H, m), 3.95-4.21 (1H, m), 4.21 (2H, d, J=5 Hz), 4.62 (1H, m), 6.92 (1H, br t, J=5 Hz), 7.08-7.42 (6H, m), 7.85 (2H, d, J=8 Hz), 7.85 (2H, d, J=8 Hz), 7.95 (2H, d, J=8 Hz) (−)ESI-MS (m/z): 629 (M−H)⁻

(5) 3-[[3-[[4-[2-[N-(tert-Butoxycarbonyl)-N-[(2R)-2-(3-chlorophenyl)-2-hydroxyethyl]amino]ethyl]phenyl]-sulfonyl]benzoyl]amino]propanoic acid

NMR (DMSO-d₆, δ): 1.02, 1.08 (total 9H, a pair of s), 2.62-2.98 (2H, m), 3.00-3.70 (8H, m), 4.73 (1H, m), 5.58 (1H, br s, OH), 7.08-7.52 (6H, m), 7.70 (1H, t, J=8 Hz), 7.89 (2H, d, J=8 Hz), 8.07 (1H, d, J=8 Hz), 8.11 (1H, d, J=8 Hz), 8.39 (1H, s), 8.87 (1H, br t, J=5 Hz), 12.30 (1H, br s) (−)ESI-MS (m/z): 629 (M−H)⁻

(6) [[3-[[4-[2-[N-(tert-Butoxycarbonyl)-N-[(2R)-2-(3-chlorophenyl)-2-hydroxyethyl]amino]ethyl]phenyl]-sulfonyl]benzoyl](methyl)amino]acetic acid

NMR (DMSO-d₆, δ): 1.05, 1.20 (total 9H, a pair of s), 2.65-3.60 (6H, m), 2.90, 2.99 (total 3H, a pair of s), 3.88, 4.15 (total 2H, a pair of s), 4.73 (1H, m), 5.58 (1H, br s, OH), 7.10-8.15 (12H, m), 13.10(1H, br s) (−)ESI-MS (m/z): 629 (M−H)⁻

(7) (2S)-2-[[3-[[4-[2-[N-(tert-Butoxycarbonyl)-N-[(2R)-2-(3-chlorophenyl)-2-hydroxyethyl]amino]ethyl]phenyl]-sulfonyl]benzoyl]amino]propanoic acid

NMR (DMSO-d₆, δ): 1.02, 1.19 (total 9H, a pair of s), 1.41 (3H, d, J=7 Hz), 2.65-3.70 (6H, m), 4.44 (1H, quintet, J=7 Hz), 4.73 (1H, m), 5.59 (1H, br s, OH), 7.10-7.55 (6H, m), 7.72 (1H, t, J=8 Hz), 7.90 (2H, d, J=8 Hz), 8.10 (1H, d, J=8 Hz), 8.17 (1H, d, J=8 Hz), 8.45 (1H, s), 9.02 (1H, br d, J=7 Hz), 12.65 (1H, br s) (−)ESI-MS (m/z): 629 (M−H)⁻

(8) (2R)-2-[[3-[[4-[2-[N-(tert-Butoxycarbonyl)-N-[(2R)-2-(3-chlorophenyl)-2-hydroxyethyl]amino]ethyl]phenyl]-sulfonyl]benzoyl]amino]propanoic acid

NMR (DMSO-d₆, δ): 1.02, 1.18 (total 9H, a pair of s), 1.41 (3H, d, J=7 Hz), 2.65-3.65 (6H, m), 4.44 (1H, quintet, J=7 Hz), 4.74 (1H, m), 5.59 (1H, br s, OH), 7.10-7.55 (6H, m), 7.72 (1H, t, J=8 Hz), 7.90 (2H, d, J=8 Hz), 8.10 (1H, d, J=8 Hz), 8.17 (1H, d, J=8 Hz), 8.45 (1H, s), 9.02 (1H, br d, J=7 Hz), 12.60 (1H, br s) (−)ESI-MS (m/z): 629 (M−H)⁻

(9) [[4-[[4-[(2R)-2-[N-(tert-Butoxycarbonyl)-N-[(2R)-2-(3-chlorophenyl)-2-hydroxyethyl]amino]propyl]phenyl]-sulfonyl]benzoyl](methyl)amino]acetic acid

NMR (DMSO-d₆, δ): 1.11, 1.21 (total 9H, a pair of s), 1.19 (3H, d, J=7 Hz), 2.60-3.70 (4H, m), 2.86, 2.97 (total 3H, a pair of s), 3.87, 4.15 (total 2H, a pair of s), 3.94 (1H, m), 4.68, 4.82 (total 1H, a pair of m), 5.55 (1H, br s, OH), 7.05-7.70 (8H, m), 7.75-8.12 (4H, m), 12.80 (1H, br s) (−)ESI-MS (m/z): 643 (M−H)⁻

### EXAMPLE 120

The following compounds were obtained according to a similar manner to that of Example 33.

(1) (2E)-3-[3-[[4-[2-[[(2R)-2-(3-Chlorophenyl)-2-hydroxyethyl]amino]ethyl]phenyl]sulfonyl]phenyl]-2-propenoic acid hydrochloride

NMR (DMSO-d₆, δ): 2.90-3.55 (6H, m), 4.99 (1H, m), 6.32 (1H, br s, OH), 6.71 (1H, d, J=16 Hz), 7.22-7.80, (8H, m), 7.80-8.15 (4H, m), 8.27 (1H, s), 9.34 (2H, br d) (−)ESI-MS (m/z): 484 (free, M−H)⁻

(2) (5Z)-5-[3-[[4-[2-[[(2R)-2-(3-chlorophenyl)-2-hydroxyethyl]amino]ethyl]phenyl]sulfonyl]benzylidene]-1,3-thiazolidine-2,4-dione hydrochloride

NMR (DMSO-d₆, δ): 2.80-3.75 (6H, m), 4.99 (1H, m), 6.32 (1H, br s, OH), 7.20-7.65 (6H, m), 7.65-8.10 (6H, m), 8.16 (1H, s), 8.93 (1H, br s), 9.20 (1H, br s), 12.77 (1H, br s) (−)ESI-MS (m/z): 541 (free, M−H)⁻

(3) 3-[[4-[2-[[(2R)-2-(3-Chlorophenyl)-2-hydroxyethyl]amino]ethyl]phenyl]sulfonyl]-N,N-dimethylbenzamide hydrochloride

NMR (DMSO-d₆, δ): 2.80-3.40 (6H, m), 2.86 (3H, s), 2.99 (3H, s), 5.02 (1H, m), 6.35 (1H, br s), 7.30-7.50 (4H, m), 7.54 (2H, d, J=8 Hz), 7.62-7.80 (2H, m), 7.85-8.10 (4H, m), 8.98 (1H, br s), 9.33 (1H, br s) (+)ESI-MS (m/z): 487 (free, M+H)+

(4) 4-[[4-[2-[[(2R)-2-(3-Chlorophenyl)-2-hydroxyethyl]amino)ethyl]phenyl]sulfonyl]-N,N-dimethylbenzamide hydrochloride

NMR (DMSO-d₆, δ): 2.83 (3H, s), 2.90-3.35 (6H, m), 2.98 (3H, s), 5.02 (1H, m), 7.26-7.54 (4H, m), 7.54 (2H, d, J=8 Hz), 7.62 (2H, d, J=8 Hz), 7.96 (2H, d, J=8 Hz), 8.01 (2H, d, J=8 Hz), 8.99 (1H, br s), 9.36 (1H, br s) (+)ESI-MS (m/z): 487 (free, M+H)⁺

(5) 3-[[4-[2-[[(2R)-2-(3-Chlorophenyl)-2-hydroxyethyl]amino]-ethyl]phenyl]sulfonyl]-N-methylbenzamide hydrochloride

NMR (DMSO-d₆, δ): 2.80 (3H, d, J=4 Hz), 2.90-3.30 (6H, m), 5.00 (1H, m), 6.35 (1H, br s, OH), 7.32-7.49 (4H, m), 7.54 (2H, d, J=8 Hz), 7.72 (1H, t, J=8 Hz), 7.97 (2H, d, J=8 Hz), 8.10 (1H, d, J=8 Hz), 8.13 (1H, d, J=8 Hz), 8.39 (1H, s), 8.83 (1H, q, J=4 Hz), 8.95 (1H, br s), 9.26 (1H, br s) (+)ESI-MS (m/z): 473 (free, M+H)⁺

(6) 4-[[4-[(2R)-2-[[(2R)-2-(3-Chlorophenyl)-2-hydroxyethyl]amino]propyl]phenyl]sulfonyl]-N-methylbenzamide hydrochloride

NMR (DMSO-d₆, δ): 1.09 (3H, d, J=6 Hz), 2.60-3.65 (5H, m), 2.78 (3H, d, J=5 Hz), 5.04 (1H, m), 6.35 (1H, br s, OH), 7.30-7.62 (6H, m), 7.96 (2H, d, J=8 Hz), 8.03 (4H, s), 8.71 (1H, br q, J=5 Hz), 8.83 (1H, br s), 9.32 (1H, br s) (+)ESI-MS (m/z): 487 (free, M+H)⁺

(7) 4-[[4-[(2R)-2-[[(2R)-2-(3-Chlorophenyl)-2-hydroxyethyl]amino]propyl]phenyl]sulfonyl]-N,N-dimethylbenzamide hydrochloride

NMR (DMSO-d₆, δ): 1.10 (3H, d, J=6 Hz), 2.63-3.67 (5H, m), 2.83 (3H, s), 2.98 (3H, s), 5.06 (1H, m), 6.36 (1H, br s, OH), 7.30-7.65 (6H, m), 7.62 (2H, d, J=8 Hz), 7.97 (2H, d, J=8 Hz), 8.01 (2H, d, J=8 Hz), 8.85 (1H, br s), 9.41 (1H, br s) (+)ESI-MS (m/z): 501 (free, M+H)⁺

(8) 1-[3-[[4-[2-[[(2R)-2-(3-Chlorophenyl)-2-hydroxyethyl]amino]ethyl]phenyl]sulfonyl]benzoyl]-4-piperidinol hydrochloride

NMR (DMSO-d₆, δ): 1.15-2.00 (4H, m), 2.70-4.10 (12H, m), 5.01 (1H, m), 6.35 (1H, br s, OH), 7.20-7.80 (8H, m), 7.80-8.15 (4H, m), 8.96 (1H, br s), 9.27 (1H, br s) (+)ESI-MS (m/z): 543 (free, M+H)⁺

(9) 1-[4-[[4-[(2R)-2-[[(2R)-2-(3-Chlorophenyl)-2-hydroxyethyl]amino]propyl]phenyl]sulfonyl]benzoyl]-4-piperidinol hydrochloride

NMR (DMSO-d₆, δ): 1.10 (3H, d, J=6 Hz), 1.15-1.95 (4H, m), 2.65-4.10 (10H, m), 4.81 (1H, br s, OH), 5.01 (1H, m), 6.34 (1H, br s, OH), 7.25-7.70 (8H, m), 7.85-8.15 (4H, m), 8.80 (1H, br s), 9.15 (1H, br s) (+)ESI-MS (m/z): 557 (free, M+H)⁺

(10) 3-[[4-[2-[[(2R)-2-(3-Chlorophenyl)-2-hydroxyethyl]-amino]ethyl]phenyl]sulfonyl]-N-(5-methyl-1,3-thiazol-2-yl)benzamide hydrochloride

NMR (DMSO-d₆, δ): 2.38 (3H, s), 2.90-3.35 (6H, m), 5.01 (1H, m), 6.56 (1H, br s), 7.26 (1H, s), 7.30-7.52 (4H, m), 7.55 (2H, d, J=8 Hz), 7.78 (1H, t, J=8 Hz), 8.01 (2H, d, J=8 Hz), 8.18 (1H, d, J=8 Hz), 8.34 (1H, d, J=8 Hz), 8.65 (1H, s), 8.98 (1H, br s), 9.32 (1H, br s) (+)ESI-MS (m/z): 556 (free, M+H)⁺

(11) 4-[[4-[2-[[(2R)-2-(3-Chlorophenyl)-2-hydroxyethyl]-amino]ethyl]phenyl]sulfonyl]-N-(5-methyl-1,3-thiazol-2-yl)benzamide hydrochloride

NMR (DMSO-d₆, δ): 2.37 (3H, s), 2.90-3.33 (6H, m), 4.97 (1H, m), 6.30 (1H, br s, OH), 7.24 (1H, s), 7.31-7.49 (4H, m), 7.55 (2H, d, J=8 Hz), 7.99 (2H, d, J=8 Hz), 8.10 (2H, d, J=8 Hz), 8.24 (2H, d, J=8 Hz), 8.89 (1H, br s), 9.11 (1H, br s) (+)ESI-MS (m/z): 556 (free, M+H)⁺

(12) 3-[[4-[2-[[(2R)-2-(3-Chlorophenyl)-2-hydroxyethyl]-amino]ethyl]phenyl]sulfonyl]-N-(1H-tetrazol-5-yl)benzamide hydrochloride

NMR (DMSO-d₆, δ): 2.90-3.30 (6H, m), 4.98 (1H, m), 6.34 (1H, br s, OH), 7.31-7.49 (4H, m), 7.55 (2H, d, J=8 Hz), 7.83 (1H, t, J=8 Hz), 8.02 (2H, d, J=8 Hz), 8.23 (1H, d, J=8 Hz), 8.34 (1H, d, J=8 Hz), 8.68 (1H, s), 8.88 (1H, br s), 9.15 (1H, br s), 12.83 (1H, br s) (−)ESI-MS (m/z): 525 (free, M−H)⁻

(13) 4-[[4-[2-[[(2R)-2-(3-Chlorophenyl)-2-hydroxyethyl]-amino]ethyl]phenyl]sulfonyl]-N-(1H-tetrazol-5-yl)benzamide hydrochloride

NMR (DMSO-d₆, δ): 2.90-3.30 (6H, m), 4.97 (1H, m), 6.34 (1H, br s, OH), 7.31-7.49 (4H, m), 7.55 (2H, d, J=8 Hz), 8.00 (2H, d, J=8 Hz), 8.14 (2H, d, J=8 Hz), 8.25 (2H, d, J=8 Hz), 8.89 (1H, br s), 9.10 (1H, br s), 12.71 (1H, br s) (−)ESI-MS (m/z): 525 (free, M−H)⁻

(14) Ethyl [[3-[[4-[2-[[(2R)-2-(3-chlorophenyl)-2-hydroxyethyl]amino]ethyl]phenyl]sulfonyl]benzoyl]-amino]acetate hydrochloride

NMR (DMSO-d₆, δ): 1.21 (3H, t, J=7 Hz), 2.93-3.30 (6H, m), 4.02 (2H, d, J=5 Hz), 4.12 (2H, q, J=7 Hz), 4.99 (1H, m), 6.33 (1H, br s, OH), 7.30-7.50 (4H, m), 7.54 (2H, d, J=8 Hz), 7.76 (1H, t, J=8 Hz), 7.98 (2H, d, J=8 Hz), 8.15 (1H, d, J=8 Hz), 8.17 (1H, d, J=8 Hz), 8.43(1H, s), 8.93(1H, br s), 9.10(1H, br s), 9.35(1H, t, J=5 Hz) (+)ESI-MS (m/z): 545 (free, M+H)⁺

(15) [[3-[[4-[2-[[(2R)-2-(3-Chlorophenyl)-2-hydroxyethyl]-amino]ethyl]phenyl]sulfonyl]benzoyl]amino]acetic acid hydrochloride

NMR (DMSO-d₆, δ): 2.90-3.30 (6H, m), 3.95 (2H, d, J=6 Hz), 4.97 (1H, m), 6.32 (1H, br s, OH), 7.31-7.49 (4H, m), 7.54 (2H, d, J=8 Hz), 7.76 (1H, t, J=8 Hz), 7.98 (2H, d, J=8 Hz), 8.14 (1H, d, J=8 Hz), 8.17 (1H, d, J=8 Hz), 8.44 (1H, s), 9.00 (2H, br s), 9.24 (1H, br t, J=6 Hz), 12.50 (1H, br s) (−)ESI-MS (m/z): 515 (free, M−H)⁻

(16) Ethyl [[4-[[4-[2-[[(2R)-2-(3-chlorophenyl)-2-hydroxyethyl]amino]ethyl]phenyl]sulfonyl]benzoyl]-amino]acetate hydrochloride

NMR (DMSO-d₆, δ): 1.19 (3H, t, J=7 Hz), 2.93-3.30 (6H, m), 4.00 (2H, d, J=6 Hz), 4.11 (2H, q, J=7 Hz), 4.98 (1H, m), 6.33 (1H, br s, OH), 7.31-7.49 (4H, m), 7.54 (2H, d, J=8 Hz), 7.97 (2H, d, J=8 Hz), 8.06 (2H, d, J=8 Hz), 8.08 (2H, d, J=8 Hz), 9.00 (2H, br s), 9.23 (1H, t, J=6 Hz) (+)ESI-MS (m/z): 545 (free, M+H)⁺

(17) [[4-[[4-[2-[[(2R)-2-(3-Chlorophenyl)-2-hydroxyethyl]amino]ethyl]phenyl]sulfonyl]benzoyl]amino]acetic acid hydrochloride

NMR (DMSO-d₆, δ): 2.92-3.32 (6H, m), 3.93 (2H, d, J=6 Hz), 4.97 (1H, m), 6.32 (1H, br s, OH), 7.31-7.49 (4H, m), 7.53 (2H, d, J=8 Hz), 7.96 (2H, d, J=8 Hz), 8.05 (2H, d, J=8 Hz), 8.07 (2H, d, J=8 Hz), 9.05 (2H, br s), 9.11 (1H, br t, J=5 Hz), 12.45 (1H, br s) (−)ESI-MS (m/z): 515 (free, M−H)⁻

(18) Ethyl [[4-[[4-[(2R)-2-[[(2R)-2-(3-chlorophenyl)-2-hydroxyethyl]amino]propyl]phenyl]sulfonyl]benzoyl]-amino]acetate hydrochloride

NMR (DMSO-d₆, δ): 1.09 (3H, d, J=6 Hz), 1.19 (3H, t, J=7 Hz), 2.65-3.65 (5H, m), 4.01 (2H, d, J=6 Hz), 4.11 (2H, q, J=7 Hz), 5.04 (1H, m), 6.25 (1H, br s, OH), 7.25-7.65 (6H, m), 7.57 (2H, d, J=8 Hz), 8.05 (2H, d, J=8 Hz), 8.10 (2H, d, J=8 Hz), 8.84 (1H, br s), 9.24 (1H, br t, J=6 Hz), 9.28 (1H, br s) (+)ESI-MS (m/z): 559 (free, M+H)⁺

(19) [[4-[[4-[(2R)-2-[[(2R)-2-(3-Chlorophenyl)-2-hydroxyethyl]amino]propyl]phenyl]sulfonyl]benzoyl]-amino]acetic acid hydrochloride

NMR (DMSO-d₆, δ): 1.09 (3H, d, J=6 Hz), 2.65-3.65 (5H, m), 3.93 (2H, d, J=6 Hz), 5.06 (1H, m), 6.35 (1H, br s, OH), 7.27-7.62 (6H, m), 7.97 (2H, d, J=8 Hz), 8.07 (4H, s), 9.14 (1H, br t, J=6 Hz), 9.30 (2H, br s) (−)ESI-MS (m/z): 529 (free, M−H)⁻

(20) Ethyl 3-[[3-[[4-[2-[[(2R)-2-(3-chlorophenyl)-2-hydroxyethyl]amino]ethyl]phenyl]sulfonyl]benzoyl]-amino]propanoate hydrochloride

NMR (DMSO-d₆, δ): 1.16 (3H, t, J=7 Hz), 2.60 (2H, t, J=6 Hz), 2.90-3.40 (6H, m), 3.51 (2H, q, J=6 Hz), 4.07 (2H, q, J=7 Hz), 5.01 (1H, m), 6.34 (1H, br s, OH), 7.20-7.53 (4H, m), 7.54 (2H, d, J=8 Hz), 7.73 (1H, t, J=8 Hz), 7.97 (2H, d, J=8 Hz), 8.10 (1H, d, J=8 Hz), 8.13 (1H, d, J=8 Hz), 8.39 (1H, s), 8.96 (1H, br t, J=6 Hz), 9.12 (2H, br s) (+)ESI-MS (m/z): 559 (free, M+H)⁺

(21) 3-[[3-[[4-[2-[[(2R)-2-(3-Chlorophenyl)-2-hydroxyethyl]-amino]ethyl]phenyl]sulfonyl]benzoyl]amino]propanoic acid hydrochloride

NMR (DMSO-d₆, δ): 2.54 (2H, t, J=6 Hz), 2.80-3.80 (8H, m), 5.01 (1H, m), 6.35 (1H, br s, OH), 7.20-7.55 (4H, m), 7.54 (2H, d, J=8 Hz), 7.72 (1H, t, J=8 Hz), 7.97 (2H, d, J=8 Hz), 8.10 (1H, d, J=8 Hz), 8.14 (1H, d, J=8 Hz), 8.40 (1H, s), 8.94 (1H, br t, J=6 Hz), 8.96 (1H, br s), 9.27 (1H, br s), 12.40 (1H, br s) (−)ESI-MS (m/z): 529 (free, M−H)⁻

(22) Ethyl [[3-[(4-[2-[[(2R)-2-(3-chlorophenyl)-2-hydroxyethyl]amino]ethyl]phenyl]sulfonyl]benzoyl]-(methyl)amino]acetate hydrochloride

NMR (DMSO-d₆, δ): 1.14, 1.24 (total 3H, a pair of t, J=7 Hz), 2.80-3.40 (6H, m), 2.92, 3.01 (total 3H, a pair of s), 4.09, 4.17 (total 2H, a pair of q, J=7 Hz), 4.02, 4.24 (total 2H, a pair of s), 5.02 (1H, m), 6.35 (1H, br s, OH), 7.20-8.20 (12H, m), 9.00 (1H, br s), 9.34 (1H, br s) (+)ESI-MS (m/z): 559 (free, M+H)⁺

(23) [[3-[[4-[2-[[(2R)-2-(3-Chlorophenyl)-2-hydroxyethyl]-amino]ethyl]phenyl]sulfonyl]benzoyl](methyl)amino]-acetic acid hydrochoride

NMR (DMSO-d₆, δ): 2.70-3.70 (6H, m), 2.91, 3.00 (3H, total 3H, a pair of s), 3.90, 4.17 (2H, total 2H, a pair of s), 5.03 (1H, m), 6.35 (1H, br s, OH), 7.15-8.30 (12H, m), 9.04 (1H, br s), 9.39 (1H, br s), 12.99 (1H, br s) (−)ESI-MS (m/z): 529 (free, M−H)⁻

(24) Ethyl (2S)-2-[[3-[[4-[2-[[(2R)-2-(3-chlorophenyl)-2-hydroxyethyl]amino]ethyl]phenyl]sulfonyl]benzoyl]amino]propanoate hydrochloride

NMR (DMSO-d₆, δ): 1.19 (3H, t, J=7 Hz), 1.42 (3H, d, J=7 Hz), 2.85-3.40 (6H, m), 4.11 (2H, q, J=7 Hz), 4.47 (1H, quintet, J=7 Hz), 5.00 (1H, m), 6.34 (1H, br s, OH), 7.20-7.55 (4H, m), 7.54 (2H, d, J=8 Hz), 7.75 (1H, t, J=8 Hz), 7.98 (2H, d, J=8 Hz), 8.14 (1H, d, J=8 Hz), 8.19 (1H, d, J=8 Hz), 8.44 (1H, s), 9.09 (2H, br s), 9.17 (1H, br d, J=7 Hz) (+)ESI-MS (m/z): 559 (free, M+H)⁺

(25) (2S)-2-[[3-[[4-[2-[[(2R)-2-(3-Chlorophenyl)-2-hydroxyethyl]amino]ethyl]phenyl]sulfonyl]benzoyl]-amino]propanoic acid hydrochloride

NMR (DMSO-d₆, δ): 1.42 (3H, d, J=7 Hz), 2.85-3.75 (6H, m), 4.44 (1H, quintet, J=7 Hz), 5.02 (1H, m), 6.35 (1H, br s, OH), 7.15-7.60 (4H, m), 7.55 (2H, d, J=8 Hz), 7.75 (1H, t, J=8 Hz), 7.98 (2H, d, J=8 Hz), 8.14 (1H, d, J=8 Hz), 8.20 (1H, d, J=8 Hz), 8.46 (1H, s), 9.08 (1H, br d, J=7 Hz), 9.13 (2H, br s), 12.65 (1H, br s) (−)ESI-MS (m/z): 629 (free, M−H)⁻

(26) Methyl (2R)-2-[[3-[[4-[2-[[(2R)-2-(3-chlorophenyl)-2-hydroxyethyl]amino]ethyl]phenyl]sulfonyl]benzoyl]-amino]propanoate hydrochloride

NMR (DMSO-d₆, δ): 1.43 (3H, d, J=7 Hz), 2.85-3.50 (6H, m), 3.65 (3H, s), 4.50 (1H, quintet, J=7 Hz), 5.02 (1H, m), 6.36 (1H, br s, OH), 7.20-7.55 (4H, m), 7.55 (2H, d, J=8 Hz), 7.75 (1H, t, J=8 Hz), 7.98 (2H, d, J=8 Hz), 8.14 (1H, d, J=8 Hz), 8.20 (1H, d, J=8 Hz), 8.46 (1H, s), 9.00 (1H, br s), 9.21 (1H, br d, J=7 Hz), 9.34 (1H, br s) (+)ESI-MS (m/z): 545 (free, M+H)⁺

(27) (2R)-2-[[3-[[4-[2-[[(2R)-2-(3-Chlorophenyl)-2-hydroxyethyl]amino]ethyl]phenyl]sulfonyl]benzoyl]-amino]propanoic acid hydrochloride

NMR (DMSO-d₆, δ): 1.42 (3H, d, J=7 Hz), 2.80-3.75 (6H, m), 4.45 (1H, quintet, J=7 Hz), 5.03 (1H, m), 6.35 (1H, br s, OH), 7.15-7.58 (4H, m), 7.55 (2H, d, J=8 Hz), 7.75 (1H, t, J=8 Hz), 7.98 (2H, d, J=8 Hz), 8.14 (1H, d, J=8 Hz), 8.20 (1H, d, J=8 Hz), 8.46 (1H, s), 9.09 (1H, br d, J=7 Hz), 9.20 (2H, br s), 12.60 (1H, br s) (−)ESI-MS (m/z): 529 (free, M−H)⁻

(28) Ethyl [[4-[[4-[(2R)-2-[[(2R)-2-(3-chlorophenyl)-2-hydroxyethyl]amino]propyl]phenyl]sulfonyl]benzoyl]-(methyl)amino]acetate hydrochloride

NMR (DMSO-d₆, δ): 1.09 (3H, d, J=6 Hz), 1.22 (3H, t, J=7 Hz), 2.65-3.65 (5H, m), 2.89, 2.98 (3H, total 3H, a pair of s), 4.01, 4.23 (2H, total 3H, a pair of s), 4.15 (2H, q, J=7 Hz), 5.01 (1H, m), 6.34 (1H, br s, OH), 7.30-7.65 (6H, m), 7.64 (2H, d, J=8 Hz), 7.97 (2H, d, J=8 Hz), 8.05 (2H, d, J=8 Hz), 8.82 (1H, br s), 9.14 (1H, br s) (+)ESI-MS (m/z): 573 (free, M+H)⁺

(29) [[4-[[4-[(2R)-2-[[(2R)-2-(3-Chlorophenyl)-2-hydroxyethyl]amino]propyl]phenyl]sulfonyl]benzoyl]-(methyl)amino]acetic acid hydrochloride

NMR (DMSO-d₆, δ): 1.10 (3H, d, J=6 Hz), 2.65-3.80 (5H, m), 2.88, 2.98 (total 3H, a pair of s), 3.91, 4.15 (total 2H, a pair of s), 5.06 (1H, m), 6.37 (1H, br s, OH), 7.25-7.75 (8H, m), 7.82-8.22 (4H, m), 8.95 (1H, br s), 9.40 (1H, br s), 12.75 (1H, br s) (−)ESI-MS (m/z): 543 (free, M−H)⁻

(30) Sodium [4-[[4-[(2S)-2-[[(2R)-2-(3-chlorophenyl)-2-hydroxyethyl]amino]-3-hydroxypropyl]phenyl]sulfonyl]-phenoxy]acetate

NMR (DMSO-d₆, δ): 2.70-2.90 (5H, m), 3.00-3.55 (2H, m), 4.17 (2H, s), 4.58 (1H, m), 4.58 (1H, br s, OH), 5.43 (1H, br s, OH), 6.82 (2H, d, J=9 Hz), 7.10-7.60 (6H, m), 7.76 (2H, d, J=9 Hz), 7.76 (2H, d, J=8 Hz) (−)ESI-MS (m/z): 518 (free, M−H)⁻

### EXAMPLE 121

A solution of (2E)-3-[3-[[4-[2-[N-(tert-butoxycarbonyl)-N-[(2R)-2-(3-chlorophenyl)-2-hydroxyethyl]-amino]ethyl]phenyl]sulfonyl]phenyl]-2-propenoic acid (99 mg) in ethanol (5 ml) was hydrogenated (2 atm) over platinum(IV) oxide (10 mg) at room temperature for 4 hours. The catalyst was filtered off and the filtrate was evaporated to give 3-[3-[[4-[2-[N-(tert-butoxycarbonyl)-N-[(2R)-2-(3-chlorophenyl)-2-hydroxyethyl]amino]ethyl]phenyl]sulfonyl]-phenyl]propanoic acid (106 mg) as a white amorphous powder.

NMR (CDCl₃, δ): 1.33 (9H, s), 2.50-3.60 (6H, m), 2.68 (2H, t, J=7 Hz), 2.98 (2H, t, J=7 Hz), 4.77 (1H, m), 7.00-7.50 (8H, m), 7.65-8.00 (4H, m) (+)ESI-MS (m/z): 610 (M+Na)⁺

### EXAMPLE 122

3-[3-[[4-[2-[N-(tert-Butoxycarbonyl)-N-[(2R)-2-(3-chlorophenyl)-2-hydroxyethyl]amino]ethyl]phenyl]sulfonyl]-phenyl]propanoic acid (87 mg) and 4N hydrogen chloride in 1,4-dioxane (1.7 ml) were mixed and stirred at room temperature for 8.5 hours. The solvent was evaporated and the residue was triturated with diisopropyl ether -hexane to give ethyl 3-[3-[[4-[2-[[(2R)-2-(3-chlorophenyl)-2-hydroxyethyl]amino]ethyl]phenyl]sulfonyl]phenyl]propanoate hydrochloride (67 mg) as a white powder.

NMR (DMSO-d₆, δ): 1.10 (3H, t, J=7 Hz), 2.65 (2H, t, J=7 Hz), 2.80-3.45 (6H, m), 2.93 (2H, t, J=7 Hz), 4.00 (2H, q, J=7 Hz), 4.98 (1H, m), 6.32 (1H, d, J=4 Hz, OH), 7.25-7.68 (8H, m), 7.68-8.05 (4H, m), 8.99 (2H, br s) (+)ESI-MS (m/z): 516 (free, M+H)⁺

### EXAMPLE 123

The following compounds were obtained according to a similar manner to that of Example 54.

(1) 3-[3-[[4-[2-[[(2R)-2-(3-Chlorophenyl)-2-hydroxyethyl]amino]ethyl]phenyl]sulfonyl]phenyl]-propanoic acid hydrochloride

NMR (DMSO-d₆, δ): 2.57 (2H, t, J=7 Hz), 2.60-3.60 (6H, m), 2.91 (2H, t, J=7 Hz), 4.97 (1H, m), 6.33 (1H, br s, OH), 7.20-8.00 (12H, m), 8.90 (1H, br s), 9.00 (1H, br s), 12.15 (1H, br s) (+)ESI-MS (m/z): 488 (free, M+H)⁺

(2) 4-[[4-[2-[[(2R)-2-(3-Chlorophenyl)-2-hydroxyethyl]amino]-2-methylpropyl]phenyl]sulfonyl]benzoic acid hydrochloride

NMR (DMSO-d₆, δ): 1.21 (6H, s), 2.90-3.35 (4H, m), 4.99 (1H, m), 6.36 (1H, br s), 7.30-7.60 (6H, m), 7.96 (2H, d, J=8 Hz), 8.00-8.22 (4H, m), 9.25 (2H, br s) (+)ESI-MS (m/z): 488 (free, M+H)⁺

(3) 3-[[4-[2-[[(2R)-2-(3-Chlorophenyl)-2-hydroxyethyl]-amino]-2-methylpropyl]phenyl]sulfonyl]benzoic acid hydrochloride

NMR (DMSO-d₆, δ): 1.21 (6H, s), 2.90-3.40 (4H, m), 5.01 (1H, m), 6.34 (1H, br s), 7.25-7.65 (6H, m), 7.78 (1H, t, J=8 Hz), 7.98 (2H, d, J=8 Hz), 8.10-8.34 (2H, m), 8.40 (1H, s), 8.70 (1H, br s), 9.30 (1H, br s), 13.63 (1H, br s) (−)ESI-MS (m/z): 486 (free, M−H)⁻

(4) 3-[[4-[(2R)-2-[[(2R)-2-(3-Chlorophenyl)-2-hydroxyethyl]amino]propyl]phenyl]sulfonyl]benzoic acid hydrochloride

NMR (DMSO-d₆, δ): 1.10 (3H, d, J=6 Hz), 2.65-3.65 (5H, m), 5.10 (1H, m), 6.39 (1H, br s), 7.22-7.65 (6H, m), 7.78 (1H, t, J=8 Hz), 7.99 (2H, d, J=8 Hz), 8.10-8.34 (2H, m), 8.40 (1H, t, J=7 Hz), 8.93 (1H, br s), 9.61 (1H, br s), 13.60 (1H, br s) (+)ESI-MS (m/z): 474 (free, M+H)⁺

(5) 4-[[4-[(2S)-2-[[(2R)-2-(3-Chlorophenyl)-2-hydroxyethyl]amino]propyl]phenyl]sulfonyl]benzoic acid hydrochloride

NMR (DMSO-d₆, δ): 0.91 (3H, d, J=6 Hz), 2.45-3.10 (5H, m), 4.66 (1H, m), 7.10-7.53 (6H, m), 7.83 (2H, d, J=8 Hz), 7.87 (2H, d, J=8 Hz), 8.02 (2H, d, J=8 Hz) (+)ESI-MS (m/z): 474 (free, M+H)⁺

(6) 4-[[4-[(2R)-2-[[(2R)-2-(3-Chlorophenyl)-2-hydroxyethyl]amino]propyl]phenyl]sulfonyl]benzoic acid hydrochloride

NMR (DMSO-d₆, δ): 1.09 (3H, d, J=6 Hz), 2.65-3.65 (5H, m), 5.02 (1H, m), 6.37 (1H, br s, OH), 7.25-7.65 (6H, m), 7.97 (2H, d, J=8 Hz), 8.00-8.21 (4H, m), 9.15 (2H, br s) (−)ESI-MS (m/z): 472 (free, M−H)⁻

### EXAMPLE 124

The mixture of tert-butyl N-[(2R)-2-(3-chlorophenyl)-2-hydroxyethyl-N-[2-[4-[(3-formylphenyl)sulfonyl]phenyl]-ethyl]carbamate (217 mg), 2,4-thiazolidinedione (60 mg), and ammonium acetate (68 mg) in acetic acid (0.23 ml)-benzene (4.4 ml) was heated to reflux for 11 hours. After being allowed to cool to room temperature, the mixture was partitioned between ethyl acetate and sodium bicarbonate solution. The organic layer was separated, washed successively with water and brine, dried over magnesium sulfate, and filtered. The filtrate was concentrated and the residue was purified by column chromatography (silica gel, hexane/ethyl acetate) to give tert-butyl N-[(2R)-2-(3-chlorophenyl)-2-hydroxyethyl]-N-[2-[4-[[3-[(Z)-(2,4-dioxo-1,3-thiazolidin-5-ylidene)methyl]phenyl]sulfonyl]phenyl]-ethyl]carbamate (161 mg) as a white amorphous powder.

NMR (CDCl₃, δ): 1.35 (9H, s), 2.60-3.60 (6H, m), 4.24 (1H, br s, OH), 4.84 (1H, m), 7.08-7.48 (6H, m), 7.50-7.72 (2H, m), 7.81 (1H, s), 7.89 (2H, d, J=8 Hz), 7.90-8.10 (2H, m), 9.60 (1H, br s) (−)ESI-MS (m/z): 641 (M−H)⁻

### EXAMPLE 125

The following compounds were obtained according to a similar manner to that of Example 57.

(1) tert-Butyl N-[(2R)-2-(3-chlorophenyl)-2-hydroxyethyl]-N-[2-[4-[[3-[(dimethylamino)carbonyl]phenyl]-sulfonyl]phenyl]ethyl]carbamate

NMR (CDCl₃, δ): 1.38 (9H, s), 2.60-3.55 (6H, m), 2.95 (3H, s), 3.11 (3H, s), 4.28 (1H, br s, OH), 4.86 (1H, br s), 7.10-7.42 (6H, m), 7.45-7.68 (2H, m), 7.84 (2H, d, J=8 Hz), 7.90-8.02 (2H, m) (+)ESI-MS (m/z): 609 (M+Na)⁺

(2) tert-Butyl N-[(2R)-2-(3-chlorophenyl)-2-hydroxyethyl]-N-[2-[4-[[4-[(dimethylamino)carbonyl]phenyl]sulfonyl]phenyl]ethyl]carbamate

NMR (DMSO-d₆, δ): 1.05, 1.19 (total 9H, a pair of s), 2.65-3.60 (6H, m), 2.82 (3H, s), 2.98 (3H, s), 4.73 (1H, m), 5.58 (1H, br s, OH), 7.10-7.50 (6H, m), 7.60 (2H, d, J=8 Hz), 7.89 (2H, d, J=8 Hz), 7.98 (2H, d, J=8 Hz) (+)ESI-MS (m/z): 609 (M+Na)⁺

(3) tert-Butyl N-[(2R)-2-(3-chlorophenyl)-2-hydroxyethyl]-N-[2-[4-[[3-[(methylamino)carbonyl]phenyl]sulfonyl]-phenyl]ethyl]carbamate

NMR (CDCl₃, δ): 1.36 (9H, s), 2.65-3.00 (2H, m), 3.00 (3H, d, J=5 Hz), 3.08-3.60 (4H, m), 4.39 (1H, br s, OH), 4.59 (1H, m), 6.36 (1H, br s), 7.05-7.40 (6H, m), 7.55 (1H, t, J=8 Hz), 7.88 (2H, d, J=8 Hz), 7.94 (1H, br q, J=8 Hz), 8.03 (1H, d, J=8 Hz), 8.19 (1H, s) (+)ESI-MS (m/z): 595 (M+Na)⁺

(4) tert-Butyl N-[(2R)-2-(3-chlorophenyl)-2-hydroxyethyl]-N-[(1R)-1-methyl-2-[4-[[4-[(methylamino)carbonyl]-phenyl]sulfonyl]phenyl]ethyl]carbamate

NMR (CDCl₃, δ): 1.24 (3H, d, J=6 Hz), 1.26 (9H, s), 2.50-3.65 (4H, m), 3.00 (3H, d, J=5 Hz), 3.92-4.28 (1H, m), 4.60 (1H, m), 5.21 (1H, br s, OH), 6.17 (1H, br q, J=5 Hz), 7.05-7.45 (6H, m), 7.80 (2H, d, J=8 Hz), 7.82 (2H, d, J=8 Hz), 7.96 (2H, d, J=8 Hz) (+)ESI-MS (m/z): 609 (M+Na)⁺

(5) tert-Butyl N-[(2R)-2-(3-chlorophenyl)-2-hydroxyethyl]-N-[(1R)-2-[4-[[4-[(dimethylamino)carbonyl]phenyl]-sulfonyl]phenyl]-1-methylethyl]carbamate

NMR (CDCl₃, δ): 1.23 (3H, d, J=7 Hz), 1.28 (9H, s), 2.50-3.70 (4H, m), 2.91 (3H, s), 3.11 (3H, s), 4.00-4.28 (1H, m), 4.73 (1H, m), 5.22 (1H, br s, OH), 7.10-7.47 (6H, m), 7.49 (2H, d, J=8 Hz), 7.84 (2H, d, J=8 Hz), 7.96 (2H, d, J=8 Hz) (+)ESI-MS (m/z): 623 (M+Na)⁺

### EXAMPLE 126

To a mixture of 3-[[4-[2-[N-(tert-butoxycarbonyl)-N-[(2R)-2-(3-chlorophenyl)-2-hydroxyethyl]amino]ethyl]phenyl]-sulfonyl]benzoic acid (168 mg), 4-hydroxypiperidine (34 mg), and 1-hydroxybenzotriazole (44 mg) in N,N-dimethylformamide (1.3 ml) was added 1-(3-dimethylaminopropyl)-3-ethylcarbodiimide hydrochloride (75 mg), and the mixture was stirred at room temperature for 48.5 hours. The mixture was partitioned between hexane/ethyl acetate and water. The organic layer was separated, washed successively with water and brine, dried over magnesium sulfate, and filtered. The filtrate was concentrated and the residue was purified by column chromatography (silica gel, ethyl acetate/methanol) to give tert-butyl N-[(2R)-2-(3-chlorophenyl)-2-hydroxyethyl]-N-[2-[4-[[3-[(4-hydroxy-1-piperidinyl)-carbonyl]phenyl]sulfonyl]phenyl]ethyl]carbamate (188 mg) as a white amorphous powder.

NMR (CDCl₃, δ): 1.38 (9H, s), 1.50-2.20 (4H, m), 2.50-3.75 (10H, m), 4.01 (1H, m), 4.12 (1H, br s, OH), 4.86 (1H, m), 7.05-8.10 (12H, m) (+)ESI-MS (m/z): 665 (M+Na)⁺

### EXAMPLE 127

The following compounds were obtained according to a similar manner to that of Example 126.

(1) tert-Butyl N-[(2R)-2-(3-chlorophenyl)-2-hydroxyethyl]-N-[(1R)-2-[4-[[4-[(4-hydroxy-1-piperidinyl)carbonyl]-phenyl]sulfonyl]phenyl]-1-methylethyl]carbamate

NMR (CDCl₃, δ): 1.24 (3H, d, J=6 Hz), 1.25 (9H, s), 1.50-2.10 (4H, m), 2.50-3.70 (8H, m), 4.00 (1H, m), 4.14 (1H, m), 4.15 (1H, br s, OH), 4.74 (1H, m), 5.26 (1H, br s, OH), 7.10-7.45 (6H, m), 7.47 (2H, d, J=8 Hz), 7.85 (2H, d, J=8 Hz), 7.96 (2H, d, J=8 Hz) (+)ESI-MS (m/z): 679 (M+Na)⁺

(2) tert-Butyl N-[(2R)-2-(3-chlorophenyl)-2-hydroxyethyl]-N-[2-[4-[[3-[[(5-methyl-1,3-thiazol-2-yl)amino]-carbonyl]phenyl]sulfonyl]phenyl]ethyl]carbamate

NMR (CDCl₃, δ): 1.37 (9H, s), 2.39 (3H, s), 2.60-3.70 (6H, m), 4.35 (1H, br s, OH), 4.68 (1H, m), 6.84 (1H, br s), 7.02-7.46 (7H, m), 7.63 (1H, t, J=8 Hz), 7.89 (2H, d, J=8 Hz), 8.10 (1H, d, J=8 Hz), 8.16 (1H, d, J=8 Hz), 8.44 (1H, s) (−)ESI-MS (m/z): 654 (M−H)⁻

(3) tert-Butyl N-[(2R)-2-(3-chlorophenyl)-2-hydroxyethyl]-N-[2-[4-[[4-[[(5-methyl-1,3-thiazol-2-yl)amino]-carbonyl]phenyl]sulfonyl]phenyl]ethyl]carbamate

NMR (CDCl₃, δ): 1.36 (9H, s), 2.38 (3H, s), 2.60-3.60 (6H, m), 4.25 (1H, br s, OH), 4.81 (1H, m), 6.77 (1H, s), 7.06-7.50 (7H, m), 7.89 (2H, d, J=8 Hz), 8.03 (4H, s) (−)ESI-MS (m/z): 654 (M−H)⁻

(4) Ethyl 4-[[4-[(2R)-2-[[(2R)-2-(3-chlorophenyl)-2-hydroxyethyl]amino]propyl]phenyl]sulfonyl]benzoate hydrochloride

NMR (DMSO-d₆, δ): 1.09 (3H, d, J=6 Hz), 1.31 (3H, t, J=7 Hz), 2.65-3.65 (5H, m), 4.34 (2H, q, J=7 Hz), 5.02 (1H, m), 6.34 (1H, br s, OH), 7.28-7.62 (6H, m), 7.96 (2H, d, J=8 Hz), 8.11 (2H, d, J=8 Hz), 8.14 (2H, d, J=8 Hz), 8.81 (1H, br s), 9.23 (1H, br s) (+)ESI-MS (m/z): 502 (free, M+H)⁺

(5) Ethyl 4-[[3-[2-[[(2R)-2-(3-chlorophenyl)-2-hydroxyethyl]amino]ethyl]phenyl]sulfonyl]benzoate hydrochloride

NMR (DMSO-d₆, δ): 1.31 (3H, t, J=7 Hz), 2.90-3.40 (6H, m), 4.34 (2H, q, J=7 Hz), 4.98 (1H, m), 6.35 (1H, br s, OH), 7.28-7.52 (4H, m), 7.55-7.73 (2H, m), 7.80-8.00 (2H, m), 8.12 (2H, d, J=8 Hz), 8.14 (2H, d, J=8 Hz), 8.91 (1H, br s), 9.12 (1H, br s) (+)ESI-MS (m/z): 488 (free, M+H)⁺

### EXAMPLE 128

To a solution of 3-[[4-[2-[N-(tert-butoxycarbonyl)-N-[(2R)-2-(3-chlorophenyl)-2-hydroxyethyl]amino]ethyl]-phenyl]sulfonyl]benzoic acid (83 mg), 5-amino-1H-tetrazole (17 mg), 1-hydroxybenzotriazole (20 mg), and 4-(dimethylamino)pyridine (18 mg) in N,N-dimethylformamide (0.84 ml) was added 1-(3-dimethylaminopropyl)-3-ethylcarbodiimide hydrochloride (46 mg), and the mixture was stirred at room temperature for 5 days. The mixture was partitioned between hexane/ethyl acetate and water. The organic layer was separated, washed successively with water and brine, dried over magnesium sulfate, and filtered. The filtrate was concentrated and the residue was purified by gel permeation chromatography to give tert-butyl N-[(2R)-2-(3-chlorophenyl)-2-hydroxyethyl]-N-[2-[4-[[3-[(1H -tetrazol-5-ylamino)carbonyl]phenyl]sulfonyl]phenyl]ethyl]carbamate (51 mg) as a white amorphous powder.

NMR (DMSO-d₆, δ): 1.03, 1.18 (total 9H, a pair of s), 2.70-2.95 (2H, m), 3.03-3.60 (4H, m), 4.74 (1H, m), 5.59 (1H, br s, OH), 7.15-7.55 (6H, m), 7.80 (1H, t, J=8 Hz), 7.94 (2H, d, J=8 Hz), 8.20 (1H, m), 8.32 (1H, d, J=8 Hz), 8.66 (1H, s), 12.60 (1H, br s) (−)ESI-MS (m/z): 625 (M−H)⁻

### EXAMPLE 129

The following compound was obtained according to a similar manner to that of Example 128.

tert-Butyl N-[(2R)-2-(3-chlorophenyl)-2-hydroxyethyl]-N-[2-[4-[[4-[(1H-tetrazol-5-ylamino)carbonyl]phenyl]-sulfonyl]phenyl]ethyl]carbamate

NMR (DMSO-d₆, δ): 1.07, 1.20 (total 9H, a pair of s), 2.70-3.00 (2H, m), 3.05-3.60 (4H, m), 4.73 (1H, m), 5.60 (1H, br s, OH), 7.15-7.55 (6H, m), 7.93 (2H, m), 8.10 (2H, m), 8.20 (2H, d, J=8 Hz) (−)ESI-MS (m/z): 625 (M−H)⁻

### EXAMPLE 130

To a mixture of 3-[[4-[2-[N-(tert-butoxycarbonyl)-N-[(2R)-2-(3-chlorophenyl)-2-hydroxyethyl]amino]ethyl]-phenyl]sulfonyl]benzoic acid (112 mg), glycine ethyl ester hydrochloride (32 mg), and 1-hydroxybenzotriazole (29 mg) in N,N-dimethylformamide (1.1 ml) was added 1-[3-(dimethylamino)propyl]-3-ethylcarbodiimide (45 mg), and the mixture was stirred at room temperature for 15 hours. The mixture was partitioned between hexane/ethyl acetate and water. The organic layer was separated, washed successively with water and brine, dried over magnesium sulfate, and filtered. The filtrate was concentrated and the residue was purified by column chromatography (silica gel, hexane/ethyl acetate) to give ethyl [[3-[[4-[2-[N-(tert-butoxycarbonyl)-N-[(2R)-2-(3-chlorophenyl)-2-hydroxyethyl]amino]ethyl]-phenyl]sulfonyl]benzoyl]amino]acetate (114 mg) as a white amorphous powder.

NMR (CDCl₃, δ): 1.32 (3H, t, J=7 Hz), 1.36 (9H, s), 2.60-3.60 (6H, m), 4.22 (2H, d, J=5 Hz), 4.27 (2H, q, J=7 Hz), 4.33 (1H, br s, OH), 4.74 (1H, m), 6.78 (1H, br t, J=5 Hz), 7.10-7.40 (6H, m), 7.57 1H, t, J=8 Hz), 7.88 (2H, d, J=8 Hz), 7.97 (1H, d, J=8 Hz), 8.07 (1H, d, J=8 Hz), 8.30 (1H, s) (+)ESI-MS (m/z): 667 (M+Na)⁺

### EXAMPLE 131

The following compounds were obtained according to a similar manner to that of Example 130.

(1) Ethyl [[4-[[4-[2-[N-(tert-butoxycarbonyl)-N-[(2R)-2-(3-chlorophenyl)-2-hydroxyethyl]amino]ethyl]phenyl]-sulfonyl]benzoyl]amino]acetate

NMR (CDCl₃, δ): 1.31 (3H, t, J=7 Hz), 1.35 (9H, s), 2.60-3.60 (6H, m), 4.22 (2H, d, J=5 Hz), 4.25 (1H, br s, OH), 4.26 (2H, q, J=7 Hz), 4.82 (1H, m), 6.67 (1H, br t, J=5 Hz), 7.08-7.48 (6H, m), 7.86 (2H, d, J=8 Hz), 7.89 (2H, d, J=8 Hz), 7.99 (2H, d, J=8 Hz) (+)ESI-MS (m/z): 667 (M+Na)⁺

(2) Ethyl [[4-[[4-[(2R)-2-[N-(tert-butoxycarbonyl)-N-[(2R)-2-(3-chlorophenyl)-2-hydroxyethyl]amino]propyl]phenyl]-sulfonyl]benzoyl]amino]acetate

NMR (CDCl₃, δ): 1.24 (3H, d, J=6 Hz), 1.26 (9H, s), 1.31 (3H, t, J=7 Hz), 2.50-3.65 (4H, m), 4.00-4.26 (1H, m), 4.21 (2H, d, J=5 Hz), 4.26 (2H, q, J=7 Hz), 4.66 (1H, m), 5.26 (1H, br s, OH), 6.65 (1H, br t, J=5 Hz), 7.05-7.50 (6H, m), 7.86 (2H, d, J=8 Hz), 7.87 (2H, d, J=8 Hz), 7.99 (2H, d, J=8 Hz) (+)ESI-MS (m/z): 681 (M+Na)⁺

(3) Ethyl 3-[[3-[[4-[2-[N-(tert-butoxycarbonyl)-N-[(2R)-2-(3-chlorophenyl)-2-hydroxyethyl]amino]ethyl]phenyl]-sulfonyl]benzoyl]amino]propanoate

NMR (CDCl₃, δ): 1.28 (3H, t, J=7 Hz), 1.36 (9H, s), 2.55-3.60 (6H, m), 2.65 (2H, t, J=6 Hz), 3.72 (2H, q, J=6 Hz), 4.19 (2H, q, J=7 Hz), 4.32 (1H, br s, OH), 4.77 (1H, m), 6.96 (1H, br t, J=6 Hz), 7.05-7.42 (6H, m), 7.55 (1H, t, J=8 Hz), 7.88 (2H, d, J=8 Hz), 7.92 (1H, d, J=8 Hz), 8.04 (1H, d, J=8 Hz), 8.26 (1H, s) (+)ESI-MS (m/z): 681 (M+Na)⁺

(4) Ethyl [[3-[[4-[2-[N-(tert-butoxycarbonyl)-N-[(2R)-2-(3-chlorophenyl)-2-hydroxyethyl]amino]ethyl]phenyl]-sulfonyl]benzoyl)(methyl)amino]acetate

NMR (CDCl₃, δ): 1.32 (3H, t, J=7 Hz), 1.37 (9H, s), 2.60-3.60 (6H, m), 3.02, 3.13 (total 3H, a pair of s), 3.91 (1H, br s, OH), 4.25 (2H, s), 4.25 (2H, q, J=7 Hz), 4.87 (1H, m), 7.05-8.10 (12H, m) (+)ESI-MS (m/z): 681 (M+Na)⁺

(5) Ethyl (2S)-2-[[3-[[4-[2-[N-(tert-butoxycarbonyl)-N-[-(2R)-2-(3-chlorophenyl)-2-hydroxyethyl]amino]ethyl]-phenyl]sulfonyl]benzoyl]amino]propanoate

NMR (CDCl₃, δ): 1.32 (3H, t, J=7 Hz), 1.36 (9H, s), 1.54 (3H, d, J=7 Hz), 2.60-3.60 (6H, m), 4.26 (2H, q, J=7 Hz), 4.27 (1H, br s, OH), 4.35 (1H, quintet, J=7 Hz), 4.36 (1H, m), 6.83 (1H, br d, J=7 Hz), 7.05-7.43 (6H, m), 7.56 (1H, t, J=8 Hz), 7.88 (2H, d, J=8 Hz), 7.97 (1H, d, J=8 Hz), 8.06 (1H, d, J=8 Hz), 8.30 (1H, s) (+)ESI-MS (m/z): 681 (M+Na)⁺

(6) Methyl (2R)-2-[[3-[[4-[2-[N-(tert-butoxycarbonyl)-N-[(2R)-2-(3-chlorophenyl)-2-hydroxyethyl]amino]ethyl]-phenyl]sulfonyl]benzoyl]amino]propanoate

NMR (CDCl₃, δ): 1.36(9H, s), 1.54(3H, d, J=7 Hz), 2.55-3.55(6H, m), 3.80(3H, s), 4.32(1H, br s, OH), 4.77(1H, quintet, J=7 Hz), 4.77(1H, m), 6.81(1H, br d, J=7 Hz), 7.10-7.42(6H, m), 7.56(1H, t, J=8 Hz), 7.88(2H, d, J=8 Hz), 7.97(1H, d, J=8 Hz), 8.06(1H, d, J=8 Hz), 8.30(1H, s) (+)ESI-MS (m/z): 667 (M+Na)⁺

(7) Ethyl [[4-[[4-[(2R)-2-[N-(tert-butoxycarbonyl)-N-[(2R)-2-(3-chlorophenyl)-2-hydroxyethyl]amino]propyl]phenyl]-sulfonyl]benzoyl](methyl)amino]acetate

NMR (CDCl₃, δ): 1.23 (3H, d, J=7 Hz), 1.26 (9H, s), 1.27 (3H, t, J=7 Hz), 2.50-3.70 (4H, m), 2.97, 3.11 (total 3H, a pair of s), 3.87, 4.25 (total 3H, a pair of s), 4.16 (1H, m), 4.24 (2H, q, J=7 Hz), 4.74 (1H, m), 5.26 (1H, br s, OH), 7.10-7.68 (8H, m), 7.75-8.10 (4H, m) (+)ESI-MS (m/z): 695 (M+Na)⁺

### EXAMPLE 132

The following compound was obtained according to a similar manner to that of Example 122.

Ethyl (2R)-2-[[3-[[4-[2-[[(2R)-2-(3-chlorophenyl)-2-hydroxyethyl]amino]ethyl]phenyl]sulfonyl]benzoyl]amino]-propanoate hydrochloride

NMR (DMSO-d₆, δ): 1.19 (3H, t, J=7 Hz), 1.42 (3H, d, J=7 Hz), 2.85-3.55 (6H, m), 4.11 (2H, q, J=7 Hz), 4.47 (1H, quintet, J=7 Hz), 4.91 (1H, m), 6.33 (1H, br s, OH), 7.20-7.55 (4H, m), 7.54 (2H, d, J=8 Hz), 7.75 (1H, t, J=8 Hz), 7.98 (2H, d, J=8 Hz), 8.14 (1H, d, J=8 Hz), 8.19 (1H, d, J=8 Hz), 8.44 (1H, s), 9.05 (2H, br s), 9.16 (1H, br d, J=7 Hz) (+)ESI-MS (m/z): 559 (free, M+H)⁺

### EXAMPLE 133

At room temperature, to a solution of ethyl (R)-[4-[[4-[2-[[2-(3-chlorophenyl)-2-hydroxyethyl]amino]ethyl)phenyl]-sulfonyl]phenoxy]acetate (296 mg) in ethanol (5 ml) was added 4N hydrogen chloride in ethanol (1 ml), and the mixture was evaporated under reduced pressure and dried in vacuo to give ethyl (R)-[4-[[4-(2-[[2-(3-chlorophenyl)-2-hydroxyethyl]amino]ethyl]phenyl]sulfonyl]phenoxy]acetate hydrochloride(296 mg), which was recrystallized from ethanol.

mp: 198-200° C. NMR (DMSO-d₆, δ): 1.20 (3H, t, J=7.1 Hz), 2.95-3.3 (6H, m), 4.16 (2H, q, J=7.1 Hz), 4.85-5.0 (1H, m), 4.91 (2H, s), 7.1-7.2 (2H, m), 7.3-7.55 (6H, m), 7.8-7.95 (4H, m) IR (KBr): 2958, 1762, 1733, 1594, 1295, 1214, 1155, 1108, 1074, 686 cm⁻¹ (+)ESI-MS (m/z): 518, 520 (M-HCl+H)⁺

### EXAMPLE 134

A solution of 4-[[4-[2-[N-benzyl-N-[(2R)-2-(3-chlorophenyl)-2-hydroxyethyl]amino]ethyl]phenyl]sulfonyl]-2-fluorobenzoate (251 mg) in 7N hydrogen chloride in ethanol (1.0 ml) was stirred at room temperature for 1 hour. The solvent was removed by evaporation and the residue was dissolved in a mixed solvent of chlorobenzene (1.75 ml) and ethanol (0.75 ml). To the solution was added 10% palladium on activated carbon (50% wet, 25 mg) and the mixture was hydrogenated (1 atm) for 2 hours. The precipitates were dissolved by addition of ethanol and the catalyst was removed by filtration and washed with ethanol. The filtrate was concentrated in vacuo to give ethyl 4-[[4-[2-[[(2R)-2-(3-chlorophenyl)-2-hydroxyethyl]amino]ethyl]phenyl]-sulfonyl]-2-fluorobenzoate hydrochloride (219 mg) as an orange solid.

(+)APCI-MS (m/z): 506 (M+H)⁺

### EXAMPLE 135

The following compounds were obtained according to a similar manner to that of Example 134.

(1) Ethyl [4-[[4-[(2S)-2-[[(2R)-2-(3-chlorophenyl)-2-hydroxyethyl]amino]-3-hydroxypropyl]phenyl]-sulfonyl]phenoxy]acetate hydrochloride

NMR (DMSO-d₆, δ): 1.20 (3H, t, J=7 Hz), 2.75-3.75 (7H, m), 4.15 (2H, q, J=7 Hz), 4.81 (2H, s), 5.02 (1H, m), 5.40 (1H, br s, OH), 6.33 (1H, br s, OH), 7.13 (2H, d, J=9 Hz), 7.25-7.65 (6H, m), 7.88 (2H, d, J=9 Hz), 7.91 (2H, d, J=8 Hz), 8.58 (1H, br s), 9.19 (1H, br s) (+)ESI-MS (m/z): 548 (free, M+H)⁺

(2) Ethyl 3-[[3-[2-[[(2R)-2-(3-chlorophenyl)-2-hydroxyethyl]amino]ethyl]phenyl]sulfonyl]benzoate hydrochloride

NMR (DMSO-d₆, δ): 1.34 (3H, t, J=7 Hz), 2.90-3.50 (6H, m), 4.36 (2H, q, J=7 Hz), 5.00 (1H, m), 6.36 (1H, br s, OH), 7.28-8.05 (9H, m), 8.12-8.32 (2H, m), 8.42 (1H, s), 8.91 (1H, br s), 9.18 (1H, br s) (+)ESI-MS (m/z): 488 (free, M+H)⁺

(3) Ethyl 4-[[4-[2-[((2R)-2-(3-chlorophenyl)-2-hydroxyethyl]amino]ethyl]phenyl]sulfonyl]-2-methylbenzoate hydrochloride

(+)APCI-MS (m/z): 502 (M+H)⁺

(4) Ethyl 2′-chloro-4′-[(4-[2-[[(2R)-2-(3-chlorophenyl)-2-hydroxyethyl]amino]ethyl]phenyl]sulfonyl]-1,1′-biphenyl-4-carboxylate hydrochloride

NMR (DMSO-d₆, δ): 1.34 (3H, t, J=7.1 Hz), 2.98-3.20 (6H, m), 4.35 (2H, q, J=7.1 Hz), 5.00-5.05 (1H, m), 6.37 (1H, d, J=4.1 Hz), 7.31-7.72 (9H, m), 7.99-8.08 (5H, m), 8.18 (1H, d, J=1.7 Hz), 9.05 (1H, br), 9.30 (1H, br) (+)APCI-MS (m/z): 598 (M+H)⁺

(5) Ethyl 2′-chloro-4′-[[4-[2-[[(2R)-2-(3-chlorophenyl)-2-hydroxyethyl]amino]ethyl]phenyl]sulfonyl]-1,1′-biphenyl-3-carboxylate hydrochloride

NMR (DMSO-d₆, δ): 1.31 (3H, t, J=7.1 Hz), 2.98-3.20 (6H, m), 4.33 (2H, q, J=7.1 Hz), 4.99-5.04 (1H, m), 6.36 (1H, m), 7.31-7.46 (5H, m), 7.55-7.77 (4H, m), 7.98-8.08 (5H, m), 8.18 (1H, d, J=1.7 Hz), 9.07 (1H, br), 9. 23 (1H, br) (+)APCI-MS (m/z): 598 (M+H)⁺

(6) Ethyl 4-[[4-[[[(2R)-2-(3-chlorophenyl)-2-hydroxyethyl]amino]methyl]phenyl]sulfonyl]benzoate hydrochloride

NMR (DMSO-d₆, δ): 1.32 (3H, t, J=7 Hz), 2.85-3.25 (2H, m), 4.27 (2H, s), 4.34 (2H, q, J=7 Hz), 5.01 (1H, m) 6.70 (1H, br s, OH), 7.20-7.50 (4H, m), 7.82 (2H, d, J=8 Hz), 8.05 (2H, d, J=8 Hz), 8.14 (4H, s), 9.41 (2H, br s) (+) ESI-MS (m/z): 474 (free, M+H)⁺

(7) Ethyl 4-[[4-[3-[[(2R)-2-(3-chlorophenyl)-2-hydroxyethyl]amino]propyl]phenyl]sulfonyl]benzoate hydrochloride

NMR (DMSO-d₆, δ): 1.31 (3H, t, J=7 Hz), 1.96 (2H, quintet, J=7 Hz), 2.74 (2H, t, J=7 Hz), 2.90-3.28 (2H, m), 2.93 (2H, t, J=7 Hz), 4.34 (2H, q, J=7 Hz), 4.96 (1H, m), 6.29 (1H, br s, OH), 7.25-7.55 (4H, m), 7.51 (2H, d, J=8 Hz), 7.93 (2H, d, J=8 Hz), 8.10 (2H, d, J=8 Hz), 8.14 (2H, d, J=8 Hz), 8.89 (2H, br s) (+)ESI-MS (m/z): 502 (free, M+H)⁺

(8) Ethyl [4-[[4-[[[(2R)-2-(3-chlorophenyl)-2-hydroxyethyl]amino]methyl]phenyl]sulfonyl]phenoxy]-acetate hydrochloride

NMR (DMSO-d₆, δ): 1.20 (3H, t, J=7 Hz), 2.80-3.35 (2H, m), 4.15 (2H, q, J=7 Hz), 4.26 (2H, br s), 4.92 (2H, s), 5.02 (1H, m), 6.30 (1H, br s, OH), 7.14 (2H, d, J=9 Hz), 7.22-7.52 (4H, m), 7.78 (2H, d, J=8 Hz), 7.90 (2H, d, J=9 Hz), 8.00 (2H, d, J=8 Hz), 9.28 (1H, br s), 9.56 (1H, br s) (+)ESI-MS (m/z): 504 (free, M+H)⁺

(9) Ethyl [4-[[4-[3-[[(2R)-2-(3-chlorophenyl)-2-hydroxyethyl]amino]propyl]phenyl]sulfonyl]phenoxy]-acetate hydrochloride

NMR (DMSO-d₆, δ): 1.20 (3H, t, J=7 Hz), 1.80-2.15 (2H, m), 2.55-3.30 (6H, m), 4.16 (2H, q, J=7 Hz), 4.91 (2H, s), 4.97 (1H, m), 6.30 (1H, br s, OH), 7.13 (2H, d, J=9 Hz), 7.25-7.60 (6H, m), 7.87 (2H, d, J=9 Hz), 7.87 (2H, d, J=8 Hz), 8.81 (1H, br s), 9.10 (1H, br s) (+)APCI-MS (m/z): 532 (free, M+H)⁺

### EXAMPLE 136

To a solution of ethyl 4-[[4-[2-[[(2R)-2-(3-chlorophenyl)-2-hydroxyethyl]amino]-2-methylpropyl]-phenyl]sulfonyl]benzoate (67 mg) in ethanol (1.3 ml) was added 4 M hydrogen chloride/ethanol (0.7 ml), and the solvent was evaporated. The residual solid was recrystallized from ethanol (0.7 ml)-hexane (2.1 ml) to give ethyl 4-[[4-[2-[[(2R)-2-(3-chlorophenyl)-2-hydroxyethyl]amino]-2-methylpropyl]phenyl]sulfonyl]benzoate hydrochloride (62 mg) as a white powder.

NMR (DMSO-d₆, δ): 1.21 (6H, s), 1.31 (3H, t, J=7 Hz), 2.90-3.30 (4H, m), 4.34 (2H, q, J=7 Hz), 4.99 (1H, m), 6.35 (1H, br s), 7.30-7.60 (6H, m), 7.96 (2H, d, J=8 Hz), 8.03-8.24 (4H, m), 8.63 (1H, br s), 9.28(1H, br s) (+)ESI-MS (m/z): 516 (free, M+H)⁺

### EXAMPLE 137

The following compounds were obtained according to a similar manner to that of Example 136.

(1) Ethyl 3-[[4-[2-[[(2R)-2-(3-chlorophenyl)-2-hydroxyethyl]amino]-2-methylpropyl]phenyl]sulfonyl]-benzoate hydrochloride

NMR (DMSO-d₆, δ): 1.22 (6H, s), 1.34 (3H, t, J=7 Hz), 2.90-3.35 (4H, m), 4.27 (2H, q, J=7 Hz), 5.04 (1H, m), 6.36 (1H, d, J=4 Hz), 7.25-7.65 (6H, m), 7.81 (1H, t, J=8 Hz), 7.99 (2H, d, J=8 Hz), 8.18-8.32 (2H, m), 8.41 (1H, s), 8.69 (1H, br s), 9.49 (1H, br s) (+)ESI-MS (m/z): 516 (free, M+H)⁺

(2) Ethyl 3-[[4-[(2R)-2-[[(2R)-2-(3-chlorophenyl)-2-hydroxyethyl]amino]propyl]phenyl]sulfonyl]benzoate hydrochloride

NMR (DMSO-d₆, δ): 1.09 (3H, d, J=6 Hz), 1.34 (3H, t, J=7 Hz), 2.70-3.65 (5H, m), 4.36 (2H, q, J=7 Hz), 5.03 (1H, m), 6.36 (1H, d, J=4 Hz), 7.28-7.65 (6H, m), 7.80 (1H, t, J=8 Hz), 7.99 (2H, d, J=8 Hz), 8.15-8.32 (2H, m), 8.40 (1H, t, J=7 Hz), 8.81 (1H, br s), 9.30 (1H, br s) (+)ESI-MS (m/z): 502 (free, M+H)⁺

(3) Ethyl 4-[[4-[(2S)-2-[[(2R)-2-(3-chlorophenyl)-2-hydroxyethyl]amino]propyl]phenyl]sulfonyl]benzoate hydrochloride

NMR (DMSO-d₆, δ): 1.10 (3H, d, J=6 Hz), 1.31 (3H, t, J=7 Hz), 2.95-3.60 (5H, m), 4.34 (2H, q, J=7 Hz), 5.03 (1H, m), 6.36 (1H, br d, J=4 Hz), 7.28-7.65 (6H, m), 7.96 (2H, d, J=8 Hz), 8.00-8.24 (4H, m), 8.81 (1H, br s), 9.34 (1H, br s) (+)ESI-MS (m/z): 502 (free, M+H)⁺

### EXAMPLE 138

To a suspension of ethyl 4-[[4-[2-[[(2R)-2-(3-chlorophenyl)-2-hydroxyethyl]amino]ethyl]phenyl]sulfonyl]-benzoate hydrochloride (550 mg) in ethanol (5.5 ml) was added 1N sodium hydroxide solution (2.3 ml), and the mixture was stirred at room temperature for 4 hours. After the solvent was evaporated, the residual solid was washed with water to give sodium 4-[[4-[2-[[(2R)-2-(3-chlorophenyl)-2-hydroxyethyl]amino]ethyl]phenyl]sulfonyl]benzoate (404 mg) as a white powder.

NMR (DMSO-d₆, δ): 1.80(1H, br s), 2.50-2.90(6H, m), 4.59(1H, m), 5.41(1H, br s), 7.15-7.50(6H, m), 7.82(4H, d, J=8 Hz), 7.98(2H, d, J=8 Hz) (−)ESI-MS (m/z): 460 (free, M−H)⁻

### EXAMPLE 139

The following compounds were obtained according to a similar manner to that of Example 138.

(1) Sodium 4-[[4-[3-[[(2R)-2-(3-chlorophenyl)-2-hydroxyethyl]amino]propyl]phenyl]sulfonyl]benzoate

NMR (DMSO-d₆, δ): 1.66 (2H, quintet, J=7 Hz), 2.35-2.80 (6H, m), 4.60 (1H, m), 5.44(1H, br s, OH), 7.15-7.55 (6H, m), 7.82 (2H, d, J=8 Hz), 7.82 (2H, d, J=8 Hz), 7.99 (2H, d, J=8 Hz) (+)ESI-MS (m/z): 474 (free, M+H)⁺

(2) Sodium 4-[[4-[(2R)-2-[[(2R)-2-(3-chlorophenyl)-2-hydroxyethyl]amino]propyl]phenyl]sulfonyl]benzoate

NMR (DMSO-d₆, δ): 0.88 (3H, d, J=6 Hz), 1.56 (1H, br s), 2.45-2.95 (5H, m), 4.55 (1H, m), 5.40 (1H, br s), 7.12-7.50 (6H, m), 7.80 (2H, d, J=8 Hz), 7.82 (2H, d, J=8 Hz), 7.99 (2H, d, J=8 Hz) (+)ESI-MS (m/z): 474 (free, M+H)⁺

(3) Sodium 3-[[3-[2-[[(2R)-2-(3-chlorophenyl)-2-hydroxyethyl]amino]ethyl]phenyl]sulfonyl]benzoate

NMR (DMSO-d₆, δ): 2.35-2.95 (6H, m), 4.61 (1H, dd, J=8 and 4 Hz), 7.00-7.60 (7H, m), 7.60-7.90 (2H, m), 7.90 (1H, d, J=8 Hz), 8.11 (1H, d, J=8 Hz), 8.37 (1H, s) (+)ESI-MS (m/z): 482 (M+H)⁺

(4) Sodium 4-[[3-[2-[[(2R)-2-(3-chlorophenyl)-2-hydroxyethyl]amino]ethyl]phenyl]sulfonyl]benzoate

NMR (DMSO-d₆, δ): 2.35-3.00 (6H, m), 4.62 (1H, m), 7.10-7.60 (6H, m), 7.60-8.00 (4H, m), 8.06 (2H, d, J=8 Hz) (+)ESI-MS (m/z): 482 (M+H)⁺

### EXAMPLE 140

To a solution of 4-[[4-[2-[[(2R)-2-(3-chlorophenyl)-2-hydroxyethyl]amino]ethyl]phenyl]sulfonyl]-2-fluorobenzoic acid hydrochloride (150 mg) in ethanol (1.5 ml) was added 1N sodium hydroxide (583 μl) and the solvent was removed by evaporation. The residue was chromatographed on ODS (Daisogel SP-120, eluent: water/methanol) to give sodium 4-[[4-[2-[[(2R)-2-(3-chlorophenyl)-2-hydroxyethyl]amino]-ethyl]phenyl]sulfonyl]-2-fluorobenzoate (132 mg) as a white solid.

(−)APCI-MS (m/z): 476 (M−Na)⁻

### EXAMPLE 141

The following compound was obtained according to a similar manner to that of Example 140.

Methyl 2-chloro-4-[[4-[2-[[(2R)-2-(3-chlorophenyl)-2-hydroxyethyl]amino]ethyl]phenyl]sulfonyl]benzoate hydrochloride

(+)APCI-MS (m/z): 508 (M+H)⁺

### EXAMPLE 142

To a suspension of ethyl 4-[[4-[2-[[(2R)-2-(3-chlorophenyl)-2-hydroxyethyl]amino]ethyl]phenyl]sulfonyl]-2-fluorobenzoate hydrochloride (204 mg) in ethanol (2.0 ml) was added 1N sodium hydroxide solution (0.94 ml) and the resulting solution was stirred at room temperature for 17 hours. To the solution were added 1N hydrochloric acid (0.94 ml) and water (4.0 ml). The resulting suspension was stirred for 1 hour and the precipitates were collected by filtration. The precipitates were washed with water and dried under reduced pressure to give 4-[[4-[2-[[(2R)-2-(3-chlorophenyl)-2-hydroxyethyl]amino]ethyl]phenyl]sulfonyl]-2-fluorobenzoic acid hydrochloride (161 mg) as a pale yellow solid.

IR (KBr): 3359, 3026, 1630, 1599, 1406, 1369, 1329, 1155, 698 cm⁻¹ NMR (DMSO-d₆, δ): 2.84-3.24 (6H, m), 4.94-4.97 (1H, m), 7.26-7.51 (6H, m), 7.67-7.81 (3H, m), 7.92 (2H, d, J=8.3 Hz) (−)APCI-MS (m/z): 476 (M−H)⁻

### EXAMPLE 143

The following compound was obtained according to a similar manner to that of Example 142.

(1) 2-Chloro-4-[[4-[2-[[(2R)-2-(3-chlorophenyl)-2-hydroxyethyl]amino]ethyl]phenyl]sulfonyl]benzoic acid hydrochloride

IR (KBr): 3421, 2952, 1724, 1593, 1576, 1385, 1363, 1308, 1157, 1109, 694 cm⁻¹ NMR (DMSO-d₆, δ): 2.95-3.22 (6H, m), 4.98-5.02 (1H, m), 7.32-7.52 (6H, m), 7.62 (1H, d, J=8.0 Hz), 7.80-7.97 (4H, m), 9.52 (2H, br) (−)APCI-MS (m/z): 492 (M−H)⁻

(2) 4-[[4-[2-[[(2R)-2-(3-Chlorophenyl)-2-hydroxyethyl]-amino]ethyl]phenyl]sulfonyl]-2-methylbenzoic acid hydrochloride

IR (KBr): 3417, 3005, 1716, 1597, 1294, 1194, 1155, 1084 cm⁻¹ NMR (DMSO-d₆, δ): 2.95-3.21 (6H, m), 4.97-5.00 (1H, m), 7.33-7.55 (6H, m), 7.81-7.97 (5H, m), 10.3 (2H, br) (−)APCI-MS (m/z): 472 (M−H)⁻

### EXAMPLE 144

To a solution of ethyl 4-[[4-[(2R)-2-[[(2R)-2-(3-chlorophenyl)-2-hydroxyethyl]amino]propyl]phenyl]sulfonyl]-2-fluorobenzoate (378 mg) in ethanol (3.8 ml) was added 1N sodium hydroxide (909 μl) and the mixture was stirred at room temperature overnight. An additional portion of 1N sodium hydroxide (363 μl) was added and the mixture was stirred at 60° C. for 3 hours. After cooling to room temperature, the solvent was removed by evaporation and the residual solid was chromatographed on ODS (Daisogel SP-120, eluent: water/methanol) to give sodium 4-[[4-[(2R)-2-[[(2R)-2-(3-chlorophenyl)-2-hydroxyethyl]amino]propyl]phenyl]sulfonyl]-2-fluorobenzoate (280 mg) as a white solid.

NMR (DMSO-d₆, δ): 1.00 (3H, d, J=6.2 Hz), 2.68 (1H, d, J=9.5, 12.6 Hz), 2.87-3.17 (4H, m), 3.30 (2H, br), 4.94-4.97 (1H, m), 7.35-7.46 (6H, m), 7.65-7.78 (3H, m), 7.89 (2H, d, J=8.3 Hz) (−)APCI-MS (m/z): 490 (M−Na)⁻

### EXAMPLE 145

The following compounds were obtained according to a similar manner to that of Example 144.

(1) Sodium 2-chloro-4-[[4-[(2R)-2-[[(2R)-2-(3-chlorophenyl)-2-hydroxyethyl]amino]propyl]phenyl]-sulfonyl]benzoate

NMR (DMSO-d₆, δ): 0.98 (3H, d, J=6.1 Hz), 2.67 (1H, dd, J=8.8, 12.7 Hz), 2.83-3.35 (6H, m), 4.86-4.89 (1H, m), 7.28-7.53 (7H, m), 7.74-7.90 (4H, m) (−)APCI-MS (m/z): 506 (M−Na)⁻

(2) Sodium 4-[[4-[(2R)-2-[[(2R)-2-(3-chlorophenyl)-2-hydroxyethyl]amino]propyl]phenyl]sulfonyl]-2-methylbenzoate

NMR (DMSO-d₆, δ): 0.94 (3H, d, J=6.0 Hz), 2.61 (1H, dd, J=8.0, 12.8 Hz), 2.80-3.17 (4H, m), 4.76 (1H, dd, J=4.2, 7.9 Hz), 7.24-7.41 (6H, m), 7.57-7.67 (3H, m), 7.82 (2H, d, J=8.2 Hz) (−)APCI-MS (m/z): 486 (M−Na)⁻

(3) Sodium 2′-chloro-4′-[[4-[2-[[(2R)-2-(3-chlorophenyl)-2-hydroxyethyl]amino]ethyl]phenyl]sulfonyl]-1,1′-biphenyl-4-carboxylate

NMR (DMSO-d₆, δ): 1.65 (1H, br), 2.61-2.78 (4H, m), 3.08-3.20 (2H, m), 4.60 (1H, br), 5.50 (1H, br), 7.23-7.67 (9H, m), 7.93-8.11 (6H, m) (−)APCI-MS (m/z): 568 (M−Na)⁻

(4) Sodium 2′-chloro-4′-[[4-[2-[[(2R)-2-(3-chlorophenyl)-2-hydroxyethyl]amino]ethyl]phenyl]sulfonyl]-1,1′-biphenyl-3-carboxylate

NMR (DMSO-d₆, δ): 2.69-2.90 (6H, m), 4.72-4.78 (1H, m), 7.24-7.71 (9H, m), 7.95-8.03 (5H, m), 7.95-8.03 (5H, m), 8.14 (1H, d, J=1.7 Hz) (+)APCI-MS (m/z): 570 (M+H)⁺

### EXAMPLE 146

To a solution of ethyl 5-[[4-(2-aminoethyl)phenyl]-sulfonyl]-2-methoxybenzoate (74.6 mg) in dimethyl sulfoxide (1.0 ml) was added N,O-bis(trimethylsilyl)acetamide (25.4 μl) and the solution was stirred at room temperature for 30 minutes. To the mixture was added (2R)-2-(3-chlorophenyl)oxirane (38.1 mg) and the whole was heated at 80° C. for 48 hours. After cooling to room temperature, the mixture was quenched by addition of 5% acetic acid in water (2.0 ml) and stirred for 30 minutes. The mixture was basified with saturated aqueous sodium bicarbonate (5.0 ml) and extracted with ethyl acetate (5.0 ml×3). The combined extracts were washed with water (10 ml×2) and brine (10 ml×1), and dried over magnesium sulfate. Filtration followed by evaporation gave a crude product, which was chromatographed on silica gel (eluent: chloroform/methanol) to give the ethyl 5-[[4-[2-[[(2R)-2-(3-chlorophenyl)-2-hydroxyethyl]amino]ethyl]phenyl]sulfonyl]-2-methoxybenzoate (49.8 mg) as a white solid.

(+)APCI-MS (m/z): 518 (M+H)⁺

### EXAMPLE 147

To a suspension of ethyl 5-[[4-[2-[[(2R)-2-(3-chlorophenyl)-2-hydroxyethyl]amino]ethyl]phenyl]sulfonyl]-2-methoxybenzoate (44.1 mg) in ethanol (0.44 ml) was added 1N sodium hydroxide (85.1 μl) and the mixture was stirred at room temperature for 5 hours. An additional portion of 1N sodium hydroxide (25.5 μl) was added and the mixture was stirred for 17 hours. The solvent was removed by evaporation and the residual solid was dried under reduced pressure to give sodium 5-[[4-[2-[[(2R)-2-(3-chlorophenyl)-2-hydroxyethyl]amino]ethyl]phenyl]sulfonyl]-2-methoxybenzoate (46.4 mg) as an orange solid.

NMR (CDCl₃, δ): 1.66 (1H, br), 2.59-2.75 (6H, m), 3.75 (3H, s), 4.59 (1H, br), 5.43 (1H, d, J=4.1 Hz), 7.04 (1H, d, J=8.7 Hz), 7.21-7.42 (6H, m), 7.58 (1H, d, J=2.5 Hz), 7.68-7.78 (3H, m) (−)APCI-MS (m/z): 488 (M−Na)⁻

### EXAMPLE 148

The following compound was obtained according to a similar manner to that of Example 147.

Sodium 4-[[4-[2-[[(2R)-2-(3-chlorophenyl)-2-hydroxyethyl]amino]ethyl]phenyl]sulfonyl]-2-methoxybenzoate

NMR (CDCl₃, δ): 1.67 (1H, br), 2.60-2.75 (6H, m), 3.76 (3H, s), 4.59 (1H, br), 5.42 (1H, d, J=3.7 Hz), 7.21-7.47 (9H, m), 7.83 (1H, d, J=8.1 Hz) (−)APCI-MS (m/z): 488 (M−Na)⁻

### EXAMPLE 149

The following compound was obtained according to a similar manner to that Example 49.

(1R)-2-[N-benzyl-N-[2-[4-[[3-(2-hydroxyethoxy)phenyl]-sulfonyl]phenyl]ethyl]amino]-1-(3-chlorophenyl)ethanol

(+)ESI-MS (m/z): 566 (M+H)⁺

### EXAMPLE 150

The following compounds were obtained according to a similar manner to that of Example 70.

(1) (1R)-2-[[2-[2-Chloro-4-[(4-methoxyphenyl)sulfonyl]-phenyl]ethyl]amino]-1-(3-chlorophenyl)ethanol hydrochloride

NMR (DMSO-d₆, δ): 3.02-3.35 (6H, m), 3.83 (3H, s), 4.95-4.99 (1H, m), 6.34-6.35 (1H, m), 7.12-7.16 (2H, m), 7.38-7.47 (4H, m), 7.68-7.99 (5H, m), 8.97 (1H, br) (+)ESI-MS (m/z): 480 (M-HCl+H)⁺

(2) N-[3-[[4-[2-[[(2R)-2-(3-Chlorophenyl)-2-hydroxyethyl]-amino]ethyl]phenyl]sulfonyl]phenyl]acetamide hydrochloride

NMR (DMSO-d₆, δ): 2.05 (3H, s), 3.0-3.4 (6H, m), 4.93-4.99 (1H, m), 6.32-6.34 (1H, m), 7.37-7.85 (12H, m), 8.32 (1H, s), 8.83-8.94 (1H, br), 10.38 (1H, s) (+)ESI-MS (m/z): 473 (M-HCl+H)⁺

(3) (1R)-1-(3-Chlorophenyl)-2-[[2-[4-[[3-(dimethylamino)-phenyl]sulfonyl]phenyl]ethyl]amino]ethanol hydrochloride

NMR (DMSO-d₆, δ): 2.48 (3H, s),2.49 (3H, s), 3.09-3.29 (6H, m), 4.98-5.04 (1H, m), 6.95-6.99 (1H, m), 7.13-7.16 (2H, m), 7.34-7.59 (7H, m), 7.82-7.88 (2H, m), 8.94 (1H, br), 9.26 (1H, br) (+)ESI-MS (m/z): 459 (M-HCl+H)⁺

(4) (1R)-1-(3-Chlorophenyl)-2-[[2-[6-[(4-methoxyphenyl)-sulfonyl]-3-pyridyl]ethyl]amino]ethanol hydrochloride

NMR (DMSO-d₆, δ): 3.00-3.47 (6H, m), 3.84 (3H, s), 4.95-5.00 (1H, m), 7.16 (2H, d, J=7.0 Hz), 7.33-7.45 (4H, m), 7.90 (2H, d, J=7.0 Hz), 8.03 (1H, d, J=8.0 Hz), 8.15 (1H, d, J=8.0 Hz), 8.60 (1H, s), 8.91 (1H, br), 9.15 (1H, br) (+)ESI-MS (m/z): 447 (M-HCl+H)⁺

### EXAMPLE 151

The following compounds were obtained according to a similar manner to that of Example 76.

(1) Ethyl [4-[[5-[2-[[(2R)-2-(3-chlorophenyl)-2hydroxyethyl]amino]ethyl]-2-pyridyl]sulfonyl]-phenoxy]acetate

(+)ESI-MS (m/z): 519 (M+H)⁺

(2) Ethyl [4-[[3-chloro-4-[2-[[(2R)-2-(3-chlorophenyl)-2-hydroxyethyl]amino]ethyl]phenyl]sulfonyl]phenoxy]-acetate

(+)ESI-MS (m/z): 552 (M+H)⁺

### EXAMPLE 152

The following compound was obtained according to a similar manner to that of Example 79.

Ethyl [4-[[5-[2-[[(2R)-2-(3-chlorophenyl)-2-hydroxyethyl]amino]ethyl]-2-pyridinyl]sulfonyl]phenoxy]-acetate hydrochloride

NMR (DMSO-d₆, δ): 1.20 (3H, t, J=7.0 Hz), 2.99-3.36 (6H, m), 4.16 (2H, q, J=7.0 Hz), 4.92 (2H, s), 4.90-4.95 (1H, m), 6.27-6.29 (1H, m), 7.14-7.17 (2H, m), 7.36-7.45 (4H, m), 7.87-7.89 (2H, m), 8.01-8.04 (1H, m), 8.16 (1H, d, J=4.0 Hz), 8.60 (1H, s), 8.78 (1H, br) (+)ESI-MS (m/z): 519 (M-HCl+H)⁺

### EXAMPLE 153

The following compounds were obtained according to a similar manner to that of Example 42.

(1) Ethyl [4-[[4-[[N-benzyl-N-[(2R)-2-(3-chlorophenyl)-2-hydroxyethyl]amino]methyl]phenyl]sulfonyl]phenoxy]-acetate

NMR (CDCl₃, δ): 1.28 (3H, t, J=7 Hz), 2.57 (1H, dd, J=13 and 9 Hz), 2.66 (1H, dd, J=13 and 4 Hz), 3.50 (1H, br s), 3.50 (1H, d, J=13 Hz), 3.55 (1H, d, J=14 Hz), 3.84 (1H, d, J=13 Hz), 3.89 (1H, d, J=14 Hz), 4.26 (2H, q, J=7 Hz), 4.65 (2H, s), 4.68 (1H, dd, J=9 and 4 Hz), 6.97 (2H, d, J=9 Hz), 7.00-7.50 (11H, m), 7.87 (2H, d, J=8 Hz), 7.89 (2H, d, J=9 Hz) (+)ESI-MS (m/z): 594 (M+H)⁺

(2) Ethyl [4-[[4-[3-[N-benzyl-N-[(2R)-2-(3-chlorophenyl)-2-hydroxyethyl]amino]propyl]phenyl]sulfonyl]phenoxy]-acetate

NMR (CDCl₃, δ): 1.29 (3H, t, J=7 Hz), 1.80 (2H, quintet, J=7 Hz), 2.35-2.80 (6H, m), 3.48 (1H, d, J=13 Hz), 3.87 (1H, d, J=13 Hz), 4.26 (2H, q, J=7 Hz), 4.60 (1H, dd, J=10 and 4 Hz), 4.65 (2H, s), 6.96 (2H, d, J=9 Hz), 7.08-7.45 (11H, m), 7.79 (2H, d, J=8 Hz), 7.87 (2H, d, J=9 Hz) (+)ESI-MS (m/z): 622 (M+H)⁺

### EXAMPLE 154

The following compound was obtained according to a similar manner to that of Example 138.

Sodium 4-[[4-[[[(2R)-2-(3-chlorophenyl)-2-hydroxyethyl]amino]methyl]phenyl]sulfonyl]benzoate

NMR (DMSO-d₆, δ): 2.60 (2H, d, AB of ABX), 3.78 (2H, s), 4.65 (1H, t, X of ABX), 5.45 (1H, br s, OH), 7.15-7.48 (4H, m), 7.52 (2H, d, J=8 Hz), 7.83 (2H, d, J=8 Hz), 7.86 (2H, d, J=8 Hz), 8.00 (2H, d, J=8 Hz) (−)ESI-MS (m/z): 444 (free, M−H)⁻

### EXAMPLE 155

To a solution of methyl [3-[[4-[2-[[(2R)-2-(3-chlorophenyl)-2-hydroxyethyl]amino]ethyl]phenyl]sulfonyl]-phenoxy]acetate (51 mg) in methanol (1.0 ml) was added 1M ammonia in methanol (2.0 ml), and the mixture was stirred at room temperature for 4 hours. The mixture was evaporated under reduced pressure. The residue was dissolved in dichloromethane (20 ml) and methanol (2.0 ml), and washed with water (5.0 ml). The aqueous layer was extracted with dichloromethane (20 ml). The combined organic layers were dried over magnesium sulfate and evaporated under reduced pressure. The residue was suspended in 4N hydrogen chloride in ethyl acetate (0.5 ml) and stirred for 5 minutes. The solvent was removed by evaporation to give 2-[3-[[4-[2-[[(2R)-2-(3-chlorophenyl)-2-hydroxyethyl]amino]ethyl]-phenyl]sulfonyl]phenoxy]acetamide hydrochloride (33 mg) as a white foam.

NMR (DMSO-d₆, δ): 3.05-3.53 (6H, m), 4.53 (2H, s), 4.93-4.98 (1H, m), 6.31-6.33 (1H, m), 7.22-7.26 (1H, m), 7.36-7.55 (9H, m), 7.92-7.96 (2H, m), 8.84-8.99 (2H, br) (+)ESI-MS (m/z): 489 (M-HCl+H)⁺

### EXAMPLE 156

Under nitrogen at room temperature, to a solution of 3-[[4-[2-[N-benzyl-N-[(2R)-2-(3-chlorophenyl)-2-hydroxyethyl]-amino]ethyl]phenyl]sulfonyl]phenol (210 mg) and β-propiolactone (40 μl) in tetrahydrofuran (2.5 ml) was added potassium tert-butoxide (50 mg) by portion, and the mixture was stirred at room temperature for 48 hours. To this one was added 3.95N hydrogen chloride in ethanol (1.5 ml), and the mixture was stirred for 12 hours. The resulting mixture was evaporated under reduced pressure. The residue was diluted with ethyl acetate and an aqueous solution of sodium hydroxide (1N). The organic layer was seperated, washed with brine, dried over magnesium sulfate and evaporated under reduced pressure. The residue was purified by column chromatography on silica gel (methanol/chloroform=1/30) to give ethyl 3-[3-[[4-[2-[N-benzyl-N-[2-(3-chlorophenyl)-2-hydroxyethyl]amino]ethyl]phenyl]sulfonyl]phenoxy]propanoate (105 mg) as a colorless oil.

(+)ESI-MS (m/z): 622 (M+H)⁺

### EXAMPLE 157

The following compounds were obtained according to a similar manner to that of Preparation 19.

(1) 4-[[4-[2-[N-(tert-Butoxycarbonyl)-N-[(2R)-2-(3-chlorophenyl)-2-hydroxyethyl]amino]ethyl]phenyl]-sulfonyl]butanoic acid

MS (m/z): 526 (M+H)

(2) Methyl 4-[[[4-[2-[N-(tert-butoxycarbonyl)-N-[(2R)-2-(3-chlorophenyl)-2-hydroxyethyl]amino]ethyl]phenyl]-sulfonyl]methyl]benzoate

MS (m/z): 588 (M+H)

### EXAMPLE 158

To a mixture of (R)-[4-[[4-[2-[[2-(3-chlorophenyl)-2-hydroxyethyl]amino]ethyl]phenyl]sulfonyl]phenoxy]acetic acid (102 mg) in a mixture of tetrahydrofuran (20 ml) and water (8 ml) was added saturated aqueous sodium bicarbonate to be adjusted to about pH 8.5, and to this one was added 1-[[[(5-methyl-2-oxo-1,3-dioxol-4-yl)methoxy]carbonyl]oxy]-2,5-pyrrolidinedione (68 mg) in tetrahydrofuran (3 ml) controlling the pH at 8.5 at room temperature. The mixture was stirred at the same temperature for 3 hours. The resulting mixture was adjusted to pH 3 with 1N hydrochloric acid, and ethyl acetate was added. After seperation, the organic layer was dried over anhydrous magnesium sulfate and evaporated under reduced pressure. The residue was purified by thin layer silica gel chromatography (chloroform methanol=3:1) to give (R)-[4-[[4-[2-[N-[2-(3-chlorophenyl)-2-hydroxyethyl]-N-[[(5-methyl-2-oxo-1,3-dioxol-4-yl)methoxy]carbonyl]amino]ethyl]phenyl]sulfonyl]-phenoxy]acetic acid (86 mg).

NMR (DMSO-d₆, δ): 2.05-2.15 (3H, m), 2.75-3.0 (2H, m), 3.15-3.5 (4H, m), 4.45 (2H, s), 4.6-4.85 (3H, m), 6.99 (2H, d, J=8.5 Hz), 7.15-7.5 (6H, m), 7.75-7.9 (4H, m) (−)ESI-MS (m/z): 644, 646 (M−H)⁻

### EXAMPLE 159

The following compound was obtained according to a similar manner to that of Example 146.

Ethyl 4-[[4-[2-[[(2R)-2-(3-chlorophenyl)-2-hydroxyethyl]amino]ethyl]phenyl]sulfonyl]-2-methoxybenzoate

(+)APCI-MS (m/z): 518 (M+H)⁺

### EXAMPLE 160

To a suspension of ethyl (R)-6-[[4-[2-[[2-(3-chlorophenyl)-2-hydroxyethyl]amino]ethyl]phenyl]sulfonyl]-nicotinate (31 mg) in ethanol (2 ml) was added 1N sodium hydroide (0.063 ml) at room temperature, and the mixture was stirred at the same temperature for 6 hours. The resulting mixture was evaporated under reduced pressure and dried in vacuo to give sodium (R)-6-[[4-[2-[[2-(3-chlorophenyl)-2-hydroxyethyl]amino]ethyl]phenyl]sulfonyl]nicotinate (31 mg).

NMR (DMSO-d₆, δ): 2.5-2.85 (6H, m), 4.55-4.7 (1H, m), 7.1-7.5 (6H, m), 7.84 (2H, d, J=8.3 Hz), 8.09 (1H, d, J=8.0 Hz), 8.34 (1H, dd, J=1.8, 7.9 Hz), 8.9 (1H, m) (−)ESI-MS (m/z): 459, 461 (M−Na−H)⁻

### EXAMPLE 161

4-[[4-[2-[[(2R)-2-(3-Chlorophenyl)-2-hydroxyethyl]-amino]ethyl]phenyl]sulfonyl]benzoic acid hydrochloride (39 mg) and 10% hydrogen chloride in methanol (2 ml) were mixed and stirred at room temperature for 11.5 days. Evaporation of the solvent gave methyl 4-[[4-[2-[[(2R)-2-(3-chlorophenyl)-2-hydroxyethyl]amino]ethyl]phenyl]-sulfonyl]benzoate hydrochloride (38 mg) as a white powder.

NMR (DMSO-d₆, δ): 2.90-3.50 (6H, m), 3.88(3H, s), 4.91 (1H, m), 6.33 (1H, br s, OH), 7.28-7.52 (4H, m), 7.54 (2H, d, J=8 Hz), 7.96 (2H, d, J=8 Hz), 7.98-8.23 (4H, m), 9.05 (2H, br s) (+)ESI-MS (m/z): 474 (free, M+H)⁺

1. A compound of the formula [I]: 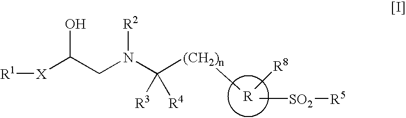
wherein
R¹ is phenyl, pyridyl, indolyl or carbazolyl, each of which may be substituted with one or two same or different substituent(s) selected from a group consisting of halogen; hydroxy; benzyloxy; nitro; cyano; mono(or di or tri)halo(lower)alkyl; and (lower alkylsulfonyl)amino,
R² is hydrogen, [5-(lower alkyl)-2-oxo-1,3-dioxol-4-yl](lower)alkoxycarbony or an amino protective group,
R³ and R⁴ are each independently hydrogen, lower alkyl or hydroxy(lower)alkyl, 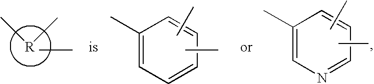
R⁵ is aryl, ar(lower)alkyl, a heterocyclic group or lower alkyl, each of which may be substituted with one, two or three same or different substituent(s) selected from a group consisting of halogen; hydroxy; cyano; amino(hydroxyimino)methyl; phenyl optionally substituted with carboxy or lower alkoxycarbonyl; phenoxy optionally substituted with halogen; lower alkoxy optionally substituted with hydroxy, amino, cyano, carboxy, carbamoyl, mono(or di)(lower)alkylcarbamoyl, lower alkoxycarbonyl, cyclo(lower)alkyloxycarbonyl, hydroxy(lower)alkoxycarbonyl, di[(lower)alkoxy](lower)alkoxycarbonyl, pyridyl(lower)alkoxycarbonyl, phenyl or tetrazolyl; mono(or di or tri)halo(lower)alkoxy; lower alkyl optionally substituted with carboxy, lower alkoxycarbonyl, dioxothiazolidinyl or dioxothiazolidinylidene; lower alkenyl optionally substituted with carboxy or lower alkoxycarbonyl; oxadiazolyl optionally substituted with lower alkyl; tetrazolyl; triazolylthio; lower alkanoyl; carboxy; lower alkoxycarbonyl; carbamoyl optionally substituted with one or two same or different substituent(s) selected from a group consisting of lower alkyl, lower alkoxy, carboxy(lower)alkyl, lower alkoxycarbonyl(lower)alkyl, tetrazolyl, thiazolyl optionally substituted with lower alkyl, oxazolyl optionally substituted with lower alkyl, oxadiazolyl, lower alkylsulfonyl and phenylsulfonyl; (hydroxypiperidino)carbonyl; (2,4-dioxo-1,3-thiazolidin-5-ylindene)methyl; and amino optionally substituted with one or two same or different substituent(s) selected from a group consisting of lower alkyl, lower alkanoyl, benzoyl, pyridylcarbonyl, lower alkylsulfonyl, phenylsulfonyl, carbamoyl, lower alkylcarbamoyl, phenylcarbamoyl, lower alkoxycarbonyl and phenoxycarbonyl, or 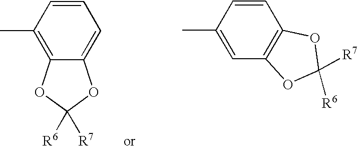
in which R⁶ and R⁷ are each independently hydrogen, carboxy or lower alkoxycarbonyl,
R⁸ is hydrogen or halogen,
X is a single bond or —O—CH₂—, and
n is 0, 1 or 2,
or a salt thereof.

2. A compound of claim 1, wherein
R² is hydrogen, [5-(lower alkyl)-2-oxo-1,3-dioxol-4-yl](lower)alkoxycarbonyl, lower alkoxycarbonyl or ar(lower)alkyl.

3. A compound of claim 2, wherein
R¹ is phenyl which may be substituted with one or two same or different substituent(s) selected from a group consisting of halogen; hydroxy; benzyloxy; nitro and (lower alkylsulfonyl)amino,
R² is hydrogen or [5-(lower alkyl)-2-oxo-1,3-dioxol-4-yl](lower)alkoxycarbonyl, and
R⁵ is phenyl, benzyl, triazolyl, tetrazolyl, quinolyl, thiazolyl, thienyl or lower alkyl, each of which may be substituted with one, two or three same or different substituent(s) selected from a group consisting of halogen; hydroxy; cyano; amino(hydroxyimino)methyl; phenyl optionally substituted with carboxy or lower alkoxycarbonyl; phenoxy optionally substituted with halogen; lower alkoxy optionally substituted with hydroxy, amino, cyano, carboxy, carbamoyl, mono(or di)(lower)alkoxycarbamoyl, lower alkoxycarbonyl, cyclo(lower)alkyloxycarbonyl, hydroxy(lower)alkoxycarbonyl, di)(lower)alkoxy)(lower)alkoxycarbonyl, pyridyl(lower)alkoxycarbonyl, phenyl or tetrazolyl; mono(or di or tri)halo(lower)alkoxy; lower alkyl optionally substituted with carboxy, lower alkoxycarbonyl, dioxothiazolidinyl or dioxothiazolidinylidene; lower alkenyl optionally substituted with carboxy or lower alkoxycarbonyl; oxadiazolyl optionally substituted with lower alkyl; tetrazolyl; triazolylthio; lower alkanoyl; carboxy; lower alkoxycarbonyl; carbamoyl optionally substituted with one or two same or different substituent(s) selected from a group consisting of lower alkyl, lower alkoxy, carboxy, lower alkoxycarbonyl, thiazolyl optionally substituted with lower alkyl, oxazolyl optionally substituted with lower alkyl, oxadiazolyl, lower alkylsulfonyl and phenylsulfonyl; (hydroxypiperidino)carbonyl; (2,4-dioxo-1,3-thiadiazolidin-5-ylidene)methyl; and amino optionally substituted with one or two same or different substituent(s) selected from a group consisting of lower alkyl, lower alkanoyl, benzoyl, pyridylcarbonyl, lower alkylsulfonyl, phenylsulfonyl, carbamoyl, lower alkylcarbamoyl, phenylcarbamoyl, lower alkoxycarbonyl and phenoxycarbonyl, or 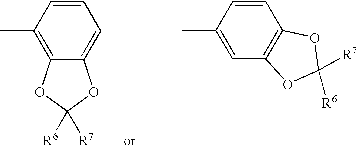
in which R⁶ and R⁷ are each independently hydrogen, carboxy or lower alkoxycarbonyl.

4. A compound of claim 3, wherein
R¹ is phenyl which may be substituted with halogen,
R² is hydrogen,
R⁵ is phenyl which may be substituted with one, two or three same or different substituent(s) selected from a group consisting of halogen; hydroxy; cyano; amino(hydroxyimino)methyl; phenyl optionally substituted with carboxy or lower alkoxycarbonyl; phenoxy optionally substituted with halogen; lower alkoxy optionally substituted with hydroxy, amino, cyano, carboxy, carbamoyl, mono(or di)(lower)alkoxycarbamoyl, lower alkoxycarbonyl, cyclo(lower)alkyloxycarbonyl, hydroxy(lower)alkoxycarbonyl, di[(lower)alkoxy](lower)alkoxycarbonyl, pyridyl(lower)alkoxycarbonyl, phenyl or tetrazolyl; mono(or di or tri)halo(lower)alkoxy; lower alkyl optionally substituted with carboxy, lower alkoxycarbonyl, dioxothiazolidinyl or dioxothiazolidinylidene; lower alkenyl optionally substituted with carboxy or lower alkoxycarbonyl; oxadiazolyl optionally substituted with lower alkyl; tetrazolyl; triazolylthio; lower alkanoyl; carboxy; lower alkoxycarbonyl; carbamoyl optionally substituted with one or two same or different substituent(s) selected from a group consisting of lower alkyl, lower alkoxy, carboxy, lower alkoxycarbonyl, thiazolyl optionally substituted with lower alkyl, oxazolyl optionally substituted with lower alkyl, oxadiazolyl, lower alkylsulfonyl or phenylsulfonyl; and amino optionally substituted with one or two same or different substituent(s) selected from a group consisting of lower alkyl and lower alkanoyl,
R⁸ is hydrogen,
X is a single bond, and
n is 1.

5. A compound of claim 4, wherein
R³ and R⁴ are each hydrogen, and
R⁵ is phenyl substituted with lower alkoxy optionally substituted with a substituent selected from a group consisting of hydroxy, amino, cyano, carboxy, carbamoyl, mono(or di)(lower)alkoxycarbamoyl, lower alkoxycarbonyl, cyclo(lower)alkyloxycarbonyl, hydroxy(lower)alkoxycarbonyl, di[(lower)alkoxy](lower)alkoxycarbonyl, pyridyl(lower)alkoxycarbonyl, phenyl and tetrazolyl.

6. A compound of claim 5, which is selected from a group consisting of
(1) Isopropyl (R)-[4-[[4-[2-[[2-(3-chlorophenyl)-2-hydroxyethyl]amino]ethyl]phenyl]sulfonyl]phenoxy]-acetate,
(2) (R)-2-[4-[[4-[2-[[2-(3-Chlorophenyl)-2-hydroxyethyl]amino]ethyl]phenyl]sulfonyl]phenoxy]-N-methylacetamide,
(3) [4-[[4-[2-[[(R)-2-(3-Chlorophenyl)-2-hydroxyethyl]-amino]ethyl]phenyl]sulfonyl]phenoxy]acetate,
(4) (1R)-2-[[2-[4-[[4-(2-Aminoethoxy)phenyl]sulfonyl]-phenyl]ethyl]amino]-1-(3-chlorophenyl)ethanol,
(5) Ethyl [4-[[4-[2-[[(2R)-2-(3-chlorophenyl)-2-hydroxy-ethyl]amino]ethyl]phenyl]sulfonyl]phenoxy]acetate,
(6) 2-Pyridylmethyl (R)-[4-[[4-[2-[[2-(3-chlorophenyl)-2-hydroxyethyl]amino]ethyl]phenyl]sulfonyl]phenoxy]-acetate,
(7) 2-Hydroxyethyl (R)-[4-[[4-[2-[[2-(3-chlorophenyl)-2-hydroxyethyl]amino]ethyl]phenyl]sulfonyl]phenoxy]-acetate,
(8) (R)-1-(3-Chlorophenyl)-2-[[2-[4-[[4-(1H-tetrazol-5-ylmethoxy)phenyl]sulfonyl]phenyl]ethyl]amino]ethanol,
(9) (R)-2-[4-[[4-[2-[[2-(3-Chlorophenyl)-2-hydroxyethyl]-amino]ethyl]phenyl]sulfonyl]phenoxy]acetamide, and
(10) (1R)-1-(3-Chlorophenyl)-2-[[2-[4-[[3-(2-hydroxy-ethoxy)phenyl]sulfonyl]phenyl]ethyl]amino]ethanol
or a pharmaceutically acceptable salt thereof.

7. A process for preparing a compound of claim 1, or a salt thereof, which comprises,
(i) reacting a compound [II] of the formula: 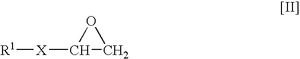
wherein R¹ and X are each as defined in claim 1, with a compound [III] of the formula: 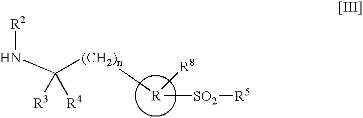
wherein R², R³, R⁴, 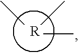
R⁵, R⁸ and n are each as defined in claim 1,
or a salt thereof, to give a compound [I] of the formula: 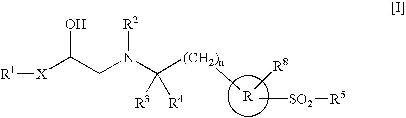
wherein R¹, R², R³, R⁴, 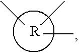
R⁵, R⁸, X and n are each as defined in claim 1,
or a salt thereof,
(ii) subjecting a compound [Ia] of the formula 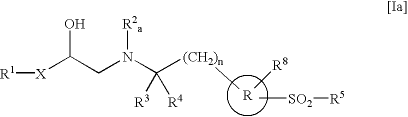
wherein R¹, R³, R⁴, 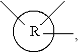
R⁵, R⁸, X and n are each as defined in claim 1, and
Rₐ² is [5-(lower alkyl)-2-oxo-1,3-dioxol-4-yl](lower)alkoxycarbonyl or an amino protective group,
or a salt thereof, to elimination reaction of the amino protective group, to give a compound [Ib] of the formula: 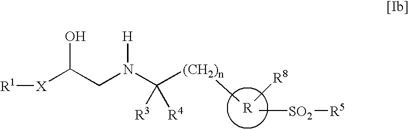
wherein R¹, R³, R⁴, 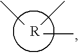
R⁵, R⁸, X and n are each as defined in claim 1,
or a salt thereof, and
(iii) reacting a compound [Ic] of the formula: 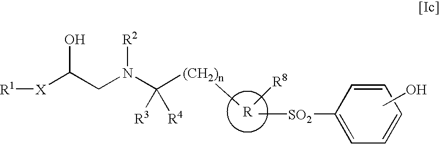
wherein R¹, R², R³, R⁴, 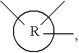
R⁸, X and n are each as defined in claim 1,
with a compound [IV] of the formula:
Y—R⁹ [IV]
wherein R⁹ is lower alkyl optionally substituted with hydroxy, amino, cyano, carboxy, carbamoyl, mono(or di)(lower)alkylcarbamoyl, lower alkoxycarbonyl, cyclo(lower)alkyloxy-carbonyl, hydroxy(lower)alkoxycarbonyl, di[(lower)alkoxy](lower)alkoxycarbonyl, pyridyl(lower)alkoxycarbonyl, phenyl or tetrazolyl, and
Y is halogen,
to give a compound [Id] of the formula: 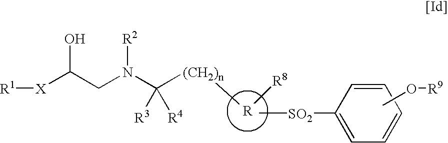
wherein R¹, R², R³, R⁴, 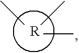
R⁸, X and n are each as defined in claim 1, and
R⁹ is as defined above,
or a salt thereof.

8. A pharmaceutical composition which comprises, as an active ingredient, a compound of claim 1 or a pharmaceutically acceptable salt thereof in admixture with pharmaceutically acceptable carriers or excipients.

9. Use of a compound of claim 1 or a pharmaceutically acceptable salt thereof for the manufacture of a medicament.

10. A compound of claim 1 or a pharmaceutically acceptable salt thereof for use as a medicament.

11. A compound,of claim 1 or a pharmaceutically acceptable salt thereof for use as selective β₃ adrenergic receptor agonists.

12. A method for the prophylactic and/or the therapeutic treatment of pollakiuria or urinary incontinence which comprises administering a compound of claim 1 or a pharmaceutically acceptable salt thereof to a human being or an animal.
